(19) United States
(12) Patent Application Publication
(10) Pub. No.: US 2024/0239813 A1
(43) Pub. Date: Jul. 18, 2024

(54) KRAS INHIBITORS AND PHARMACEUTICAL USES THEREOF

(71) Applicants: RISEN (SUZHOU) PHARMA TECH CO., LTD., Suzhou (CN); RISEN (SHANGHAI) PHARMACEUTICAL ENGINEERING CO LTD, Shanghai (CN)

(72) Inventors: Jiasheng LU, Suzhou (CN); Xiang JI, Suzhou (CN); Mengchao SHI, Suzhou (CN); Qiguo ZHANG, Suzhou (CN); Xiaolin HE, Suzhou (CN); Yanpeng WU, Suzhou (CN); Bin ZONG, Suzhou (CN); Gang WU, Suzhou (CN); Xianchao DU, Suzhou (CN); Linxin WANG, Suzhou (CN); Tianlun ZHOU, Suzhou (CN); Jian GE, Suzhou (CN); Xianqi KONG, Dollard-des-Ormeaux (CA); Xiangsheng YE, Suzhou (CN); Dawei CHEN, Suzhou (CN)

(21) Appl. No.: 18/533,332

(22) Filed: Dec. 8, 2023

(30) Foreign Application Priority Data
Dec. 8, 2022 (CN) 202211574036.1

(51) Int. Cl. C07D 519/00; A61K 45/06; C07K 16/28

(52) U.S. Cl. CPC C07D 519/00; A61K 45/06; C07K 16/2818

(57) ABSTRACT

The disclosure relates to KRAS inhibitor compounds having the structure of Formula (I), pharmaceutical compositions thereof, and methods of use thereof for inhibiting, treating, and/or preventing KRAS-associated diseases, disorders and conditions.

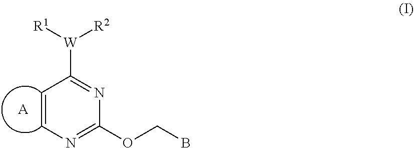

### CROSS-REFERENCE TO RELATED APPLICATIONS

This application claims the benefit of priority from Chinese patent application no. CN202211574036.1, filed Dec. 8, 2022, which is incorporated by reference herein in its entirety.

### FIELD

The present disclosure relates to KRAS inhibitors and pharmaceutically acceptable salts, esters, hydrates, solvates and stereoisomers thereof, as well as pharmaceutical compositions thereof and methods of use thereof for inhibiting, treating and/or preventing KRAS-related diseases.

### BACKGROUND

The Kirsten Rat Sarcoma Viral Oncogene Homolog (K-Ras) gene belongs to the Ras family of oncogenes and is one of the most common gene mutations in human cancers. Its encoded protein (KRAS) is part of the RAS/MAPK signal transduction pathway which regulates cell growth and differentiation. KRAS is a small GTPase, a class of enzymes which convert the nucleotide guanosine triphosphate (GTP) into guanosine diphosphate (GDP). It is turned on (activated) by binding to GTP and turned off (inactivated) by converting the GTP to GDP. In this way KRAS acts as a molecular on/off switch. In most cells, KRAS is inactivated. When activated, it can activate several downstream signaling pathways including the MAPK signal transduction pathway, the PI3K signal transduction pathway and the Ral-GEFs signal transduction pathway. These signal transduction pathways play an important role in promoting cell survival, proliferation, and cytokine release, thus affecting tumor occurrence and development.

In human cancers, K-Ras gene mutations occur in nearly 90% of pancreatic cancers, approximately 30-40% of colon cancers, approximately 17% of endometrial cancers, and approximately 15-20% of lung cancers (mostly non-small cell lung cancer, NSCLC). K-Ras gene mutations also occur in bile duct cancers, cervical cancers, bladder cancers, liver cancers, and breast cancers, as well as leukemias. K-Ras gene mutations are thus found at high rates in many different types of cancer.

Most K-Ras missense mutations occur in Codon 12, which results in changing the glycine at position 12 (G12) to another amino acid. Among these mutations, G12C, G12D, G12R and G12V are the most common KRAS mutations in patients. For instance, KRASG12D and KRASG12V mutations are found in approximately 90% of pancreatic cancers, whereas KRASG12D is the most common KRAS mutation in colon cancer. KRASG12C mutant protein has gained significant attention recently as a prominent target for research. In 2021, The U.S. Food and Drug Administration (FDA) approved sotorasib as the first KRASG12C blocking drug for the treatment of adult patients with NSCLC. The KRASG12C inhibitor adagrasib was also approved by the U.S. FDA in 2022 for treatment of NSCLC. KRAS gene mutations also include KRAS G12A, KRAS G12S, KRAS G13D, and KRAS Q61H, among others (Liu, Pingyu et al., Acta Pharmaceutica Sinica. B (2019), 9(5), 871-879).

However, existing KRAS inhibitors face significant limitations. One of the biggest obstacles to KRAS inhibitor treatment is the emergence of drug resistance. While the biological basis of acquired drug resistance is not well understood, it has been suggested that several factors may play a role, including cellular heterogeneity in tumors; the activation of wild-type RAS by multiple receptor tyrosine kinases (RTKs) rather than a single RTK; and secondary gene mutations (see, e.g., Liu et al., Cancer Gene Therapy 2022, 29:875-878). There are few reports on inhibitor compounds involving KRASG12, primarily due to the chemical challenge of effectively binding to the specific amino acid residue. Some KRASG12D inhibitors have been disclosed in International (PCT) Application Publication Nos. WO2021041671 and WO2021106231. However, their clinical use is limited.

There is a need for new KRAS inhibitors with higher activity and improved therapeutic effect for clinical use.

### SUMMARY

The present disclosure relates to KRAS inhibitor compounds, compositions thereof, and methods of use thereof for inhibiting, treating or preventing a KRAS-associated disease, disorder or condition such as a hyperproliferative disorder. Specifically, the disclosure provides KRAS inhibitor compounds having the structure shown in Formula (I), as well as pharmaceutically acceptable salts, esters, hydrates, solvates or stereoisomers thereof. As reported hereinbelow, inhibitor compounds of the disclosure demonstrate favorable anti-tumor activity and are useful therapeutically for treatment or prevention of KRAS-associated cancers and tumors and related conditions.

In a first broad aspect, there are provided compounds of Formula (I) and pharmaceutically acceptable salts, esters, hydrates, solvates or stereoisomers thereof:

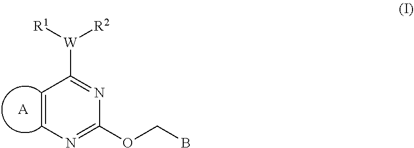

where:
A is a substituted or unsubstituted aromatic ring, a heteroaromatic ring, a carboatomic ring or a carbon heteroatomic ring;
B is a substituted or unsubstituted alkyl, a substituted or unsubstituted alkylaminoacyl, a carboatomic ring, a carbon heteroatomic ring, a substituted or unsubstituted aromatic ring, a heteroaromatic ring, a substituted or unsubstituted condensed ring, or a group shown below:

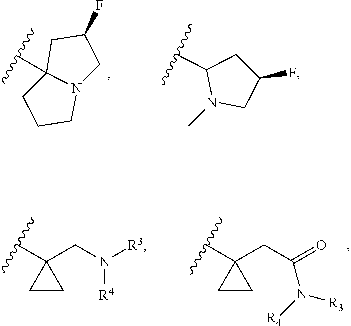

or a combination thereof,
wherein: R³ and R⁴ are independently H or substituted or unsubstituted alkyl, wherein the substitutions in substituted alkyl are halogen, C₁-C₄ alkyl, —OH, C₁-C₄ alkoxy, C₁-C₄ carboxyl, C₁-C₄ ester group, C₁-C₄ acylamino group, or substituted or unsubstituted C₃-C₇ carbon heteroatomic ring, wherein the heteroatom is N, O or S, and the number of heteroatoms can be 1, 2, 3 or 4; or,
R³ and R⁴ and the N atom to which they are attached form a substituted or unsubstituted heterocyclic ring, a substituted or unsubstituted spiro-heterocycle ring, or a substituted or unsubstituted heterobridged ring; or,
R³ and R⁴ and the N atom to which they are attached form a substituted or unsubstituted C₃-C₁₂ heteroaryl (such as, for example and without limitation, azetidinyl, pyrrolyl, pyridyl, pyrimidinyl, pyrazinyl, indolyl, quinolyl, isoquinolyl, purinyl, carbazolyl, etc.);
W is C, O or N, wherein: if W is O, then R¹ is absent, and R² is independently H or substituted or unsubstituted alkyl or substituted or unsubstituted cycloalkyl; or, if W is C, then R¹ and R² are independently H, hydroxyl, halogen, alkyl, alkoxy, or alkanoyl, or R¹ and R² together form substituted or unsubstituted C₅-C₈ aryl or C₅-C₈ bicyclic; or, if W is N, then R¹ and R² are independently H, substituted or unsubstituted alkyl spiro ring, or substituted or unsubstituted alkanoyl, or R¹ and R² and the W to which they are linked form a substituted or unsubstituted aryl, heteroaryl, cycloalkyl, heterocycloalkyl or group shown below:

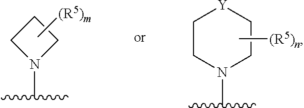

wherein Y is O, N, —CH₂—, —CH₂CH₂—, —CH═CH—, —OCH₂— or absent, and the H on Y and the substitutable sites on the ring can be arbitrarily substituted by R⁵;
m is an integer of from 0 to 6 (i.e., r is 0, 1, 2, 3, 4, 5 or 6);
n is an integer of from 0 to 8 (i.e., s is 0, 1, 2, 3, 4, 5, 6, 7 or 8);
R⁵ is independently H, alkyl, hydroxyl, halogen, amino, —CF₃, —NH(C₁-C₃ alkyl), —N(C₁-C₃ alkyl)₂, ═O, —CN, —O—(C₁-C₃ alkyl), —(C₁-C₃ alkyl)-OH, —C(═O)OH, —C(═O)(C₁-C₃ alkyl), —C(═O)O(C₁-C₃ alkyl), aryl, arylalkyl, cycloalkyl or heterocycloalkyl; or,
m and/or n is 2, and the two R⁵s are attached to the same atom on the ring and linked together to form a spiro ring or a condensed ring, wherein R¹ and R² and the N atom to which they are linked form a first ring, and the two R⁵s form a second ring.

In some embodiments, the second ring formed by the two R⁵s is an alkyl ring or a heteroalkyl ring, and the spiro ring is optionally substituted by alkyl, hydroxyl, halogen, amino, ═O or —CN.

In other embodiments, the second ring formed by the two R⁵s is a condensed ring, where R¹ and R² and the N atom to which they are linked form a first ring, and the two R⁵s form the second ring. In some embodiments, the ring formed by two arbitrary adjacent R⁵s is an alkyl ring or a heteroalkyl ring, and the condensed ring described can be substituted by alkyl, hydroxyl, halogen, amino, ═O or —CN.

In other embodiments, two arbitrary non-adjacent R⁵s and the ring to which they are linked form a bridged ring of C₁-C₂ bridge, where R¹ and R² and the N atom to which they are linked form the ring, and the two non-adjacent R⁵s bind to form the bridged bond.

In some embodiments, R⁵ is independently H; an amino-protecting group (e.g., Boc); a C₁₁-C₃₀ alkyl group; a C₁₁-C₃₀ alkenyl with at least one olefinic bond; —C(═O)(C₁-C₃ alkyl); —C(═O)O(C₁-C₃ alkyl); —C(═O)N(C₁-C₃ alkyl); C₆-C₃₀ aryl; C₅-C₃₀ heteroaryl with N atom; C₃-C₈ heterocycloalkyl with O atom; C₇-C₂₀ aralkyl (e.g., benzyl, naphthylmethyl); C₆-C₃₀ cycloalkyl of double ring, triple ring, spiro ring or bridged ring; or, C₆-C₁₀ heterocycloalkyl of double ring, triple ring, spiro ring or bridged ring with at least one N atom.

In some embodiments of compounds of the disclosure, a C₁₁-C₃₀ alkyl is a C₁₁-C₂₀ alkyl. In some embodiments, a C₁₁-C₃₀ alkyl is a C₁₂-C₁₈ alkyl. In some embodiments, a C₁₁-C₃₀ alkyl is a dodecyl, tetradecyl, hexadecyl, octadecyl, or nonadecyl, etc.

In some embodiments of compounds of the disclosure, R⁵ is C₁₁-C₃₀ alkenyl with at least one olefinic bond. In some such embodiments, R⁵ is C₁₁-C₂₀ alkenyl with at least one olefinic bond. In some such embodiments, R⁵ is C₁₂-C₁₈ alkenyl with at least one olefinic bond.

In some embodiments of compounds of the disclosure, R⁵ is a C₆-C₂₀ aryl. In some such embodiments, R⁵ is a C₁₀-C₁₈ aryl. In some such embodiments, R⁵ is a C₆-C₃₀ aryl. In some such embodiments, R⁵ is a phenyl, naphthyl, anthryl, fluorenyl, fluorenonyl or pyrenyl, etc.

In some embodiments of compounds of the disclosure, R⁵ is a C₅-C₂₀ N-heteroaryl. In some embodiments, R⁵ is a C₅-C₁₄ N-heteroaryl. In some embodiments, R⁵ is a C₄-C₃₀ N-heteroaryl. In some such embodiments, R⁵ is indolyl, carbazolyl, pyrazine, or pyridazine, etc.

In some embodiments of compounds of the disclosure, R⁵ is a C₃-C₈ heterocycloalkyl with an O atom, such as furan or pyran, etc.

In some embodiments of compounds of the disclosure, a C₇-C₂₀ aralkyl is a C₇-C₁₄ aralkyl. In some embodiments, a C₇-C₂₀ aralkyl is benzyl, phenethyl, phenylpropyl, menaphthyl, naphthylethyl, etc.

In some embodiments of compounds of the disclosure, a C₆-C₃₀ cycloalkyl of dual ring, triple ring, spiro ring or bridged ring is a C₆-C₂₀ cycloalkyl. In some embodiments, a C₆-C₃₀ cycloalkyl of dual ring, triple ring, spiro ring or bridged ring is a C₇-C₁₅ cycloalkyl. In some embodiments, a cycloalkyl is a bridged ring. Examples of bridged rings include, without limitation, adamantyl,

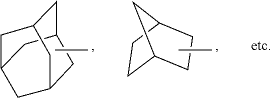

In some embodiments of compounds of the disclosure, the C₆-C₁₀ heterocycloalkyl of dual ring, triple ring, spiro ring or bridged ring with at least one N atom, is a bridged ring. Examples of heterocycloalkyl bridged rings include, without limitation,

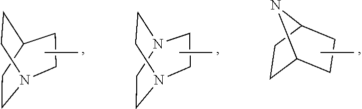

etc. In other embodiments of compounds of the disclosure, the two R⁵s and the ring formed by R¹ and R² form a C₄-C₁₀ heterocycloalkyl of dual ring, triple ring, spiro ring or bridged ring (optionally further with at least one N atom). In some such embodiments, the C₄-C₁₀ heterocycloalkyl is a N-containing spirocycloalkyl. Examples of such N-containing spirocycloalkyls include, without limitation,

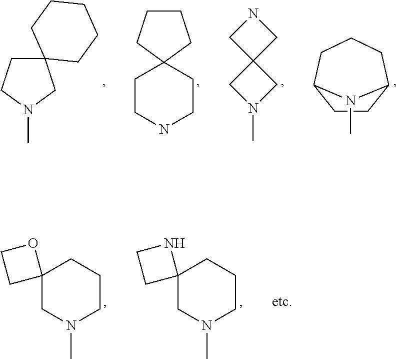

In some embodiments of compounds of the disclosure: (1) a substituted or unsubstituted phenyl, a substituted or unsubstituted nitrogen-containing six-membered aromatic heterocycle, a substituted or unsubstituted five-membered aromatic heterocycle, where the substituent is selected from:
(a) C₁₋₈ straight or branched hydrocarbyl, halogen-substituted C₁₋₈ straight or branched hydrocarbyl, F, Cl, Br, NO₂, CN, methylenedioxy, OR¹, SR², NR³R¹, NR⁴COR², COOR⁵, CONR⁶R³, NR⁷COOR⁴, SO2NR⁸R⁵, (CH₂)_{1,2,3}NR⁹R⁶, (CH₂)_{1,2,3} OR¹⁰, where R¹, R², R³, R⁴, R⁵, R⁶, R⁷, R⁸, R⁹ and R¹⁰ are independently selected from H, substituted or unsubstituted C₁₋₈ straight or branched hydrocarbyl, substituted or unsubstituted C₂₋₈ straight or branched alkenyl, substituted or unsubstituted C₂₋₈ straight or branched alkynyl, substituted or unsubstituted 3-7 membered cyclohydrocarbyl, substituted or unsubstituted 3-8 membered oxygen-containing cycloheterohydrocarbyl, substituted or unsubstituted 3-8 membered nitrogen-containing cycloheterohydrocarbyl, substituted or unsubstituted phenyl, substituted or unsubstituted six-membered aromatic heterocycle, substituted or unsubstituted five-membered aromatic heterocycle, where the substituent is selected from F, Cl, Br, CN, OR^{a1}, SR^{a2}, NR^{a3}R^{b1}, COOR^{a4}, CONR^{a5}R^{b2}, NR^{a6}COOR^{b3}, SO₂NR^{a7}R^{b4} and NR^{a8}COR^{b5}, where R^{a1}, R^{a2}, R^{a3}, R^{b1}, R^{a4}, R^{a5}, R^{b2}, R^{a6}, R^{b3}, R^{a7}, R^{b4}, R^{a8} and R^{b5} are independently selected from H, C₁₋₄ straight or branched hydrocarbyl, cyclopropyl, cyclopropylmethylene, cyclobutyl and cyclopentyl; the 3-8 membered oxygen-containing cycloheterohydrocarbyl or nitrogen-containing cycloheterohydrocarbyl may contain 1 heteroatom, or may simultaneously contain multiple heteroatoms, where the heteroatoms are selected from O, N and S; and the halogens are selected from F, Cl and Br;
(b) substituted or unsubstituted C₃₋₇ cycloalkyl, substituted or unsubstituted 3-8 membered oxygen-containing cycloheterohydrocarbyl, substituted or unsubstituted 3-8 membered nitrogen-containing cycloheterohydrocarbyl, where the substituent is selected from C₁₋₅ straight or branched hydrocarbyl, F, Cl, Br, CN, OR^{a1}, SR^{a2}, NR^{a3}R^{b1}, COOR^{a4}, CONR^{a5}R^{b2}, NR^{a6}COOR^{b3}, SO₂NR^{a7}R^{b4} and NR^{a8}COR^{b5}, where R^{a1}, R^{a2}, R^{a3}, R^{b1}, R^{a4}, R^{a5}, R^{b2}, R^{a6}, R^{b3}, R^{a7}, R^{b4}, R^{a8} and R^{b5} are independently selected from H, C₁₋₄ straight or branched hydrocarbyl, cyclopropyl, cyclopropylmethylene, cyclobutyl, cyclopentyl and cyclohexyl; the 3-8 membered oxygen-containing cycloheterohydrocarbyl or nitrogen-containing cycloheterohydrocarbyl may contain 1 heteroatom, or may simultaneously contain multiple heteroatoms, where the heteroatoms are selected from O, N and S; n is selected from 1, 2 and 3; and the halogens are selected from F, Cl and Br;
(c) substituted or unsubstituted phenyl, substituted or unsubstituted six-membered aromatic heterocycle, substituted or unsubstituted five-membered aromatic heterocycle, where the substituent is selected from F, Cl, Br, CN, OR^{a1}, SR^{a2}, NR^{a3}R^{b1}, COOR^{a4}, CONR^{a5}R^{b2}, NR^{a6}COOR^{b3}, SO₂NR^{a7}R^{b4} and NR^{a8}COR^{b5}, where R^{a1}, R^{a2}, R^{a3}, R^{b1}, R^{a4}, R^{a5}, R^{b2}, R^{a6}, R^{b3}, R^{a7}, R^{b4}, R^{a8} and R^{b5} are independently selected from H, C₁₋₄ straight or branched hydrocarbyl, cyclopropyl, cyclopropylmethylene, cyclobutyl and cyclopentyl; the 3-8 membered oxygen-containing cycloheterohydrocarbyl or nitrogen-containing cycloheterohydrocarbyl may contain 1 heteroatom, or may simultaneously contain multiple heteroatoms; the phenyl ring, nitrogen-containing six-membered aromatic heterocycle, or five-membered aromatic heterocycle can be mono-substituted or poly-substituted; the six-membered aromatic heterocycle may contain 1 N atom, or may contain multiple nitrogen atoms; the five-membered aromatic heterocycle may contain 1 heteroatom, or may contain multiple heteroatoms, where the heteroatoms are selected from O, N and S; n is selected from 1, 2 and 3; and the halogens are selected from F, Cl and Br;
(2) substituted or unsubstituted aromatic fused rings or fused heterocycles, substituted or unsubstituted non-aromatic fused rings or fused heterocycles, including substituted or unsubstituted naphthalene ring, substituted or unsubstituted six-membered benzoheterocycle, substituted or unsubstituted five-membered benzoheterocycle, where the substituent described is selected from C₁₋₄ straight or branched hydrocarbyl, halogen-substituted C₁₋₄ straight or branched hydrocarbyl, F, Cl, Br, NO₂, CN, methylenedioxy, OR^{s1}, SR^{s2}, NR^{s3}R^{t1}, NR^{s4}COR^{t2}, COOR^{s5}, CONR^{s6}R^{t3}, NR^{s7}COOR^{t4}, SO₂NR^{s8}R^{t5}, (CH₂)_{1,2,3}NR^{s9}R^{t6} and (CH₂)_{1,2,3}OR^{s10}, where R^{s1}, R^{s2}, R^{s3}, R^{t1}, R^{s4}, R^{t2}, R^{s5}, R^{s6}, R^{t3}, R^{s7}, R^{t4}, R^{s8}, R^{t5}, R^{s9}, R^{t6} and R^{s10} are independently selected from H, C₁₋₄ straight or branched hydrocarbyl, cyclopropyl, cyclopropylmethylene, cyclobutyl and cyclopentyl; the naphthalene ring, six-membered benzoheterocycle, or five-membered benzoheterocycle can be mono-substituted or poly-substituted; the six-membered benzoheterocycle or five-membered benzoheterocycle may contain 1 heteroatom, or may contain multiple heteroatoms, where the heteroatoms are selected from O, N and S; n is selected from 1, 2 and 3; and the halogens are selected from F, Cl and Br.

In some embodiments, the compound represented by Formula (I) is a compound represented by Formula (I-a) or a pharmaceutically acceptable salt, ester, hydrate, solvate, or stereoisomer thereof:

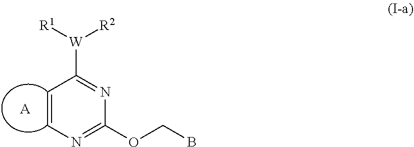

where:
the A ring is a substituted or unsubstituted aromatic ring, heteroaromatic ring, carboatomic ring or carbon heteroatomic ring;
W is C, O or N, wherein when W is O, R¹ is absent;
B is a substituted or unsubstituted alkyl, alkylamino, carboatomic ring, carbon heteroatomic ring, aromatic ring, heteroaromatic ring, condensed ring, or group which is

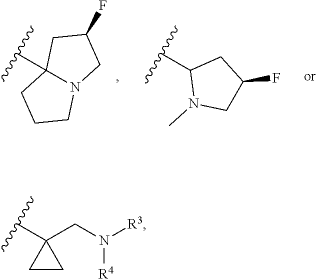

wherein B is optionally substituted by one or more R⁸,
wherein two arbitrary R⁸s linked to one atom form a first ring and the ring linked to them forms a spiro ring, wherein the first ring formed by the two arbitrary R⁸s is a carboatomic ring or carboheteroatomic ring, and the spiro ring is optionally substituted by alkyl, hydroxyl, halogen, amino, ═O or —CN; or,
two arbitrary R⁸s linked to one atom form a first ring and the ring linked to them forms a condensed ring, wherein the first ring formed by the two arbitrary R⁸s is a carboatomic ring or carboheteroatomic ring, and the condensed ring is optionally substituted by alkyl, hydroxyl, halogen, amino, ═O or —CN;
wherein, when W is C, R¹ and R² are independently H, hydroxy, halogen, alkyl, alkoxy, or alkanoyl; when W is N, R¹ and R² are independently H, alkyl, or alkanoyl; and when W is O, R² is independently H or alkyl; or, R¹ and R² and the W linked to them form a substituted or unsubstituted aryl, heteroaryl, cycloalkyl, heterocycloalkyl, or group which is:

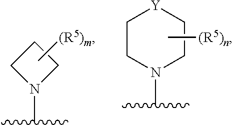

wherein:
R³ and R⁴ are independently H or alkyl group; or, R³ and R⁴ and the N atom to which they are attached form a substituted or unsubstituted heterocyclic ring;
R⁵ is independently H, alkyl, hydroxyl, halogen, amino, —NH(C1-C₃ alkyl), —N(C₁-C₃ alkyl)₂, ═O, —CN, —O—(C₁-C₃ alkyl), —(C₁-C₃ alkyl)-OH, —C(═O)OH, —C(═O)(C₁-C₃ alkyl), —C(═O)O(C₁-C₃ alkyl), aryl, arylalkyl, cycloalkyl or heterocycloalkyl; or,
two arbitrary R⁵s linked to one atom and the ring linked to them form a spiro ring, where R¹ and R² and the N atom linked to them form one ring, and the two R⁵s linked to one atom form another ring, wherein the ring formed by the two arbitrary R⁵s linked to one atom is an alkyl ring or heteroalkyl ring, and the spiro ring is optionally substituted by alkyl, hydroxyl, halogen, amino, ═O or —CN; or,
two arbitrary adjacent R⁵s and the ring linked to them form a condensed ring, where R¹ and R² and the N atom linked to them form one ring, and the two adjacent R⁵s form another ring, wherein the ring formed by the two arbitrary adjacent R⁵s is an alkyl ring or heteroalkyl ring, and the condensed ring is optionally substituted by alkyl, hydroxyl, halogen, amino, ═O or —CN; or,
two arbitrary non-adjacent R⁵s and the ring linked to them form a bridged ring of C₁-C₂ bridge, where R¹ and R² and the N atom linked to them form the ring, and the two non-adjacent R⁵s bind to form the bridged bond;
Y is O, N, —CH₂—, —CH₂CH₂—, —CH═CH—, —OCH₂— or absent;
m is an integer of 0 to 6 (i.e., m is 0, 1, 2, 3, 4, 5 or 6); and
n is an integer of 0 to 8 (i.e., n is 0, 1, 2, 3, 4, 5, 6, 7 or 8).

In some embodiments of compounds of Formula (I-a), R⁵ is independently H; amino-protecting group (e.g., Boc), a C₁₁-C₃₀ alkyl group; a C₁₁-C₃₀ alkenyl with at least one olefinic bond; —C(═O)(C₁-C₃ alkyl); —C(═O)O(C₁-C₃ alkyl); —C(═O)N(C₁-C₃ alkyl); a C₆-C₃₀ aryl; a C₅-C₃₀ heteroaryl with N atom; a C₇-C₂₀ aralkyl (benzyl, naphthylmethyl); a C₆-C₃₀ cycloalkyl of dual ring, triple ring, spiro ring or bridged ring; or a C₆-C₁₀ heterocycloalkyl of dual ring, triple ring, spiro ring or bridged ring with at least one N atom.

In some such embodiments of compounds of Formula (I-a), the C₁₁-C₃₀ alkyl is a C₁₁-C₂₀ alkyl. In some such embodiments, the C₁₁-C₃₀ alkyl is a C₁₂-C₁₈ alkyl. In some embodiments, the C₁₁-C₃₀ alkyl is a dodecyl, tetradecyl, hexadecyl, octadecyl, etc.

In some embodiments of compounds of Formula (I-a), the C₁₁-C₃₀ alkenyl with at least one olefinic bond is a C₁₁-C₂₀ alkyl with at least one olefinic bond. In some embodiments, the C₁₁-C₃₀ alkenyl with at least one olefinic bond is a C₁₂-C₁₈ alkyl with at least one olefinic bond.

In some embodiments of compounds of Formula (I-a), the C₆-C₃₀ aryl is a C₆-C₂₀ aryl. In some embodiments, the C₆-C₃₀ aryl is a C₁₀-C₁₈ aryl. In some embodiments, the C₆-C₃₀ aryl is phenyl, naphthyl, anthryl, fluorenyl, fluorenonyl, pyrenyl, etc.

In some embodiments of compounds of Formula (I-a), the C₅-C₃₀ heteroaryl with N atom is a C₅-C₂₀ N-heteroaryl. In some embodiments, the C₅-C₃₀ heteroaryl with N atom is a C₅-C₁₄ N-heteroaryl. In some embodiments, the C₅-C₃₀ heteroaryl with N atom is indolyl, carbazolyl, etc.

In some embodiments of compounds of Formula (I-a), the C₇-C₂₀ aralkyl is a C₇-C₁₄ aralkyl. In some embodiments, the C₇-C₂₀ aralkyl is benzyl, phenethyl, phenylpropyl, menaphthyl, naphthylethyl, etc.

In some embodiments of compounds of Formula (I-a), the C₆-C₃₀ cycloalkyl of dual ring, triple ring, spiro ring or bridged ring is a C₆-C₂₀ cycloalkyl. In some embodiments, the C₆-C₃₀ cycloalkyl of dual ring, triple ring, spiro ring or bridged ring is a C₇-C₁₅ cycloalkyl. In some embodiments, the C₆-C₃₀ cycloalkyl is a bridged ring. Non-limiting examples include adamantyl

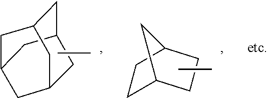

In some embodiments of compounds of Formula (I-a), the C₆-C₁₀ heterocycloalkyl of dual ring, triple ring, spiro ring or bridged ring with at least one N atom is a bridged ring. Non-limiting examples include

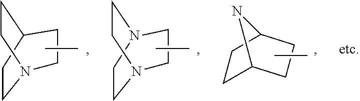

In some embodiments of compounds of Formula (I-a), two R⁵s and the ring formed by R¹ and R² linked to them form a C₄-C₁₀ heterocycloalkyl of dual ring, triple ring, spiro ring or bridged ring, optionally further with at least one N atom. In some such embodiments, the C₄-C₁₀ heterocycloalkyl is a C₄-C₁₀ N-containing spirocycloalkyl. Non-limiting examples include

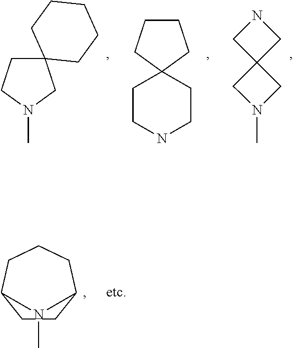

In some embodiments of compounds of Formula (I-a), R³ and R⁴ and the N atom linked to them form a C₄-C₁₂ heteroaryl. In some embodiments, the C₄-C₁₂ heteroaryl is pyrrolyl, pyridyl, pyrimidinyl, pyrazinyl, indolyl, quinolyl, isoquinolyl, purinyl, carbazolyl, etc.

In some embodiments, the compound represented by Formula (I) is a compound represented by Formula (I-b) or a pharmaceutically acceptable salt, ester, hydrate, solvate, or stereoisomer thereof:

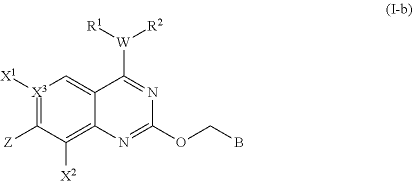

where:
X¹ and X² are independently H, OH, halogen (e.g., F, Cl), CF₃, NH₂, substituted or unsubstituted C₁-C₄ alkyl (e.g., C₁, C₂, C₃ or C₄ alkyl), or absent;
X³ is C or N;
Z is a substituted or unsubstituted cycloalkyl, substituted or unsubstituted aryl or substituted or unsubstituted polycycloaryl; and
R¹, R², W and B are as described above.

In some embodiments of compounds of Formula (I-b), Z is

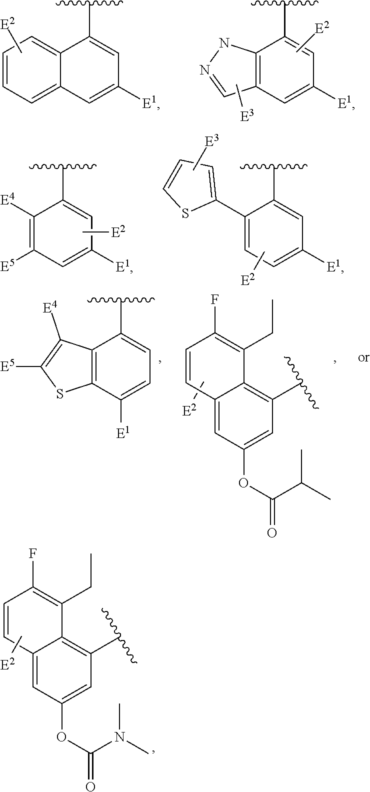

where E¹, E², E³, E⁴ and E⁵ are independently H, halogen (e.g., F, Cl), CF₃, NH₂, OH, CN, substituted or unsubstituted C₁-C₄ hydrocarbyl (e.g., C₁, C₂, C₃ or C₄ hydrocarbyl), or absent; wherein E² and E³ are optionally substituted at any substitutable site on the ring; wherein, when E² and E³ are absent or H, E¹ is Cl or methyl.

In some embodiments, the compound of the disclosure is a compound represented by Formula (II-a) or a pharmaceutically acceptable salt, ester, hydrate, solvate, or stereoisomer thereof:

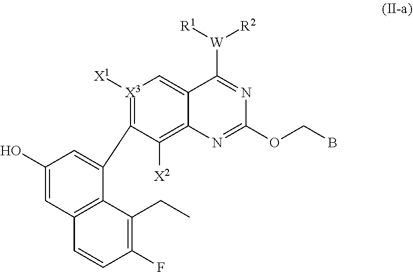

where:
X¹ and X² are independently H, OH, F, Cl, CF₃, NH₂, or substituted or unsubstituted C₁-C₄ alkyl (e.g., C₁, C₂, C₃ or C₄ alkyl);
X³ is C or N; and
R¹, R², W and B are as described above.

In some embodiments of compounds of Formula (II-a), X³ is N and X¹ is absent.

In some embodiments, the compound of the disclosure is a compound represented by Formula (II-b) or a pharmaceutically acceptable salt, ester, hydrate, solvate, or stereoisomer thereof:

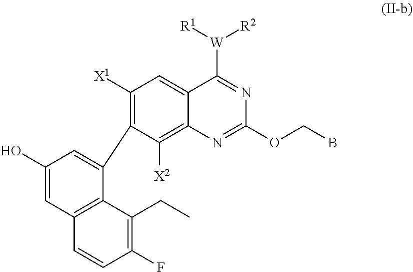

where:
X¹ and X² are independently H, F, Cl, CF₃, NH₃, or substituted or unsubstituted C₁-C₄ alkyl (e.g., C₁, C₂, C₃ or C₄ alkyl); and
R¹, R², W and B are as described above.

In some embodiments of compounds of Formula (II-a) and Formula (II-b), B is

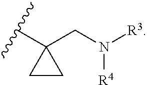

In some embodiments of compounds of Formula (II-a) and Formula (II-b), B is

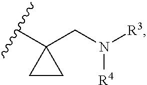

and R³ and R⁴ are substituted or unsubstituted C₁-C₅ alkyl group (e.g., substituted or unsubstituted C₁, C₂, C₃, C₄ or C₅ alkyl group) or absent, respectively.

In some embodiments of compounds of Formula (II-a) and Formula (II-b), B is

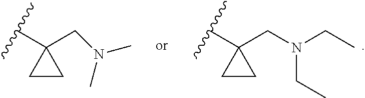

In some embodiments of compounds of Formula (II-a) and Formula (II-b), R³ and R⁴ are substituted C₁-C₅ alkyl group, wherein the substitutions are 3- to 7-membered carbon heteroatomic ring, wherein the heteroatom is N, O or S, wherein the number of heteroatoms is 1, 2, or 3.

In some embodiments of compounds of Formula (II-a) and Formula (II-b), R³ and R⁴ are

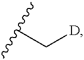

respectively; wherein D is H or 3- to 7-membered carbon heteroatomic ring (e.g., the ring is 3-, 4-, 5-, 6-, or 7-membered), pyrrole, furan, thiophene, oxazole, thiazole, pyrazole, imidazole, pyran, pyridine, piperidine, pyrimidine, pyrazine, oxacyclobutane, or azacyclobutane.

In some embodiments of compounds of Formula (II-a) and Formula (II-b), R³ and R⁴ and the N linked to them form a substituted or unsubstituted six-membered heterocyclic ring, five-membered heterocyclic ring or four-membered heterocyclic ring. In some embodiments, the heterocyclic ring has the structure:

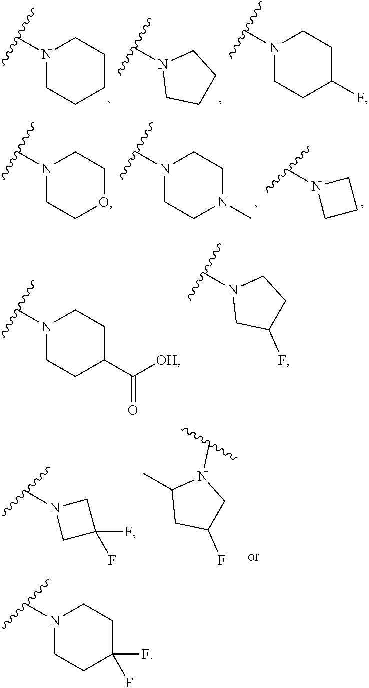

In some embodiments of compounds of Formula (II-a) and Formula (II-b), R³ and R⁴ and the N linked to them form a multiple-membered heterocyclic ring. In some embodiments, the heterocyclic ring has the structure:

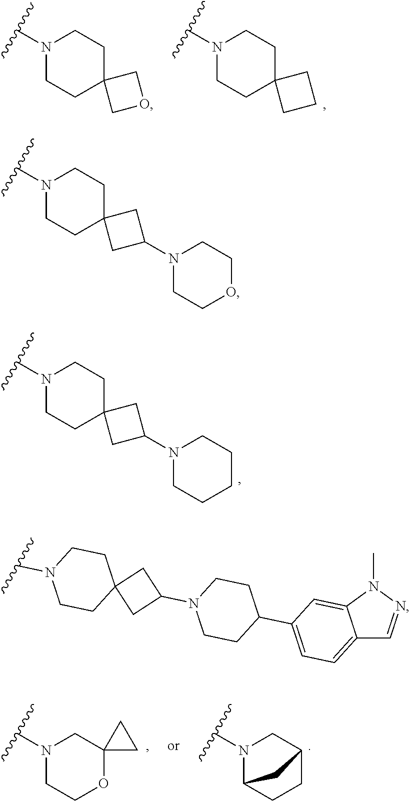

In some embodiments of compounds of Formula (II-a) and Formula (II-b), B is

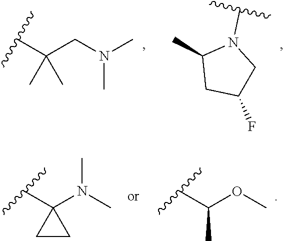

In some embodiments of compounds of Formula (II-a) and Formula (II-b), W is N, and R¹ and R² and the W linked to them form

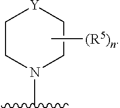

In some such embodiments, Y is O, N, —CH₂—, —CH₂CH₂—, —OCH₂— or absent. In some specific embodiments, Y is absent, and R¹ and R² and the W linked to them form

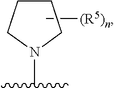

wherein R⁵ is selected from one or more of —OH, hydroxy-substituted C₁-C₂ alkyl group, amino group, amide group, C₁-C₂ alkyl group, halogen, C(═O)O C₁-C₂ alkyl group, C₁-C₂ alkoxy group, or carboxyl group. In some such embodiments,

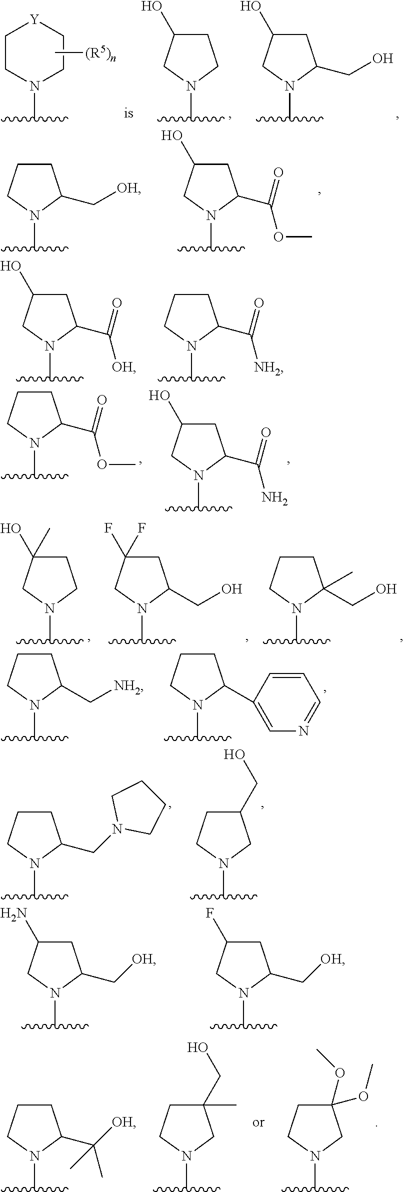

In some embodiments, Y is absent, R¹, R² and the W linked to them form a substituted or unsubstituted 5-membered nitrogen heterocyclic ring, and together they form a bridged ring, condensed ring or multi-carbon ring, which is

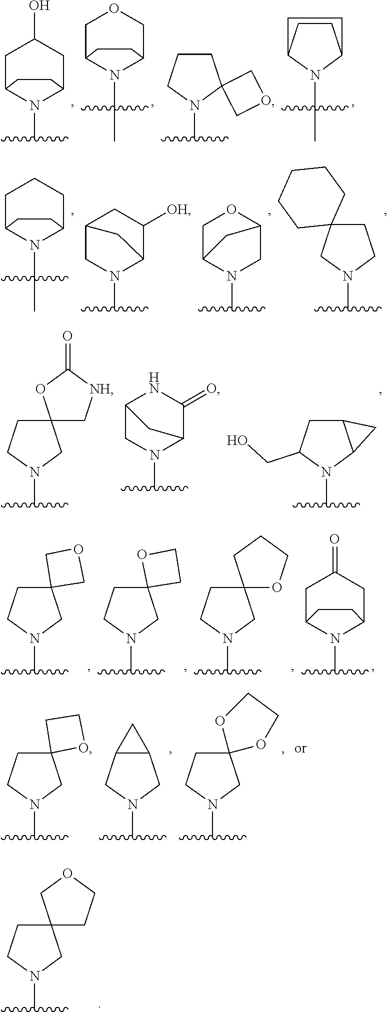

In some embodiments, Y is O, R¹, R² and the W linked to them form

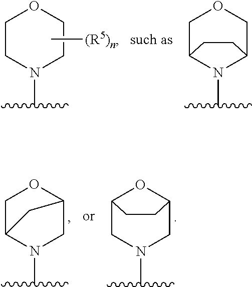

In some embodiments, Y is N, R¹, R² and the W linked to them form

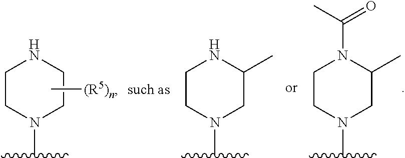

In some embodiments, Y is —CH₂, and R¹, R² and the W linked to them form a substituted or unsubstituted 6-membered nitrogen heterocyclic ring, which is

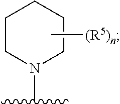

wherein R⁵ is —OH, —C═O—, C₁-C₂ alkyl substituted by —COOH, C₁-C₂ alkyl, C₁-C₂ alkoxy, amino, penyl, pyridyl; or

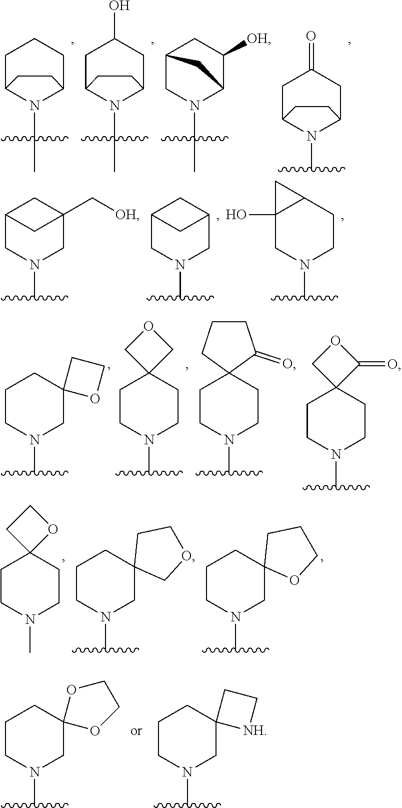

In some embodiments, R⁵ and the atoms linked to it form one of the following structures:

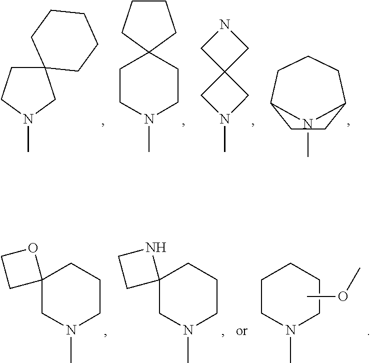

In some embodiments, the compound of the disclosure is a compound represented by Formula (III-a) or Formula (III-b) or a pharmaceutically acceptable salt, ester, hydrate, solvate, or stereoisomer thereof:

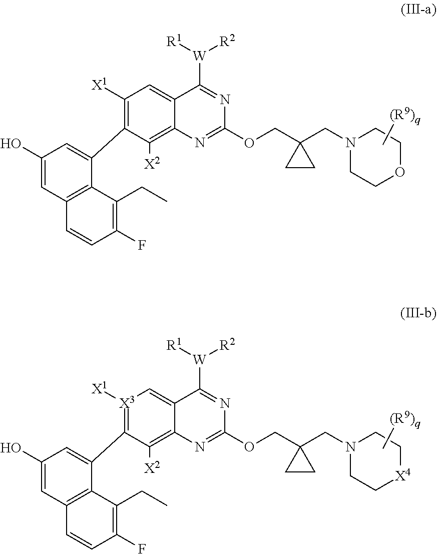

where:
any arbitrary R⁹ is independently H, F, Cl, CF₃, NH₃ or substituted or unsubstituted C₁-C₄ alkyl (e.g., substituted or unsubstituted C₁, C₂, C₃ or C₄ alkyl); or,
two arbitrary R⁹s that are linked to one atom and a heterocyclic ring linked to them form a spiro ring; or,
two adjacent R⁹s and a heterocyclic ring linked to them form a condensed ring; or,
two non-adjacent R⁹s and ae heterocyclic ring linked to them form a bridged ring; and
X⁴ is C, N, O.
q is an integer of 0 to 4, i.e., q is 0, 1, 2, 3 or 4.

In some embodiments, the compound of the disclosure is a compound represented by Formula (IV-a) or a pharmaceutically acceptable salt, ester, hydrate, solvate, or stereoisomer thereof:

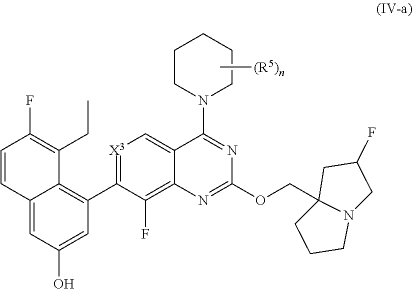

where:
two arbitrary R⁵s linked to one atom and a ring linked to them form a spiro ring, where the two R⁵s linked to one atom form one ring and R¹ and R² and N atom form another ring; wherein the ring formed by the two R⁵s linked to one atom is an oxaalkyl ring, and the spiro ring is optionally substituted by alkyl, hydroxyl, halogen, amino, ═O or —CN; and
X³ is as described above.

In some embodiments of compounds of Formula (IV-a), the ring formed by the two R⁵s linked to one atom is a three-membered oxacyclic ring, four-membered oxacyclic ring, five-membered oxacyclic ring or six-membered oxacyclic ring.

In some embodiments of compounds of Formula (IV-a), R⁵ and the ring linked to it form the following structure:

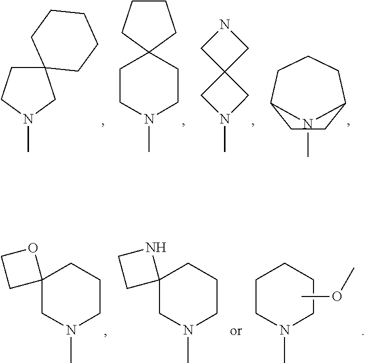

In some embodiments, the compound of the disclosure is a compound represented by Formula (IV-b) or a pharmaceutically acceptable salt, ester, hydrate, solvate, or stereoisomer thereof:

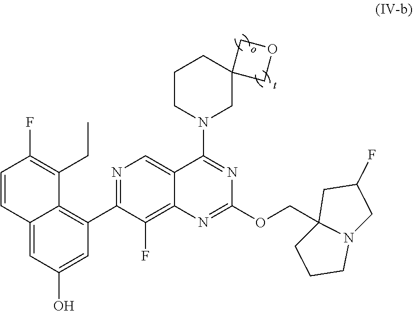

where:
is an integer of 0 to 3, i.e., o is 0, 1, 2 or 3; and
t is an integer of 2 to 6, i.e., t is 2, 3, 4, 5 or 6.

In some embodiments, the compound of the disclosure is a compound represented by Formula (IV-c) or a pharmaceutically acceptable salt, ester, hydrate, solvate, or stereoisomer thereof:

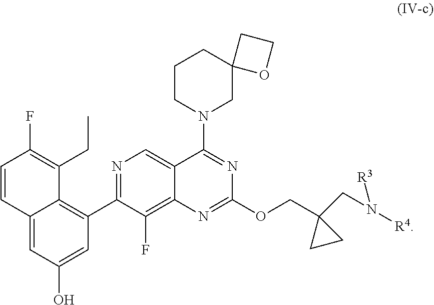

In some embodiments, the compound of the disclosure is a compound shown by Formula (V) or a pharmaceutically acceptable salt, ester, hydrate, solvate, or stereoisomer thereof:

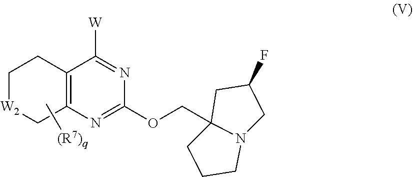

where:
W₂ is —CH₂— or —NH—;
R⁷ is H, F, Cl, CF₃, NH₂, substituted or unsubstituted aryl, substituted or unsubstituted cycloalkyl, or substituted or unsubstituted heterocycloalkyl; or,
any two R⁷s linked to the same atom and the ring they are linked to form a substituted or unsubstituted spiro ring (e.g., any two R⁷s linked to the same atom combine to form a substituted or unsubstituted cycloalkyl or substituted or unsubstituted heterocycloalkyl);
q is an integer of 0 to 4 (i.e., q is 0, 1, 2, 3 or 4); and
W is as defined above.

In some embodiments, the compound of the disclosure is a compound shown by Formula (VI) or a pharmaceutically acceptable salt, ester, hydrate, solvate, or stereoisomer thereof:

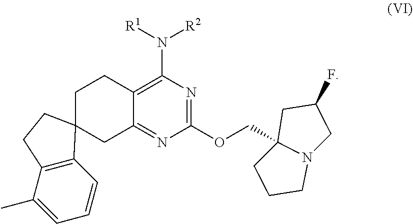

In some embodiments, the compound of the disclosure is a compound shown in Table 1 or Table 2, or a pharmaceutically acceptable salt, ester, hydrate, solvate or stereoisomer thereof.

TABLE 1
Structures of exemplary compounds in accordance with certain embodiments of
the disclosure.

| Cpd | |
|---|---|
| No. | Structure |
| 1 | 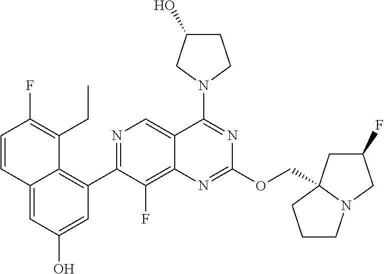 [SMILES: CCc1c(F)ccc2cc(O)cc(-c3ncc4c(N5CC[C@@H](O)C5)nc(OC[C@@]56CCCN5C[C@H](F)C6)nc4c3F)c12] |
| 2 | 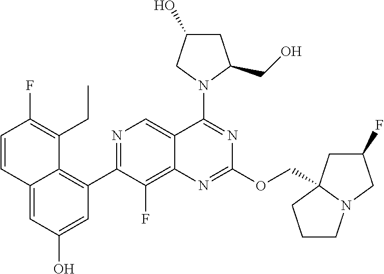 [SMILES: CCc1c(F)ccc2cc(O)cc(-c3ncc4c(N5C[C@H](O)C[C@H]5CO)nc(OC[C@@]56CCCN5C[C@H](F)C6)nc4c3F)c12] |
| 3 | 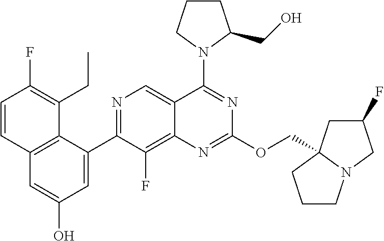 [SMILES: CCc1c(F)ccc2cc(O)cc(-c3ncc4c(N5CCC[C@H]5CO)nc(OC[C@@]56CCCN5C[C@H](F)C6)nc4c3F)c12] |
| 4 | 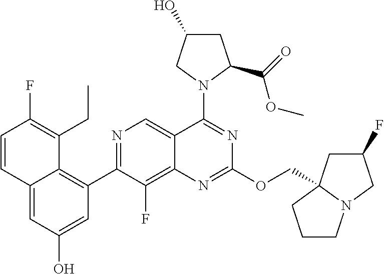 [SMILES: CCc1c(F)ccc2cc(O)cc(-c3ncc4c(N5C[C@H](O)C[C@H]5C(=O)OC)nc(OC[C@@]56CCCN5C[C@H](F)C6)nc4c3F)c12] |
| 5 | 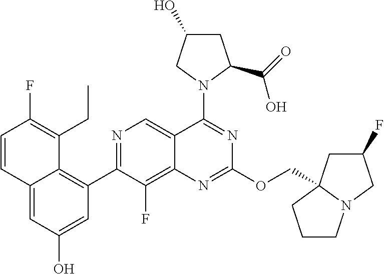 [SMILES: CCc1c(F)ccc2cc(O)cc(-c3ncc4c(N5C[C@H](O)C[C@H]5C(=O)O)nc(OC[C@@]56CCCN5C[C@H](F)C6)nc4c3F)c12] |
| 6 | 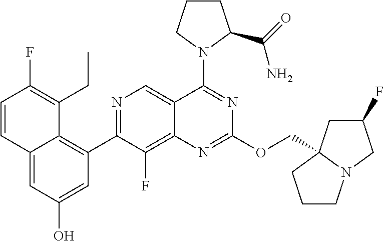 [SMILES: CCc1c(F)ccc2cc(O)cc(-c3ncc4c(N5CCC[C@H]5C(N)=O)nc(OC[C@@]56CCCN5C[C@H](F)C6)nc4c3F)c12] |
| 7 | 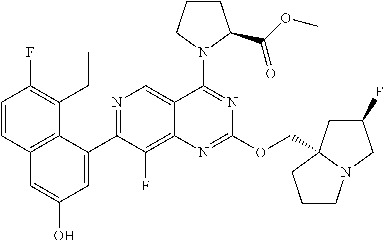 [SMILES: CCc1c(F)ccc2cc(O)cc(-c3ncc4c(N5CCC[C@H]5C(=O)OC)nc(OC[C@@]56CCCN5C[C@H](F)C6)nc4c3F)c12] |
| 8 | 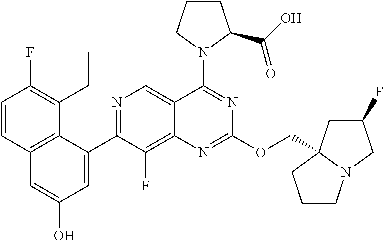 [SMILES: CCc1c(F)ccc2cc(O)cc(-c3ncc4c(N5CCC[C@H]5C(=O)O)nc(OC[C@@]56CCCN5C[C@H](F)C6)nc4c3F)c12] |
| 9 | 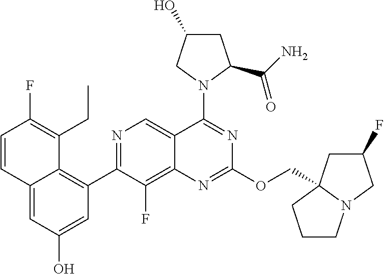 [SMILES: CCc1c(F)ccc2cc(O)cc(-c3ncc4c(N5C[C@H](O)C[C@H]5C(N)=O)nc(OC[C@@]56CCCN5C[C@H](F)C6)nc4c3F)c12] |
| 10 | 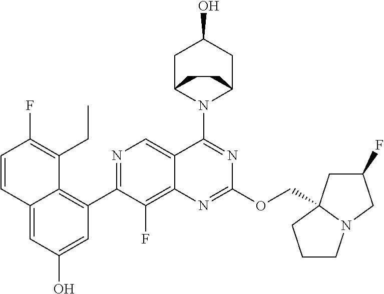 [SMILES: CCc1c(F)ccc2cc(O)cc(-c3ncc4c(N5[C@@H]6CC[C@H]5C[C@@H](O)C6)nc(OC[C@@]56CCCN5C[C@H](F)C6)nc4c3F)c12] |
| 11 | 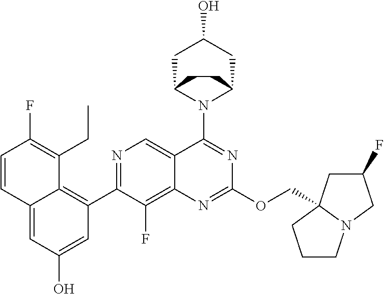 [SMILES: CCc1c(F)ccc2cc(O)cc(-c3ncc4c(N5[C@@H]6CC[C@H]5C[C@H](O)C6)nc(OC[C@@]56CCCN5C[C@H](F)C6)nc4c3F)c12] |
| 12 | 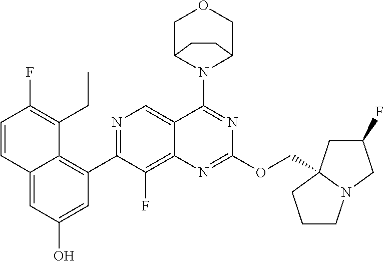 [SMILES: CCc1c(F)ccc2cc(O)cc(-c3ncc4c(N5C6CCC5COC6)nc(OC[C@@]56CCCN5C[C@H](F)C6)nc4c3F)c12] |
| 13 | 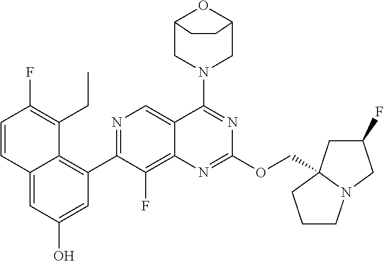 [SMILES: CCc1c(F)ccc2cc(O)cc(-c3ncc4c(N5CC6CCC(C5)O6)nc(OC[C@@]56CCCN5C[C@H](F)C6)nc4c3F)c12] |
| 14 | 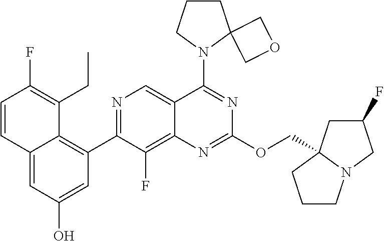 [SMILES: CCc1c(F)ccc2cc(O)cc(-c3ncc4c(N5CCCC56COC6)nc(OC[C@@]56CCCN5C[C@H](F)C6)nc4c3F)c12] |
| 15 | 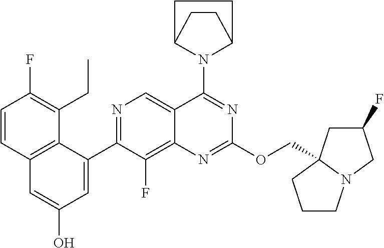 [SMILES: CCc1c(F)ccc2cc(O)cc(-c3ncc4c(N5C6CCC5CC6)nc(OC[C@@]56CCCN5C[C@H](F)C6)nc4c3F)c12] |
| 16 | 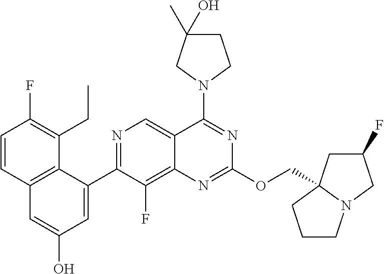 [SMILES: CCc1c(F)ccc2cc(O)cc(-c3ncc4c(N5CCC(C)(O)C5)nc(OC[C@@]56CCCN5C[C@H](F)C6)nc4c3F)c12] |
| 17 | 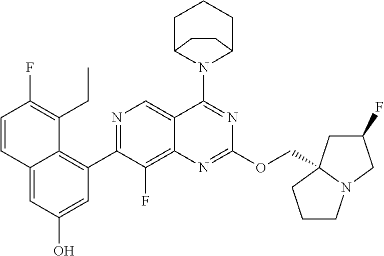 [SMILES: CCc1c(F)ccc2cc(O)cc(-c3ncc4c(N5C6CCCC5CC6)nc(OC[C@@]56CCCN5C[C@H](F)C6)nc4c3F)c12] |
| 18 | 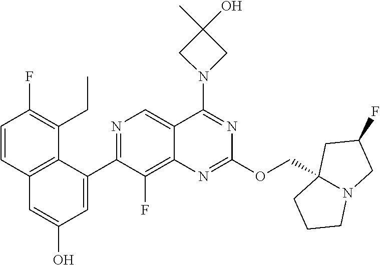 [SMILES: CCc1c(F)ccc2cc(O)cc(-c3ncc4c(N5CC(C)(O)C5)nc(OC[C@@]56CCCN5C[C@H](F)C6)nc4c3F)c12] |
| 19 | 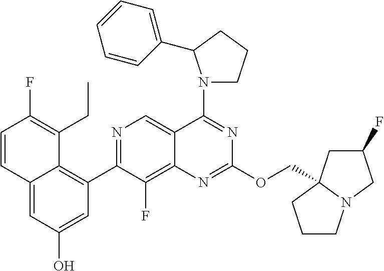 [SMILES: CCc1c(F)ccc2cc(O)cc(-c3ncc4c(N5CCCC5c5ccccc5)nc(OC[C@@]56CCCN5C[C@H](F)C6)nc4c3F)c12] |
| 20 | 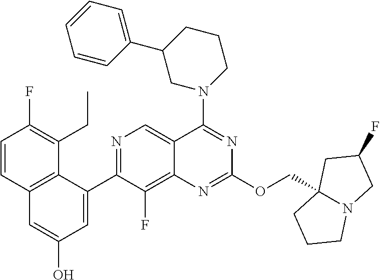 [SMILES: CCc1c(F)ccc2cc(O)cc(-c3ncc4c(N5CCCC(c6ccccc6)C5)nc(OC[C@@]56CCCN5C[C@H](F)C6)nc4c3F)c12] |
| 21 | 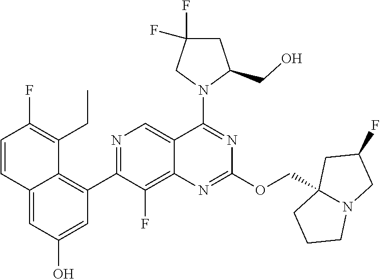 [SMILES: CCc1c(F)ccc2cc(O)cc(-c3ncc4c(N5CC(F)(F)C[C@H]5CO)nc(OC[C@@]56CCCN5C[C@H](F)C6)nc4c3F)c12] |
| 22 | 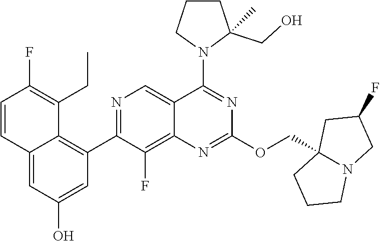 [SMILES: CCc1c(F)ccc2cc(O)cc(-c3ncc4c(N5CCC[C@@]5(C)CO)nc(OC[C@@]56CCCN5C[C@H](F)C6)nc4c3F)c12] |
| 23 | 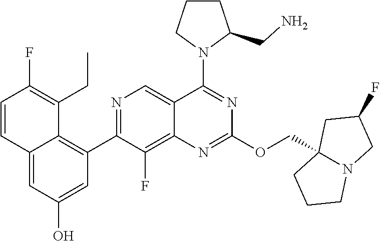 [SMILES: CCc1c(F)ccc2cc(O)cc(-c3ncc4c(N5CCC[C@H]5CN)nc(OC[C@@]56CCCN5C[C@H](F)C6)nc4c3F)c12] |
| 24 | 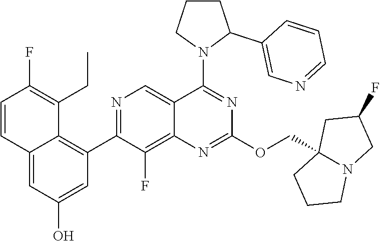 [SMILES: CCc1c(F)ccc2cc(O)cc(-c3ncc4c(N5CCCC5c5cccnc5)nc(OC[C@@]56CCCN5C[C@H](F)C6)nc4c3F)c12] |
| 25 | 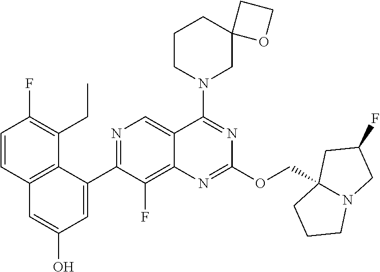 [SMILES: CCc1c(F)ccc2cc(O)cc(-c3ncc4c(N5CCCC6(CCO6)C5)nc(OC[C@@]56CCCN5C[C@H](F)C6)nc4c3F)c12] |
| 26 | 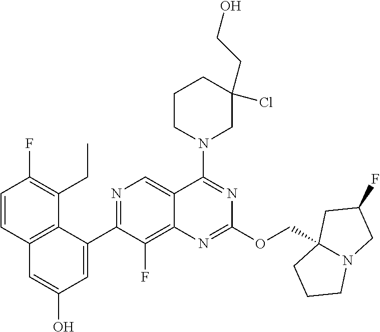 [SMILES: CCc1c(F)ccc2cc(O)cc(-c3ncc4c(N5CCCC(Cl)(CCO)C5)nc(OC[C@@]56CCCN5C[C@H](F)C6)nc4c3F)c12] |
| 27 | 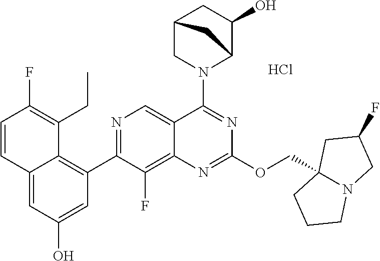 [SMILES: CCc1c(F)ccc2cc(O)cc(-c3ncc4c(N5C[C@@H]6C[C@@H](O)[C@H]5C6)nc(OC[C@@]56CCCN5C[C@H](F)C6)nc4c3F)c12 ; Cl] |
| 28 | 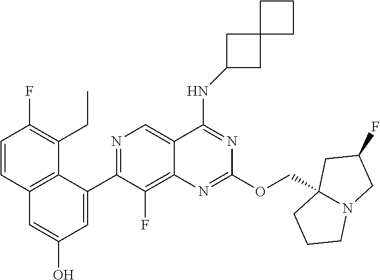 [SMILES: CCc1c(F)ccc2cc(O)cc(-c3ncc4c(NC5CC6(CCC6)C5)nc(OC[C@@]56CCCN5C[C@H](F)C6)nc4c3F)c12] |
| 29 | 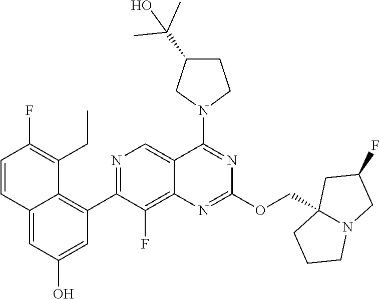 [SMILES: CCc1c(F)ccc2cc(O)cc(-c3ncc4c(N5CC[C@@H](C(C)(C)O)C5)nc(OC[C@@]56CCCN5C[C@H](F)C6)nc4c3F)c12] |
| 30 | 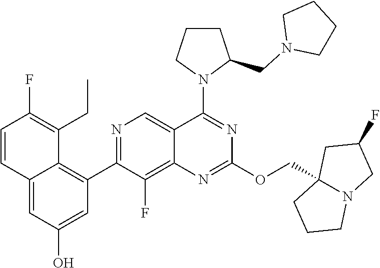 [SMILES: CCc1c(F)ccc2cc(O)cc(-c3ncc4c(N5CCC[C@H]5CN5CCCC5)nc(OC[C@@]56CCCN5C[C@H](F)C6)nc4c3F)c12] |
| 31 | 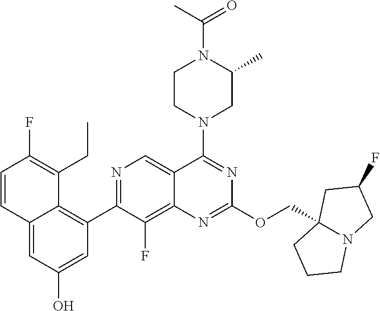 [SMILES: CCc1c(F)ccc2cc(O)cc(-c3ncc4c(N5CCN(C(C)=O)[C@H](C)C5)nc(OC[C@@]56CCCN5C[C@H](F)C6)nc4c3F)c12] |
| 32 | 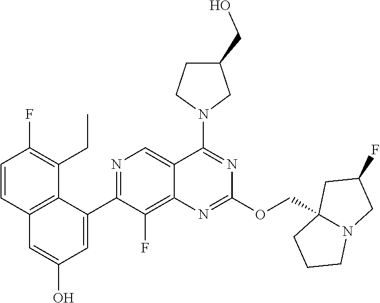 [SMILES: CCc1c(F)ccc2cc(O)cc(-c3ncc4c(N5CC[C@@H](CO)C5)nc(OC[C@@]56CCCN5C[C@H](F)C6)nc4c3F)c12] |
| 33 | 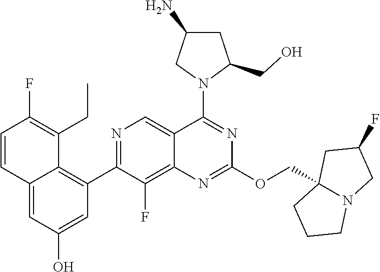 [SMILES: CCc1c(F)ccc2cc(O)cc(-c3ncc4c(N5C[C@@H](N)C[C@H]5CO)nc(OC[C@@]56CCCN5C[C@H](F)C6)nc4c3F)c12] |
| 34 | 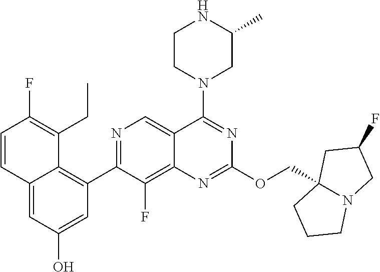 [SMILES: CCc1c(F)ccc2cc(O)cc(-c3ncc4c(N5CCN[C@H](C)C5)nc(OC[C@@]56CCCN5C[C@H](F)C6)nc4c3F)c12] |
| 35 | 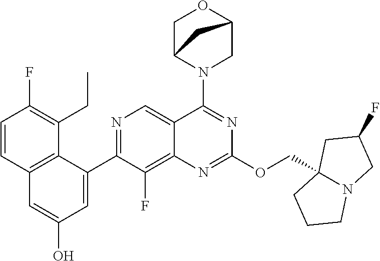 [SMILES: CCc1c(F)ccc2cc(O)cc(-c3ncc4c(N5C[C@@H]6C[C@H]5CO6)nc(OC[C@@]56CCCN5C[C@H](F)C6)nc4c3F)c12] |
| 36 | 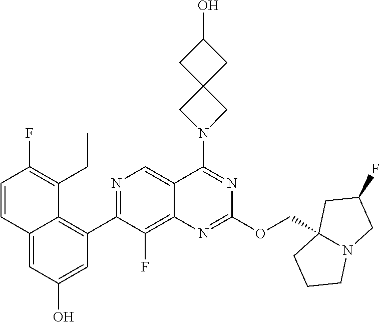 [SMILES: CCc1c(F)ccc2cc(O)cc(-c3ncc4c(N5CC6(CC(O)C6)C5)nc(OC[C@@]56CCCN5C[C@H](F)C6)nc4c3F)c12] |
| 37 | 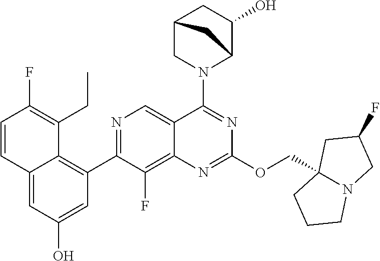 [SMILES: CCc1c(F)ccc2cc(O)cc(-c3ncc4c(N5C[C@H]6C[C@@H]5[C@@H](O)C6)nc(OC[C@@]56CCCN5C[C@H](F)C6)nc4c3F)c12] |
| 38 | 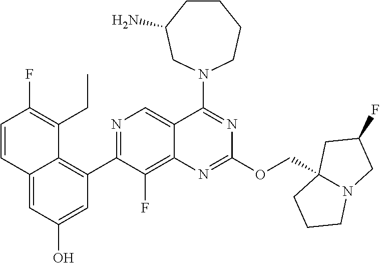 [SMILES: CCc1c(F)ccc2cc(O)cc(-c3ncc4c(N5CCCC[C@@H](N)C5)nc(OC[C@@]56CCCN5C[C@H](F)C6)nc4c3F)c12] |
| 39 | 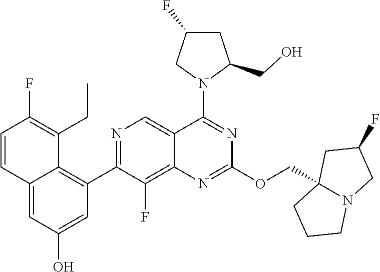 [SMILES: CCc1c(F)ccc2cc(O)cc(-c3ncc4c(N5C[C@H](F)C[C@H]5CO)nc(OC[C@@]56CCCN5C[C@H](F)C6)nc4c3F)c12] |
| 40 | 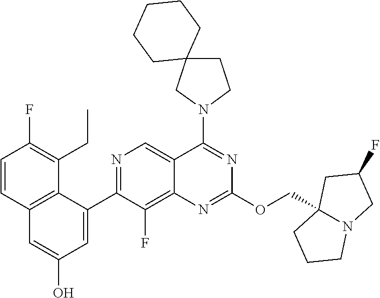 [SMILES: CCc1c(F)ccc2cc(O)cc(-c3ncc4c(N5CCC6(CCCCC6)C5)nc(OC[C@@]56CCCN5C[C@H](F)C6)nc4c3F)c12] |
| 41 | 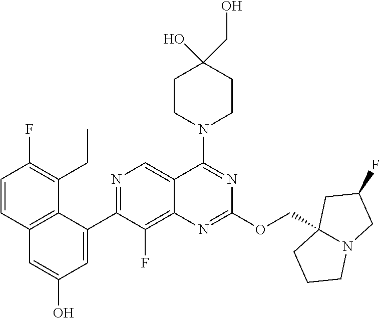 [SMILES: CCc1c(F)ccc2cc(O)cc(-c3ncc4c(N5CCC(O)(CO)CC5)nc(OC[C@@]56CCCN5C[C@H](F)C6)nc4c3F)c12] |
| 42 | 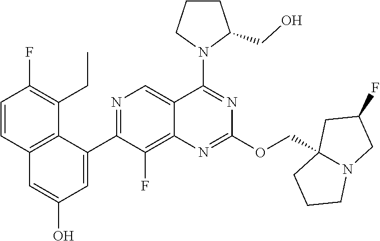 [SMILES: CCc1c(F)ccc2cc(O)cc(-c3ncc4c(N5CCC[C@@H]5CO)nc(OC[C@@]56CCCN5C[C@H](F)C6)nc4c3F)c12] |
| 43 | 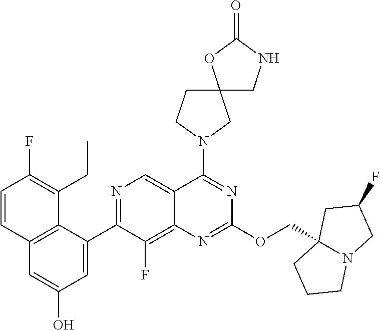 [SMILES: CCc1c(F)ccc2cc(O)cc(-c3ncc4c(N5CCC6(CNC(=O)O6)C5)nc(OC[C@@]56CCCN5C[C@H](F)C6)nc4c3F)c12] |
| 44 | 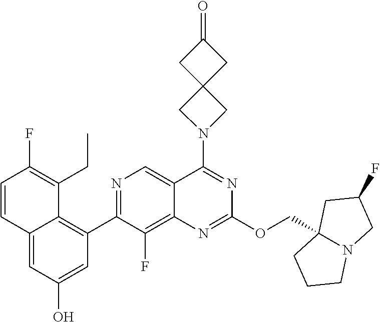 [SMILES: CCc1c(F)ccc2cc(O)cc(-c3ncc4c(N5CC6(CC(=O)C6)C5)nc(OC[C@@]56CCCN5C[C@H](F)C6)nc4c3F)c12] |
| 45 | 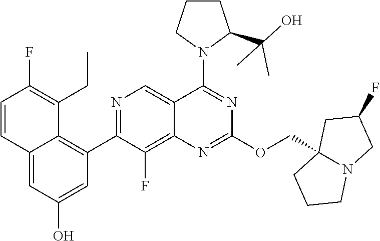 [SMILES: CCc1c(F)ccc2cc(O)cc(-c3ncc4c(N5CCC[C@H]5C(C)(C)O)nc(OC[C@@]56CCCN5C[C@H](F)C6)nc4c3F)c12] |
| 46 | 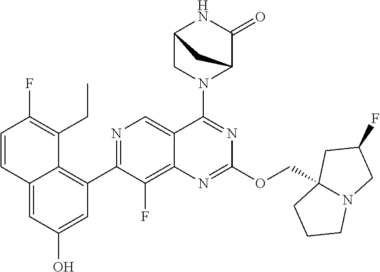 [SMILES: CCc1c(F)ccc2cc(O)cc(-c3ncc4c(N5C[C@H]6C[C@@H]5C(=O)N6)nc(OC[C@@]56CCCN5C[C@H](F)C6)nc4c3F)c12] |
| 47 | 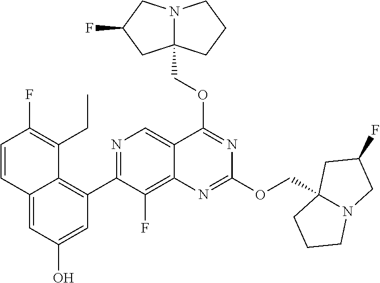 [SMILES: CCc1c(F)ccc2cc(O)cc(-c3ncc4c(OC[C@@]56CCCN5C[C@H](F)C6)nc(OC[C@@]56CCCN5C[C@H](F)C6)nc4c3F)c12] |
| 48 | 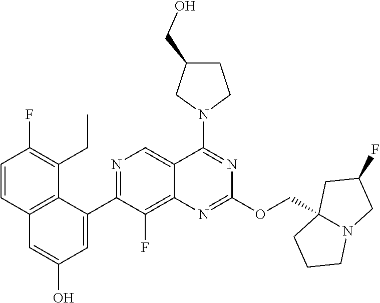 [SMILES: CCc1c(F)ccc2cc(O)cc(-c3ncc4c(N5CC[C@H](CO)C5)nc(OC[C@@]56CCCN5C[C@H](F)C6)nc4c3F)c12] |
| 49 | 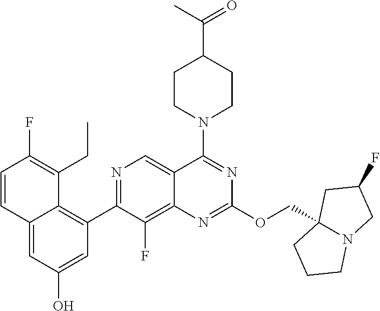 [SMILES: CCc1c(F)ccc2cc(O)cc(-c3ncc4c(N5CCC(C(C)=O)CC5)nc(OC[C@@]56CCCN5C[C@H](F)C6)nc4c3F)c12] |
| 50 | 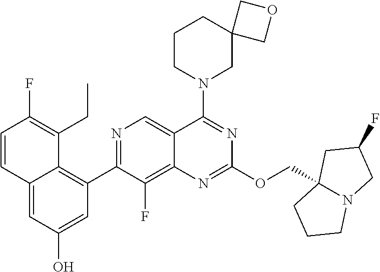 [SMILES: CCc1c(F)ccc2cc(O)cc(-c3ncc4c(N5CCCC6(COC6)C5)nc(OC[C@@]56CCCN5C[C@H](F)C6)nc4c3F)c12] |
| 51 | 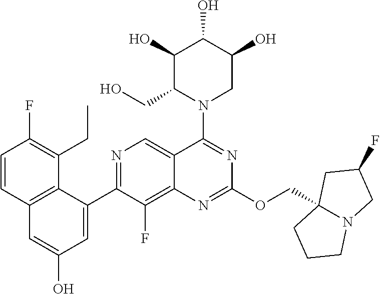 [SMILES: CCc1c(F)ccc2cc(O)cc(-c3ncc4c(N5C[C@H](O)[C@@H](O)[C@H](O)[C@H]5CO)nc(OC[C@@]56CCCN5C[C@H](F)C6)nc4c3F)c12] |
| 52 | 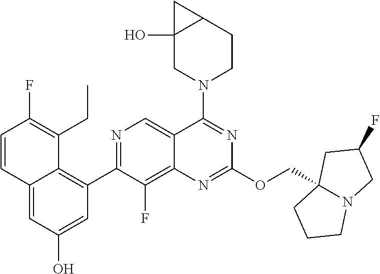 [SMILES: CCc1c(F)ccc2cc(O)cc(-c3ncc4c(N5CCC6CC6(O)C5)nc(OC[C@@]56CCCN5C[C@H](F)C6)nc4c3F)c12] |
| 53 | 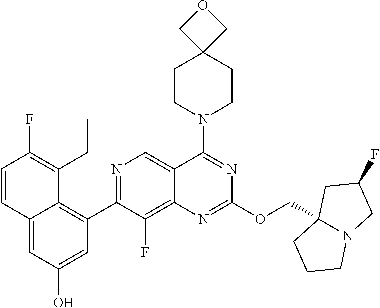 [SMILES: CCc1c(F)ccc2cc(O)cc(-c3ncc4c(N5CCC6(CC5)COC6)nc(OC[C@@]56CCCN5C[C@H](F)C6)nc4c3F)c12] |
| 54 | 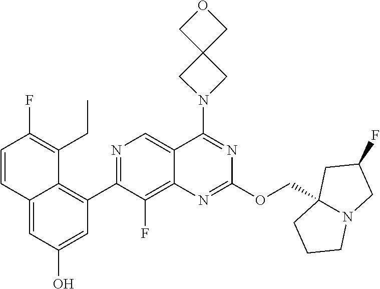 [SMILES: CCc1c(F)ccc2cc(O)cc(-c3ncc4c(N5CC6(COC6)C5)nc(OC[C@@]56CCCN5C[C@H](F)C6)nc4c3F)c12] |
| 55 | 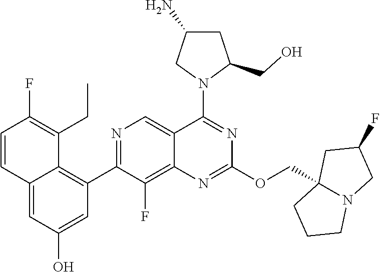 [SMILES: CCc1c(F)ccc2cc(O)cc(-c3ncc4c(N5C[C@H](N)C[C@H]5CO)nc(OC[C@@]56CCCN5C[C@H](F)C6)nc4c3F)c12] |
| 56 | 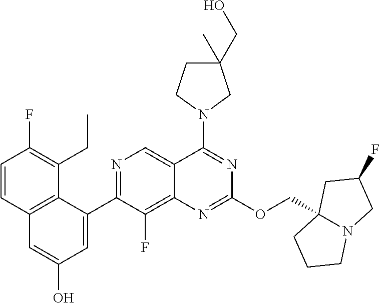 [SMILES: CCc1c(F)ccc2cc(O)cc(-c3ncc4c(N5CCC(C)(CO)C5)nc(OC[C@@]56CCCN5C[C@H](F)C6)nc4c3F)c12] |
| 57 | 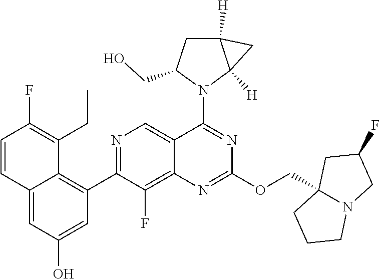 [SMILES: CCc1c(F)ccc2cc(O)cc(-c3ncc4c(N5[C@H](CO)C[C@H]6C[C@H]65)nc(OC[C@@]56CCCN5C[C@H](F)C6)nc4c3F)c12] |
| 58 | 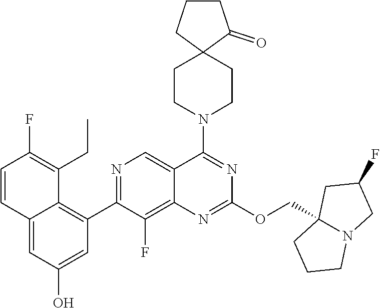 [SMILES: CCc1c(F)ccc2cc(O)cc(-c3ncc4c(N5CCC6(CCCC6=O)CC5)nc(OC[C@@]56CCCN5C[C@H](F)C6)nc4c3F)c12] |
| 59 | 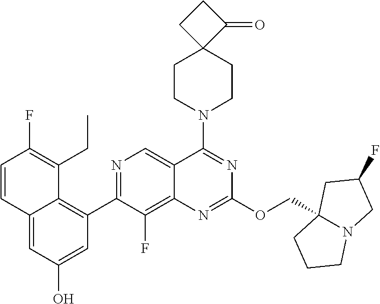 [SMILES: CCc1c(F)ccc2cc(O)cc(-c3ncc4c(N5CCC6(CCC6=O)CC5)nc(OC[C@@]56CCCN5C[C@H](F)C6)nc4c3F)c12] |
| 60 | 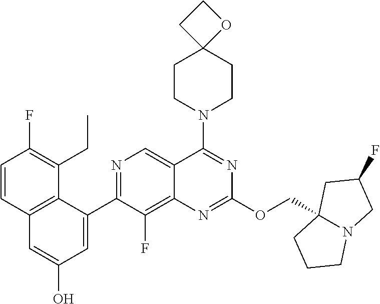 [SMILES: CCc1c(F)ccc2cc(O)cc(-c3ncc4c(N5CCC6(CCO6)CC5)nc(OC[C@@]56CCCN5C[C@H](F)C6)nc4c3F)c12] |
| 61 | 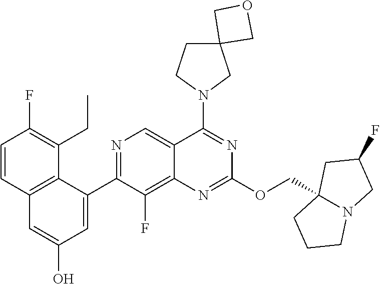 [SMILES: CCc1c(F)ccc2cc(O)cc(-c3ncc4c(N5CCC6(COC6)C5)nc(OC[C@@]56CCCN5C[C@H](F)C6)nc4c3F)c12] |
| 62 | 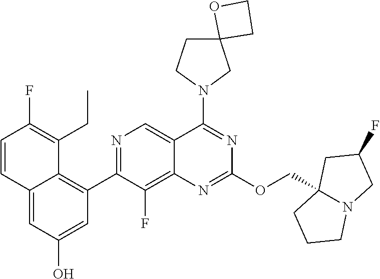 [SMILES: CCc1c(F)ccc2cc(O)cc(-c3ncc4c(N5CCC6(CCO6)C5)nc(OC[C@@]56CCCN5C[C@H](F)C6)nc4c3F)c12] |
| 63 | 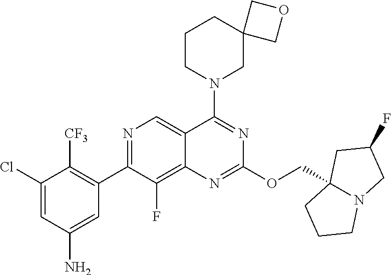 [SMILES: Nc1cc(Cl)c(C(F)(F)F)c(-c2ncc3c(N4CCCC5(COC5)C4)nc(OC[C@@]45CCCN4C[C@H](F)C5)nc3c2F)c1] |
| 64 | 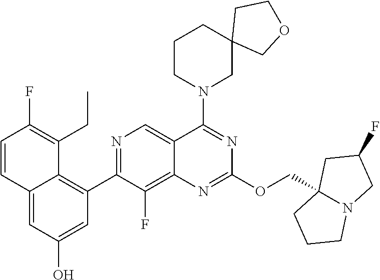 [SMILES: CCc1c(F)ccc2cc(O)cc(-c3ncc4c(N5CCCC6(CCOC6)C5)nc(OC[C@@]56CCCN5C[C@H](F)C6)nc4c3F)c12] |
| 65 | 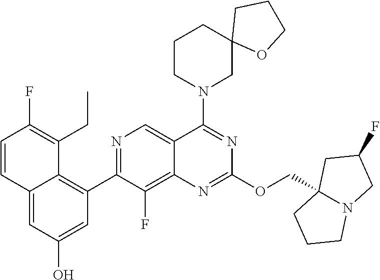 [SMILES: CCc1c(F)ccc2cc(O)cc(-c3ncc4c(N5CCCC6(CCCO6)C5)nc(OC[C@@]56CCCN5C[C@H](F)C6)nc4c3F)c12] |
| 66 | 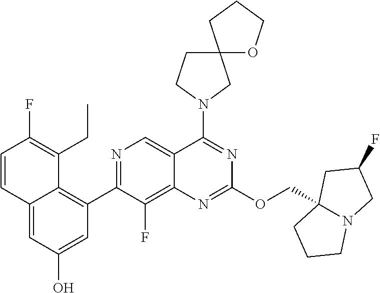 [SMILES: CCc1c(F)ccc2cc(O)cc(-c3ncc4c(N5CCC6(CCCO6)C5)nc(OC[C@@]56CCCN5C[C@H](F)C6)nc4c3F)c12] |
| 67 | 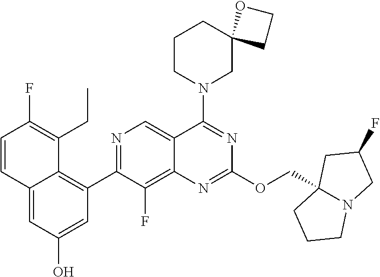 [SMILES: CCc1c(F)ccc2cc(O)cc(-c3ncc4c(N5CCC[C@@]6(CCO6)C5)nc(OC[C@@]56CCCN5C[C@H](F)C6)nc4c3F)c12] |
| 68 | 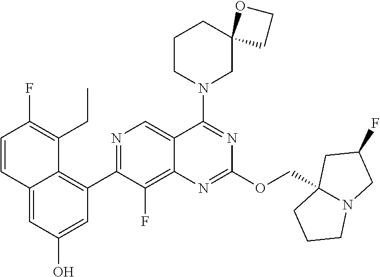 [SMILES: CCc1c(F)ccc2cc(O)cc(-c3ncc4c(N5CCC[C@]6(CCO6)C5)nc(OC[C@@]56CCCN5C[C@H](F)C6)nc4c3F)c12] |
| 69 | 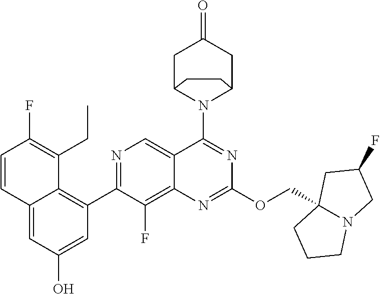 [SMILES: CCc1c(F)ccc2cc(O)cc(-c3ncc4c(N5C6CCC5CC(=O)C6)nc(OC[C@@]56CCCN5C[C@H](F)C6)nc4c3F)c12] |
| 70 | 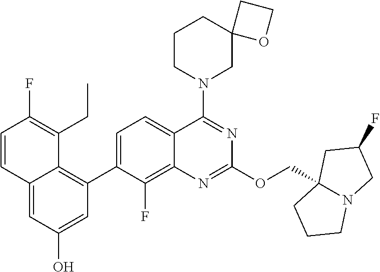 [SMILES: CCc1c(F)ccc2cc(O)cc(-c3ccc4c(N5CCCC6(CCO6)C5)nc(OC[C@@]56CCCN5C[C@H](F)C6)nc4c3F)c12] |
| 71 | 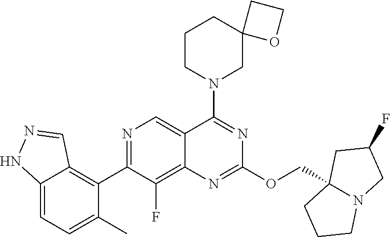 [SMILES: Cc1ccc2[nH]ncc2c1-c1ncc2c(N3CCCC4(CCO4)C3)nc(OC[C@@]34CCCN3C[C@H](F)C4)nc2c1F] |
| 72 | 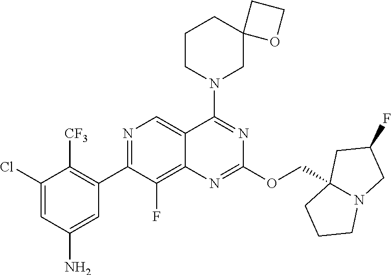 [SMILES: Nc1cc(Cl)c(C(F)(F)F)c(-c2ncc3c(N4CCCC5(CCO5)C4)nc(OC[C@@]45CCCN4C[C@H](F)C5)nc3c2F)c1] |
| 73 | 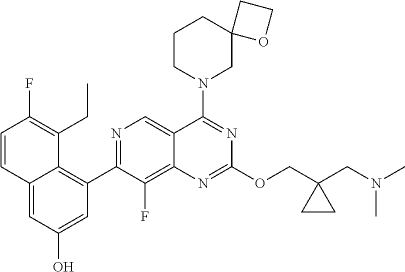 [SMILES: CCc1c(F)ccc2cc(O)cc(-c3ncc4c(N5CCCC6(CCO6)C5)nc(OCC5(CN(C)C)CC5)nc4c3F)c12] |
| 74 | 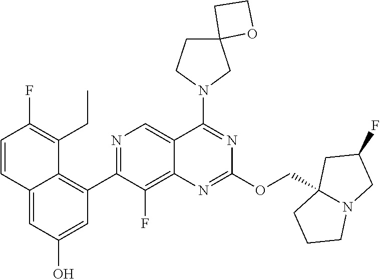 [SMILES: CCc1c(F)ccc2cc(O)cc(-c3ncc4c(N5CCC6(CCO6)C5)nc(OC[C@@]56CCCN5C[C@H](F)C6)nc4c3F)c12] |
| 75 | 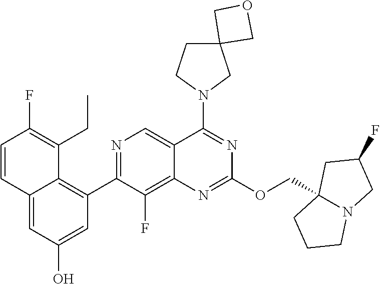 [SMILES: CCc1c(F)ccc2cc(O)cc(-c3ncc4c(N5CCC6(COC6)C5)nc(OC[C@@]56CCCN5C[C@H](F)C6)nc4c3F)c12] |
| 76 | 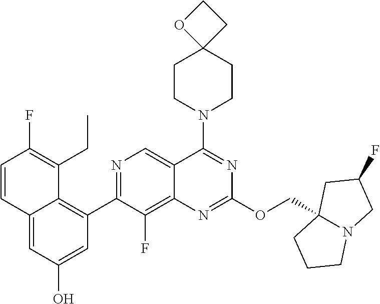 [SMILES: CCc1c(F)ccc2cc(O)cc(-c3ncc4c(N5CCC6(CCO6)CC5)nc(OC[C@@]56CCCN5C[C@H](F)C6)nc4c3F)c12] |
| 77 | 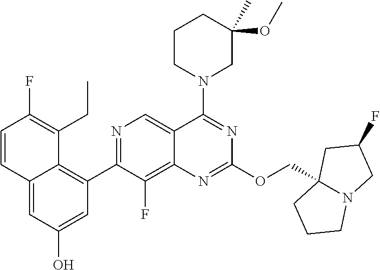 [SMILES: CCc1c(F)ccc2cc(O)cc(-c3ncc4c(N5CCC[C@@](C)(OC)C5)nc(OC[C@@]56CCCN5C[C@H](F)C6)nc4c3F)c12] |
| 78 | 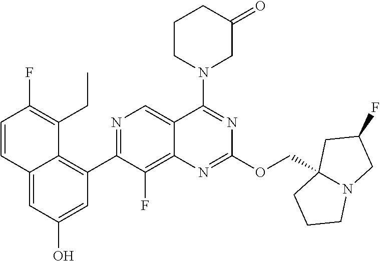 [SMILES: CCc1c(F)ccc2cc(O)cc(-c3ncc4c(N5CCCC(=O)C5)nc(OC[C@@]56CCCN5C[C@H](F)C6)nc4c3F)c12] |
| 79 | 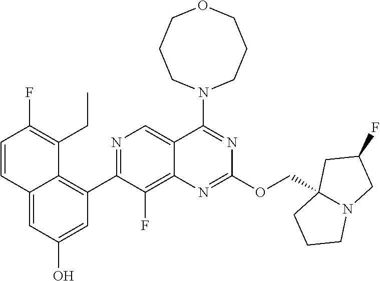 [SMILES: CCc1c(F)ccc2cc(O)cc(-c3ncc4c(N5CCCOCCC5)nc(OC[C@@]56CCCN5C[C@H](F)C6)nc4c3F)c12] |
| 80 | 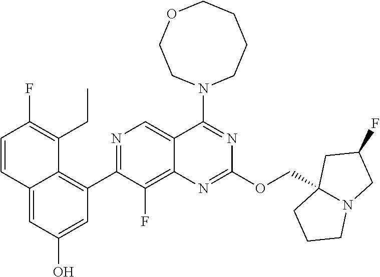 [SMILES: CCc1c(F)ccc2cc(O)cc(-c3ncc4c(N5CCCCOCC5)nc(OC[C@@]56CCCN5C[C@H](F)C6)nc4c3F)c12] |
| 81 | 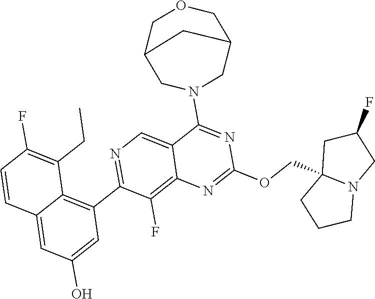 [SMILES: CCc1c(F)ccc2cc(O)cc(-c3ncc4c(N5CC6COCC(C6)C5)nc(OC[C@@]56CCCN5C[C@H](F)C6)nc4c3F)c12] |
| 82 | 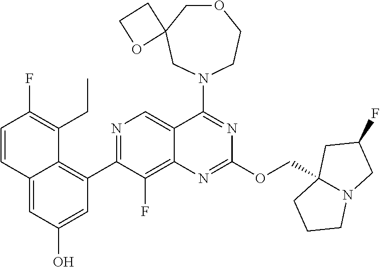 [SMILES: CCc1c(F)ccc2cc(O)cc(-c3ncc4c(N5CCOCC6(CCO6)C5)nc(OC[C@@]56CCCN5C[C@H](F)C6)nc4c3F)c12] |
| 83 | 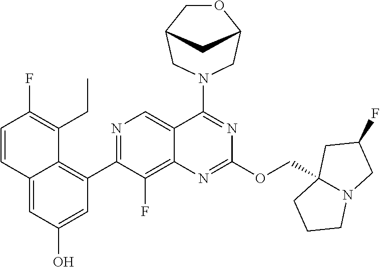 [SMILES: CCc1c(F)ccc2cc(O)cc(-c3ncc4c(N5C[C@@H]6CO[C@@H](C6)C5)nc(OC[C@@]56CCCN5C[C@H](F)C6)nc4c3F)c12] |
| 84 | 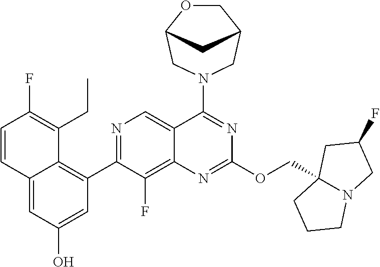 [SMILES: CCc1c(F)ccc2cc(O)cc(-c3ncc4c(N5C[C@H]6CO[C@H](C6)C5)nc(OC[C@@]56CCCN5C[C@H](F)C6)nc4c3F)c12] |
| 85 | 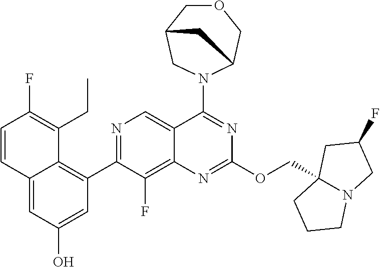 [SMILES: CCc1c(F)ccc2cc(O)cc(-c3ncc4c(N5C[C@@H]6COC[C@H]5C6)nc(OC[C@@]56CCCN5C[C@H](F)C6)nc4c3F)c12] |
| 86 | 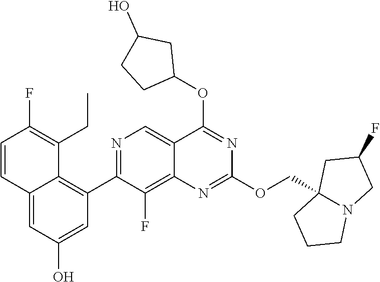 [SMILES: CCc1c(F)ccc2cc(O)cc(-c3ncc4c(OC5CCC(O)C5)nc(OC[C@@]56CCCN5C[C@H](F)C6)nc4c3F)c12] |
| 87 | 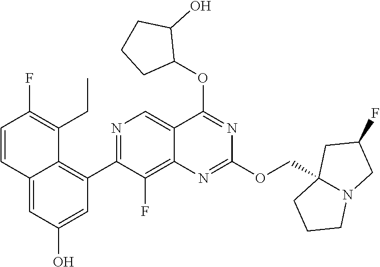 [SMILES: CCc1c(F)ccc2cc(O)cc(-c3ncc4c(OC5CCCC5O)nc(OC[C@@]56CCCN5C[C@H](F)C6)nc4c3F)c12] |
| 88 | 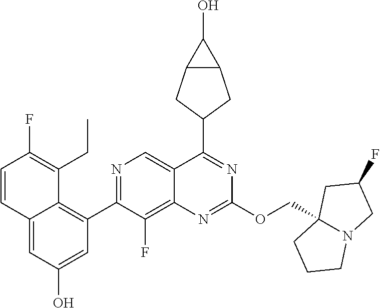 [SMILES: CCc1c(F)ccc2cc(O)cc(-c3ncc4c(C5CC6C(O)C6C5)nc(OC[C@@]56CCCN5C[C@H](F)C6)nc4c3F)c12] |
| 89 | 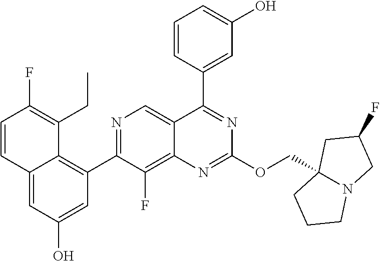 [SMILES: CCc1c(F)ccc2cc(O)cc(-c3ncc4c(-c5cccc(O)c5)nc(OC[C@@]56CCCN5C[C@H](F)C6)nc4c3F)c12] |
| 90 | 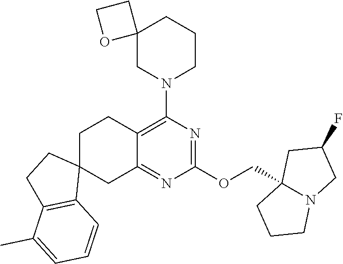 [SMILES: Cc1cccc2c1CCC21CCc2c(nc(OC[C@@]34CCCN3C[C@H](F)C4)nc2N2CCCC3(CCO3)C2)C1] |
| 91 | 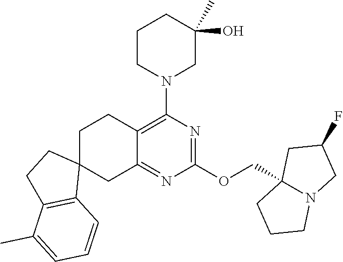 [SMILES: Cc1cccc2c1CCC21CCc2c(nc(OC[C@@]34CCCN3C[C@H](F)C4)nc2N2CCC[C@@](C)(O)C2)C1] |
| 92 | 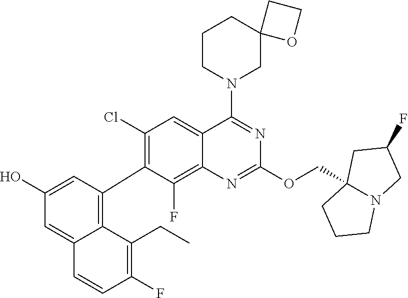 [SMILES: CCc1c(F)ccc2cc(O)cc(-c3c(Cl)cc4c(N5CCCC6(CCO6)C5)nc(OC[C@@]56CCCN5C[C@H](F)C6)nc4c3F)c12] |
| 93 | 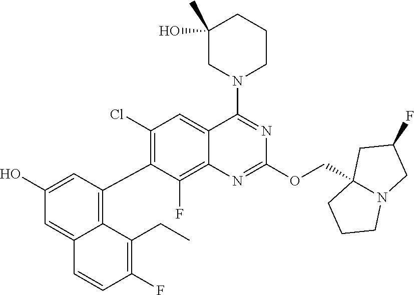 [SMILES: CCc1c(F)ccc2cc(O)cc(-c3c(Cl)cc4c(N5CCC[C@@](C)(O)C5)nc(OC[C@@]56CCCN5C[C@H](F)C6)nc4c3F)c12] |
| 94 | 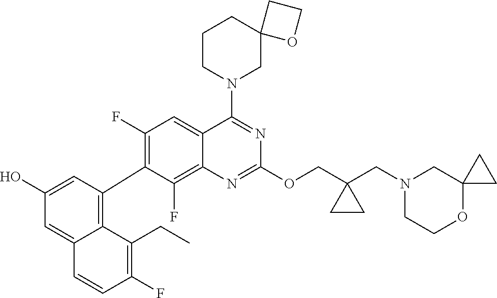 [SMILES: CCc1c(F)ccc2cc(O)cc(-c3c(F)cc4c(N5CCCC6(CCO6)C5)nc(OCC5(CN6CCOC7(CC7)C6)CC5)nc4c3F)c12] |
| 95 | 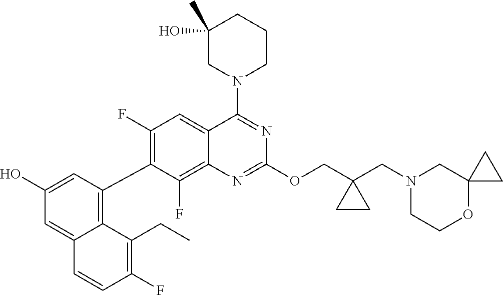 [SMILES: CCc1c(F)ccc2cc(O)cc(-c3c(F)cc4c(N5CCC[C@@](C)(O)C5)nc(OCC5(CN6CCOC7(CC7)C6)CC5)nc4c3F)c12] |
| 96 | 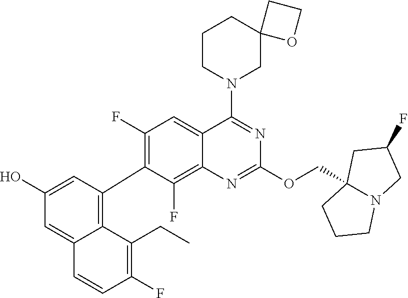 [SMILES: CCc1c(F)ccc2cc(O)cc(-c3c(F)cc4c(N5CCCC6(CCO6)C5)nc(OC[C@@]56CCCN5C[C@H](F)C6)nc4c3F)c12] |
| 97 | 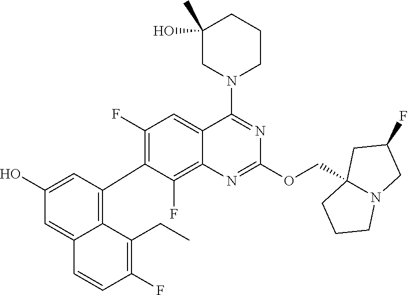 [SMILES: CCc1c(F)ccc2cc(O)cc(-c3c(F)cc4c(N5CCC[C@@](C)(O)C5)nc(OC[C@@]56CCCN5C[C@H](F)C6)nc4c3F)c12] |
| 98 | 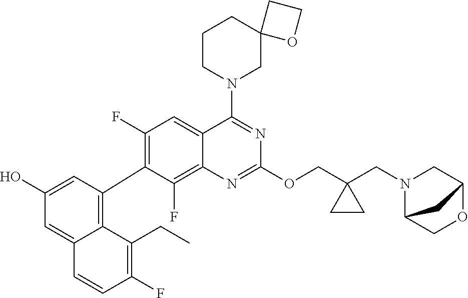 [SMILES: CCc1c(F)ccc2cc(O)cc(-c3c(F)cc4c(N5CCCC6(CCO6)C5)nc(OCC5(CN6C[C@H]7C[C@@H]6CO7)CC5)nc4c3F)c12] |
| 99 | 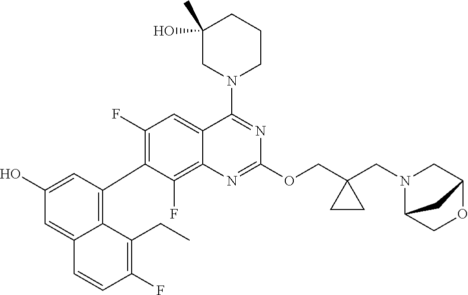 [SMILES: CCc1c(F)ccc2cc(O)cc(-c3c(F)cc4c(N5CCC[C@@](C)(O)C5)nc(OCC5(CN6C[C@H]7C[C@@H]6CO7)CC5)nc4c3F)c12] |
| 100 | 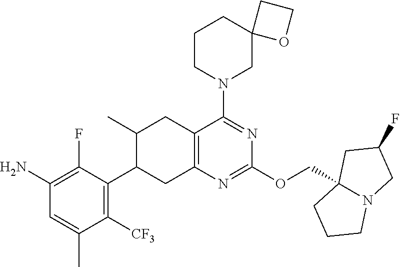 [SMILES: Cc1cc(N)c(F)c(C2Cc3nc(OC[C@@]45CCCN4C[C@H](F)C5)nc(N4CCCC5(CCO5)C4)c3CC2C)c1C(F)(F)F] |
| 101 | 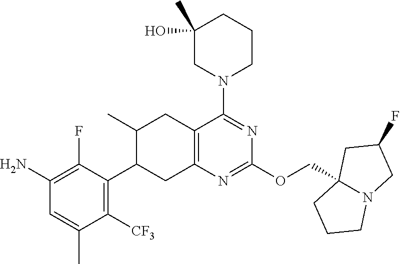 [SMILES: Cc1cc(N)c(F)c(C2Cc3nc(OC[C@@]45CCCN4C[C@H](F)C5)nc(N4CCC[C@@](C)(O)C4)c3CC2C)c1C(F)(F)F] |
| 102 | 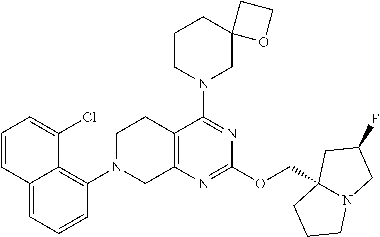 [SMILES: F[C@H]1CN2CCC[C@@]2(COc2nc3c(c(N4CCCC5(CCO5)C4)n2)CCN(c2cccc4cccc(Cl)c24)C3)C1] |
| 103 | 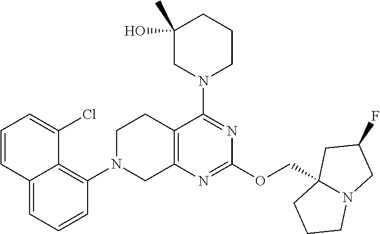 [SMILES: C[C@@]1(O)CCCN(c2nc(OC[C@@]34CCCN3C[C@H](F)C4)nc3c2CCN(c2cccc4cccc(Cl)c24)C3)C1] |
| 104 | 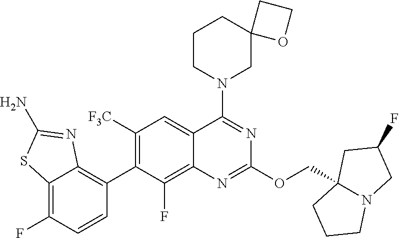 [SMILES: Nc1nc2c(-c3c(C(F)(F)F)cc4c(N5CCCC6(CCO6)C5)nc(OC[C@@]56CCCN5C[C@H](F)C6)nc4c3F)ccc(F)c2s1] |
| 105 | 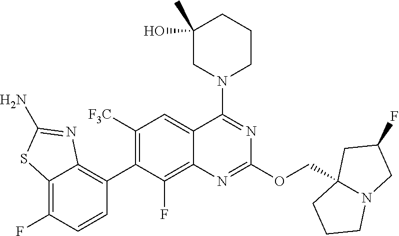 [SMILES: C[C@@]1(O)CCCN(c2nc(OC[C@@]34CCCN3C[C@H](F)C4)nc3c(F)c(-c4ccc(F)c5sc(N)nc45)c(C(F)(F)F)cc23)C1] |
| 106 | 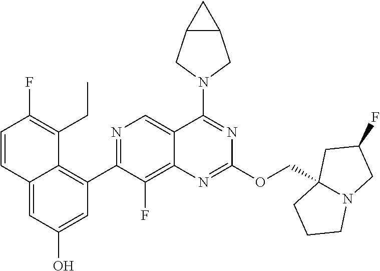 [SMILES: CCc1c(F)ccc2cc(O)cc(-c3ncc4c(N5CC6CC6C5)nc(OC[C@@]56CCCN5C[C@H](F)C6)nc4c3F)c12] |
| 107 | 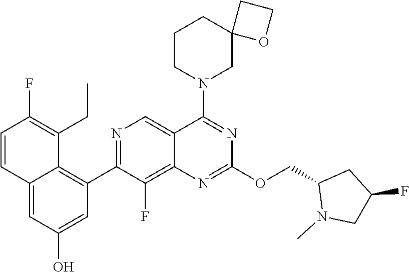 [SMILES: CCc1c(F)ccc2cc(O)cc(-c3ncc4c(N5CCCC6(CCO6)C5)nc(OC[C@@H]5C[C@@H](F)CN5C)nc4c3F)c12] |
| 108 | 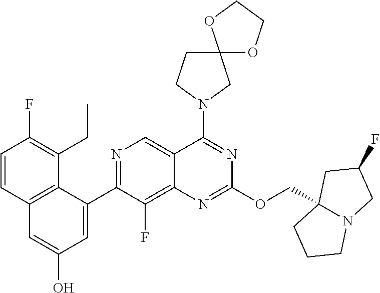 [SMILES: CCc1c(F)ccc2cc(O)cc(-c3ncc4c(N5CCC6(C5)OCCO6)nc(OC[C@@]56CCCN5C[C@H](F)C6)nc4c3F)c12] |
| 109 | 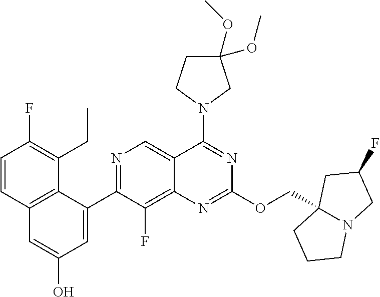 [SMILES: CCc1c(F)ccc2cc(O)cc(-c3ncc4c(N5CCC(OC)(OC)C5)nc(OC[C@@]56CCCN5C[C@H](F)C6)nc4c3F)c12] |
| 110 | 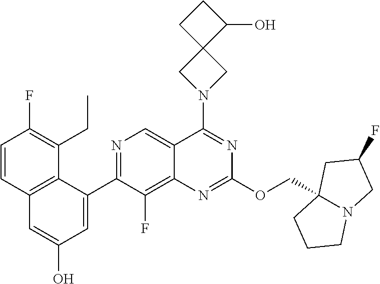 [SMILES: CCc1c(F)ccc2cc(O)cc(-c3ncc4c(N5CC6(CCC6O)C5)nc(OC[C@@]56CCCN5C[C@H](F)C6)nc4c3F)c12] |
| 111 | 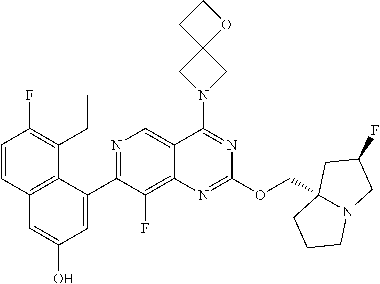 [SMILES: CCc1c(F)ccc2cc(O)cc(-c3ncc4c(N5CC6(CCO6)C5)nc(OC[C@@]56CCCN5C[C@H](F)C6)nc4c3F)c12] |
| 112 | 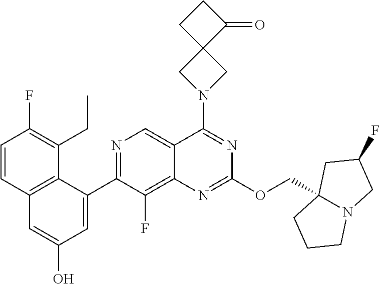 [SMILES: CCc1c(F)ccc2cc(O)cc(-c3ncc4c(N5CC6(CCC6=O)C5)nc(OC[C@@]56CCCN5C[C@H](F)C6)nc4c3F)c12] |
| 113 | 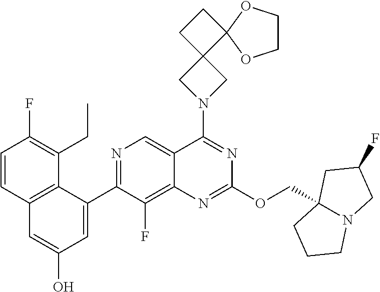 [SMILES: CCc1c(F)ccc2cc(O)cc(-c3ncc4c(N5CC6(CCC67OCCO7)C5)nc(OC[C@@]56CCCN5C[C@H](F)C6)nc4c3F)c12] |
| 114 | 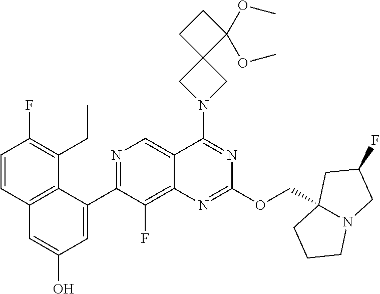 [SMILES: CCc1c(F)ccc2cc(O)cc(-c3ncc4c(N5CC6(CCC6(OC)OC)C5)nc(OC[C@@]56CCCN5C[C@H](F)C6)nc4c3F)c12] |
| 115 | 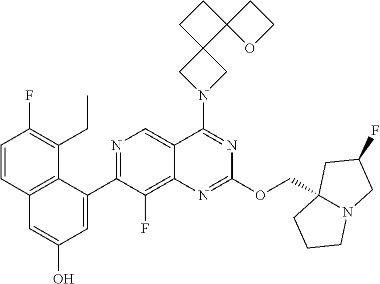 [SMILES: CCc1c(F)ccc2cc(O)cc(-c3ncc4c(N5CC6(CCC67CCO7)C5)nc(OC[C@@]56CCCN5C[C@H](F)C6)nc4c3F)c12] |
| 116 | 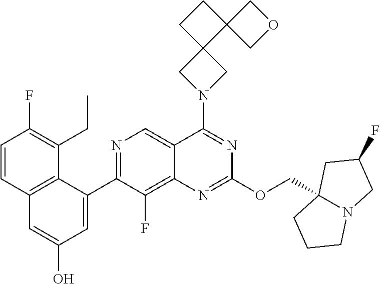 [SMILES: CCc1c(F)ccc2cc(O)cc(-c3ncc4c(N5CC6(CCC67COC7)C5)nc(OC[C@@]56CCCN5C[C@H](F)C6)nc4c3F)c12] |
| 117 | 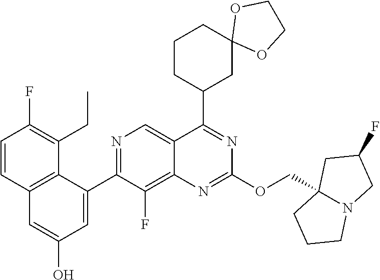 [SMILES: CCc1c(F)ccc2cc(O)cc(-c3ncc4c(C5CCCC6(C5)OCCO6)nc(OC[C@@]56CCCN5C[C@H](F)C6)nc4c3F)c12] |
| 118 | 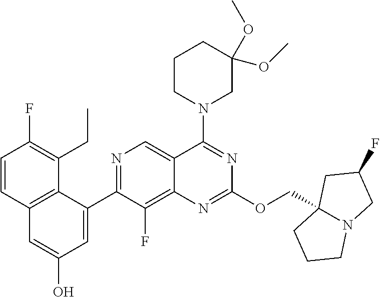 [SMILES: CCc1c(F)ccc2cc(O)cc(-c3ncc4c(N5CCCC(OC)(OC)C5)nc(OC[C@@]56CCCN5C[C@H](F)C6)nc4c3F)c12] |

TABLE 2
Structures of exemplary compounds in accordance with certain embodiments of
the disclosure.

| Cpd | |
|---|---|
| No. | Structure |
| 1a | 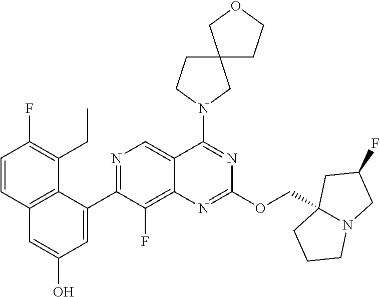 [SMILES: CCc1c(F)ccc2cc(O)cc(-c3ncc4c(N5CCC6(CCOC6)C5)nc(OC[C@@]56CCCN5C[C@H](F)C6)nc4c3F)c12] |
| 2a | 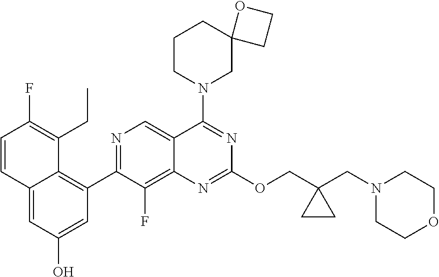 [SMILES: CCc1c(F)ccc2cc(O)cc(-c3ncc4c(N5CCCC6(CCO6)C5)nc(OCC5(CN6CCOCC6)CC5)nc4c3F)c12] |
| 3a | 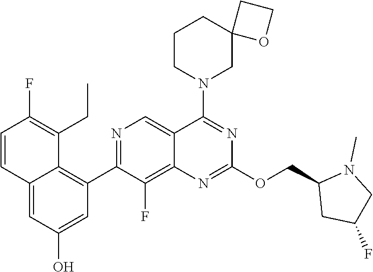 [SMILES: CCc1c(F)ccc2cc(O)cc(-c3ncc4c(N5CCCC6(CCO6)C5)nc(OC[C@@H]5C[C@@H](F)CN5C)nc4c3F)c12] |
| 4a | 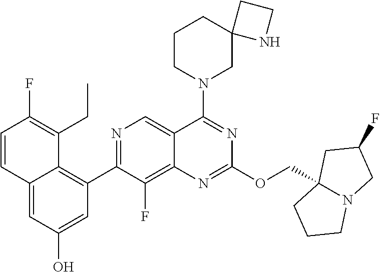 [SMILES: CCc1c(F)ccc2cc(O)cc(-c3ncc4c(N5CCCC6(CCN6)C5)nc(OC[C@@]56CCCN5C[C@H](F)C6)nc4c3F)c12] |
| 5a | 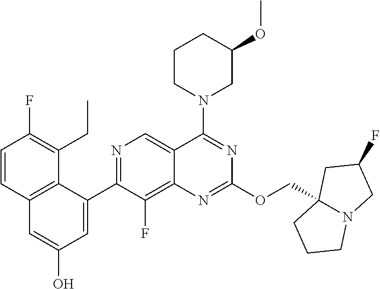 [SMILES: CCc1c(F)ccc2cc(O)cc(-c3ncc4c(N5CCC[C@@H](OC)C5)nc(OC[C@@]56CCCN5C[C@H](F)C6)nc4c3F)c12] |
| 6a | 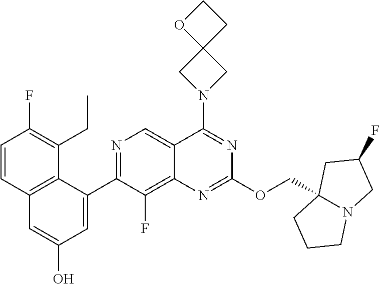 [SMILES: CCc1c(F)ccc2cc(O)cc(-c3ncc4c(N5CC6(CCO6)C5)nc(OC[C@@]56CCCN5C[C@H](F)C6)nc4c3F)c12] |
| 7a | 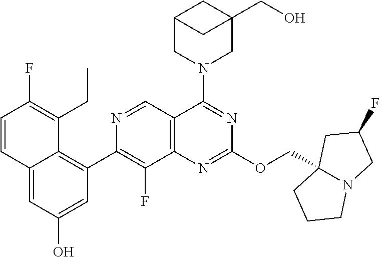 [SMILES: CCc1c(F)ccc2cc(O)cc(-c3ncc4c(N5CC6CC(CO)(C6)C5)nc(OC[C@@]56CCCN5C[C@H](F)C6)nc4c3F)c12] |
| 8a | 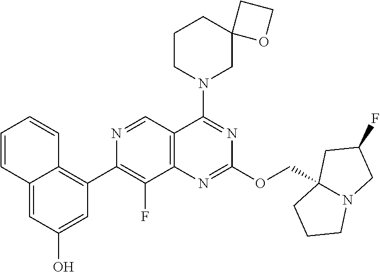 [SMILES: Oc1cc(-c2ncc3c(N4CCCC5(CCO5)C4)nc(OC[C@@]45CCCN4C[C@H](F)C5)nc3c2F)c2ccccc2c1] |
| 9a | 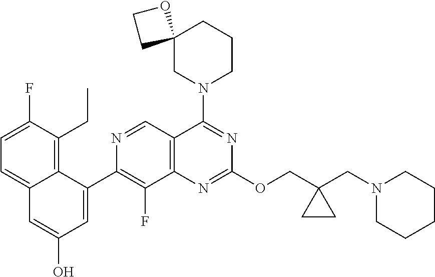 [SMILES: CCc1c(F)ccc2cc(O)cc(-c3ncc4c(N5CCC[C@]6(CCO6)C5)nc(OCC5(CN6CCCCC6)CC5)nc4c3F)c12] |
| 10a | 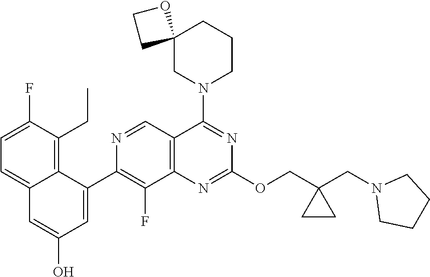 [SMILES: CCc1c(F)ccc2cc(O)cc(-c3ncc4c(N5CCC[C@]6(CCO6)C5)nc(OCC5(CN6CCCC6)CC5)nc4c3F)c12] |
| 11a | 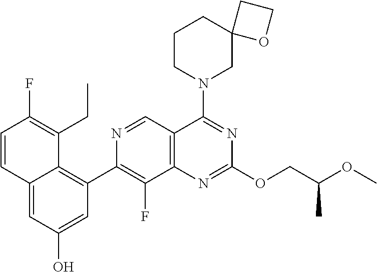 [SMILES: CCc1c(F)ccc2cc(O)cc(-c3ncc4c(N5CCCC6(CCO6)C5)nc(OC[C@H](C)OC)nc4c3F)c12] |
| 12a | 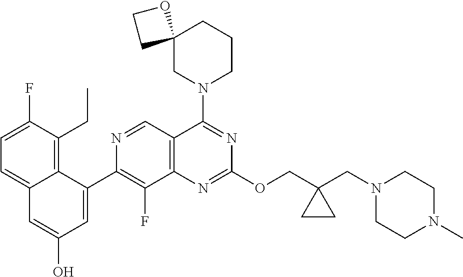 [SMILES: CCc1c(F)ccc2cc(O)cc(-c3ncc4c(N5CCC[C@]6(CCO6)C5)nc(OCC5(CN6CCN(C)CC6)CC5)nc4c3F)c12] |
| 13a | 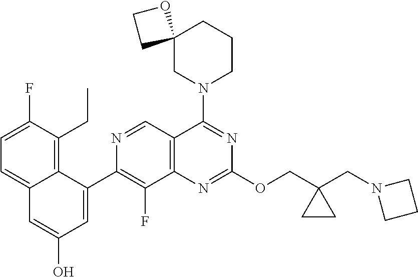 [SMILES: CCc1c(F)ccc2cc(O)cc(-c3ncc4c(N5CCC[C@]6(CCO6)C5)nc(OCC5(CN6CCC6)CC5)nc4c3F)c12] |
| 14a | 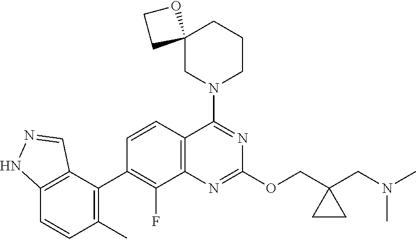 [SMILES: Cc1ccc2[nH]ncc2c1-c1ccc2c(N3CCC[C@]4(CCO4)C3)nc(OCC3(CN(C)C)CC3)nc2c1F] |
| 15a | 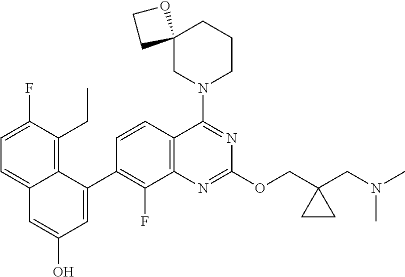 [SMILES: CCc1c(F)ccc2cc(O)cc(-c3ccc4c(N5CCC[C@]6(CCO6)C5)nc(OCC5(CN(C)C)CC5)nc4c3F)c12] |
| 16a | 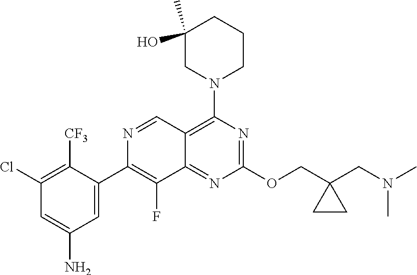 [SMILES: CN(C)CC1(COc2nc(N3CCC[C@](C)(O)C3)c3cnc(-c4cc(N)cc(Cl)c4C(F)(F)F)c(F)c3n2)CC1] |
| 17a | 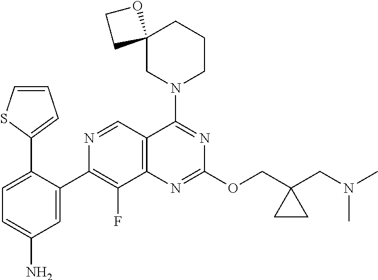 [SMILES: CN(C)CC1(COc2nc(N3CCC[C@]4(CCO4)C3)c3cnc(-c4cc(N)ccc4-c4cccs4)c(F)c3n2)CC1] |
| 18a | 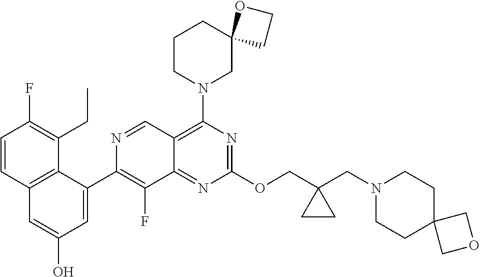 [SMILES: CCc1c(F)ccc2cc(O)cc(-c3ncc4c(N5CCC[C@]6(CCO6)C5)nc(OCC5(CN6CCC7(CC6)COC7)CC5)nc4c3F)c12] |
| 19a | 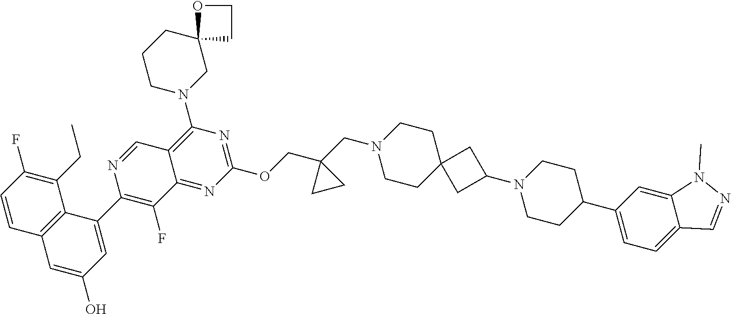 [SMILES: CCc1c(F)ccc2cc(O)cc(-c3ncc4c(N5CCC[C@]6(CCO6)C5)nc(OCC5(CN6CCC7(CC6)CC(N6CCC(c8ccc9cnn(C)c9c8)CC6)C7)CC5)nc4c3F)c12] |
| 20a | 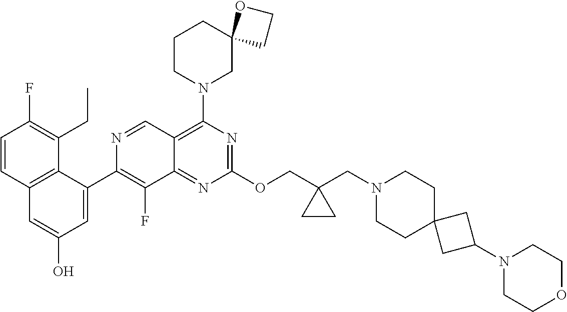 [SMILES: CCc1c(F)ccc2cc(O)cc(-c3ncc4c(N5CCC[C@]6(CCO6)C5)nc(OCC5(CN6CCC7(CC6)CC(N6CCOCC6)C7)CC5)nc4c3F)c12] |
| 21a | 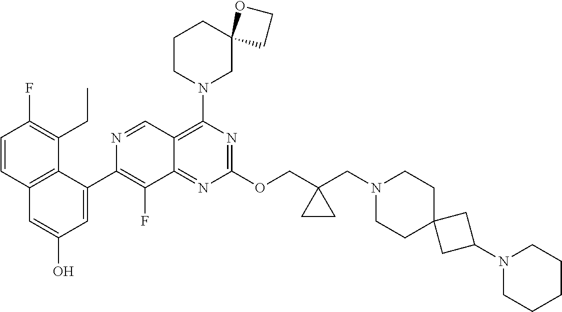 [SMILES: CCc1c(F)ccc2cc(O)cc(-c3ncc4c(N5CCC[C@]6(CCO6)C5)nc(OCC5(CN6CCC7(CC6)CC(N6CCCCC6)C7)CC5)nc4c3F)c12] |
| 22a | 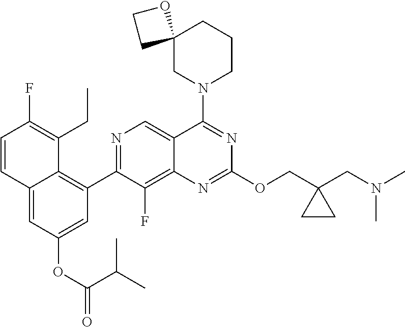 [SMILES: CCc1c(F)ccc2cc(OC(=O)C(C)C)cc(-c3ncc4c(N5CCC[C@]6(CCO6)C5)nc(OCC5(CN(C)C)CC5)nc4c3F)c12] |
| 23a | 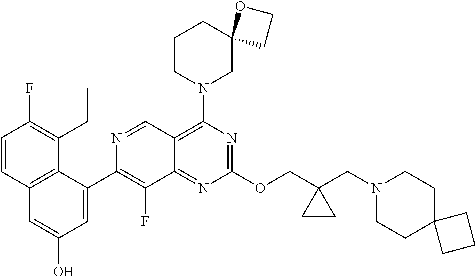 [SMILES: CCc1c(F)ccc2cc(O)cc(-c3ncc4c(N5CCC[C@]6(CCO6)C5)nc(OCC5(CN6CCC7(CCC7)CC6)CC5)nc4c3F)c12] |
| 24a | 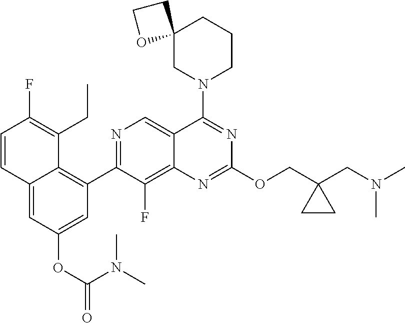 [SMILES: CCc1c(F)ccc2cc(OC(=O)N(C)C)cc(-c3ncc4c(N5CCC[C@]6(CCO6)C5)nc(OCC5(CN(C)C)CC5)nc4c3F)c12] |
| 25a | 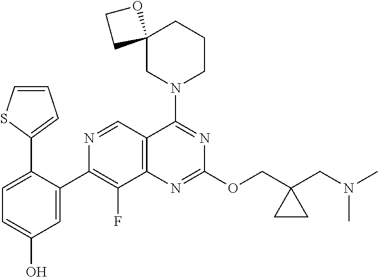 [SMILES: CN(C)CC1(COc2nc(N3CCC[C@]4(CCO4)C3)c3cnc(-c4cc(O)ccc4-c4cccs4)c(F)c3n2)CC1] |
| 26a | 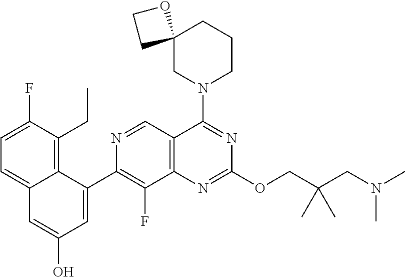 [SMILES: CCc1c(F)ccc2cc(O)cc(-c3ncc4c(N5CCC[C@]6(CCO6)C5)nc(OCC(C)(C)CN(C)C)nc4c3F)c12] |
| 27a | 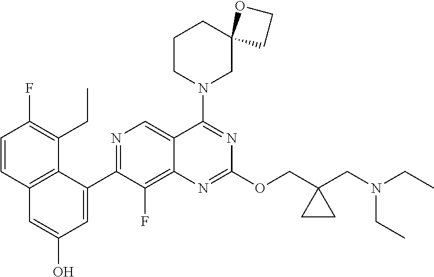 [SMILES: CCc1c(F)ccc2cc(O)cc(-c3ncc4c(N5CCC[C@]6(CCO6)C5)nc(OCC5(CN(CC)CC)CC5)nc4c3F)c12] |
| 28a | 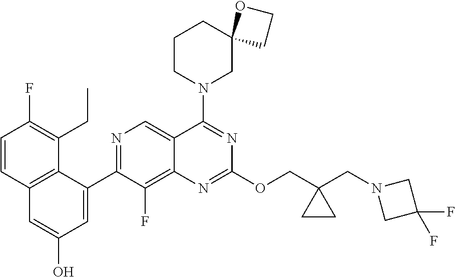 [SMILES: CCc1c(F)ccc2cc(O)cc(-c3ncc4c(N5CCC[C@]6(CCO6)C5)nc(OCC5(CN6CC(F)(F)C6)CC5)nc4c3F)c12] |
| 29a | 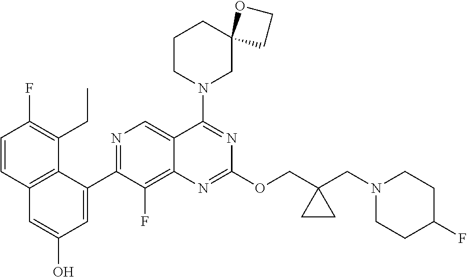 [SMILES: CCc1c(F)ccc2cc(O)cc(-c3ncc4c(N5CCC[C@]6(CCO6)C5)nc(OCC5(CN6CCC(F)CC6)CC5)nc4c3F)c12] |
| 30a | 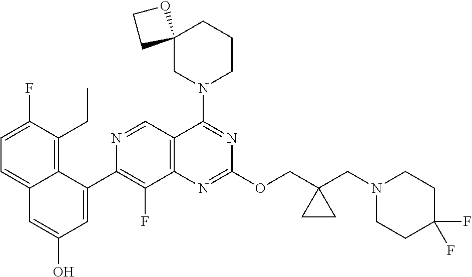 [SMILES: CCc1c(F)ccc2cc(O)cc(-c3ncc4c(N5CCC[C@]6(CCO6)C5)nc(OCC5(CN6CCC(F)(F)CC6)CC5)nc4c3F)c12] |
| 31a | 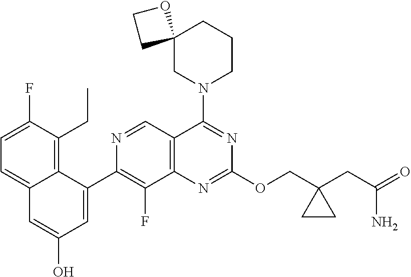 [SMILES: CCc1c(F)ccc2cc(O)cc(-c3ncc4c(N5CCC[C@]6(CCO6)C5)nc(OCC5(CC(N)=O)CC5)nc4c3F)c12] |
| 32a | 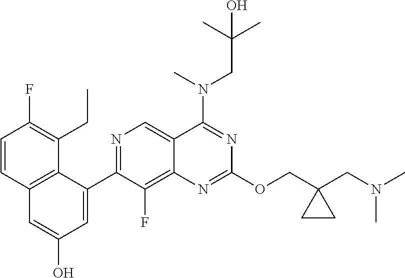 [SMILES: CCc1c(F)ccc2cc(O)cc(-c3ncc4c(N(C)CC(C)(C)O)nc(OCC5(CN(C)C)CC5)nc4c3F)c12] |
| 33a | 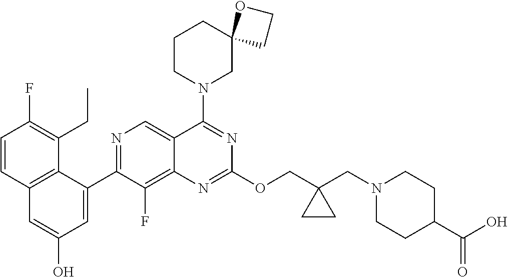 [SMILES: CCc1c(F)ccc2cc(O)cc(-c3ncc4c(N5CCC[C@]6(CCO6)C5)nc(OCC5(CN6CCC(C(=O)O)CC6)CC5)nc4c3F)c12] |
| 34a | 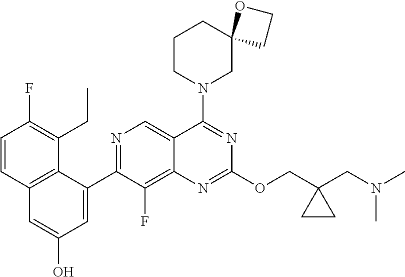 [SMILES: CCc1c(F)ccc2cc(O)cc(-c3ncc4c(N5CCC[C@]6(CCO6)C5)nc(OCC5(CN(C)C)CC5)nc4c3F)c12] |
| 35a | 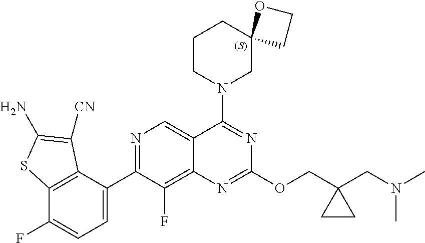 |
| 36a | 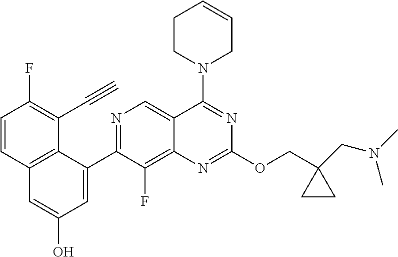 [SMILES: C#Cc1c(F)ccc2cc(O)cc(-c3ncc4c(N5CC=CCC5)nc(OCC5(CN(C)C)CC5)nc4c3F)c12] |
| 37a | 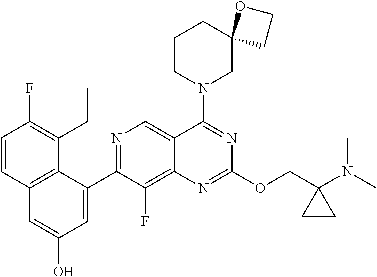 [SMILES: CCc1c(F)ccc2cc(O)cc(-c3ncc4c(N5CCC[C@]6(CCO6)C5)nc(OCC5(N(C)C)CC5)nc4c3F)c12] |
| 38a | 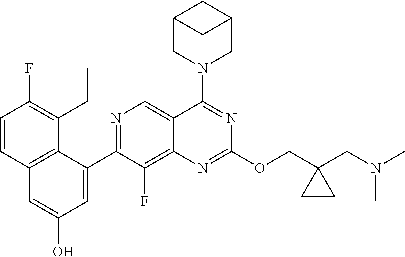 [SMILES: CCc1c(F)ccc2cc(O)cc(-c3ncc4c(N5CC6CC(C6)C5)nc(OCC5(CN(C)C)CC5)nc4c3F)c12] |
| 39a | 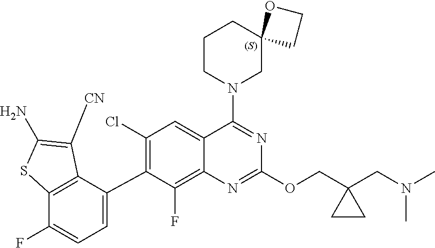 |
| 40a | 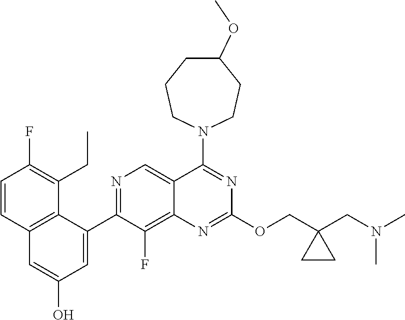 [SMILES: CCc1c(F)ccc2cc(O)cc(-c3ncc4c(N5CCCC(OC)CC5)nc(OCC5(CN(C)C)CC5)nc4c3F)c12] |
| 41a | 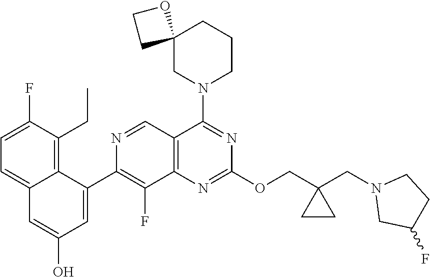 [SMILES: CCc1c(F)ccc2cc(O)cc(-c3ncc4c(N5CCC[C@]6(CCO6)C5)nc(OCC5(CN6CCC(F)C6)CC5)nc4c3F)c12] |
| 42a | 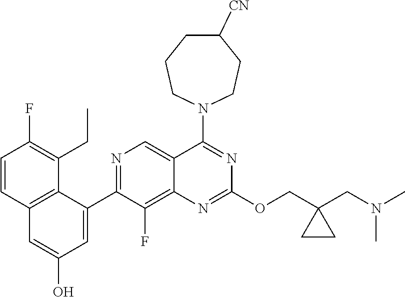 [SMILES: CCc1c(F)ccc2cc(O)cc(-c3ncc4c(N5CCCC(C#N)CC5)nc(OCC5(CN(C)C)CC5)nc4c3F)c12] |
| 43a | 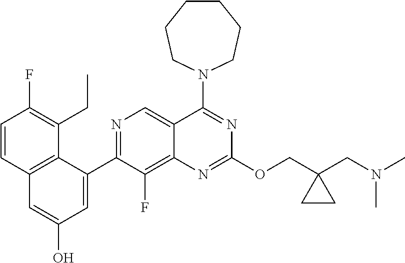 [SMILES: CCc1c(F)ccc2cc(O)cc(-c3ncc4c(N5CCCCCC5)nc(OCC5(CN(C)C)CC5)nc4c3F)c12] |
| 44a | 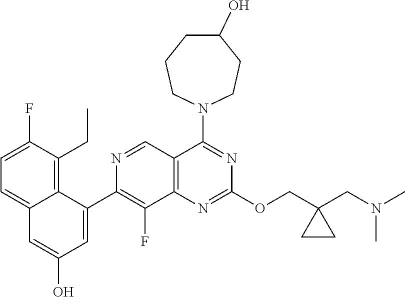 [SMILES: CCc1c(F)ccc2cc(O)cc(-c3ncc4c(N5CCCC(O)CC5)nc(OCC5(CN(C)C)CC5)nc4c3F)c12] |
| 45a | 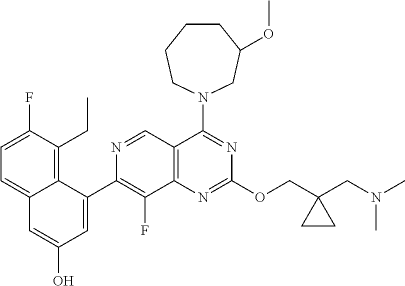 [SMILES: CCc1c(F)ccc2cc(O)cc(-c3ncc4c(N5CCCCC(OC)C5)nc(OCC5(CN(C)C)CC5)nc4c3F)c12] |
| 46a | 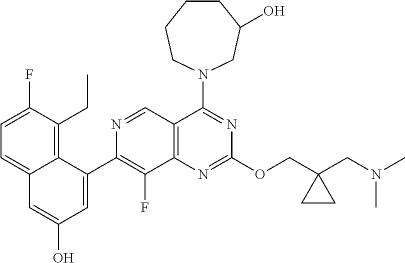 [SMILES: CCc1c(F)ccc2cc(O)cc(-c3ncc4c(N5CCCCC(O)C5)nc(OCC5(CN(C)C)CC5)nc4c3F)c12] |
| 47a | 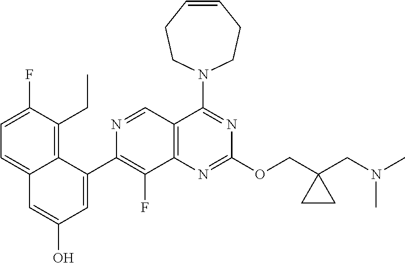 [SMILES: CCc1c(F)ccc2cc(O)cc(-c3ncc4c(N5CCC=CCC5)nc(OCC5(CN(C)C)CC5)nc4c3F)c12] |
| 48a | 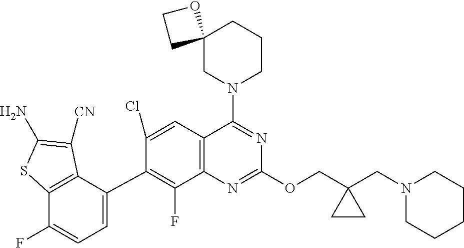 [SMILES: N#Cc1c(N)sc2c(F)ccc(-c3c(Cl)cc4c(N5CCC[C@]6(CCO6)C5)nc(OCC5(CN6CCCCC6)CC5)nc4c3F)c12] |
| 49a | 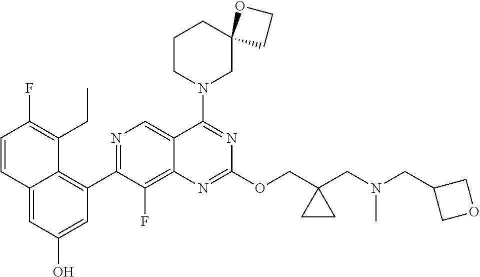 [SMILES: CCc1c(F)ccc2cc(O)cc(-c3ncc4c(N5CCC[C@]6(CCO6)C5)nc(OCC5(CN(C)CC6COC6)CC5)nc4c3F)c12] |
| 50a | 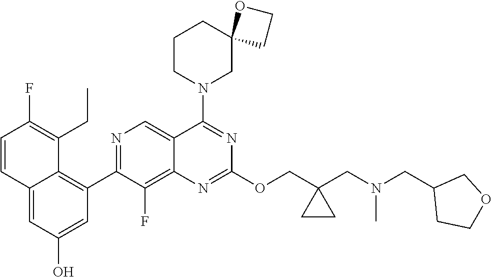 [SMILES: CCc1c(F)ccc2cc(O)cc(-c3ncc4c(N5CCC[C@]6(CCO6)C5)nc(OCC5(CN(C)CC6CCOC6)CC5)nc4c3F)c12] |

Without wishing to be limited by theory, the compounds of the disclosure, as well as pharmaceutically acceptable salts, esters, hydrates, solvates or stereoisomers thereof, can act as KRAS inhibitors and can be used effectively to treat diseases associated with KRAS (including wild-type KRAS and KRAS mutations). In some embodiments, the compounds of the disclosure have anti-tumor/anti-cancer activity and can be used effectively for the inhibition, treatment or prevention of a hyperproliferative disorder, such as a KRAS-associated cancer or tumor.

In some embodiments, the compounds of the disclosure may be naturally abundant or isotopically substituted compounds, and the isotopes may be D, T, ¹⁸O, ¹⁷O, ¹⁵N or ¹³C etc.

In some embodiments, the compound of the disclosure can be used for the inhibition, treatment or prevention of wild-type KRAS-related diseases or symptoms.

In some embodiments, the compound of the disclosure can be used for the inhibition, treatment or prevention of KRAS G12A-related diseases or symptoms.

In some embodiments, the compound of the disclosure can be used for the inhibition, treatment or prevention of KRAS G12C-related diseases or symptoms.

In some embodiments, the compound of the disclosure can be used for the inhibition, treatment or prevention of KRAS G12D-related diseases or symptoms.

In some embodiments, the compound of the disclosure can be used for the inhibition, treatment or prevention of KRAS G12R-related diseases or symptoms.

In some embodiments, the compound of the disclosure can be used for the inhibition, treatment or prevention of KRAS G12S-related diseases or symptoms.

In some embodiments, the compound of the disclosure can be used for the inhibition, treatment or prevention of KRAS G12V-related diseases or symptoms.

In some embodiments, the compound of the disclosure can be used for the inhibition, treatment or prevention of KRAS G13D-related diseases or symptoms.

In some embodiments, the compound of the disclosure can be used for the inhibition, treatment or prevention of KRAS Q61H-related diseases or symptoms.

In some embodiments, the compound of the disclosure can be used for the simultaneous inhibition, treatment or prevention of two or more KRAS proteins, e.g., two or more KRAS proteins selected from wild-type (WT) and KRAS mutant proteins, e.g., selected from KRAS WT, KRAS G12A, KRAS G12C, KRAS G12D, KRAS G12R, KRAS G12S, KRAS G12V, KRAS G13D and KRAS Q61H.

In some embodiments, the compound of the disclosure can be used for the simultaneous inhibition, treatment or prevention of three or more KRAS proteins, e.g., three or more KRAS proteins selected from wild-type (WT) and KRAS mutant proteins, e.g., selected from KRAS WT, KRAS G12A, KRAS G12C, KRAS G12D, KRAS G12R, KRAS G12S, KRAS G12V, KRAS G13D and KRAS Q61H.

In some embodiments, the compound of the disclosure can be used for the simultaneous inhibition, treatment or prevention of four or more KRAS proteins, e.g., four or more KRAS proteins selected from wild-type (WT) and KRAS mutant proteins, e.g., selected from KRAS WT, KRAS G12A, KRAS G12C, KRAS G12D, KRAS G12R, KRAS G12S, KRAS G12V, KRAS G13D and KRAS Q61H.

In some embodiments, the compound disclosed herein may be administered to a subject in the form of a prodrug that is metabolized after administration into biologically active constituents, thereby effecting treatment or prevention of KRAS-related diseases or symptoms.

In another broad aspect, there are provided pharmaceutical compositions comprising a compound described herein, or a pharmaceutically acceptable salt, ester, hydrate, solvate or stereoisomer thereof. In some embodiments, the pharmaceutical compositions further comprise a pharmaceutically acceptable excipient, carrier or diluent.

In some embodiments, there are provided pharmaceutical compositions comprising a compound of Formula (I), or a pharmaceutically acceptable salt, ester, hydrate, solvate or stereoisomer thereof, and a pharmaceutically acceptable carrier.

In some embodiments, there are provided pharmaceutical compositions comprising a compound of Formula (I-a), or a pharmaceutically acceptable salt, ester, hydrate, solvate or stereoisomer thereof, and a pharmaceutically acceptable carrier.

In some embodiments, there are provided pharmaceutical compositions comprising a compound of Formula (I-b), or a pharmaceutically acceptable salt, ester, hydrate, solvate or stereoisomer thereof, and a pharmaceutically acceptable carrier.

In some embodiments, there are provided pharmaceutical compositions comprising a compound of Formula (II-a), or a pharmaceutically acceptable salt, ester, hydrate, solvate or stereoisomer thereof, and a pharmaceutically acceptable carrier.

In some embodiments, there are provided pharmaceutical compositions comprising a compound of Formula (II-b), or a pharmaceutically acceptable salt, ester, hydrate, solvate or stereoisomer thereof, and a pharmaceutically acceptable carrier.

In some embodiments, there are provided pharmaceutical compositions comprising a compound of Formula (III-a), or a pharmaceutically acceptable salt, ester, hydrate, solvate or stereoisomer thereof, and a pharmaceutically acceptable carrier.

In some embodiments, there are provided pharmaceutical compositions comprising a compound of Formula (III-b), or a pharmaceutically acceptable salt, ester, hydrate, solvate or stereoisomer thereof, and a pharmaceutically acceptable carrier.

In some embodiments, there are provided pharmaceutical compositions comprising a compound of Formula (IV-a), or a pharmaceutically acceptable salt, ester, hydrate, solvate or stereoisomer thereof, and a pharmaceutically acceptable carrier.

In some embodiments, there are provided pharmaceutical compositions comprising a compound of Formula (IV-b), or a pharmaceutically acceptable salt, ester, hydrate, solvate or stereoisomer thereof, and a pharmaceutically acceptable carrier.

In some embodiments, there are provided pharmaceutical compositions comprising a compound of Formula (IV-c), or a pharmaceutically acceptable salt, ester, hydrate, solvate or stereoisomer thereof, and a pharmaceutically acceptable carrier.

In some embodiments, there are provided pharmaceutical compositions comprising a compound of Formula (V), or a pharmaceutically acceptable salt, ester, hydrate, solvate or stereoisomer thereof, and a pharmaceutically acceptable carrier.

In some embodiments, there are provided pharmaceutical compositions comprising a compound of Formula (VI), or a pharmaceutically acceptable salt, ester, hydrate, solvate or stereoisomer thereof, and a pharmaceutically acceptable carrier.

In some embodiments, there are provided pharmaceutical compositions comprising a compound of Table 1, or a pharmaceutically acceptable salt, ester, hydrate, solvate or stereoisomer thereof, and a pharmaceutically acceptable carrier.

In some embodiments, there are provided pharmaceutical compositions comprising a compound of Table 2, or a pharmaceutically acceptable salt, ester, hydrate, solvate or stereoisomer thereof, and a pharmaceutically acceptable carrier.

In some such embodiments, the pharmaceutical composition comprises a pharmaceutically acceptable excipient comprising one or more adhesive, binder, filler, disintegrant, lubricant, glidant and/or dispersant. In some embodiments, the pharmaceutically acceptable carrier comprises one or more of a cream, an emulsion, a gel, a liposome, and a nanoparticle.

In some embodiments, the pharmaceutical composition is suitable for administration orally or by injection.

In one embodiment, the pharmaceutical composition is suitable for oral administration. In some such embodiments, the composition is in the form of a hard-shell gelatin capsule, a soft-shell gelatin capsule, a cachet, a pill, a tablet, a lozenge, a powder, a granule, a pellet, a pastille, or a dragee. In some embodiments, the composition is in the form of a solution, an aqueous liquid suspension, a non-aqueous liquid suspension, an oil-in-water liquid emulsion, a water-in-oil liquid emulsion, an elixir, or a syrup. In some embodiments, the composition is enteric coated. In some embodiments, the composition is formulated for controlled release.

In another embodiment, the pharmaceutical composition is suitable for administration by injection. For example, the pharmaceutical composition may be administered subcutaneously, intravenously, intramuscularly, or intraperitoneally. In one embodiment, the pharmaceutical composition is suitable for intravenous administration.

In some embodiments, the pharmaceutical composition is suitable for parenteral, intraperitoneal, intradermal, intracardiac, intraventricular, intracranial, cerebrospinal, intrasynovial, intrathecal, intramuscular, intravitreal, intravenous, intra-arterial, oral, intraoral, sublingual, transdermal, intratracheal, intrarectal, subcutaneous, or topical administration, or for administration by injection.

In another broad aspect, there are provided methods of inhibiting KRAS activity in a subject in need thereof, comprising administering to the subject an effective amount of a compound and/or a pharmaceutical composition described herein.

In certain embodiments, there are provided methods of treating or preventing a KRAS-associated disease, disorder or condition in a subject in need thereof, comprising administering an effective amount of a compound and/or a pharmaceutical composition described herein, such that the KRAS-associated disease, disorder or condition is treated or prevented in the subject.

In particular embodiments, the compounds described herein act to inhibit KRAS and are useful as therapeutic or prophylactic therapy when such inhibition is desired, e.g., for the prevention or treatment of KRAS-associated diseases, conditions and/or disorders. Unless otherwise indicated, when uses of the compounds of the present disclosure are described herein, it is to be understood that such compounds may be in the form of a composition (e.g., a pharmaceutical composition).

As used herein, the term “KRAS inhibitor” refers to a compound of the disclosure capable of inhibiting the KRAS protein (wild-type or mutant, e.g., KRAS WT, KRAS G12A, KRAS G12C, KRAS G12D, KRAS G12R, KRAS G12S, KRAS G12V, KRAS G13D and/or KRAS Q61H) in a cellular assay, an in vivo model, and/or other assay means indicative of KRAS inhibition and potential therapeutic or prophylactic efficacy. The terms also refer to compounds that exhibit at least some therapeutic or prophylactic benefit in a human subject. Although the compounds of the present disclosure are believed to have effect by inhibiting KRAS activity in a cell, a precise understanding of the compounds' underlying mechanism of action is not required to practice the technology.

In some embodiments, there are provided methods for inhibiting, treating or preventing a KRAS-associated disease, disorder or condition in a subject in need thereof. The KRAS-associated disease, disorder or condition may be, for example and without limitation, a cancer or tumor or hyperplastic or hyperproliferative disease or disorder related to or associated with the KRAS protein (wild-type or mutant). In some embodiments, the KRAS-associated disease, disorder or condition is a hyperproliferative disorder. In some embodiments, the KRAS-associated disease, disorder or condition is a hyperplastic disorder. In some embodiments, the KRAS-associated disease, disorder or condition is a malignant cancer or tumor. In some embodiments, the KRAS-associated disease, disorder or condition is a cardiac, lung, gastrointestinal, genitourinary tract, biliary tract, large intestine, small intestine, liver, bone, nervous system, gynecological, hematologic, skin, or adrenal gland cancer or tumor. In some embodiments, the KRAS-associated disease, disorder or condition is a non-small-cell lung cancer (NSCLC), a small cell lung cancer, a pancreatic cancer, a colorectal cancer, a colon cancer, a bile duct cancer, a cervical cancer, a bladder cancer, a liver cancer or a breast cancer.

In some embodiments, there are provided methods for treating or preventing a cancer or a hyperplastic condition in a subject (e.g., a human) comprising administering to the subject a therapeutically effective amount of at least one KRAS inhibitor compound (e.g., compound of the disclosure) or composition described herein. In some embodiments of such methods, the subject is administered at least one KRAS inhibitor compound or composition in an amount effective to reverse, slow or stop the progression of a KRAS-associated disease, disorder or condition, e.g., the cancer or hyperplastic condition. As used herein, the term “hyperplastic condition” refers to a malignant tumor or cancer, e.g., which is related to KRAS expression and/or related to at least one KRAS mutation.

The type of cancer or tumor that can be treated or prevented using the compounds and compositions described herein is not meant to be particularly limited. Examples of cancers and tumors that can be treated or prevented using the compounds and compositions described herein include, but are not limited to, cancers of the: (i) cardiac tissue or heart (including sarcoma, angiosarcoma, hemangiosarcoma, fibrosarcoma, rhabdomyosarcoma, liposarcoma, myxoma, rhabdomyoma, fibroma, lipoma, teratoma); (ii) lung (including bronchogenic carcinoma, squamous cell carcinoma, undifferentiated small cell carcinoma, undifferentiated large cell carcinoma, adenocarcinoma, alveolar (bronchiolar) carcinoma, bronchial adenoma, sarcoma, lymphoma, chondromatous hamartoma, mesothelioma); (iii) gastrointestinal system (including esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vasodilator tumors, Kaposi's sarcoma, leiomyoma, hemangioma, lipoma, neurofibroma, fibroma), small intestine (adenocarcinoma, lymphoma, carcinoid tumor, Kaposi's sarcoma, leiomyoma, hemangioma, lipoma, neurofibroma, fibroma), large intestine (adenocarcinoma, tubular adenoma, villous adenoma, hematoma, leiomyoma), large bowel (adenocarcinomas, tubular adenoma, villous adenoma, hamartoma, leiomyoma)); (iv) genitourinary tract (including kidney (adenocarcinoma, WiIm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, stromal cell carcinoma, interstitial cell carcinoma, fibroma, fibroadenoma, adenoid tumors, lipoma); (v) liver (including hepatoma (hepatocellular carcinoma), cholangiocarcinoma, hepatoblastoma, angiosarcoma, hepatocellular adenoma, hemangioma); (vi) biliary tract (including gallbladder cancer, ampule cancer, bile duct carcinoma); (vii) bone (including osteogenic sarcoma (osteosarcoma), fibrosarcoma, malignant fibrous histocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors); (viii) nervous system (including skull (osteoma, angioma, hemangioma, granuloma, xanthoma, osteitis deformans, scleromalacia), meninges (meningioma, meningiosarcoma, gliomatosis), brain (astrocytoma, medulloblastoma, myeloblastoma, glioma, ependymoma, epididymis tumor, germinoma (pinealoma), glioblastoma multiform, oligodendroglioma, schwannoma, retinoblastoma, congenitial neoplasm, congential tumors), spinal cord, (neurofibroma, meningioma, glioma, sarcoma)); (ix) gynecological tissues (including uterus (endometrial carcinoma, serous bladder cancer, mucinous bladder cancer, carcinoma unclassified), granular sheath cell tumor, serous stromal cell tumor, dysplasia, dysgerminoma, malignant teratoma), cervix (cervical carcinoma, pre-tumor cervical dsplasia), ovaries (ovarian carcinoma [serous cystadenocarcinoma, mucinous cystadenocarcinoma], granulose-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma) vagina (clear cell carcinoma, squamous cell carcinoma, uveal or botryoid sarcoma (embryonal rhabdomyosarcoma), fallopian tubes carcinoma)); (x) hematologic system (including blood (myeloid leukemia (acute and chronic), (myelomatosis (acute and chronic), acute lymphocytic leukemia, chronic lymphocytic leukemia, myeloproliferative disorders, acute lymphoblastic leukemia, chronic lymphocytic leukemia, myeloproliferative diseases, multiple myeloma, myelodysplastic syndrome), Hodgkin's disease, non-Hodgkins's lymphoma, malignant lymphoma); (xi) skin (including malignant melanoma, basal cell carcinoma, squamous cell carcinoma, Kaposi's sarcoma, moles dysplastic nevus, lipoma, angioma, hemangioma, dermatofibroma, keloids, psoriasis, or adrenal neuroblastoma); and (xii) adrenal glands (including neuroblastoma).

In some embodiments of methods of the present disclosure, the cancer is non-small cell lung cancer (NSCLC), small cell lung cancer, pancreatic cancer, colorectal cancer, colon cancer, bile duct cancer, cervical cancer, bladder cancer, liver cancer or breast cancer.

In certain embodiments, there are provided methods for treating or preventing a hyperplastic or hyperproliferative disease or disorder (e.g., a cancer or a tumor) in a subject (e.g., a human) comprising administering to the subject a therapeutically effective amount of at least one KRAS inhibitor compound or composition provided herein. In some embodiments, the hyperplastic disorder is a cancer or a tumor, such as without limitation non-small cell lung cancer (NSCLC), pancreatic cancer, colorectal cancer, colon cancer, bile duct cancer, cervical cancer, bladder cancer, liver cancer or breast cancer.

Other diseases, disorders and conditions that can be treated or prevented, in whole or in part, by inhibition of KRAS activity are candidate indications for the KRAS inhibitor compounds and compositions provided herein and are encompassed by methods of the disclosure.

In some embodiments, the present disclosure provides a method for treating and/or preventing an immune-related disease, disorder, or condition, or symptoms thereof, in a subject comprising administering using at least one KRAS inhibitor compound or composition of the present disclosure to the subject.

In some embodiments, the present disclosure provides a method for treating and/or preventing an inflammatory disorder in a subject, comprising administering using at least one KRAS inhibitor compound or composition of the present disclosure to the subject.

In some embodiments, there is further provided the use of the KRAS inhibitor compounds and compositions described herein in combination with one or more additional agents. The one or more additional agents may have some KRAS-modulating activity and/or they may function through distinct mechanisms of action. In some embodiments, such agents comprise radiation (e.g., localized radiation therapy or total body radiation therapy) and/or other treatment modalities of a non-pharmacological nature. When combination therapy is utilized, the KRAS inhibitor(s) and one additional agent(s) may be in the form of a single composition or multiple compositions, and the treatment modalities can be administered concurrently, sequentially, or through some other regimen. By way of example, in some embodiments there is provided a treatment regimen wherein a radiation phase is followed by a chemotherapeutic phase. A combination therapy can have an additive or synergistic effect.

In some embodiments, there is provided the use of a KRAS inhibitor compound or composition described herein in combination with bone marrow transplantation, peripheral blood stem cell transplantation, or other types of transplantation therapy.

In particular embodiments, there is provided the use of the inhibitors of KRAS function described herein in combination with immune checkpoint inhibitors. The blockade of immune checkpoints, which results in the amplification of antigen-specific T cell responses, has been shown to be a promising approach in human cancer therapeutics. Non-limiting examples of immune checkpoints (ligands and receptors), some of which are selectively upregulated in various types of tumor cells, that are candidates for blockade include PD1 (programmed cell death protein 1); PDL1 (PD1 ligand); BTLA (B and T lymphocyte attenuator); CTLA4 (cytotoxic T-lymphocyte associated antigen 4); TIM3 (T-cell membrane protein 3); LAG3 (lymphocyte activation gene 3); A2aR (adenosine A2a receptor A2aR); and Killer Inhibitory Receptors. Non-limiting examples of immune checkpoint inhibitors include ipulimumab, nivolumab and lambrolizumab.

In other embodiments, there are provided methods for treating a cancer in a subject, comprising administering to the subject a therapeutically effective amount of at least one KRAS inhibitor compound or composition thereof and at least one chemotherapeutic agent, such agents including, but not limited to alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nucleoside analogs (e.g., gemcitabine); nitroso ureas such as carmustine, lomustine, and streptozocin; topoisomerase 1 inhibitors (e.g., irinotecan); platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, estramustine, vinblastine, docetaxol, epothilone derivatives, and paclitaxel); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide). There is also provided the use of the KRAS inhibitors in combination with other agents known in the art (e.g., arsenic trioxide) and other chemotherapeutic or anti-cancer agents that may be appropriate for treatment.

In some embodiments drawn to methods of treating cancer, the administration of a therapeutically effective amount of a KRAS inhibitor in combination with at least one chemotherapeutic agent results in a cancer survival rate greater than the cancer survival rate observed by administering either agent alone. In further embodiments drawn to methods of treating cancer, the administration of a therapeutically effective amount of a KRAS inhibitor in combination with at least one chemotherapeutic agent results in a reduction of tumor size or a slowing of tumor growth greater than reduction of the tumor size or slowing of tumor growth observed by administration of either agent alone.

In further embodiments, there are provided methods for treating or preventing cancer in a subject, comprising administering to the subject a therapeutically effective amount of at least one KRAS inhibitor compound or composition and at least one signal transduction inhibitor (STI). In a particular embodiment, the at least one STI is selected from the group consisting of bcr/abl kinase inhibitors, epidermal growth factor (EGF) receptor inhibitors, her-2/neu receptor inhibitors, and farnesyl transferase inhibitors (FTIs).

In other embodiments, there are provided methods of augmenting the rejection of tumor cells in a subject comprising administering an KRAS inhibitor compound or composition in conjunction with at least one chemotherapeutic agent and/or radiation therapy, wherein the resulting rejection of tumor cells is greater than that obtained by administering either the KRAS inhibitor, the chemotherapeutic agent or the radiation therapy alone.

In further embodiments, there are provided methods for treating cancer in a subject, comprising administering to the subject a therapeutically effective amount of at least one KRAS inhibitor and at least one anti-cancer agent other than a KRAS inhibitor. It should be understood that, as used herein, a “KRAS inhibitor” refers to compounds of the disclosure, e.g., a compound of Formula (I), a compound of Formula (I-a), a compound of Formula (I-b), a compound of any one of Formulae (II-a), (II-b), (III-a), (III-b), (IV-a), (IV-b), (IV-c), (V), (VI), a compound of Table 1 or 2, or a pharmaceutically acceptable salt, ester, hydrate, or solvate thereof, or a stereoisomer thereof, and to pharmaceutical compositions thereof.

In some embodiments, there are provided methods of treating or preventing a KRAS-associated disease, disorder or condition in a subject in need thereof, comprising administering a therapeutically effective amount of at least one KRAS inhibitor or a pharmaceutical composition thereof to the subject, such that the KRAS-associated disease, disorder or condition is treated or prevented in the subject. In some embodiments, the compound is administered in an amount effective to reverse, slow or stop the progression of a KRAS-mediated cancer in the subject.

In some embodiments, the KRAS-associated disease, disorder or condition is a KRAS-related cancer, tumor or hyperplastic or hyperproliferative disorder, such as, for example and without limitation, a cancer of the cardiac system, heart, lung, gastrointestinal system, genitourinary tract, biliary tract, small intestine, large intestine, liver, bone, nervous system, brain, gynecological system, hematologic tissues, skin, or adrenal glands, as described herein. In certain embodiments, the cancer, tumor or hyperplastic or hyperproliferative disorder is non-small cell lung cancer (NSCLC), small cell lung cancer, pancreatic cancer, colorectal cancer, colon cancer, bile duct cancer, cervical cancer, bladder cancer, liver cancer or breast cancer.

In some embodiments, methods provided herein further comprise administration of at least one additional therapeutic agent to the subject. The at least one additional therapeutic agent may be administered concomitantly or sequentially with the compound or composition described herein. In some embodiments, the at least one additional therapeutic agent is a chemotherapeutic agent or an anti-cancer agent. In an embodiment, the at least one additional therapeutic agent is an immune checkpoint inhibitor, such as, without limitation, ipulimumab, nivolumab or lambrolizumab.

In additional embodiments, methods provided herein further comprise administration of a tumor vaccine (e.g., a vaccine effective against melanoma); the tumor vaccine can comprise genetically modified tumor cells or a genetically modified cell line, including genetically modified tumor cells or a genetically modified cell line that has been transfected to express granulocyte-macrophage stimulating factor (GM-CSF). In particular embodiments, the vaccine includes one or more immunogenic peptides and/or dendritic cells.

In another broad aspect, there are provided kits comprising the compound or composition of the disclosure. Kits may include a compound described herein, or a pharmaceutically acceptable salt, ester, hydrate, solvate or stereoisomer thereof, for use to treat, prevent or inhibit a KRAS-associated disease, disorder or condition. Kits may further comprise a buffer or excipient, and/or instructions for use. In some embodiments, kits further comprise at least one additional therapeutic agent, such as without limitation a chemotherapeutic agent, an immune- and/or inflammation-modulating agent, an anti-hypercholesterolemia agent, an anti-infective agent, or an immune checkpoint inhibitor.

### DETAILED DESCRIPTION

### Definitions

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this invention pertains.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The terms “about” and “approximately” are used to indicate that a value includes an inherent variation of error for the device or the method being employed to determine the value.

The term “derivative” as used herein, is understood as being a substance similar in structure to another compound but differing in some slight structural detail.

The distribution of the positions of amino acid codons and residues of human KRAS is determined based on the P01116 amino acid sequence in UniProtKB/Swiss-Prot.

As used herein, the terms “wild-type KRAS” or “KRAS WT” refer to the non-mutated form of the mammalian KRAS protein. The term “wild-type KRAS inhibitor” as used herein refers to compounds of the disclosure, e.g., as represented by Formula (I) herein, which are capable of negatively regulating or inhibiting the enzyme activity of wild-type KRAS in whole or in part. As used herein, “wild-type KRAS-related diseases or conditions” refers to diseases or conditions that are associated with, mediated by, or involve wild-type KRAS. Non-limiting examples of wild-type KRAS-related diseases or conditions include wild-type KRAS-related cancers.

The term “KRAS G12A” as used herein refers to a mutated form of the mammalian KRAS protein, which contains an amino acid substitution of alanine for glycine at amino acid position 12. The term “KRAS G12A inhibitor” as used herein refers to compounds of the disclosure, e.g., as represented in Formula (I) herein, which are capable of negatively regulating or inhibiting the enzyme activity of KRAS G12A in whole or in part. As used herein, “KRAS G12A-related diseases or conditions” refers to diseases or conditions that are associated with, mediated by, or involve the KRAS G12A mutation. Non-limiting examples of KRAS G12A-related diseases or conditions include KRAS G12A-related cancers.

The term “KRAS G12C” as used herein refers to a mutated form of the mammalian KRAS protein, which contains an amino acid substitution of cysteine for glycine at amino acid position 12. The term “KRAS G12C inhibitor” as used herein refers to compounds of the disclosure, e.g., as represented in Formula (I) herein, which are capable of negatively regulating or inhibiting the enzyme activity of KRAS G12C in whole or in part. The term “KRAS G12C-related diseases or conditions” as used herein refers to diseases or conditions that are associated with or mediated by or involve the KRAS G12C mutation. Non-limiting examples of KRAS G12C-related diseases or conditions include KRAS G12C-related cancers.

The term “KRAS G12D” as used herein refers to a mutated form of the mammalian KRAS protein, which contains an amino acid substitution of aspartic acid for glycine at amino acid position 12. As used herein, a “KRAS G12D inhibitor” refers to compounds compounds of the disclosure, e.g., as represented in Formula (I), which are capable of negatively regulating or inhibiting the enzymatic activity of KRAS G12D in whole or in part. The term “KRAS G12D-related diseases or conditions” as used herein refers to diseases or conditions that are associated with or mediated by or involve the KRAS G12D mutation. Non-limiting examples of KRAS G12D-related diseases or conditions include KRAS G12D-related cancers.

The term “KRAS G12R” as used herein refers to a mutated form of the mammalian KRAS protein, which contains an amino acid substitution of arginine for glycine at amino acid position 12. The term “KRAS G12R inhibitor” as used herein refers to compounds of the disclosure, e.g., as represented in Formula (I) herein, which are capable of negatively regulating or inhibiting the enzyme activity of KRAS G12R in whole or in part. The term “KRAS G12R-related diseases or conditions” as used herein refers to diseases or conditions that are associated with or mediated by or involve the KRAS G12R mutation. Non-limiting examples of KRAS G12R-related diseases or conditions include KRAS G12R-related cancers.

The term “KRAS G12S” as used herein refers to a mutated form of the mammalian KRAS protein, which contains an amino acid substitution of serine for glycine at amino acid position 12. The term “KRAS G12S inhibitor” as used herein refers to compounds of the disclosure, e.g., as represented in Formula (1) herein, which are capable of negatively regulating or inhibiting the enzyme activity of KRAS G12S in whole or in part. The term “KRAS G12S-related diseases or conditions” as used herein refers to diseases or conditions that are associated with or mediated by or involve the KRAS G12S mutation. Non-limiting examples of KRAS G12S-related diseases or conditions include KRAS G12S-related cancers.

The term “KRAS G12V” as used herein refers to a mutated form of the mammalian KRAS protein, which contains an amino acid substitution of valine for glycine at amino acid position 12. The term “KRAS G12V inhibitor” as used herein refers to compounds of the disclosure, e.g., as represented in Formula (I), which are capable of negatively regulating or inhibiting the full or partial enzymatic activity of KRAS G12V The term “KRAS G12-related diseases or conditions” as used herein refers to diseases or conditions that are associated with or mediated by or involve the KRAS G12V mutation. Non-limiting examples of KRAS G12V-related diseases or conditions include KRAS G12V-related cancers.

The term “KRAS G13D” as used herein refers to a mutated form of the mammalian KRAS protein, which contains an amino acid substitution of aspartic acid for glycine at amino acid position 13. The term “KRAS G13D inhibitor” as used herein refers to compounds of the disclosure, e.g., as represented in Formula (I) herein, which are capable of negatively regulating or inhibiting the enzyme activity of KRAS G13D in whole or in part. The term “KRAS G13D-related diseases or conditions” as used herein refers to diseases or conditions associated with, mediated by, or involve KRAS G13D. Non-limiting examples of KRAS G13D-related diseases or conditions include KRAS G13D-related cancers.

The term “KRAS Q61H” as used herein refers to a mutated form of the mammalian KRAS protein, which contains an amino acid substitution of histidine for glutamine at amino acid position 61. The term “KRAS Q61H inhibitor” as used herein refers to compounds of the disclosure, e.g., as represented in Formula (I) herein, which are capable of negatively regulating or inhibiting the enzyme activity of KRAS Q61H in whole or in part. The term “KRAS Q61H-related diseases or conditions” as used herein refers to diseases or conditions that are associated with or mediated by or involve the KRAS Q61H mutation. Non-limiting examples of KRAS Q61H-related diseases or conditions include KRAS Q61H-related cancers.

The term “prodrug” or its equivalent refers to a reagent that is directly or indirectly converted into an active form in vitro or in vivo (see, for example, R. B. Silverman, 1992, “The Organic Chemistry of Drug Design and Drug Action,” Academic Press, Chap. 8; Bundgaard, Hans; Editor. Neth. (1985), “Design of Prodrugs” 360 pp. Elsevier, Amsterdam; Stella, V.; Borchardt, R.; Hageman, M.; Oliyai, R.; Maag, H.; Tilley, J. (Eds.) (2007), “Prodrugs: Challenges and Rewards, XVIII, 1470 p. Springer). A prodrug can be used to change the biological distribution of specific drugs (for example, to make the drug usually not enter the protease reaction site) or its pharmacokinetics. A variety of groups have been used to modify compounds to form prodrugs, such as esters, ethers, phosphate esters/salts, etc. When a prodrug is administered to a subject, the group is cleaved in the subject by an enzymatic or non-enzymatic process, e.g., by reduction, oxidation or hydrolysis, or in another way, to release the active compound. As used herein, “prodrug” may include pharmaceutically acceptable salts or esters, or pharmaceutically acceptable solvates or chelates, as well as crystalline forms of a compound.

The term “pharmaceutically acceptable”, as used in the present disclosure, means drugs, pharmaceutical products, inert ingredients etc., described by the term, which are suitable for use in contact with tissues of humans and lower animals without abnormal toxicity, incompatibility, instability, irritation, allergic reactions etc., proportional to a reasonable benefit/risk ratio.

A “pharmaceutically acceptable stereoisomer” of a compound refers to the isomer produced by the different spatial arrangement of atoms or groups in a molecule. Isomers caused by the same order of atoms or atomic groups in the molecule but with different spatial arrangement are called stereoisomers. Stereoisomers are mainly divided into two categories: stereoisomers caused by bond length, bond angle, intramolecular double bond, ring, and the like are called configuration stereoisomers. In general, isomers cannot or are difficult to convert into each other. Stereoisomers caused only by the rotation of a single bond are called conformational stereoisomers, sometimes also known as rotational isomers. When the rotation in the rotating isomer is blocked and cannot rotate, it becomes a “stereoisomer”, for example, in the biphenyl structure, when α- and α′-positions bear large and different substituents, the rotation of the single bond between the two phenyl rings stops due to the hindrance between the substituents, producing two stereoisomers.

The terms “substituted”, “with substituent” and “with substitution” mean that the parent compound or part thereof has at least one substituent group. Unless otherwise indicated, a “substituent” group can be at one or more substitutable positions of the parent group, and when there is more than one substituent present at different positions of a given structure, the substituents can be the same or different at each position. In certain embodiments, the terms “substituent” and “substituted group” include, but are not limited to, halogen (F, Cl, Br or I), hydroxyl, sulfhydryl, mercapto, amino, nitro, carbonyl, carboxyl, alkyl, alkoxy, alkylamino, aryl, aryloxy, arylamino, acyl, sulfinyl, sulfonyl, phosphonyl and other organic parts routinely used and accepted in organic chemistry.

Where multiple substituents are indicated as being attached to a structure, it is to be understood that the substituents can be the same or different. Thus for example “Rₘ optionally substituted with 1, 2 or 3 R_{q} groups” indicates that Rₘ is substituted with 1, 2, or 3 R_{q} groups where the R_{q} groups can be the same or different.

The terms “unsubstituted” and “without substitution” mean that a compound or part thereof has no substituent except the undetermined chemical saturation of hydrogen atom.

In some embodiments, alkyl, heteroazanyl, acyl, cycloalkyl, heterocycloalkyl, alkoxy, aryloxy, heteroalkoxy, heteroaryloxy, aryl, heteroaryl group, amino acid residues, oligopeptide (dipeptide, tripeptide, tetrapeptide) residues, phosphoryl, phosphonyl, aminophosphonyl, sulfonyl, thioacyl, benzyl, alkoxycarbonyl, aminocarbonyl, mercaptothiocarbonyl, alkylthio, thiocarbonyl, benzyloxycarbonyl, glucoside, and glucuronide, as mentioned in the present disclosure, may be optionally substituted (e.g., “substituted” or “unsubstituted” alkyl, “substituted” or “unsubstituted” heterocyclyl, “substituted” or “unsubstituted” aryl, or “substituted” or “unsubstituted” heteroaryl group).

Unless otherwise specified, a “substituted” group has one substituent at one or more substitutable positions, and the substituent is the same or different at each position when replacing more positions in any given structure.

As used herein, the term “hydrocarbyl” means a group only containing carbon and hydrogen atoms, which may be saturated or unsaturated. For example, alkyl, alkenyl, and alkynyl are all examples of “hydrocarbyl”. Non-limiting examples of hydrocarbyl include methyl, ethyl, propyl, n-butyl, isobutyl, vinyl, propynyl, etc. The term “hydrocarbyl” includes, but is not limited to saturated hydrocarbyl, unsaturated hydrocarbyl, aromatic hydrocarbyl, oxyhydrocarbyl, azahydrocarbyl, thiahydrocarbyl, phosphahydrocarbyl, as well as mixed heterohydrocarbyl with various heteroatoms. The chain length of the hydrocarbyl or heterohydrocarbyl ranges from 1 to 20 atoms. When it's hydrocarbyl, it contains 1 to 5 heteroatoms, and the chemical valence of these heteroatoms can be satisfied by hydrogen, oxygen, nitrogen, etc., as needed, through appropriate bonding.

Unless the number of carbons is otherwise specified, “lower” as in “lower aliphatic group”, “lower hydrocarbyl”, “lower alkyl”, “lower alkenyl”, and “lower alkynyl”, as used herein, means that the moiety has at least one (at least two for alkenyl and alkynyl) and not more than 6 (≤6) carbon atoms.

The terms “cycloalkyl”, “alicyclic group”, “carbocycle”, “cyclic group”, “cyclohydrocarbyl” and equivalent expressions mean a group containing saturated or partially unsaturated carbon rings in a monocyclic, bicyclic (sharing a common atom), spiro (sharing one atom), polycyclic (sharing at least one bond), or fused (sharing at least one bond) carbocyclic system, wherein the carbocyclic system has 3-15 carbon atoms. Examples of cycloalkyl include, but are not limited to, cyclopropyl, cyclobutyl, cyclopentyl, cyclopenten-1-yl, cyclopenten-2-yl, cyclopenten-3-yl, cyclohexyl, cyclohexen-1-yl, cyclohexen-2-yl, cyclohexen-3 cycloheptyl, bicyclo[4,3,0]nonyl, norbornyl, etc. The term “cycloalkyl” includes both unsubstituted and substituted cycloalkyl. The term “cyclohydrocarbyl” refers to the combination of a cyclo group and a hydrocarbyl group.

The term “heterocyclic ring” and equivalent descriptions used in the present invention refer to the group containing saturated or unsaturated carbon ring in a monocyclic, spiro (sharing one atom) or fused (sharing at least one bond) carbon ring system, which contains 3 to 15 carbon atom groups, including 1 to 6 heteroatoms (e.g., N, O, S and P) or groups containing heteroatoms (e.g., NH, NRx (Rx is alkyl, acyl, aryl, heteroaryl or cycloalkyl), PO₂, SO, SO₂, etc.). Heterocyclohydrocarbyls can be linked to C or heteroatoms (e.g., via N atoms). “Heterocycle” or “heterocyclic” covers heterocycloalkyl and heteroaryl. The examples of heterocyclic ring include but are not limited to acridinyl, azocinyl, benzimidazolyl, benzofuranyl, benzothiophenyl, benzothiophenyl, benzoxazolyl, benzothiazolyl, benzotriazolyl, benzotetrazolyl, benzoisoxazolyl, benzoisothiazolyl, 4αH-carbazolyl, carbolinyl, chromanyl, chromenyl, cinnolinyl, decahydroquinolinyl, 2H, 6H-1, 5, 2-dithiazinyl, dihydrofurano[2, 3-b]tetrahydrofuran, furanyl, furazanyl, imidazolidinyl, imidazolinyl, imidazolyl, 1H-indazolyl, dihydroindolyl, 3H-indazolyl, isoquinolinyl, isothiazolyl, isoxazolyl, methylenedioxybenzyl, morpholinyl, naphthyridinyl, octahydroisoquinolinyl, oxadiazolyl, 1, 2, 3-oxadiazoyl, 1, 2, 4-oxadiazoyl, 1, 2, 5-oxadiazoyl, 1, 3, 4-oxadiazoyl, oxazolidinyl, oxazolyl, oxazolidinyl, pyrimidinyl, phenanthridinyl, phenanthrolinyl, phenazinyl, phenothiazinyl, phenoxazinyl, phthalazinyl, piperazinyl, piperidyl, piperidonyl, 4-piperidonyl, piperonyl, pteridyl, purinyl, pyranyl, pyrazinyl, pyrazolidinyl, pyrazolinyl, pyrazolyl, pyridazinyl, pyrido-oxazole, pyrido-imidazole, pyrido-thiazole, pyridyl, pyridyl, pyrryl, pyrryl, quinazolinyl, quinolyl, 4H-quinolizinyl, quinoxalinyl, quinuclidinyl, tetrahydrofuranyl, tetrahydroisoquinolinyl, tetrahydroquinolinyl, tetrazolyl, 6H-1, 2, 5-thiadiazinyl, 1, 2, 3-thiadiazolyl, 1, 2, 4-thiadiazolyl, 1, 2, 5-thiadiazolyl, 1, 3, 4-thiadiazolyl, thianthrenyl, thiazolyl, thiophenyl, thieno-thiazolyl, thieno-oxazolyl, thieno-imidazolyl, thiophenyl, triazinyl, 1, 2, 3-triazolyl 1, 2, 4-triazolyl, 1, 2, 5-triazolyl, 3, 4-triazolyl, xanthenyl, etc. The term “cycloalkyl” covers unsubstituted heterocyclyls and substituted heterocyclyls. A heterocyclic ring can be linked via hydrocarbyls, also called heterocyclohydrocarbyls.

The terms “aryl” and “aromatic”, as used in the present disclosure, refers to aryl groups having “4n+2” (π) electrons and 6-14 ring atoms in a conjugated mono- or polycyclic system (fused or non-fused), wherein n is an integer from 1 to 3. The polycyclic system includes at least one aromatic ring. The aryl can be linked directly or via C₁-C₃ alkyl (also known as arylalkyl or alkylaryl or aralkyl). Examples of aryl include, but are not limited to, phenyl, benzyl, phenethyl, 1-phenylethyl, tolyl, naphthyl, biphenyl, terphenyl, indenyl, benzocyclooctenyl, benzocycloheptenyl, azulenyl, acenaphthyl, fluorenyl, phenanthryl, anthryl, etc. The term “aryl” includes unsubstituted and substituted aryl. When the aryl group is linked by hydrocarbyl, it is also known as aryl hydrocarbyl group.

The term “heterocycle” and equivalents, as used in the present disclosure, means a group comprising a saturated or partially unsaturated carbocyclic ring in a monocyclic, spiro (sharing one atom) or fused (sharing at least one bond) carbocyclic system, which has 3-15 carbon atoms, including 1-6 heteroatoms (e.g., N, O, S, P) or groups containing heteroatoms (e.g., NH, NRx (where Rx is alkyl, acyl, aryl, heteroaryl or cycloalkyl), PO₂, SO, SO₂, etc.). Heterocyclic hydrocarbyl groups may be linked to C or with heteroatoms (e.g., via nitrogen atoms). The term “heterocycle” or “heterocyclic” includes heterocycloalkyl and heteroaryl. Examples of heterocycles include, but are not limited to, acridinyl, azocinelyl, benzimidazolyl, benzofuranyl, benzothienyl, benzoxazolyl, benzothiazolyl, benzotriazolyl, benzotetrazolyl, benzisoxazolyl, benzisothiazolyl, 4αH-carbazolyl, carbolinyl, chromanyl, chromenyl, misolinyl, decahydroquinolinyl, 2H, 6H-1, 5, 2-dithiazinyl, dihydrofuro [2, 3-b] tetrahydrofuran, furanyl, furazanyl, imidazolidinyl, imidazolinyl, 1H-indazolyl, dihydroindolyl, 3H-indolyl, isoquinolinyl, isothiazolyl, isoxazolyl, methylenedioxyphenyl, morpholinyl, naphthyridinyl, octahydroisoquinolinyl, oxadiazolyl, 1, 2, 3-oxadiazolyl, 1, 2, 4-oxadiazolyl, 1, 2, 5-oxadiazolyl, 1, 3, 4-oxadiazolyl, oxazolyl, oxazolidinyl, pyrimidinyl, phenanthridinyl, phenanthroline, phenazinyl, phenothiazinyl, phenoxazinyl, phthalazinyl, piperazinyl, piperidinyl, piperidinyl, 4-piperidinyl, piperonyl, pteridinyl, purinyl, pyranyl, pyrazolinyl, pyrazolidinyl, pyrazolinyl, pyridazinyl, pyridoxazole, pyridimidazole, pyridothiazole, pyridyl, pyridyl, pyrrolyl, pyrrolyl, quinazolinyl, quinolinyl, 4H-quinazinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthryl, thiazolyl, thienyl, thienothiazolyl, thienoxazolyl, thienoimidazolyl, thienyl, triazinyl, 1,2,3-triazolyl, 1,2,4-triazolyl, 1,2,5-triazolyl, 3,4-triazolyl, xanthenyl etc. The term “heterocycle” includes unsubstituted and substituted heterocyclyl. Heterocycles linked by hydrocarbyl are also known as heterocyclic hydrocarbyl.

The term “spiro” or “spirocyclic” refers to organic compounds that exhibit a twisted structure involving two or more rings (ring systems) where 2 or 3 rings are linked through one shared atom. Spirocyclic compounds can consist of entirely carbon rings (all-carbon), such as spiro [5.5] undecane, or heterocyclic compounds (containing one or more non-carbon atoms), including but not limited to carbon spirocyclic compounds, heterocyclic spirocyclic compounds, and polycyclic compounds.

The term “bridged ring” or “bridged” refers to carbon or heterocyclic moieties sharing two or more atoms in two or more ring structures, where the shared atoms can be C, N, S, or other heteroatoms arranged in chemically reasonable substitution patterns. Alternatively, “bridged ring” compounds also refer to carbon or heterocyclic structures in which an atom at any position on the main ring is bonded to a second atom on the main ring via a chemical bond or an atom other than a bond, and it does not actually form a part of the main ring structure. The first and second atoms can be adjacent to each other or non-adjacent within the main ring. In addition to examples of bridged ring structures provided herein, other carbon ring or heterocyclic bridged ring structures are also foreseeable, including bridged rings where the bridging atoms are either C or heteroatoms arranged in chemically reasonable substitution patterns, as known in the field.

The term “fused ring” or “fused-ring system” means a polycyclic system containing fused rings. The fused ring system typically contains 2 or 3 rings and/or up to 18 ring atoms. As described above, cycloalkyl, aryl, and heterocyclic can form a fused ring system. Thus, the fused ring system may be aromatic, partially or non-aromatic, and may contain heteroatoms. According to this definition, spiro ring systems are not fused polycyclic systems. However, the fused polycyclic systems of the present disclosure may have a spiro ring linked thereto through a single ring atom of the system. Examples of fused ring systems include, but are not limited to, naphthyl (e.g., 2-naphthyl), indenyl, phenanthryl, anthryl, pyrenyl, benzimidazole, benzothiazole, and so on.

As used herein, the term “heteroaromatic ring” refers to substituted or unsubstituted N-containing six-membered heteroaromatic rings and substituted or unsubstituted five-membered heteroaromatic rings, where the substituent groups are selected from the C₁₋₄ linear or branched alkyls, halogen-substituted C₁₋₄ linear or branched alkyls, F, Cl, Br, NO₂, CN, methylenedioxy, cyclopropyl, cyclopropylmethylene, substituted or unsubstituted cyclobutyl, substituted or unsubstituted cyclopentyl, and the benzene ring. N-containing six-membered heteroaromatic rings and five-membered heteroaromatic rings can be monosubstituted or polysubstituted. Six-membered heteroaromatic rings can contain one N atom or multiple N atoms. In some embodiments, five-membered heteroaromatic rings can contain one heteroatom or multiple heteroatoms, with the heteroatom(s) selected from O, N and S, with halogens selected from F, Cl and Br.

The term “acyl”, as used in the present disclosure, refers to the fragment —C(═O)Rₐ obtained after the removal of a hydroxy from a carboxylic acid molecule. The term “acyl” refers to a compound or fragment in which at least one carbon or heteroatom is covalently bonded to the carbon atom of —C═O. Acyl includes substituted and unsubstituted groups.

The term “amine” or “amino”, as used in the present disclosure, refers to unsubstituted or substituted fragments of the general Formula —NRₐR_{b} in which Rₐ and R_{b} are independently substituted or unsubstituted hydrogen, alkyl, hydrocarbyl, aryl, cyclic or heterocyclic, etc., or Rₐ and R_{b} together form a heterocyclic ring with the nitrogen atom to which they are linked. The term “amide” refers to the structure —C(═O)NR_{b}R_{c} in which the amino is directly linked to the acyl. The term “acylamino” means the covalent bonding of at least one carbon or heteroatom in a compound or fragment to a carbon atom on acylamino.

The term “alkanoyloxy” means that Ra on an acyl is an alkyl, and the oxygen atom of the alkyl is connected to a carbon atom in the acyl, while the other end is covalently bonded to at least one carbon or heteroatom in the compound or fragment.

The term “thioacyl” refers to a fragment of —C(═S)Rₐ, formed by substituting the oxygen atom on the acyl group with a sulfur atom.

The term “aliphatic acyl” means an acyl group to which an aliphatic group is linked to a carbon atom on the acyl, i.e., Rₐ is aliphatic. The term “aroyl” refers to an acyl to which the aryl is linked to a carbon atom on the acyl, i.e., Rₐ is aryl.

The term “phosphonyl” or “phosphoryl” means the fragment —P(═O)(OR_{d})Rₑ left after dehydroxylation of monomolecular phosphoric acid. The term “phosphonyl” means the covalent bonding of at least one carbon or heteroatom in a compound or fragment to a phosphorus atom on the phosphonyl. R_{d} is substituted or unsubstituted hydrogen, hydrocarbyl, aryl, cyclic or heterocyclic group, etc. The term “aminophosphonyl” means the linkage of amine to phosphonyl, i.e., Rₑ is amine.

The term “sulfonyl” refers to the fragment left after the dehydroxylation of monomolecular sulfonic acid, and the term “sulfonyl” refers to the covalent bonding of at least one carbon or heteroatom in a compound or fragment to a sulfur atom on the sulfonyl.

The term “carbonyl” refers to a C═OR_{f} fragment formed by carbon and oxygen atoms linked by a double bond, and the term “carbonyl” is a constituent of functional groups such as aldehydes, ketones, acids, etc. The term “carbonyl” refers to the covalent bonding of at least one carbon or heteroatom of a compound or fragment to a carbon atom on C═OR_{f}, and R_{f} is a substituted or unsubstituted hydrogen, hydrocarbyl, aryl, cyclic or heterocycloalkyl, etc. The term “alkoxycarbonyl” means that R_{f} is an alkoxy, wherein the oxygen atom of the alkoxy is linked to the carbon atom of the carbonyl. The term “aminocarbonyl” means that R_{f} is an amine, wherein the nitrogen atom of the amine is linked to the carbon atom of the carbonyl. The term “benzyloxycarbonyl” means the linkage of the oxygen atom of the benzyloxy to the carbon atom of the carbonyl.

The term “thiocarbonyl” refers to a fragment of —C(═S)R_{f} formed after the substitution of an oxygen atom on the carbonyl by a sulfur atom. The term “mercaptothiocarbonyl” means that R_{f} is a sulfhydryl, wherein the carbon atom of the thiocarbonyl is linked to the sulfur atom of the sulfhydryl.

The term “alkylthio” refers to an alkyl linked to a sulfhydryl thereon. A suitable alkylthio includes 1 to about 20 carbon atoms, preferably 1 to about 15 carbon atoms.

The term “alkoxy” or “lower alkoxy”, as used in the present disclosure, refers to a structure in which the alkyl is linked to an oxygen atom. A representative alkoxy includes a group having 1 to about 6 carbon atoms, such as methoxy, ethoxy, propoxy, t-butoxy, etc. Examples of alkoxy include, but are not limited to, methoxy, ethoxy, isopropoxy, propoxy, butoxy, pentoxy, fluoromethoxy, difluoromethoxy, trifluoromethoxy, chloromethoxy, dichloromethoxy, trichloromethoxy, etc. The term “alkoxy” includes unsubstituted or substituted alkoxy, as well as perhaloalkoxy groups.

“Cholic acid substituents” in the present disclosure refer to bile acids synthesized by liver cells, also called primary bile acids, including cholic acid, ursodeoxycholic acid, chenodeoxycholic acid, glycocholic acid, taurocholic acid, glycochenodeoxycholic acid, taurochenodeoxycholic acid, especially substituted chenodeoxycholic acid and substituted ursodeoxycholic acid.

The term “base compound” or “base molecule”, as used in the present disclosure, refers to a particular compound or drug molecule with desirable biological activity. In addition to being a drug molecule by itself, it can also be further modified or derivatized to form new compounds, such as prodrug compounds or derivatized compounds.

The term “ester-forming group” or “ester”, as used in the present disclosure, refers to a structure in which the fragment contains an ester functional group —RCOOR′ (where R′ is generally another non-H group such as alkyl). This group is typically obtained by the reaction of a carboxylic acid with an alcohol (eliminating one molecule of water). Non-limiting examples for R include a lower alkyl or aryl, such as methylene, ethylene, isopropylene, isopropylidene, phenylene, etc. Non-limiting examples for R′ include a lower alkyl or aryl, such as methyl, ethyl, propyl, isopropyl, butyl, phenyl, etc. The term “ester alkyl” means that R′ is an alkyl, one end of which is directly connected with the oxygen on the ester, and the other end is covalently bonded with at least one carbon or heteroatom in a compound or fragment.

The term “amino acid” generally refers to an organic compound that contains both a carboxylic acid group and an amino group. The term “amino acid” includes both “natural” and “unnatural” amino acids. In addition, the term “amino acid” includes an O-alkylated amino acid or an N-alkylated amino acid, as well as an amino acid with a nitrogen-, sulfur-, or oxygen-containing side chain (e.g., Lys, Cys, or Ser), wherein the nitrogen, sulfur, or oxygen atom may or may not be acylated or alkylated. The amino acid may be a pure L-isomer or D-isomer, or a mixture of L-isomer and D-isomer, including (but not limited to) a racemic mixture.

The term “natural amino acid” and equivalent refers to L-amino acids normally found in naturally occurring proteins. Examples of natural amino acids include, but are not limited to, alanine (Ala), cysteine (Cys), aspartic acid (Asp), glutamic acid (Glu), phenylalanine (Phe), glycine (Gly), histidine (His), isoleucine (Ile), lysine (Lys), leucine (Leu), methionine (Met), asparagine (Asn), proline (Pro), glutamine (Gln), arginine (Arg), serine (Ser), threonine (Thr), valine (Val), tryptophan (Trp), tyrosine (Tyr), β-alanine (beta-Ala) and γ-aminobutyric acid (GABA).

The term “unnatural amino acid” refers to any derivative of a natural amino acid, including D-amino acid, as well as α- and β-amino acid derivatives. The terms “unnatural amino acid” and “not natural amino acid” are used interchangeably herein. It should be noted that certain amino acids (e.g., hydroxyproline), which may be classified as unnatural amino acids in the present disclosure, may also be present in certain biological tissues or specific proteins in nature. The amino acids with many different protecting groups and suitable for direct application in solid-phase peptide synthesis are available to purchase. In addition to twenty of the most common natural amino acids, the following exemplary unnatural amino acids and amino acid derivatives (common abbreviations in parentheses) may be used according to the present disclosure: 2-aminoadipic acid (Aad), 3-aminoadipic acid (β-Aad), 2-aminobutyric acid (2-Abu), α,β-dehydro-2-aminobutyric acid (8-AU), 1-aminocyclopropane-1-carboxylic acid (ACPC), aminoisobutyric acid (Aib), 3-aminoisobutyric acid (β-Aib), 2-amino-thiazoline-4-carboxylic acid, 5-aminopentanoic acid (5-Ava), 6-aminohexanoic acid (6-Ahx), 2-aminoheptanoic acid (Ahe), 8-aminooctanoic acid (8-Aoc), 11-aminoundecanoic acid (11-Aun), 12-aminododecanoic acid (12-Ado), 2-aminobenzoic acid (2-Abz), 3-aminobenzoic acid (3-Abz), 4-aminobenzoic acid (4-Abz), 4-amino-3-hydroxy-6-methylheptanoic acid (statine, Sta), aminooxyacetic acid (Aoa), 2-aminotetraline-2-carboxylic acid (ATC), 4-amino-5-cyclohexyl-3-hydroxyvaleric acid (ACHPA), P-aminophenylalanine (4-NH₂-Phe), 2-aminopimelic acid (Apm), biphenylalanine (Bip), P-bromophenylalanine (4-Br-Phe), O-chlorophenylalanine (2-Cl-Phe), M-chlorophenylalanine (3-Cl-Phe), P-chlorophenylalanine (4-Cl-Phe), M-chlorotyrosine (3-Cl-Tyr), P-benzoylphenylalanine (Bpa), t-butylglycine (TLG), cyclohexylalanine (Cha), cyclohexylglycine (Chg), desmosine (Des), 2,2-diaminopimelic acid (Dpm), 2,3-diaminopropionic acid (Dpr), 2,4-diaminobutyric acid (Dbu), 3,4-dichlorophenylalanine (3,4-Cl2-Phe), 3,4-difluorophenylalanine (3,4-F2-Phe), 3,5-diiodotyrosine (3,5-I2-Tyr), N-ethylglycine (EtGly), N-ethylasparagine (EtAsn), o-fluorophenylalanine (2-F-Phe), m-fluorophenylalanine (3-F-Phe), p-fluorophenylalanine (4-F-Phe), M-fluorotyrosine (3-F-Tyr), homoserine (Hse), homophenylalanine (Hfe), homotyrosine (Htyr), hydroxylysine (Hyl), isohydroxylysine (aHyl), 5-hydroxytryptophan (5-OH-Trp), 3- or 4-hydroxyproline (3- or 4-Hyp), p-iodophenylalanine-isotyrosine (3-I-Tyr), dihydroindole-2-carboxylic acid (Idc), isoiduramycin (Ide), isoleucine (α-Ile), isopipecolic Acid (Inp), N-methylisoleucine (MeLys), m-methyltyrosine (3-Me-Tyr), N-methylvaline (MeVal), 1-naphthylalanine (1-Nal), 2-naphthylalanine (2-Nal), p-nitrophenylalanine (4-NO2-Phe), 3-nitrotyrosine (3-NO2-Tyr), norleucine (Nle), norvaline (Nva), ornithine (Orn), O-phosphotyrosine (H2PO3-Tyr), octahydroindole-2-carboxylic acid (Oic), penicillamine (Pen), pentafluorophenylalanine (F5-Phe), phenylglycine (Phg), pipecolic acid (Pip), propargylglycine (Pra), pyroglutamic acid (PGLU), sarcosine (Sar), tetrahydroisoquinoline-3-carboxylic acid (Tic), and thiazolidine-4-carboxylic acid (thioproline, Th).

The term “peptide” or “oligopeptide” refers to a compound formed by the intermolecular dehydration condensation of two or more amino acids linked together by an amide bond. In general, the number of amino acids constituting a peptide ranges from 2 (dipeptide) to 20 (eicosapeptide).

The term “residue” refers to the major part of the molecule after the removal of a group, such as amino acid residue (e.g., the structure H₂NCH₂CO—, i.e., glycyl, the part after the removal of hydroxyl from glycine) and peptide residue.

The term “solvate” refers to a physical association of a compound with one or more solvent molecules, whether organic or inorganic. This physical association includes hydrogen bonding. In certain instances, a solvate will be capable of isolation, for example when one or more solvent molecules are incorporated in the crystal lattice of a crystalline solid. “Solvate” encompasses both solution-phase and isolable solvates. Exemplary solvates include, without limitation, hydrates, ethanolates, methanolates, hemiethanolates, and the like.

The term “hydrate” refers to a compound that is bonded to one or more water (H₂O) molecule, e.g., by a hydrogen bond.

The term “salt-forming moiety”, as used in the present disclosure, refers to a moiety capable of forming a salt with an acidic group, such as a carboxyl, including but not limited to, sodium, potassium, tetraethylamine, tetrabutylamine, tetraethylammonium, tetrabutylammonium, etc.

As used herein, a “pharmaceutically acceptable salt” of a compound means a salt of a compound that is pharmaceutically acceptable. Desirable are salts of a compound that retain or improve the biological effectiveness and desired biology activities or properties of the free acids and bases of the parent compound as defined herein, or that take advantage of an intrinsically basic, acidic or charged functionality on the molecule and that are not biologically or otherwise undesirable. Examples of pharmaceutically acceptable salts are also described, for example, in Berge et al., “Pharmaceutical Salts”, J. Pharm. Sci. 66, 1-19 (1977). Examples of pharmaceutically acceptable salts include but are not limited to:
(1) A salt formed by adding an acid to a basic or positively charged functional group. Inorganic acids include hydrochloric acid, hydrobromic acid, hydroiodic acid, sulfuric acid, sulfamic acid, nitric acid, phosphoric acid, carbonate, etc. Organic acids include acetic acid, propionic acid, lactic acid, oxalic acid, glycolic acid, pivalic acid, t-butyl acetic acid, β-hydroxybutyric acid, valeric acid, caproic acid, cyclopentane propionic acid, pyruvic acid, malonic acid, succinic acid, malic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1, 2-ethanedisulfonic acid, 2-hydroxyethanesulfonic acid, cyclohexylsulfamic acid, benzenesulfonic acid, sulfanilic acid, 4-chlorobenzenesulfonic acid, 2-naphthalenesulfonic acid, 4-toluenesulfonic acid, camphorsulfonic acid, 3-phenylpropionic acid, laurylsulfonic acid, laurylsulfuric acid, oleic acid, palmitic acid, stearic acid, lauric acid, fluric acid, pantothenic acid, lactobionic acid, alginic acid, galactonic acid, galacturonic acid, gluconic acid, glucoheptonic acid, glutamic acid, naphthoic acid, hydroxynaphthoic acid, salicylic acid, ascorbic acid, stearic acid, muconic acid, etc.
(2) When an acidic proton is present in the parent compound, or a metal ion replaces it, a base may be added to give a salt. The metal ions include alkaline metal ions (such as lithium, sodium, and potassium), alkaline earth metal ions (magnesium, calcium, barium), or other metal ions such as aluminum, zinc, iron, etc. Organic bases include, but are not limited to, N,N′-dibenzylethylenediamine, ethanolamine, diethanolamine, triethanolamine, tromethamine, N-methylglucamine, piperazine, chloroprocaine, procaine, choline, lysine, etc.

Pharmaceutically acceptable salts may be synthesized from a parent compound containing basic or acidic fragments by conventional chemical methods. Typically, such salts are prepared by reacting a compound (free acid or base) with an equal stoichiometric amount of a base or acid in water, an organic solvent, or a mixture of the two. Salts may be prepared in situ during the final isolation or purification of a compound or by separately reacting a compound in its free acid or base form alone with the desired corresponding base or acid and isolating the salt thus formed. The term “pharmaceutically acceptable salt” also includes zwitterionic compounds comprising a cationic group covalently bonded to an anionic group, called “inner salt” or “internal salt”. It should be understood that all acid, salt, base, and other ionic and non-ionic forms of compounds described herein are intended to be encompassed. For example, if a compound is shown as an acid herein, the salt forms of the compound are also encompassed. Likewise, if a compound is shown as a salt, the acid and/or basic forms are also encompassed.

### Compounds

As used herein, the term “compounds of the disclosure” and equivalent expressions refers to KRAS inhibitor compounds provided herein as being useful for at least one purpose of the disclosure, e.g., those encompassed by structural Formula (I), (I-a), (I-b), (II-a), (II-b), (III-a), (III-b), (IV-a), (IV-b), (IV-c), (V) and (VI), and includes specific compounds mentioned herein such as those in Tables 1 and 2 as well as their pharmaceutically acceptable salts, esters, hydrates, solvates and stereoisomers.

As would be understood by a person of ordinary skill in the art, the recitation of “a compound” is intended to include salts, esters, solvates, hydrates, oxides, and inclusion complexes of that compound as well as any stereoisomeric form or polymorphic form, or a mixture of any such forms of that compound in any ratio. Thus, in accordance with some embodiments, a compound as described herein, including in the contexts of pharmaceutical compositions and methods of treatment, is provided as the salt form.

It should be understood that compounds described herein may contain one or more chiral centers and/or double bonds and therefore, may exist as stereoisomers, such as double-bond isomers (i.e., geometric isomers), enantiomers, or diastereomers. Chemical structures disclosed herein are intended to encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan, e.g., chiral chromatography (such as chiral HPLC), immunoassay techniques, or the use of covalently (such as Mosher's esters) and non-covalently (such as chiral salts) bound chiral reagents to respectively form a diastereomeric mixture which can be separated by conventional methods, such as chromatography, distillation, crystallization or sublimation, the chiral salt or ester is then exchanged or cleaved by conventional means, to recover the desired isomers. The compounds may also exist in several tautomeric forms including the enol form, the keto form, and mixtures thereof. The chemical structures depicted herein are also intended to encompass all possible tautomeric forms of the illustrated compounds.

Compounds may exist in unsolvated forms as well as solvated forms, including hydrated forms. In general, compounds may be hydrated or solvated. Certain compounds may exist in multiple crystalline or amorphous forms. In general, all physical forms are intended to be encompassed herein.

Compounds described herein include, but are not limited to, their optical isomers, racemates, and other mixtures thereof. In those situations, the single enantiomers or diastereomer, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. In addition, such compounds include Z- and E-forms (or cis- and trans-forms) of compounds with carbon-carbon double bonds. Where compounds described herein exist in various tautomeric forms, the term “compound” is intended to include all tautomeric forms of the compound. Such compounds also include crystal forms including polymorphs and clathrates. Similarly, the term “salt” is intended to include all tautomeric forms and crystal forms of the compound.

The configuration of any carbon-carbon double bond appearing herein is selected for convenience only and is not intended to designate a particular configuration; thus a carbon-carbon double bond depicted arbitrarily herein as E may be Z, E, or a mixture of the two in any proportion.

For compounds provided herein, it is intended that, in some embodiments, salts thereof are also encompassed, including pharmaceutically acceptable salts. Those skilled in the art will appreciate that many salt forms (e.g., TFA salt, tetrazolium salt, sodium salt, potassium salt, etc) are possible; appropriate salts are selected based on considerations known in the art. The term “pharmaceutically acceptable salt” refers to salts prepared from pharmaceutically acceptable non-toxic acids or bases including inorganic acids and bases and organic acids and bases. For example, for compounds that contain a basic nitrogen, salts may be prepared from pharmaceutically acceptable non-toxic acids including inorganic and organic acids. Suitable pharmaceutically acceptable acid addition salts for the compounds of the present disclosure include without limitation acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. When the compounds contain an acidic side chain, suitable pharmaceutically acceptable base addition salts for the compounds of the present disclosure include without limitation metallic salts made from aluminum, calcium, lithium, magnesium, potassium, sodium and zinc or organic salts made from lysine, N, N′-dibenzylethylenediamine, chloroprocaine, choline, diethanolamine, ethylenediamine, meglumine (N-methylglucamine), and procaine.

For compounds provided herein, it is intended that, in some embodiments, compounds may contain unnatural proportions of atomic isotopes at one or more of the atoms that constitute such compounds. Unnatural proportions of an isotope may be defined as ranging from the amount found in nature to an amount consisting of 100% of the atom in question. For example, compounds may incorporate radioactive isotopes, such as for example tritium (³H), iodine-125 (¹²⁵I) or carbon-14 (¹⁴C), or non-radioactive isotopes, such as deuterium (²H) or carbon-13 (¹³C). Such isotopic variations can provide additional utilities to those described elsewhere within this application. For instance, isotopic variants of the compounds of the disclosure may find additional utility, including but not limited to, as diagnostic and/or imaging reagents, or as cytotoxic/radiotoxic therapeutic agents. Additionally, isotopic variants can have altered pharmacokinetic and pharmacodynamic characteristics which can contribute to enhanced safety, tolerability or efficacy during treatment. All isotopic variations of compounds provided herein, whether radioactive or not, are intended to be encompassed herein.

Isotopic enrichment is a process by which the relative abundance of the isotopes of a given element are altered, thus producing a form of the element that has been enriched (i.e., increased) in one particular isotope and reduced or depleted in its other isotopic forms. As used herein, an “isotope-enriched” compound or derivative refers to a compound in which one or more specific isotopic form has been increased, i.e., one or more of the elements has been enriched (i.e., increased) in one or more particular isotope. Generally, in an isotope-enriched compound or derivative, a specific isotopic form of an element at a specific position of the compound is increased. It should be understood however that isotopic forms of two or more elements in the compound may be increased. Further, an isotope-enriched compound may be a mixture of isotope-enriched forms that are enriched for more than one particular isotope, more than one element, or both. As used herein, an “isotope-enriched” compound or derivative possesses a level of an isotopic form that is higher than the natural abundance of that form. The level of isotope-enrichment will vary depending on the natural abundance of a specific isotopic form. In some embodiments, the level of isotope-enrichment for a compound, or for an element in a compound, may be from about 2 to about 100 molar percent (%), e.g., about 2%, about 5%, about 17%, about 30%, about 51%, about 83%, about 90%, about 95%, about 96%, about 97%, about 98%, greater than about 98%, about 99%, or 100%.

As used herein, an “element of natural abundance” and an “atom of natural abundance” refers to the element or atom respectively having the atomic mass most abundantly found in nature. For example, hydrogen of natural abundance is ¹H (protium); nitrogen of natural abundance is ¹⁴N; oxygen of natural abundance is ¹⁶O; carbon of natural abundance is ¹²C; and so on. A “non-isotope enriched” compound is a compound in which all the atoms or elements in the compound are isotopes of natural abundance, i.e., all the atoms or elements have the atomic mass most abundantly found in nature.

In some embodiments of compounds of the disclosure, one or more C, H, O, and/or N atoms in the compound are each independently selected from atoms of natural abundance and isotope-enriched atoms. Examples of isotopes of natural abundance include ¹²C, ¹H, ¹⁶O and ¹⁴N. Examples of isotope-enriched atoms include, without limitation, ¹³C and ¹⁴C for carbon; ²H (D) and ³H (T) for hydrogen; ¹⁷O and ¹⁸O for oxygen; and ¹⁵N for nitrogen. In some embodiments of compounds of the disclosure, all the elements or atoms in a compound are isotopes of natural abundance. In other embodiments, one or more elements or atoms in a compound are isotope-enriched.

### Compositions

In certain embodiments, there are provided pharmaceutical compositions comprising a compound of the disclosure, e.g., a compound of Formula (I), Formula (I-a), Formula (I-b), Formula (II-a), Formula (II-b), Formula (III-a), Formula (III-b), Formula (IV-a), Formula (IV-b), Formula (IV-c), (Formula V), or Formula (VI), or a pharmaceutically acceptable salt, ester, hydrate, solvate or stereoisomer thereof, and a pharmaceutically acceptable excipient, carrier or diluent. In an embodiment, there is provided a pharmaceutical composition comprising a compound of Formula (I), Formula (I-a), Formula (I-b), Formula (II-a), Formula (II-b), Formula (III-a), Formula (III-b), Formula (IV-a), Formula (IV-b), Formula (IV-c), (Formula V), or Formula (VI), or a compound in any one of Tables 1 and 2, or a pharmaceutically acceptable salt thereof, and a pharmaceutically acceptable excipient, carrier, or diluent.

The preparation of pharmaceutical compositions can be carried out as known in the art (see, for example, Remington: The Science and Practice of Pharmacy, 20th Edition, 2000). For example, a therapeutic compound and/or composition, together with one or more solid or liquid pharmaceutical carrier substances and/or additives (or auxiliary substances) and, if desired, in combination with other pharmaceutically active compounds having therapeutic or prophylactic action, are brought into a suitable administration form or dosage form which can then be used as a pharmaceutical in human or veterinary medicine. Pharmaceutical preparations can also contain additives, of which many are known in the art, for example fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents or antioxidants.

The term “pharmaceutical composition” means a composition comprising a compound as described herein and at least one component comprising pharmaceutically acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents, dispersants and dispensing agents, depending on the nature of the mode of administration and dosage forms. It should be understood that, as used herein, a pharmaceutical composition comprises a compound disclosed herein (or a pharmaceutically acceptable salt, ester, hydrate, solvate, or stereoisomer thereof) and a pharmaceutically acceptable excipient, carrier, diluent, adjuvant, or vehicle. In certain embodiments, the amount of a compound in a composition is such that it is effective as an inhibitor of KRAS in a biological sample (e.g., in a cellular assay, in an in vivo model, etc.) or in a subject. In certain embodiments, the composition is formulated for administration to a subject in need of such composition. In some embodiments, the composition is an injectable formulation. In other embodiments, the composition is formulated for oral administration to a subject.

The term “pharmaceutically acceptable carrier” is used to mean any carrier, diluent, adjuvant, excipient, or vehicle, as described herein. Examples of suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monosterate and gelatin. Examples of suitable carriers, diluents, solvents, or vehicles include water, ethanol, polyols, suitable mixtures thereof, vegetable oils (such as olive oil), and injectable organic esters such as ethyl oleate. Examples of excipients include lactose, milk sugar, sodium citrate, calcium carbonate, and dicalcium phosphate. Examples of disintegrating agents include starch, alginic acids, and certain complex silicates. Examples of lubricants include magnesium stearate, sodium lauryl sulphate, talc, as well as high molecular weight polyethylene glycols.

A pharmaceutical composition provided herein can be administered orally, for example in the form of pills, tablets, lacquered tablets, sugar-coated tablets, granules, hard and soft gelatin capsules, aqueous, alcoholic or oily solutions, syrups, emulsions or suspensions, or rectally, for example in the form of suppositories. Administration can also be carried out parenterally, for example subcutaneously, intramuscularly or intravenously in the form of solutions for injection or infusion. Other suitable administration forms are, for example, percutaneous or topical administration, for example in the form of ointments, creams, tinctures, sprays or transdermal therapeutic systems, or the inhalative administration in the form of nasal sprays or aerosol mixtures, or, for example, microcapsules, implants or wafers.

In some embodiments, pharmaceutical compositions provided herein are suitable for oral administration. For example, a pharmaceutical composition may be in the form of a hard shell gelatin capsule, a soft shell gelatin capsule, a cachet, a pill, a tablet, a lozenge, a powder, a granule, a pellet, a pastille, or a dragee. Alternatively, a pharmaceutical composition may be in the form of a solution, an aqueous liquid suspension, a non-aqueous liquid suspension, an oil-in-water liquid emulsion, a water-in-oil liquid emulsion, an elixir, or a syrup. Pharmaceutical compositions may or may not be enteric coated. In some embodiments, pharmaceutical compositions are formulated for controlled release, such as delayed or extended release.

In further embodiments, compounds and compositions thereof may be formulated in multi-dose forms, i.e., in the form of multi-particulate dosage forms (e.g., hard gelatin capsules or conventional tablets prepared using a rotary tablet press) comprising one or more bead or minitab populations for oral administration. The conventional tablets rapidly disperse on entry into the stomach. The one or more coated bead or minitab populations may be compressed together with appropriate excipients into tablets (for example, a binder, a diluent/filler, and a disintegrant for conventional tablets.

Tablets, pills, beads, or minitabs of the compounds and compositions of the compounds may be coated or otherwise compounded to provide a dosage form affording the advantage of controlled release, including delayed or extended release, or to protect from the acid conditions of the stomach. For example, the tablet or pill can include an inner dosage and an outer dosage component, the latter being in the form of a coating over the former. The two components can be separated by a polymer layer that controls the release of the inner dosage.

In certain embodiments, the layer may comprise at least one enteric polymer. In further embodiments, the layer may comprise at least one enteric polymer in combination with at least one water-insoluble polymer. In still further embodiments, the layer may comprise at least one enteric polymer in combination with at least one water-soluble polymer. In yet further embodiments, the layer may comprise at least one enteric polymer in combination with a pore-former.

In certain embodiments, the layer may comprise at least one water-insoluble polymer. In still further embodiments, the layer may comprise at least one water-insoluble polymer in combination with at least one water-soluble polymer. In yet further embodiments, the layer may comprise at least one water-insoluble polymer in combination with a pore-former.

Representative examples of water-soluble polymers include polyvinylpyrrolidone (PVP), hydroxypropyl methylcellulose (HPMC), hydroxypropylcellulose (HPC), polyethylene glycol, and the like.

Representative examples of enteric polymers include esters of cellulose and its derivatives (cellulose acetate phthalate, hydroxypropyl methylcellulose phthalate, hydroxypropyl methylcellulose acetate succinate), polyvinyl acetate phthalate, pH-sensitive methacrylic acid-methylmethacrylate copolymers and shellac. These polymers may be used as a dry powder or an aqueous dispersion. Some commercially available materials that may be used are methacrylic acid copolymers sold under the trademark Eudragit (LI 00, S I 00, L30D) manufactured by Rohm Pharma, Cellacefate (cellulose acetate phthalate) from Eastman Chemical Co., Aquateric (cellulose acetate phthalate aqueous dispersion) from FMC Corp. and Aqoat (hydroxypropyl methylcellulose acetate succinate aqueous dispersion) from Shin Etsu K.K.

Representative examples of useful water-insoluble polymers include ethylcellulose, polyvinyl acetate (for example, Kollicoat SR #30D from BASF), cellulose acetate, cellulose acetate butyrate, neutral copolymers based on ethyl acrylate and methylmethacrylate, copolymers of acrylic and methacrylic acid esters with quaternary ammonium groups such as Eudragit NE, RS and RS30D, RL or RL30D and the like.

Any of the above polymers may be further plasticized with one or more pharmaceutically acceptable plasticizers. Representative examples of plasticizers include triacetin, tributyl citrate, triethyl citrate, acetyl tri-n-butyl citrate diethyl phthalate, castor oil, dibutyl sebacate, acetylated monoglycerides and the like or mixtures thereof. The plasticizer, when used, may comprise about 3 to 30 wt. % and more typically about 10 to 25 wt. % based on the polymer. The type of plasticizer and its content depends on the polymer or polymers and nature of the coating system (e.g., aqueous or solvent based, solution or dispersion based and the total solids).

Pharmaceutical compositions typically must be sterile and stable under the conditions of manufacture and storage. A composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of injectable compositions can be brought about by including in the composition an agent which delays absorption, for example, monostearate salts and gelatin. Moreover, a compound can be administered in a time release formulation, for example in a composition which includes a slow release polymer. The compound can be prepared with carriers that will protect against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG).

Pharmaceutical compositions can also include carriers to protect the composition against rapid degradation or elimination from the body, such as a controlled release formulation, including liposomes, hydrogels, and microencapsulated delivery systems. For example, a time delay material such as glyceryl monostearate or glyceryl stearate alone, or in combination with a wax, may be employed. Any drug delivery apparatus may be used to deliver compounds and compositions of the disclosure, including implants (e.g., implantable pumps) and catheter systems, slow injection pumps and devices, all of which are well known to the skilled artisan.

Pharmaceutical compositions may also be in the form of a sterile injectable aqueous or oleagenous (oily) suspension. This suspension may be formulated according to the known art using those suitable dispersing or wetting agents and suspending agents mentioned herein. The sterile injectable preparation may also be a sterile injectable solution or suspension in a non-toxic parenterally acceptable diluent or solvent, for example, as a solution in 1,3-butane diol. Acceptable diluents, solvents and dispersion media that may be employed include water, Ringer's solution, isotonic sodium chloride solution, Cremophor EL™ (BASF, Parsippany, NJ) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose any bland fixed oil may be employed, including synthetic mono- or diglycerides. Moreover, fatty acids such as oleic acid, can be used in the preparation of injectables. Prolonged absorption of particular injectable formulations can be achieved by including an agent that delays absorption (e.g., aluminum monostearate or gelatin).

Many methods for the preparation of such formulations are generally known to those skilled in the art. Sterile injectable solutions can be prepared by incorporating an active compound, such as a compound of Formula (A) provided herein, in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, common methods of preparation are vacuum drying and freeze-drying which yields a powder of the active ingredient plus any additional desired ingredient from a previously sterile-filtered solution thereof. Compounds may also be formulated with one or more additional compounds that enhance their solubility.

It is often advantageous to formulate compositions (such as parenteral compositions) in dosage unit form for ease of administration and uniformity of dosage. The term “unit dosage form” refers to a physically discrete unit suitable as unitary dosages for human subjects and other animals, each unit containing a predetermined quantity of active material calculated to produce the desired therapeutic effect, in association with a suitable pharmaceutical carrier. The specification for the dosage unit forms of the disclosure may vary and are dictated by and directly dependent on (a) the unique characteristics of the therapeutic compound and the particular therapeutic effect to be achieved, and (b) the limitations inherent in the art of compounding such a therapeutic compound for the prevention or treatment of a KRAS-associated disease, disorder or condition, such as a hyperplastic disease, e.g., a cancer or a tumor.

In some embodiments, the pharmaceutical composition is provided in a single-use container (e.g., a single-use vial, ampoule, syringe, or autoinjector), whereas a multi-use container (e.g., a multi-use vial) is provided in other embodiments.

In some embodiments, the pharmaceutical composition is provided in a disposable container, such as a disposable vial, ampoule, syringe, or auto-injector. In other embodiments, the pharmaceutical composition is provided in a repeatedly usable container, such as repeatedly usable vial.

Pharmaceutical compositions provided herein can be formulated to be compatible with the intended method or route of administration; exemplary routes of administration are set forth herein. Furthermore, the pharmaceutical compositions may be used in combination with other therapeutically active agents or compounds as described herein in order to treat or prevent the KRAS-associated diseases, disorders and conditions as contemplated herein.

Pharmaceutical compositions containing the active ingredient (e.g., a KRAS inhibitor compound) may be in a form suitable for oral use, for example, as tablets, capsules, troches, lozenges, aqueous or oily suspensions, dispersible powders or granules, emulsions, hard or soft capsules, or syrups, solutions, beads, microbeads or elixirs. Pharmaceutical compositions intended for oral use may be prepared according to any method known in the art for the manufacture of pharmaceutical compositions, and such compositions may contain one or more agents such as, for example, sweetening agents, flavoring agents, coloring agents and preserving agents in order to provide pharmaceutically acceptable preparations. Tablets, capsules and the like generally contain the active ingredient in admixture with non-toxic pharmaceutically acceptable carriers or excipients which are suitable for the manufacture of tablets. These carriers or excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin, gum arabic or acacia, and lubricating agents, for example magnesium stearate, stearic acid or talc.

Tablets, capsules and the like suitable for oral administration may be uncoated or coated using known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylenevinylacetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly (methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil. Aqueous suspensions contain the active materials in admixture with excipients suitable for the manufacture thereof. Such excipients can be suspending agents, for example sodium carboxymethylcellulose, methykellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are known in the art.

Pharmaceutical compositions of the present disclosure may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soybean, lecithin, and esters or partial esters derived from fatty acids; hexitol anhydrides, for example, sorbitan monooleate; and condensation products of partial esters with ethylene oxide, for example, polyoxyethylene sorbitan monooleate.

Pharmaceutical compositions typically comprise a therapeutically effective amount of a KRAS inhibitor compound provided herein and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bi sulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-MoqJholino)ethanesulfonic acid (MES), 2-(N-Morpholino)ethanesulfonic acid sodium salt (MES), 3-(N-Morpholino)propanesulfonic acid (MOPS), and Ntris[Hydroxymethyl]methyl-3-aminopropanesulfonic acid (TAPS). After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form.

In some embodiments, there are provided pharmaceutical compositions that comprise an effective amount of a compound and/or composition described herein, and a pharmaceutically acceptable excipient, carrier or diluent. In an embodiment, there are provided pharmaceutical compositions for the treatment or prevention of a KRAS-associated disease, disorder or condition, such as a cancer or a tumor, comprising a compound described herein, or a pharmaceutically acceptable salt, ester, hydrate, solvate or stereoisomer thereof, and a pharmaceutically acceptable carrier. In another embodiment, there is provided a pharmaceutical composition for the prevention or treatment of a KRAS-associated disease, disorder or condition, such as a cancer or a tumor, the composition comprising a compound described herein, or a pharmaceutically acceptable salt thereof, and a pharmaceutically acceptable carrier.

### Methods of Use of Compounds and Compositions

In certain embodiments, there are provided methods for inhibition, prevention or treatment of a KRAS-associated disease, disorder or condition in a subject by administering an effective amount of a compound or composition described herein. In a related aspect, there are provided methods for prevention or treatment of a KRAS-associated hyperplastic or hyperproliferative disorder, e.g., a cancer or a tumor, in a subject in need thereof by administering an effective amount of a compound or composition described herein.

In an embodiment, there is provided herein a method of treating a subject (e.g., a human) with cancer or a disorder mediated by KRAS comprising the step of administering to the subject a therapeutically effective amount of an KRAS inhibitor compound provided herein or a pharmaceutically acceptable composition thereof.

There is also provided a method of treating a subject (e.g., a human) with cancer or a hyperproliferative disorder mediated by KRAS (e.g., by a KRAS mutation) comprising the step of administering to the subject a therapeutically effective amount of a compound provided herein, e.g., a compound provided herein or a pharmaceutically acceptable composition thereof. In certain embodiments, the amount of a compound in a composition is such that it is effective as an inhibitor of KRAS in a biological sample (e.g., in a cellular assay, in an in vivo model, etc.) or in a subject. In certain embodiments, the composition is formulated for administration to a subject in need of such composition. In some embodiments, the composition is an injectable formulation. In some embodiments, the composition is formulated for intravenous administration. In other embodiments, the composition is formulated for oral administration to a subject. In some embodiments, the composition is in the form of a hard shell gelatin capsule, a soft shell gelatin capsule, a cachet, a pill, a tablet, a lozenge, a powder, a granule, a pellet, a pastille, or a dragee. In some embodiments, the composition is in the form of a solution, an aqueous liquid suspension, a non-aqueous liquid suspension, an oil-in-water liquid emulsion, a water-in-oil liquid emulsion, an elixir, or a syrup. In some embodiments, the composition is enteric coated. In some embodiments, the composition is formulated for controlled release.

In further embodiments, there are provided methods for treating or preventing cancer in a subject, comprising administering to the subject a therapeutically effective amount of at least one compound of the disclosure and at least one additional signal transduction inhibitor (STI). In a particular embodiment, the at least one STI is selected from the group consisting of bcr/abl kinase inhibitors, epidermal growth factor (EGF) receptor inhibitors, her-2/neu receptor inhibitors, and farnesyl transferase inhibitors (FTIs). There are also provided methods of augmenting the rejection of tumor cells in a subject comprising administering a compound of the disclosure in conjunction with at least one chemotherapeutic agent and/or radiation therapy, wherein the resulting rejection of tumor cells is greater than that obtained by administering either the compound, the chemotherapeutic agent or the radiation therapy alone. In further embodiments, there are provided methods for treating cancer in a subject, comprising administering to the subject a therapeutically effective amount of at least one compound of the disclosure and at least one immunomodulator.

In further embodiments, there are provided methods for treating, inhibiting or preventing a hyperproliferative or hyperplastic disease or disorder in a subject, comprising administering to the subject an effective amount of at least one compound or pharmaceutical composition of the disclosure.

The terms “patient” and “subject” are used interchangeably herein to refer to a human or a non-human animal (e.g., a mammal). Non-limiting examples of subjects include humans, monkeys, cows, rabbits, sheep, goats, pigs, dogs, cats, rats, mice, and transgenic species thereof. In some embodiments, a subject is in need of treatment by the methods provided herein, and is selected for treatment based on this need. A subject in need of treatment is art-recognized, and includes subjects that have been identified as having a disease or condition (e.g., cancer, tumor, hyperproliferative disorder), or having a symptom of such a disease or condition, or being at risk of such a disease or condition, and would be expected, based on diagnosis, e.g., medical diagnosis, to benefit from treatment (e.g., curing, healing, preventing, alleviating, relieving, altering, remedying, ameliorating, improving, or affecting the disease or disorder, the symptom of the disease or disorder, or the risk of the disease or disorder). In certain embodiments, a subject is a human. In some embodiments, a subject has a cancer or tumor carrying a KRAS mutation.

The term “in need of treatment” as used herein refers to a judgment made by a physician or other caregiver that a subject requires or will benefit from treatment. This judgment is made based on a variety of factors that are in the realm of the physician's or caregiver's expertise.

The terms “administration”, “administer” and the like, as they apply to, for example, a subject, cell, tissue, organ, or biological fluid, refer to contact of, for example, an inhibitor of KRAS (wild-type or mutated), a pharmaceutical composition comprising same, or a diagnostic agent to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

The terms “treat”, “treating”, “treatment” and the like refer to a course of action (such as administering an inhibitor of KRAS (wild-type or mutated) or a pharmaceutical composition comprising same) initiated after a disease, disorder or condition, or a symptom thereof, has been diagnosed, observed, and the like, so as to eliminate, alleviate, reduce, suppress, mitigate, improve, or ameliorate, either temporarily or permanently, at least one of the underlying causes of a disease, disorder, or condition afflicting a subject, or at least one of the symptoms associated with a disease, disorder, condition afflicting a subject. Thus, treatment includes inhibiting (e.g., arresting the development or further development of the disease, disorder or condition or clinical symptoms association therewith) an active disease. Specifically, the term “treatment”, as used in the present application, means that a therapeutic substance including a compound or composition according to the present disclosure is administered to a patient in need thereof. In certain embodiments, the term “treatment” also relates to the use of a compound or composition according to the present disclosure, optionally in combination with one or more anticancer agents, to alleviate one or more symptoms associated with KRAS (e.g., with wild-type or a KRAS mutation), to slow down the development of one or more symptoms related to KRAS (wild-type or mutated), to reduce the severity of one or more symptoms related to KRAS (wild-type or mutated), to inhibit the clinical manifestations related to KRAS mutation, and/or to inhibit the expression of adverse symptoms associated with the KRAS mutation. In certain embodiments, “treating” any disease or condition means alleviating the disease or condition; treating may refer to physical (e.g., stabilization of distinguishable symptom) or physiological (e.g., stabilization of a physical parameter), inhibition of a disease or condition, or both. In certain embodiments, “treatment” refers to improving the quality of life or side effects of the disease in a subject in need.

The terms “prevent”, “preventing”, “prevention”, “prophylaxis” and the like refer to a course of action (such as administering a KRAS inhibitor or a pharmaceutical composition comprising same) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof: generally in the context of a subject predisposed to having a particular disease, disorder or condition. In certain instances, the terms also refer to slowing the progression of the disease, disorder or condition or inhibiting progression thereof to a harmful or otherwise undesired state. Specifically, the term “prevention”, as used in the present application, means that a therapeutic substance including a compound or composition according to the present disclosure is administered to a subject to prevent the occurrence of diseases related to a KRAS mutation, e.g., to prevent the clinical symptoms of at least one disease from developing in patients who may be exposed to or at risk of the disease but have not yet experienced or displayed symptoms of the disease.

The term “in need of prevention” as used herein refers to a judgment made by a physician or other caregiver that a subject requires or will benefit from preventative care. This judgment is made based on a variety of factors that are in the realm of a physician's or caregiver's expertise.

The terms “therapeutically effective amount” and “effective amount” are used interchangeably herein to refer to the administration of an agent to a subject, either alone or as part of a pharmaceutical composition and either in a single dose or as part of a series of doses, in an amount capable of having any detectable, positive effect on any symptom, aspect, or characteristic of a disease, disorder or condition when administered to the subject. The therapeutically effective amount can be ascertained by measuring relevant physiological effects, and it can be adjusted in connection with the dosing regimen and diagnostic analysis of the subject's condition, and the like. By way of example, measurement of the serum level of a KRAS inhibitor (or, e.g., a metabolite thereof) at a particular time post-administration may be indicative of whether a therapeutically effective amount has been used. In some embodiments, the terms “therapeutically effective amount” and “effective amount” refer to the amount or dose of a therapeutic agent, such as a compound, upon single or multiple dose administration to a subject, which provides the desired therapeutic, diagnostic, or prognostic effect in the subject. An effective amount can be readily determined by an attending physician or diagnostician using known techniques and by observing results obtained under analogous circumstances. In determining the effective amount or dose of compound administered, a number of factors are considered including, but not limited to: the size, age, and general health of the subject; the specific disease involved; the degree of or involvement or the severity of the disease or condition to be treated; the response of the individual subject; the particular compound administered; the mode of administration; the bioavailability characteristics of the preparation administered; the dose regimen selected; the use of concomitant medication(s); and other relevant considerations.

The term “substantially pure” is used herein to indicate that a component makes up greater than about 50% of the total content of the composition, and typically greater than about 60% of the total content. More typically, “substantially pure” refers to compositions in which at least 75′%, at least 85%), at least 90% or more of the total composition is the component of interest. In some cases, the component of interest will make up greater than about 90%), or greater than about 95%) of the total content of the composition.

As used herein, the terms “KRAS-associated disease, disorder or condition” and “disease, disorder or condition mediated by KRAS” and “KRAS-related disease” are used interchangeably to refer to any disease, disorder or condition for which a KRAS mutation is known to play a role, and/or for which treatment with a KRAS inhibitor may be beneficial. A KRAS-associated disease, disorder or condition may be associated with or mediated by, for example and without limitation, the KRAS G12A, KRAS G12C, KRAS G12D, KRAS G12R, KRAS G12S, KRAS G12V, KRAS G13D and/or KRAS Q61H mutation, or the wild-type KRAS. In general, KRAS-associated or mediated diseases, disorders and conditions are those in which KRAS activity plays a biological, mechanistic, or pathological role. Non-limiting examples of KRAS-associated diseases, disorders and conditions include oncology-related disorders (cancers, tumors, etc.), including hyperproliferative disorders, hyperplastic diseases, and malignant tumors, such as without limitation lung cancer, non-small cell lung cancer (NSCLC), pancreatic cancer, colorectal cancer, colon cancer, cholangiocarcinoma, cervical cancer, bladder cancer, liver cancer or breast cancer. For example, a KRAS inhibitor (i.e., a compound or composition of the disclosure) may be used to prevent or treat a proliferative condition, cancer or tumor.

In some embodiments, a KRAS inhibitor is used to prevent or treat one or more of non-small cell lung cancer, pancreatic cancer, colorectal cancer, bile duct cancer, cervical cancer, bladder cancer, liver cancer and breast cancer.

In certain embodiments, a KRAS inhibitor is used to prevent or treat an immune-related and/or an inflammatory disease, disorder or condition in a subject.

KRAS inhibitor compounds and compositions provided herein may be administered to a subject in any appropriate manner known in the art. Suitable routes of administration include, without limitation: oral, parenteral (e.g., intramuscular, intravenous, subcutaneous (e.g., injection or implantation), intraperitoneal, intracisternal, intraarticular, intracerebral (intraparenchymal, intraventricular, and intracerebroventricular), extra-gastrointestinal, nasal, vaginal, sublingual, intraocular, rectal, topical (e.g., transdermal), buccal and inhalation. Depot injections, which are generally administered subcutaneously or intramuscularly, may also be utilized to release the KRAS inhibitors disclosed herein over a defined period of time. In certain embodiments, KRAS inhibitor compounds and compositions are administered orally to a subject in need thereof. In certain embodiments, KRAS inhibitor compounds and compositions are administered intravenously to a subject in need thereof.

KRAS inhibitor compounds and compositions provided herein may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan. In general, dosing parameters dictate that the dosage amount be less than an amount that could be irreversibly toxic to the subject (the maximum tolerated dose (MTD)) and not less than an amount required to produce a measurable effect on the subject. Such amounts are determined by, for example, the pharmacokinetic and pharmacodynamic parameters associated with ADME, taking into consideration the route of administration and other factors.

In some embodiments, an KRAS inhibitor may be administered (e.g., orally) at dosage levels of about 0.01 mg/kg to about 50 mg/kg, or about 1 mg/kg to about 25 mg/kg, of subject body weight per day, one or more times a day, to obtain the desired therapeutic effect. For administration of an oral agent, the compositions can be provided in the form of tablets, capsules and the like containing from 1.0 to 1000 milligrams of the active ingredient, particularly 1, 3, 5, 10, 15, 20, 25, 50, 75, 100, 150, 200, 250, 300, 400, 500, 600, 750, 800, 900, or 1000 milligrams of the active ingredient.

In some embodiments, the dosage of the desired KRAS inhibitor is contained in a “unit dosage form”. The phrase “unit dosage form” refers to physically discrete units, each unit containing a predetem1ined amount of the KRAS inhibitor, either alone or in combination with one or more additional agents, sufficient to produce the desired effect. It will be appreciated that the parameters of a unit dosage form will depend on the particular agent(s) and the effect to be achieved.

### Kits

There are also provided herein kits comprising a KRAS inhibitor compound or composition of the disclosure. Kits are generally in the form of a physical structure housing various components and may be used, for example, in practicing the methods provided herein. For example, a kit may include one or more KRAS inhibitor disclosed herein (provided in, e.g., a sterile container), which may be in the form of a pharmaceutical composition suitable for administration to a subject. The KRAS inhibitor can be provided in a form that is ready for use (e.g., a tablet or capsule) or in a form requiring, for example, reconstitution or dilution (e.g., a powder) prior to administration. When the KRAS inhibitors are in a form that needs to be reconstituted or diluted by a user, the kit may also include diluents (e.g., sterile water), buffers, pharmaceutically acceptable excipients, and the like, packaged with or separately from the KRAS inhibitors. When combination therapy is contemplated, the kit may contain several therapeutic agents separately or they may already be combined in the kit. Each component of the kit may be enclosed within an individual container, and all of the various containers may be within a single package. A kit of the present disclosure may be designed for conditions necessary to properly maintain the components housed therein (e.g., refrigeration or freezing).

A kit may also contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial).

For a better understanding of the present invention and to demonstrate its implementation more clearly, the features of the embodiments according to the present disclosure are described in detail through the examples.

### EXAMPLES

The present invention will be more readily understood by referring to the following examples, which are provided to illustrate the invention and are not to be construed as limiting the scope thereof in any manner.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the invention. Unless otherwise stated, the materials and instruments used in this invention are commercially available.

### Compound Synthesis

### Synthesis of Compound 1

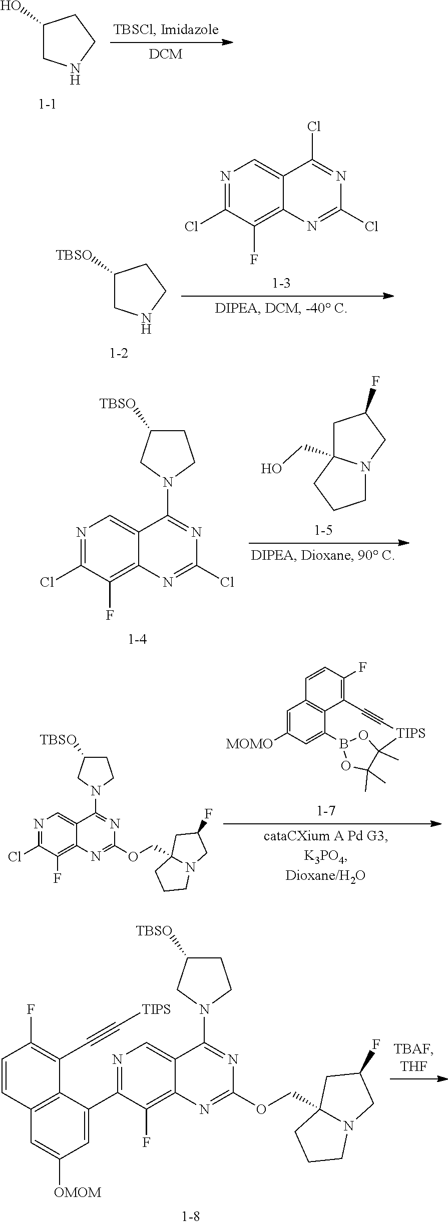

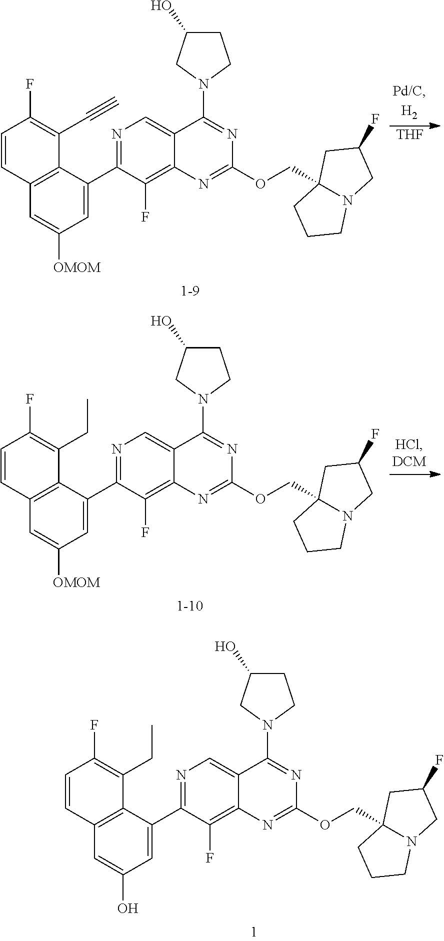

To a stirred solution of intermediate 1-1 (500 mg, 5.74 mmol, 1 eq) in dichloromethane (10 mL), imidazole (585.40 mg, 8.61 mmol, 1.5 eq) and tert-Butyldimethylsilyl chloride (708.93 mg, 8.61 mmol, 1.5 eq) were added subsequently. The reaction mixture was stirred overnight. Upon the completion of conversion, the solvent was removed in vacuo, water was poured onto the residue and extracted with ethyl acetate. The organic layer was washed with water and brine, dried over sodium sulfate and then filtered. The filtrate was concentrated in vacuo to afford crude product, which was subjected to silica gel chromatography eluted with dichloromethane/methanol (v/v=30/1) to give purified compound 1-2 (230 mg, Yield 19.90%).

The suspension of intermediate 1-3 (200 mg, 792.20 μmol, 1 eq) in dichloromethane (2 mL) was purged with nitrogen gas and cooled to −40° C. Intermediate 1-2 (223.35 mg, 1.11 mmol, 1.4 eq) and Diisopropylethylamine (307.15 mg, 2.38 mmol, 413.95 μL, 3 eq) were added subsequently. The reaction mixture was stirred at −40° C. for 1 hour then was gradually warmed up to room temperature. The solvent was removed in vacuo to afford crude residue, which was subjected to silica gel chromatography eluted with Petroleum Ether/Ethyl Acetate (v/v=10/1) to afford compound 1-4 (280 mg, Yield 84.68%).

To a stirred solution of intermediate 1-4 (280 mg, 670.85 μmol, 1 eq) in 1,4-dioxane (5 mL), intermediate 1-5 (160.20 mg, 1.01 mmol, 1.5 eq) and Diisopropylethylamine (173.40 mg, 1.34 mmol, 233.70 μL, 2 eq) were added subsequently. The reaction mixture was warmed to 90° C. and stirred overnight. Upon the completion of conversion, the reaction mixture was cooled to room temperature and the solvent was removed in vacuo to afford crude product, which was subjected to silica gel chromatography eluted with dichloromethane/methanol (v/v=20/1) to give purified compound 1-6 (180 mg, Yield 49.68%).

Compound 1-7 (247.22 mg, 499.89 μmol, 1.5 eq) was added to a stirred solution of compound 1-6 (180 mg, 333.26 μmol, 1 eq) in dioxane/water (5 mL/2 mL). Potassium phosphate (212.21 mg, 999.78 μmol, 3 eq) and cataCXium APd G3 (48.54 mg, 66.65 μmol, 0.2 eq) were added subsequently. The reaction mixture was purged with nitrogen gas three times, then warmed to 100° C. and kept on stirring for 3 h. Upon the completion, the reaction mixture was cooled to room temperature, diluted with water and extracted with ethyl acetate. The organic layer was washed with water and brine, dried over sodium sulfate and then filtered. The filtrate was concentrated in vacuo to afford crude residue, which was subjected to silica gel column chromatography eluted with dichloromethane/methanol (v/v=30/1) to afford compound 1-8 (230 mg, Yield 77.53%).

To a stirred solution of intermediate 1-8 (230 mg, 258.36 μmol, 1 eq) in tetrahydrofuran (5 mL), tetrabutylammonium fluoride (160.20 mg, 1.01 mmol, 1.5 eq) were added in one portion. The reaction mixture was stirred 1 hour at room temperature. Upon the completion of conversion, the reaction mixture was cooled to room temperature and the solvent was removed in vacuo to afford crude residue, which was subjected to silica gel chromatography eluted with dichloromethane/methanol (v/v=30/1) to obtain purified compound 1-9 (140 mg, Yield 87.45%).

To a round bottom flask, intermediate 1-9 (140 mg, 225.94 μmol, 1 eq) was dissolved in tetrahydrofuran (5 mL). Palladium 10% on carbon (14 mg) was added in one portion. The reaction mixture was purged with hydrogen gas and stirred for 24 hours at room temperature. Upon the completion of conversion, the reaction mixture was filtered, and the filtrate was concentrated in vacuo to afford compound 1-10, which was directly subjected to the next transformation without further purification (130 mg, Yield 92.30%).

To a stirred solution of intermediate 1-10 (60 mg, 96.21 μmol, 1 eq) in dichloromethane (2 mL), hydrogen chloride solution 4.0 M in dioxane (196.2 μL, 384.82 μmol, 4 eq) was added in one portion. The reaction mixture was stirred 10 minutes at room temperature. Upon the completion of conversion, the solvent was removed in vacuo to afford crude residue, which was subjected to a reverse phase preparative HPLC eluted with 0.05% ammonia hydroxide aqueous solution/acetonitrile to give the compound 1 (30 mg, Yield 52.92%). ¹H NMR (500 MHz, CD₃OD) δ ppm 0.88 (dd, J=18.0, 7.3 Hz, 3H), 1.83-2.55 (m, 11H), 3.01 (dd, J=14.5, 9.3 Hz, 1H), 3.15-3.28 (m, 3H), 3.96-4.16 (m, 3H), 4.35 (d, J=10.0 Hz, 1H), 4.25 (d, J=10.4 Hz, 1H), 4.62 (s, 1H), 5.32 (s, 0.5H), 5.36 (s, 0.5H), 7.06 (d, J=10.7 Hz, 1H), 7.25 (t, J=9.4 Hz, 1H), 7.30 (d, J=2.1 Hz, 1H), 7.67 (dd, J=8.8, 5.8 Hz, 1H), 9.26 (s, 1H). m/z (ESI): 580.4 [M+H]⁺.

### Synthesis of Compound 2

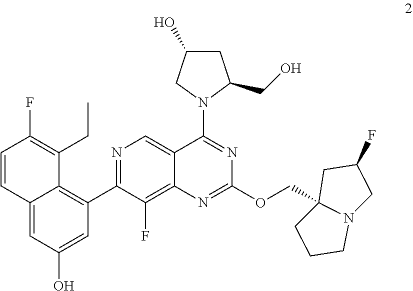 [SMILES: CCc1c(F)ccc2cc(O)cc(-c3ncc4c(N5C[C@H](O)C[C@H]5CO)nc(OC[C@@]56CCCN5C[C@H](F)C6)nc4c3F)c12]

Title compound was synthesized from (3R,5S)-5-(hydroxymethyl)pyrrolidin-3-ol in a manner essentially analogous to the synthesis of compound 1. ¹H NMR (500 MHz, CD₃OD) δ ppm 0.80 (dt, J=21.2, 7.2 Hz, 3H), 1.85-2.05 (m, 3H), 2.11-2.56 (m, 7H), 3.03 (dd, J=15.5, 9.5 Hz, 1H), 3.19-3.27 (m, 2H), 3.83 (d, J=10.9 Hz, 1H), 4.02 (d, J=11.2 Hz, 1H), 4.15-4.25 (m, 2H), 4.29 (s, 1H), 4.38 (d, J=10.3 Hz, 1H), 4.62 (d, J=12.0 Hz, 3H), 5.26 (s, 0.5H), 5.37 (s, 0.5H), 7.06 (d, J=16.2 Hz, 1H), 7.25 (t, J=9.2 Hz, 1H), 7.31 (s, 1H), 7.65-7.71 (m, 1H), 9.13 (d, J=10.6 Hz, 1H). m/z (ESI): 610.5 [M+H]⁺

### Synthesis of Compound 3

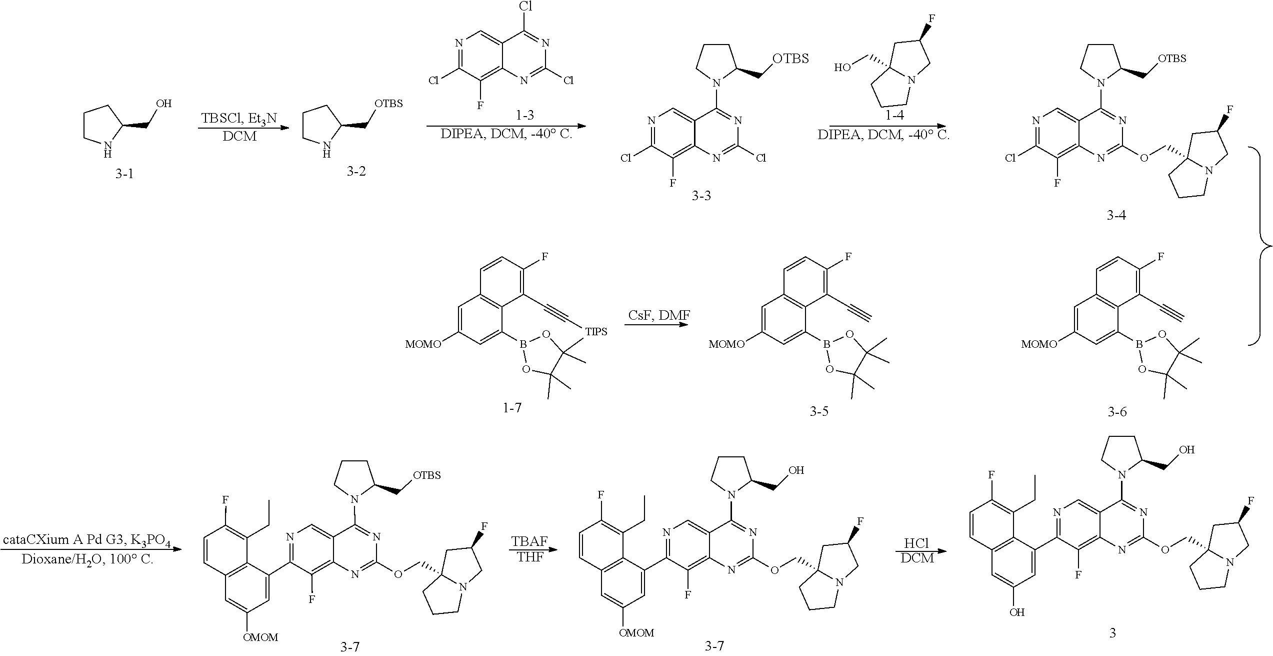

To a stirred suspension of intermediate 3-1 (500 mg, 4.94 mmol, 1 eq) in dichloromethane (5 mL), triethyl amine (700.30 mg, 6.92 mmol, 1.4 eq) and tert-butyldimethylsilyl chloride (1.04 g, 6.92 mmol, 1.4 eq) were added subsequently. The reaction mixture was stirred for 5 hours. Upon the completion of conversion, the reaction was quenched by adding brine. The organic layer was washed with water and brine, dried over sodium sulfate and then filtered. The filtrated was concentrated in vacuo to afford compound 3-2, which was directly subjected to the next transformation without further purification (700 mg, Yield 65.74%).

Intermediate 1-3 (255.97 mg, 1.19 mmol, 1.5 eq) was dissolved in dichloromethane (5 mL), then purged with nitrogen gas and cooled to −40° C. Intermediate 3-2 (200 mg, 792.20 μmol, 1 eq) and Diisopropylethylamine (409.54 mg, 3.17 mmol, 551.94 μL, 4 eq) were added subsequently. The reaction mixture was stirred at −40° C. for 30 minutes then was gradually warmed to room temperature. The solvent was removed in vacuo to give crude residue, which was subjected to silica gel chromatography eluted with Petroleum Ether/Ethyl Acetate (v/v=100/0 to 94/6) to afford compound 3-3 (285 mg, Yield 83.39%).

To a stirred solution of intermediate 3-3 (285 mg, 660.63 μmol, 1 eq) in 1,4-dioxane (5 mL), compound 1-5 (157.76 mg, 990.95 μmol, 1.5 eq), Diisopropylethylamine (256.14 mg, 1.98 mmol, 345.20 μL, 3 eq) and 4 Å molecular sieves were added subsequently. The reaction mixture was warmed to 90° C. and stirred overnight. Upon the completion of conversion, the reaction mixture was cooled to room temperature and the solvent was removed in vacuo to afford crude product, which was subjected to silica gel chromatography eluted with dichloromethane/methanol (v/v=20/1) to give purified compound 3-4 (120 mg, Yield 32.78%).

To a stirred solution of intermediate 1-7 (1.0 g, 1.95 mmol, 1 eq) in dimethylformamide (10 mL), cesium fluoride (2.96 g, 19.51 mmol, 10 eq) was added in one portion. The reaction mixture was stirred for 2 hours at room temperature. Upon the completion of conversion, the reaction was quenched by adding water then diluted with ethyl acetate. The organic layer was washed with water and brine, dried over sodium sulfate and then filtered. The filtrate was concentrated in vacuo to afford compound 3-5, which was directly subjected to the next transformation without further purification (694 mg, Yield 99.8%).

To a round bottom flask, intermediate 3-5 (600 mg, 1.68 mmol, 1 eq) was dissolved in tetrahydrofuran (10 mL). Palladium 10% on carbon powder (100 mg) was added in one portion. The reaction mixture was purged with hydrogen gas and stirred for 4 hours at room temperature. Upon the completion of conversion, the reaction mixture was filtered and the filtrate was concentrated in vacuo to afford crude product, which was subjected to silica gel chromatography eluted with petroleum ether/ethyl acetate (v/v=100/0 to 94/6) to give purified compound 3-6 (600 mg, Yield 98.80%).

Compound 3-6 (117.01 mg, 324.82 μmol, 1.5 eq) was added to a stirred solution of compound 3-4 (120 mg, 216.55 μmol, 1 eq) in dioxane/water (5 mL/1.5 mL). Potassium phosphate (137.89 mg, 649.65 μmol, 3 eq) and cataCXium A Pd G3 (31.54 mg, 43.31 μmol, 0.2 eq) were added subsequently. The reaction mixture was purged with nitrogen gas three times, then warmed up to 100° C. and stirred for 4 hours. Upon the completion, the reaction mixture was cooled to room temperature and the reaction mixture was concentrated in vacuo to afford crude residue, which was subjected to silica gel column chromatography eluted with dichloromethane/methanol (v/v=100/0 to 95/5) to give compound 3-7 (100 mg, Yield 61.41%).

To a stirred solution of intermediate 3-7 (100 mg, 132.99 μmol, 1 eq) in tetrahydrofuran (5 mL), tetrabutylammonium fluoride solution 1.0 M in THF (1 mmol, 1 mL) was added through a syringe. The reaction mixture was stirred for 1 hour at room temperature. Upon the completion of conversion, the solvent was removed in vacuo to afford crude residue, which was subjected to silica gel chromatography eluted with dichloromethane/methanol (v/v=100/0 to 96/4) to give purified compound 3-8 (70 mg, Yield 82.54%).

To a stirred solution of intermediate 3-8 (70 mg, 109.77 μmol, 1 eq) in dichloromethane (10 mL), hydrogen chloride solution 4.0 M in dioxane (1.0 mL, 4.0 mmol) was added through a syringe. The reaction mixture was stirred for 10 minutes at room temperature. Upon the completion of conversion, the solvent was removed in vacuo to afford crude residue, which was subjected to a reverse phase preparative HPLC eluted with 0.05% ammonia hydroxide aqueous solution/acetonitrile to give title compound 3 (22.6 mg, Yield 34.31%). ¹H NMR (500 MHz, CD₃OD) δ ppm 9.30-9.23 (m, 1H), 7.72-7.64 (m, 1H), 7.30 (s, 1H), 7.25 (t, J=9.3 Hz, 1H), 7.05 (d, J=9.1 Hz, 1H), 5.37 (s, 0.5H), 5.26 (s, 0.5H), 4.59 (s, 2H), 4.39-4.32 (m, 1H), 4.26-4.07 (m, 3H), 3.98-3.88 (m, 2H), 3.29-3.14 (m, 3H), 3.08-2.97 (m, 1H), 2.56-2.44 (m, 1H), 2.36-1.86 (m, 11H), 0.85-0.74 (m, 3H). m/z (ESI): 594.5 [M+H]⁺.

### Synthesis of Compound 4

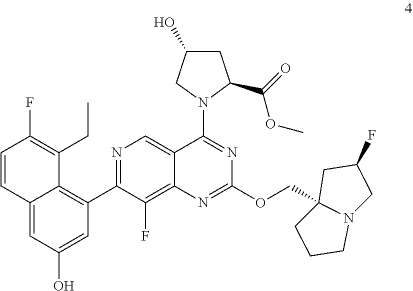 [SMILES: CCc1c(F)ccc2cc(O)cc(-c3ncc4c(N5C[C@H](O)C[C@H]5C(=O)OC)nc(OC[C@@]56CCCN5C[C@H](F)C6)nc4c3F)c12]

Title compound was synthesized from (S)-4-hydroxy-D-proline methyl ester in a manner essentially analogous to the synthesis of compound 1. ¹H NMR (500 MHz, CD₃OD) δ ppm 9.25 (d, J=5.5 Hz, 1H), 7.67 (dd, J=8.7, 6.0 Hz, 1H), 7.30 (s, 1H), 7.24 (t, J=9.0 Hz, 1H), 7.06 (d, J=22.1 Hz, 1H), 5.38 (s, 0.5H), 5.27 (s, 0.5H), 5.12-5.01 (m, 1H), 4.71 (s, 1H), 4.66-4.55 (m, 1H), 4.55-4.44 (m, 1H), 4.37 (d, J=10.7 Hz, 1H), 4.10 (d, J=10.7 Hz, 2H), 3.79 (d, J=9.9 Hz, 3H), 3.46-3.19 (m, 2H), 3.09-3.00 (m, 1H), 2.58-2.39 (m, 2H), 2.38-2.07 (m, 5H), 2.06-1.99 (m, 2H), 1.96-1.86 (m, 1H), 0.78 (q, J=7.1 Hz, 3H). m/z, (ESI): 638.5 [M+H]⁺.

### Synthesis of Compound 5

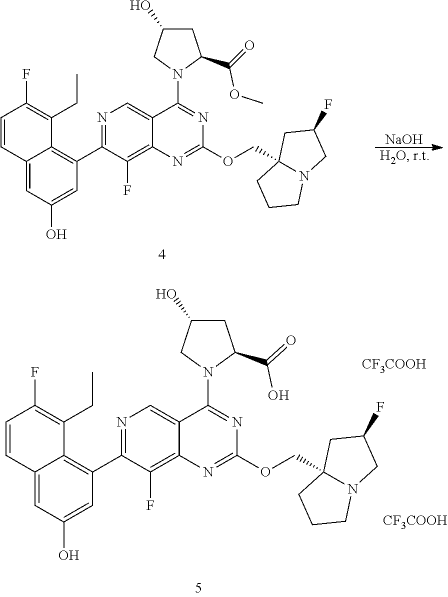

To a stirred suspension of compound 4 (40 mg, 62.73 μmol, 1 eq) in water (3 mL), sodium hydroxide (25.09 mg, 627.31 μmol, 10 eq) was added in one portion. The reaction mixture was stirred for 30 minutes at room temperature. Upon the completion of conversion, the solvent was removed in vacuo to afford crude residue, which was subjected to a reverse phase preparative HPLC eluted with 0.05% trifluoroacetic acid aqueous solution/acetonitrile to give compound 5 as a pale-yellow powder (17.1 mg, Yield 31.77%). ¹H NMR (500 MHz, CD₃OD) δ ppm 9.32 (d, J=6.3 Hz, 1H), 7.69 (dd, J=8.9, 5.9 Hz, 1H), 7.32 (d, J=2.2 Hz, 1H), 7.26 (t, J=9.4 Hz, 1H), 7.06 (d, J=19.3 Hz, 1H), 5.63 (s, 0.5H), 5.53 (s, 0.5H), 5.01 (t, J=8.6 Hz, 1H), 4.78-4.70 (m, 2H), 4.61-4.46 (m, 2H), 4.14 (d, J=10.9 Hz, 1H), 4.09-3.83 (m, 3H), 3.52-3.42 (m, 1H), 2.76-2.41 (m, 4H), 2.39-2.24 (m, 4H), 2.23-2.06 (m, 2H), 0.79 (dt, J=17.6, 7.5 Hz, 3H). m/z, (ESI): 624.5 [M+H]⁺.

### Synthesis of Compound 6

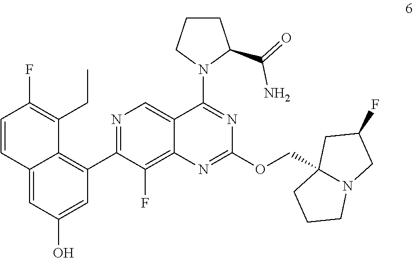 [SMILES: CCc1c(F)ccc2cc(O)cc(-c3ncc4c(N5CCC[C@H]5C(N)=O)nc(OC[C@@]56CCCN5C[C@H](F)C6)nc4c3F)c12]

Title compound was synthesized from L-prolinamide in a manner essentially analogous to the synthesis of compound 1. ¹H NMR (500 MHz, CD₃OD) δ ppm 9.33 (s, 1H), 7.68 (dd, J=8.5, 6.1 Hz, 1H), 7.31 (d, J=1.7 Hz, 1H), 7.25 (t, J=9.3 Hz, 1H), 7.05 (d, J=12.1 Hz, 1H), 5.37 (s, 0.5H), 5.26 (s, 0.5H), 4.95-4.90 (m, 1H), 4.39-4.19 (m, 4H), 3.29-3.13 (m, 3H), 3.09-2.97 (m, 1H), 2.58-2.40 (m, 2H), 2.39-2.07 (m, 7H), 2.04-1.88 (m, 3H), 0.80 (t, J=7.3 Hz, 3H). m/z, (ESI): 607.4 [M+H]⁺.

### Synthesis of Compound 7

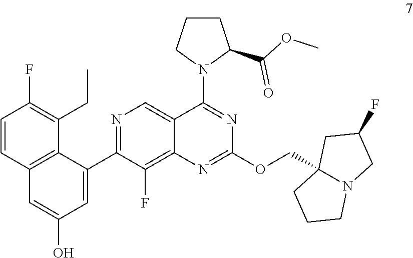 [SMILES: CCc1c(F)ccc2cc(O)cc(-c3ncc4c(N5CCC[C@H]5C(=O)OC)nc(OC[C@@]56CCCN5C[C@H](F)C6)nc4c3F)c12]

Title compound was synthesized from L-proline methyl ester in a manner essentially analogous to the synthesis of compound 3. ¹H NMR (500 MHz, CD₃OD) δ ppm 9.33 (s, 1H), 7.74-7.63 (m, 1H), 7.34-7.21 (m, 2H), 7.06 (d, J=15.6 Hz, 1H), 5.37 (s, 0.5H), 5.27 (s, 0.5H), 4.97 (s, 1H), 4.37-4.23 (m, 3H), 4.14-4.02 (m, 1H), 3.83-3.75 (m, 3H), 3.27-3.12 (m, 3H), 3.10-2.98 (m, 1H), 2.55-2.41 (m, 1H), 2.40-2.12 (m, 5H), 2.12-1.76 (m, 4H), 1.39-1.26 (m, 2H), 0.85-0.75 (m, 3H). m/z (ESI): 622.5 [M+H]⁺.

### Synthesis of Compound 8

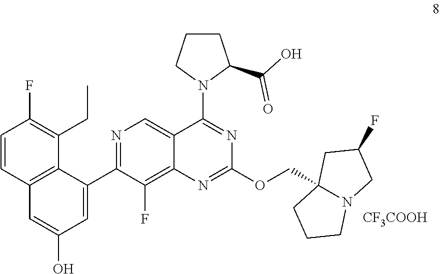

Title compound was synthesized from compound 7 in a manner essentially analogous to the synthesis of compound 8. ¹H NMR (500 MHz, CD₃OD) δ ppm 9.40 (s, 1H), 7.74-7.65 (m, 1H), 7.35-7.23 (m, 2H), 7.10-7.02 (m, 1H), 5.64 (s, 0.5H), 5.53 (s, 0.5H), 4.74-4.68 (m, 1H), 4.60-4.52 (m, 1H), 4.43-4.25 (m, 2H), 4.10-3.84 (m, 3H), 3.77-3.68 (m, 1H), 3.53-3.43 (m, 1H), 3.26-3.20 (m, 1H), 2.74-2.44 (m, 3H), 2.41-2.32 (m, 3H), 2.21-2.10 (m, 1H), 1.37-1.34 (m, 4H), 0.84-0.74 (m, 3H). m/z (ESI): 608.4 [M+H]⁺.

### Synthesis of Compound 9

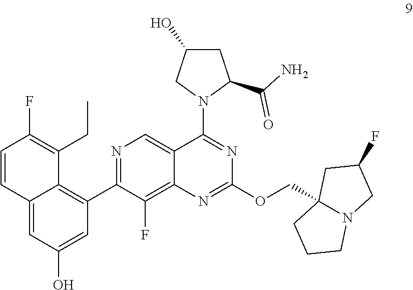 [SMILES: CCc1c(F)ccc2cc(O)cc(-c3ncc4c(N5C[C@H](O)C[C@H]5C(N)=O)nc(OC[C@@]56CCCN5C[C@H](F)C6)nc4c3F)c12]

Title compound was synthesized from (2S,4R)-4-hydroxypyrrolidine-2-carboxamide in a manner essentially analogous to the synthesis of compound 3. ¹H NMR (500 MHz, CD₃OD) δ ppm 0.80 (dt, J=14.8, 7.4 Hz, 3H), 1.89-2.53 (m, 10H), 3.01 (dd, J=14.0, 8.4 Hz, 1H), 3.19 (dd, J=33.7, 13.7 Hz, 3H), 4.09 (d, J=11.0 Hz, 1H), 4.29 (dd, J=22.6, 10.6 Hz, 2H), 4.49 (s, 1H), 4.71 (s, 1H), 5.04 (t, J=8.5 Hz, 1H), 5.25 (s, 0.5H), 5.36 (s, 0.5H), 7.06 (dd, J=20.8, 2.0 Hz, 1H), 7.25 (t, J=9.2 Hz, 1H), 7.30 (s, 1H), 7.68 (dd, J=8.7, 6.0 Hz, 1H), 9.25 (d, J=7.7 Hz, 1H). m/z (ESI): 623.5 [M+H]⁺.

### Synthesis of Compound 10

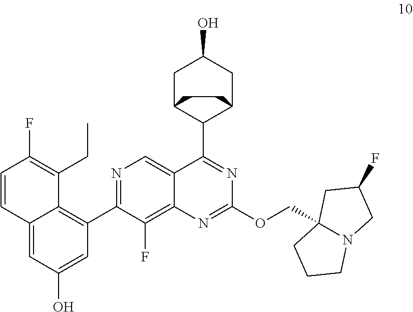 [SMILES: CCc1c(F)ccc2cc(O)cc(-c3ncc4c(C5[C@@H]6CC[C@H]5C[C@@H](O)C6)nc(OC[C@@]56CCCN5C[C@H](F)C6)nc4c3F)c12]

Title compound was synthesized from nortropine in a manner essentially analogous to the synthesis of compound 3. ¹H NMR (500 MHz, CD₃OD) δ ppm 9.07 (s, 1H), 7.67 (dd, J=8.4, 6.0 Hz, 1H), 7.30 (s, 1H), 7.25 (t, J=9.5 Hz, 1H), 7.05 (s, 1H), 5.36 (s, 0.5H), 5.31-5.15 (m, 2.5H), 4.30 (d, J=10.9 Hz, 1H), 4.25-4.15 (m, 2H), 3.28-3.16 (m, 3H), 3.08-2.96 (m, 1H), 2.56-2.43 (m, 3H), 2.41-2.03 (m, 2H), 2.03-1.81 (m, 11H), 0.81 (t, J=7.0 Hz, 3H). m/z, (ESI): 620.5 [M+H]⁺.

### Synthesis of Compound 11

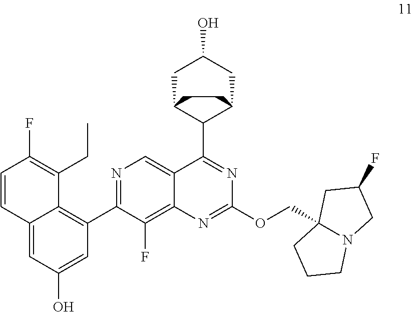 [SMILES: CCc1c(F)ccc2cc(O)cc(-c3ncc4c(C5[C@@H]6CC[C@H]5C[C@H](O)C6)nc(OC[C@@]56CCCN5C[C@H](F)C6)nc4c3F)c12]

Title compound was synthesized from exo-8-azabicyclo[3.2.1]octan-3-ol in a manner essentially analogous to the synthesis of compound 3. ¹H NMR (500 MHz, CD₃OD) δ ppm 9.12 (s, 1H), 7.67 (dd, J=8.7, 6.2 Hz, 1H), 7.30 (s, 1H), 7.24 (t, J=9.5 Hz, 1H), 7.06 (s, 1H), 5.35 (s, 0.5H), 5.32-5.22 (m, 2.5H), 4.36-4.27 (m, 2H), 4.23 (d, J=10.4 Hz, 1H), 3.29-3.13 (m, 3H), 3.05-2.97 (m, 1H), 2.56-2.42 (m, 1H), 2.37-2.10 (m, 8H), 2.04-1.78 (m, 7H), 0.80 (t, J=7.2 Hz, 3H). m/z, (ESI): 620.5 [M+H]⁺.

### Synthesis of Compound 12

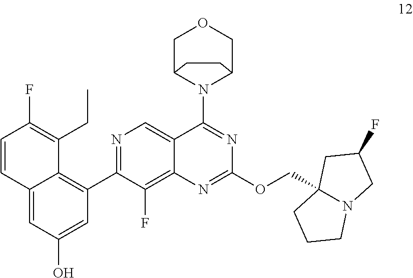 [SMILES: CCc1c(F)ccc2cc(O)cc(-c3ncc4c(N5C6CCC5COC6)nc(OC[C@@]56CCCN5C[C@H](F)C6)nc4c3F)c12]

Title compound was synthesized from 3-Oxa-8-azabicyclo[3.2.1]octane in a manner essentially analogous to the synthesis of compound 3. ¹H NMR (500 MHz, CD₃OD) δ ppm 9.10 (s, 1H), 7.68 (dd, J=8.7, 5.9 Hz, 1H), 7.31 (s, 1H), 7.26 (t, J=9.3 Hz, 1H), 7.06 (s, 1H), 5.36 (s, 0.5H), 5.25 (s, 0.5H), 5.12 (s, 2H), 4.31 (d, J=10.6 Hz, 1H), 4.24 (d, J=10.5 Hz, 1H), 4.01 (d, J=11.0 Hz, 2H), 3.84 (d, J=10.9 Hz, 2H), 3.29-3.14 (m, 3H), 3.07-2.97 (m, 1H), 2.54-2.43 (m, 1H), 2.40-2.06 (m, 8H), 2.05-1.83 (m, 3H), 0.81 (t, J=7.2 Hz, 3H). m/z, (ESI): 606.4 [M+H]⁺.

### Synthesis of Compound 13

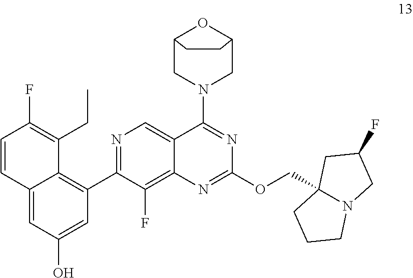 [SMILES: CCc1c(F)ccc2cc(O)cc(-c3ncc4c(N5CC6CCC(C5)O6)nc(OC[C@@]56CCCN5C[C@H](F)C6)nc4c3F)c12]

Title compound was synthesized from 8-Oxa-3-azabicyclo[3.2.1]octane in a manner essentially analogous to the synthesis of compound 3. ¹H NMR (500 MHz, CD₃OD) δ ppm 9.07 (s, 1H), 7.68 (dd, J=9.1, 5.6 Hz, 1H), 7.30 (s, 1H), 7.25 (t, J=9.1 Hz, 1H), 7.05 (s, 1H), 5.36 (s, 0.5H), 5.25 (s, 0.5H), 4.63-4.48 (m, 4H), 4.31 (d, J=10.4 Hz, 1H), 4.24 (d, J=10.1 Hz, 1H), 3.88-3.74 (m, 2H), 3.29-3.15 (m, 3H), 3.06-2.98 (m, 1H), 2.54-2.43 (m, 1H), 2.39-2.10 (m, 4H), 2.06-1.81 (m, 7H), 0.80 (t, J=6.8 Hz, 3H). m/z, (ESI): 606.4 [M+H]⁺.

### Synthesis of Compound 14

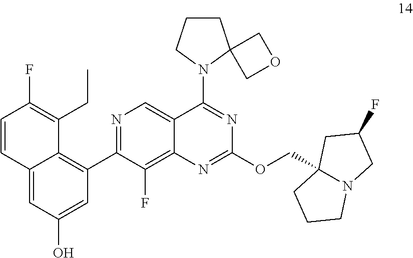 [SMILES: CCc1c(F)ccc2cc(O)cc(-c3ncc4c(N5CCCC56COC6)nc(OC[C@@]56CCCN5C[C@H](F)C6)nc4c3F)c12]

Title compound was synthesized from 2-Oxa-5-azaspiro[3.4]octane in a manner essentially analogous to the synthesis of compound 3. ¹H NMR (500 MHz, CD₃OD) δ ppm 0.80 (t, J=6.3 Hz, 3H), 1.86-2.56 (m, 13H), 3.01 (dd, J=15.5, 9.2 Hz, 1H), 3.13-3.27 (m, 2H), 4.15 (t, J=6.2 Hz, 2H), 4.47-4.53 (m, 3H), 4.63 (t, J=11.0 Hz, 1H), 5.26 (s, 0.5H), 5.37 (s, 0.5H), 5.90 (d, J=5.4 Hz, 2H), 7.04 (s, 1H), 7.23 (d, J=9.4 Hz, 1H), 7.28 (d, J=2.1 Hz, 1H), 7.65 (dd, J=8.7, 6.0 Hz, 1H), 9.27 (s, 1H). m/z (ESI): 606.4 [M+H]⁺.

### Synthesis of Compound 15

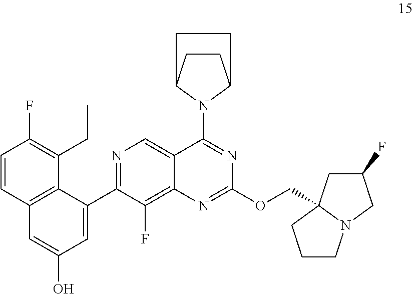 [SMILES: CCc1c(F)ccc2cc(O)cc(-c3ncc4c(N5C6CCC5CC6)nc(OC[C@@]56CCCN5C[C@H](F)C6)nc4c3F)c12]

Title compound was synthesized from 7-azabicyclo[2.2.1]heptane in a manner essentially analogous to the synthesis of compound 3. ¹H NMR (500 MHz, CD₃OD) δ ppm 9.33-9.06 (m, 1H), 7.78-7.63 (m, 1H), 7.40-7.21 (m, 2H), 7.11-7.09 (m, 1H), 5.36 (d, J=53.0 Hz, 1H), 5.12 (s, 3H), 4.64 (s, 2H), 4.47-4.17 (m, 2H), 3.29-3.21 (m, 1H), 3.07 (s, 1H), 2.51 (s, 1H), 2.41-2.35 (m, 1H), 2.24-2.12 (m, 3H), 2.11-2.00 (m, 6H), 1.94 (s, 1H), 1.80-1.70 (m, 4H), 0.88-0.78 (m, 3H). m/z (ESI): 590.5 [M+H]⁺.

### Synthesis of Compound 16

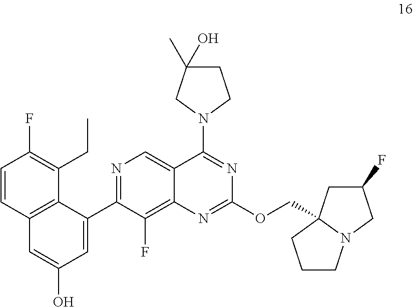 [SMILES: CCc1c(F)ccc2cc(O)cc(-c3ncc4c(N5CCC(C)(O)C5)nc(OC[C@@]56CCCN5C[C@H](F)C6)nc4c3F)c12]

Title compound was synthesized from 3-Methylpyrrolidin-3-ol in a manner essentially analogous to the synthesis of compound 3. ¹H NMR (500 MHz, CD₃OD) δ ppm 9.28 (s, 1H), 7.70 (dd, J=9.1, 5.8 Hz, 1H), 7.33 (d, J=2.6 Hz, 1H), 7.28 (t, J=9.4 Hz, 1H), 7.09 (d, J=12.0, 2.5 Hz, 1H), 5.34 (d, J=53.7 Hz, 1H), 4.41-4.34 (m, 1H), 4.30 (t, J=10.4 Hz, 1H), 4.17 (s, 1H), 4.05-4.01 (m, 1H), 3.33-3.21 (m, 4H), 3.08-3.03 (m, 1H), 2.51 (dd, J=14.9, 7.7 Hz, 1H), 2.43-2.32 (m, 1H), 2.31-2.23 (m, 2H), 2.23-2.12 (m, 3H), 2.08-2.00 (m, 2H), 1.98-1.88 (m, 1H), 1.56 (s, 3H), 0.87-0.78 (m, 3H). m/z (ESI⁺): 594.4 [M+H]⁺.

### Synthesis of Compound 17

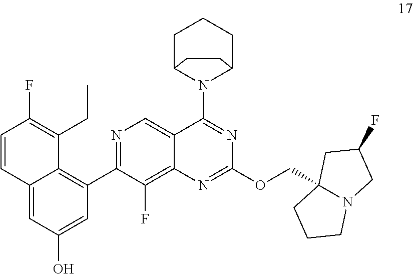 [SMILES: CCc1c(F)ccc2cc(O)cc(-c3ncc4c(N5C6CCCC5CC6)nc(OC[C@@]56CCCN5C[C@H](F)C6)nc4c3F)c12]

Title compound was synthesized from 8-Aza-bicyclo[3.2.1]octane in a manner essentially analogous to the synthesis of compound 3. ¹H NMR (400 MHz, CD₃OD) δ ppm 0.81 (dd, J=7.4, 6.0 Hz, 3H), 1.75-2.55 (m, 20H), 3.08-3.19 (m, 1H), 3.37-3.56 (m, 3H), 4.37 (dd, J=34.6, 11.1 Hz, 2H), 5.31 (s, 0.5H), 5.44 (s, 0.5H), 7.06 (d, J=2.5 Hz, 1H), 7.25 (t, J=9.4 Hz, 1H), 7.31 (d, J=2.6 Hz, 1H), 7.68 (dd, J=9.0, 5.8 Hz, 1H), 9.11 (s, 1H). m/z (ESI⁺): 604.5 [M+H]⁺.

### Synthesis of Compound 18

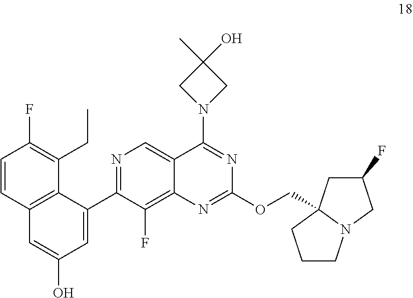 [SMILES: CCc1c(F)ccc2cc(O)cc(-c3ncc4c(N5CC(C)(O)C5)nc(OC[C@@]56CCCN5C[C@H](F)C6)nc4c3F)c12]

Title compound was synthesized from 3-methylazetidin-3-ol in a manner essentially analogous to the synthesis of compound 3. ¹H NMR (400 MHz, CD₃OD) δ ppm 8.94 (s, 1H), 7.69 (dd, J=9.1, 5.8 Hz, 1H), 7.32 (d, J=2.6 Hz, 1H), 7.26 (t, J=9.3 Hz, 1H), 7.06 (d, J=2.6 Hz, 1H), 5.31 (d, J=54.0 Hz, 1H), 4.77-4.36 (m, 4H), 4.30 (q, J=10.0 Hz, 2H), 3.31-3.26 (m, 1H), 3.25-3.22 (m, 1H), 3.21-3.18 (m, 1H), 3.06-2.98 (m, 1H), 2.56-2.43 (m, 1H), 2.41-2.27 (m, 1H), 2.26-2.20 (m, 1H), 2.18-2.10 (m, 2H), 2.05-1.95 (m, 2H), 1.94-1.84 (m, 1H), 1.64 (s, 3H), 0.81 (t, J=7.4 Hz, 3H). m/z (ESI): 580.4 [M+H]⁺.

### Synthesis of Compound 19

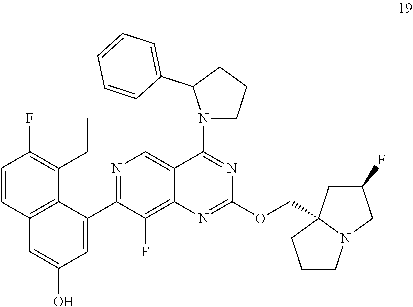 [SMILES: CCc1c(F)ccc2cc(O)cc(-c3ncc4c(N5CCCC5c5ccccc5)nc(OC[C@@]56CCCN5C[C@H](F)C6)nc4c3F)c12]

Title compound was synthesized from 2-phenylpyrrolidine in a manner essentially analogous to the synthesis of compound 3. ¹H NMR (400 MHz, CD₃OD) δ ppm 9.28 (s, 1H), 7.70-7.66 (m, 1H), 7.37 (d, J=4.2 Hz, 2H), 7.34 (d, J=4.8 Hz, 2H), 7.31 (d, J=2.6 Hz, 1H), 7.29-7.22 (m, 2H), 7.04 (d, J=14.5 Hz, 1H), 5.68 (s, 1H), 5.24 (dd, J=54.0, 14.4 Hz, 1H), 4.54 (d, J=44.2 Hz, 1H), 4.33 (d, J=9.4 Hz, 1H), 4.09 (s, 1H), 3.91-3.35 (m, 1H), 3.30-3.05 (m, 3H), 2.98 (s, 1H), 2.70-2.34 (m, 2H), 2.32-1.50 (m, 10H), 0.95-0.53 (m, 3H). m/z (ESI): 640.4 [M+H]⁺.

### Synthesis of Compound 20

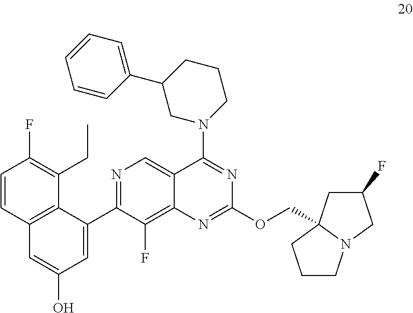 [SMILES: CCc1c(F)ccc2cc(O)cc(-c3ncc4c(N5CCCC(c6ccccc6)C5)nc(OC[C@@]56CCCN5C[C@H](F)C6)nc4c3F)c12]

Title compound was synthesized from 3-phenylpiperidine in a manner essentially analogous to the synthesis of compound 3. ¹H NMR (400 MHz, CD₃OD) δ ppm 9.06 (d, J=3.3 Hz, 1H), 7.67 (dd, J=9.0, 5.8 Hz, 1H), 7.38-7.32 (m, 4H), 7.31 (d, J=2.6 Hz, 1H), 7.29-7.21 (m, 2H), 7.08 (dd, J=6.2, 2.6 Hz, 1H), 5.29 (d, J=53.6 Hz, 1H), 4.79-4.70 (m, 2H), 4.48-4.12 (m, 2H), 3.60-3.37 (m, 2H), 3.36-3.26 (m, 2H), 3.25-3.17 (m, 2H), 3.15-2.96 (m, 2H), 2.54-2.39 (m, 1H), 2.27-2.18 (m, 2H), 2.16-2.07 (m, 2H), 2.05-1.95 (m, 4H), 1.94-1.81 (m, 2H), 0.80 (t, J=7.4 Hz, 3H). m/z (ESI): 654.4 [M+H]⁺.

### Synthesis of Compound 21

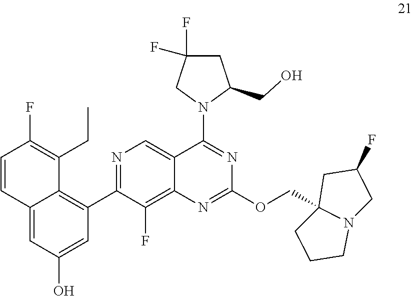 [SMILES: CCc1c(F)ccc2cc(O)cc(-c3ncc4c(N5CC(F)(F)C[C@H]5CO)nc(OC[C@@]56CCCN5C[C@H](F)C6)nc4c3F)c12]

Title compound was synthesized from (S)-(4,4-Difluoropyrrolidin-2-yl)methanol in a manner essentially analogous to the synthesis of compound 3. ¹H NMR (500 MHz, CD₃OD) δ ppm 9.15 (d, J=16.7 Hz, 1H), 7.67 (dd, J=9.1, 5.7 Hz, 1H), 7.29 (s, 1H), 7.24 (t, J=9.3 Hz, 1H), 7.04 (s, 1H), 5.35 (s, 0.5H), 5.24 (s, 0.5H), 5.18-5.05 (m, 1H), 4.67-4.43 (m, 2H), 4.34 (d, J=10.2 Hz, 1H), 4.22 (d, J=10.4 Hz, 1H), 4.11-3.99 (m, 1H), 3.89-3.82 (m, 1H), 3.27-3.17 (m, 3H), 3.05-2.93 (m, 1H), 2.84-2.60 (m, 2H), 2.56-1.72 (m, 8H), 0.85-0.71 (m, 3H). m/z (ESI): 630.4 [M+H]⁺.

### Synthesis of Compound 22

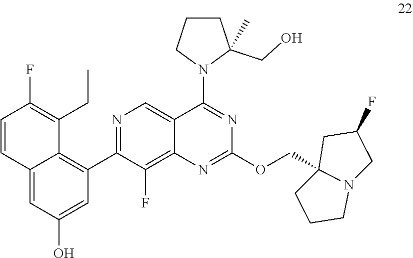 [SMILES: CCc1c(F)ccc2cc(O)cc(-c3ncc4c(N5CCC[C@@]5(C)CO)nc(OC[C@@]56CCCN5C[C@H](F)C6)nc4c3F)c12]

Title compound was synthesized from (S)-2-methylprolinol in a manner essentially analogous to the synthesis of compound 3. ¹H NMR (500 MHz, CD₃OD) δ ppm 0.77-0.84 (m, 3H), 1.68 (d, J=6.8 Hz, 3H), 1.86-2.53 (m, 13H), 3.04-3.11 (m, 1H), 3.85 (dd, J=11.5, 3.3 Hz, 1H), 4.21 (ddd, J=17.2, 16.8, 8.9 Hz, 3H), 4.33 (d, J=10.7 Hz, 1H), 4.51 (dd, J=22.4, 11.5 Hz, 1H), 4.60 (s, 2H), 5.29 (s, 0.5H), 5.40 (s, 0.5H), 7.04 (s, 1H), 7.25 (t, J=9.4 Hz, 1H), 7.30 (d, J=2.0 Hz, 1H), 7.68 (dd, J=8.6, 6.0 Hz, 1H), 9.30 (s, 1H). m/z (ESI): 608.5 [M+H]⁺.

### Synthesis of Compound 23

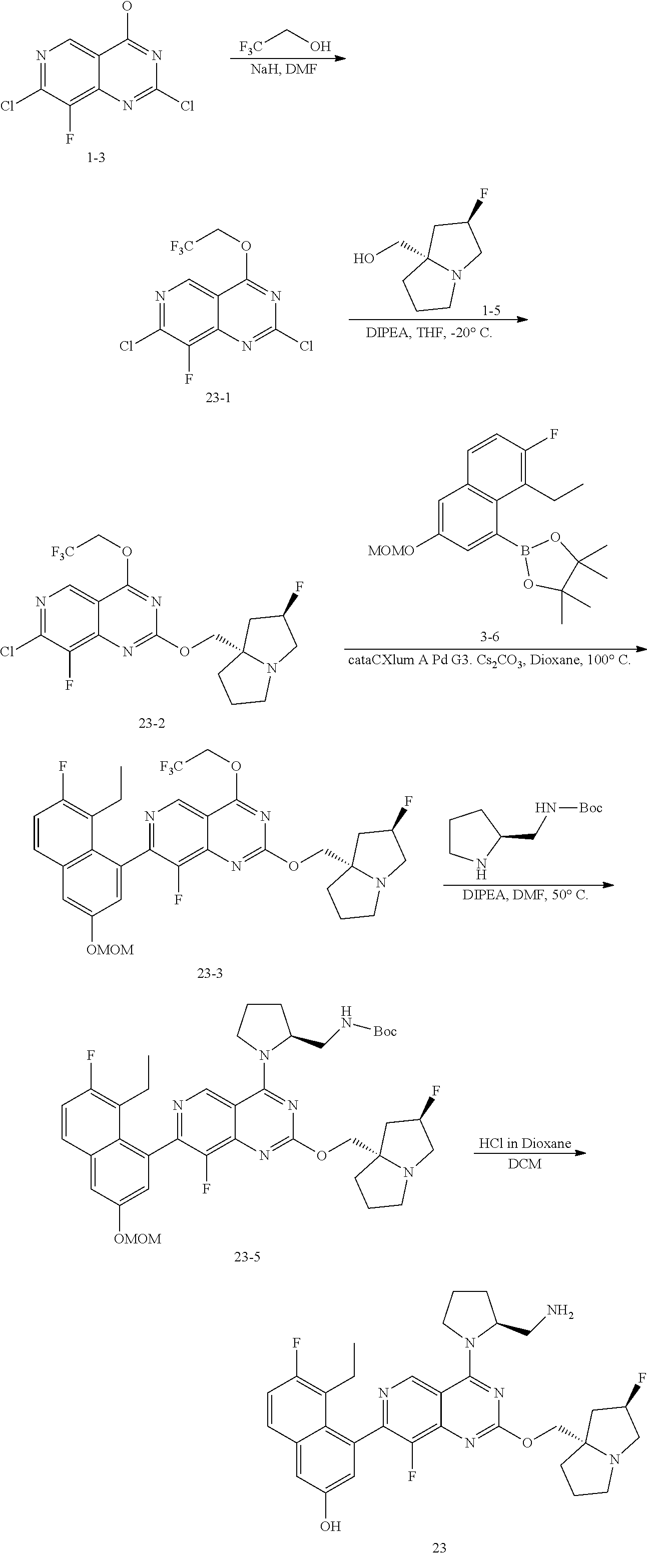

The solution of 2,2,2-trifluoroethanol (1.43 g, 14.26 mmol, 1.2 eq) in anhydrous dimethylformamide (20 mL) was purged with nitrogen gas and cooled to −40° C. 60% sodium hydride (w/w) in mineral oil (522.8 mg, 13.07 mmol, 1.1 eq) was added at −40° C. and stirred for 30 minutes to reach full deprotonation. Intermediate 1-3 (3.0 g, 11.88 mmol, 1.0 eq) was added, the reaction mixture was stirred at −40° C. for another 1 hour, then was quenched by adding water. Ethyl acetate was poured into the reaction mixture and the organic layer was collected, washed with water and brine, dried over sodium sulfate then filtered. The filtrate was concentrated in vacuo to give crude residue, which was subjected to silica gel chromatography eluted with Petroleum Ether/Ethyl Acetate (v/v=10/1) to afford compound 23-1 (2.5 g, Yield 66.57%).

The solution of intermediate 23-1 (1.0 g, 3.16 mmol, 1.0 eq) in anhydrous tetrahydrofuran (20 mL) was purged with nitrogen gas and cooled to −20° C. Intermediate 1-5 (1.01 g, 6.33 mmol, 2 eq) and diisopropylethylamine (817.87 mg, 6.33 mmol, 1.10 mL, 2 eq) were added subsequently at −20° C. and the reaction mixture was stirred at −20° C. for 5 hours. Upon the completion of conversion, the reaction was quenched by adding water, then ethyl acetate was poured into the reaction mixture. The organic layer was separated, washed with water and brine, and dried over sodium sulfate then filtered. The filtrate was concentrated in vacuo to give crude residue, which was subjected to silica gel chromatography eluted with Petroleum Ether/Ethyl Acetate (v/v=1/1) to afford compound 23-2 (917 mg, Yield 66.05%).

Intermediate 3-6 (779.97 mg, 2.28 mmol, 2.0 eq) was added to a stirred solution of compound 23-2 (500 mg, 1.14 mmol, 1.0 eq) in dioxane (5 mL). Cesium carbonate (1.11 g, 3.42 mmol, 3 eq) and cataCXium A Pd G3 (248.87 mg, 341.86 μmol, 0.3 eq) were added subsequently. The reaction mixture was purged with nitrogen gas three times, then warmed up to 100° C. and stirred for 3 hours. Upon completion, the reaction mixture was cooled to room temperature, diluted with water and extracted with ethyl acetate. The organic layer was washed with water and brine, dried over sodium sulfate and then filtered. The filtrate was concentrated in vacuo to afford crude residue, which was subjected to silica gel column chromatography eluted with Petroleum Ether/Ethyl Acetate (v/v=1/1) to afford compound 23-3 (200 mg, Yield 27.57%).

To a stirred solution of intermediate 23-2 (280 mg, 670.85 μmol, 1 eq) in dimethylformamide (5 mL), reagent 23-4 (14.16 mg, 70.69 μmol, 1.5 eq) and Diisopropylethylamine (18.27 mg, 141.38 μmol, 24.63 μL, 3 eq) were added subsequently. The reaction mixture was warmed to 50° C. and stirred for 20 hours. Upon the completion of conversion, the reaction mixture was quenched with water and the mixture was diluted with ethyl acetate. The organic layer was washed with water and brine, dried over sodium sulfate and then filtered. The filtrate was concentrated in vacuo to afford crude product, which was subjected to silica gel chromatography eluted with dichloromethane/methanol (v/v=10/1) to give purified compound 23-5 (27 mg, Yield 77.76%).

To a stirred solution of intermediate 23-5 (27 mg, 36.64 μmol, 1 eq) in dichloromethane (1 mL), hydrogen chloride solution 4.0 M in dioxane (36.6 μL, 146.58 μmol, 4 eq) was added in one portion. The reaction mixture was stirred for 10 minutes at room temperature. Upon the completion of conversion, the solvent was removed in vacuo to afford crude residue, which was subjected to a reverse phase preparative HPLC eluted with 0.05% ammonia hydroxide aqueous solution/acetonitrile to give the compound 23 (12 mg, Yield 50.86%). ¹H NMR (500 MHz, CD₃OD) δ ppm 0.76-0.84 (m, 3H), 1.97 (dd, J=42.8, 25.2 Hz, 3H), 2.23 (ddd, J=52.9, 37.8, 19.0 Hz, 9H), 2.91-2.97 (m, 1H), 3.02 (d, J=5.7 Hz, 1H), 3.22 (dd, J=36.8, 15.7 Hz, 5H), 4.12 (dt, J=10.1, 6.7 Hz, 1H), 4.21 (d, J=10.5 Hz, 2H), 4.34 (d, J=10.6 Hz, 1H), 5.25 (s, 0.5H), 5.36 (s, 0.5H), 7.05 (d, J=11.8 Hz, 1H), 7.25 (t, J=9.3 Hz, 1H), 7.30 (d, J=2.1 Hz, 1H), 7.68 (dd, J=8.8, 5.9 Hz, 1H), 9.24 (d, J=6.5 Hz, 1H). m/z (ESI): 593.4 [M+H]⁺.

### Synthesis of Compound 24

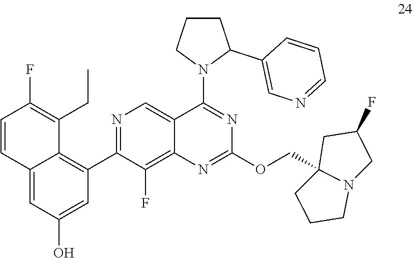 [SMILES: CCc1c(F)ccc2cc(O)cc(-c3ncc4c(N5CCCC5c5cccnc5)nc(OC[C@@]56CCCN5C[C@H](F)C6)nc4c3F)c12]

Title compound was synthesized from 3-(pyrrolidin-2-yl)pyridine in a manner essentially analogous to the synthesis of compound 3. ¹H NMR (500 MHz, CD₃OD) δ ppm 0.79 (dd, J=10.8, 7.0 Hz, 3H), 1.79 (s, 1H), 1.92-2.35 (m, 10H), 2.44-2.67 (m, 2H), 2.97 (d, J=6.3 Hz, 1H), 3.13-3.20 (m, 3H), 4.06 (dd, J=26.4, 11.7 Hz, 1H), 4.37 (s, 1H), 4.58 (s, 1H), 5.20 (s, 0.5H), 5.30 (s, 0.5H), 5.66 (s, 1H), 7.04 (d, J=14.3 Hz, 1H), 7.25 (t, J=9.2 Hz, 1H), 7.31 (s, 1H), 7.42 (dd, J=7.2, 4.2 Hz, 1H), 7.68 (dd, J=8.6, 6.1 Hz, 1H), 7.87 (dd, J=19.1, 7.5 Hz, 1H), 8.45 (t, J=5.3 Hz, 1H), 8.64 (d, J=12.6 Hz, 1H), 9.38 (s, 1H). m/z (ESI): 641.5 [M+H]⁺.

### Synthesis of Compound 25

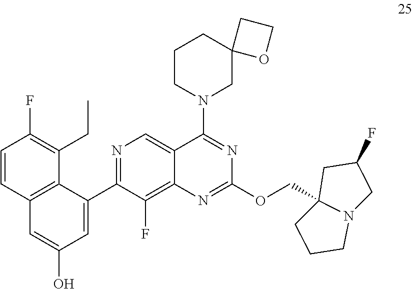 [SMILES: CCc1c(F)ccc2cc(O)cc(-c3ncc4c(N5CCCC6(CCO6)C5)nc(OC[C@@]56CCCN5C[C@H](F)C6)nc4c3F)c12]

Title compound was synthesized from 1-Oxa-6-azaspiro[3.5]nonane in a manner essentially analogous to the synthesis of compound 23. ¹H NMR (500 MHz, CD₃OD) δ ppm 9.29 (d, J=5.6 Hz, 1H), 7.71 (dd, J=9.1, 5.7 Hz, 1H), 7.34 (d, J=2.7 Hz, 1H), 7.28 (t, J=9.3 Hz, 1H), 7.12 (s, 1H), 5.34 (d, J=53.9 Hz, 1H), 4.66-4.57 (m, 3H), 4.40-4.25 (m, 2H), 3.93-3.78 (m, 1H), 3.62-3.41 (m, 1H), 3.31-3.14 (m, 3H), 3.09-3.00 (m, 1H), 2.59-2.46 (m, 3H), 2.43-2.12 (m, 6H), 2.10-1.78 (m, 6H), 0.84 (q, J=7.3 Hz, 3H). m/z (ESI): 620.4 [M+H]⁺.

### Synthesis of Compound 26

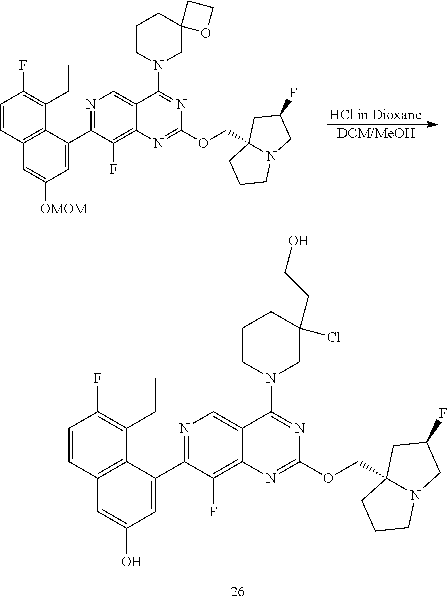

Title compound was a byproduct isolated in the synthesis of compound 25 by preparative HPLC eluted with 0.05% ammonia hydroxide aqueous solution/acetonitrile. ¹H NMR (400 MHz, CD₃OD) δ ppm 9.26 (dd, J=12.3, 3.1 Hz, 1H), 7.71 (dd, J=9.0, 5.9 Hz, 1H), 7.34 (s, 1H), 7.28 (t, J=9.4 Hz, 1H), 7.10 (s, 1H), 5.34 (d, J=52.7 Hz, 1H), 4.66-4.55 (m, 1H), 4.47-4.33 (m, 2H), 4.33-4.26 (m, 1H), 3.84-3.75 (m, 2H), 3.73-3.60 (m, 1H), 3.57-3.42 (m, 1H), 3.29-3.22 (m, 2H), 3.10-3.01 (m, 1H), 2.56-2.45 (m, 1H), 2.41-2.14 (m, 5H), 2.11-1.99 (m, 5H), 1.92-1.76 (m, 4H), 0.89-0.79 (m, 3H). m/z (ESI): 656.5 [M+H]⁺.

### Synthesis of Compound 27

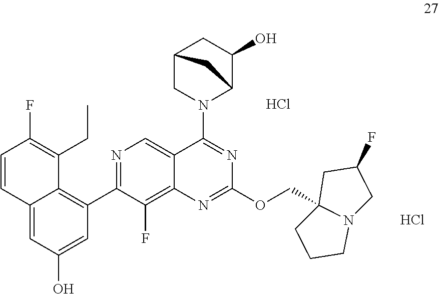 [SMILES: CCc1c(F)ccc2cc(O)cc(-c3ncc4c(N5C[C@@H]6C[C@@H](O)[C@H]5C6)nc(OC[C@@]56CCCN5C[C@H](F)C6)nc4c3F)c12 ; Cl ; Cl]

Title compound was synthesized from (1R,4S,6R)-2-azabicyclo[2.2.1]heptan-6-ol in a manner essentially analogous to the synthesis of compound 23. ¹H NMR (500 MHz, CD₃OD) δ ppm 9.33 (s, 1H), 7.68 (dd, J=9.1, 5.2 Hz, 1H), 7.34 (s, 1H), 7.25 (t, J=9.2 Hz, 1H), 7.10 (s, 1H), 5.62 (s, 0.5H), 5.52 (s, 0.5H), 5.19 (s, 1H), 4.48-3.65 (m, 6H), 3.43 (s, 1H), 3.01-2.39 (m, 6H), 2.38-1.98 (m, 7H), 1.97-1.85 (m, 1H), 1.65-1.51 (m, 1H), 0.93-0.70 (m, 4H). m/z (ESI): 606.4 [M+H]⁺.

### Synthesis of Compound 28

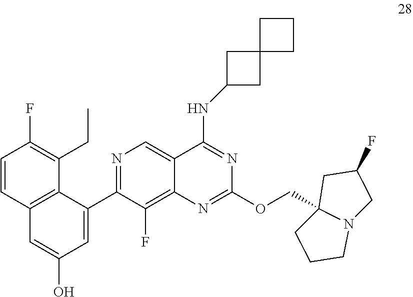 [SMILES: CCc1c(F)ccc2cc(O)cc(-c3ncc4c(NC5CC6(CCC6)C5)nc(OC[C@@]56CCCN5C[C@H](F)C6)nc4c3F)c12]

Title compound was synthesized from 2-aminospiro[3.3]heptane in a manner essentially analogous to the synthesis of compound 23. ¹H NMR (500 MHz, CD₃OD) δ ppm 9.25 (s, 1H), 7.71 (dd, J=9.0, 5.8 Hz, 1H), 7.34 (s, 1H), 7.28 (t, J=9.4 Hz, 1H), 7.09 (s, 1H), 5.40 (d, J=53.4 Hz, 1H), 4.72-4.63 (m, 1H), 4.51-4.31 (m, 2H), 3.52-3.37 (m, 3H), 3.26-3.08 (m, 1H), 2.67-2.56 (m, 2H), 2.53-2.30 (m, 3H), 2.27-2.19 (m, 5H), 2.16-2.02 (m, 5H), 2.00-1.88 (m, 3H), 0.81 (t, J=7.4 Hz, 3H). m/z (ESI): 604.5 [M+H]⁺.

### Synthesis of Compound 29

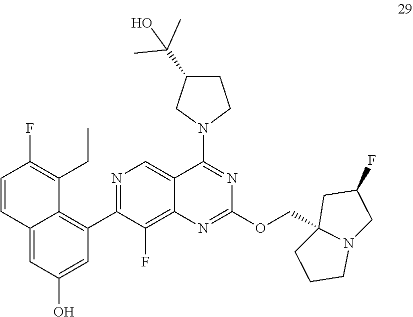 [SMILES: CCc1c(F)ccc2cc(O)cc(-c3ncc4c(N5CC[C@@H](C(C)(C)O)C5)nc(OC[C@@]56CCCN5C[C@H](F)C6)nc4c3F)c12]

Title compound was synthesized from (R)-2-(pyrrolidin-3-yl)propan-2-ol in a manner essentially analogous to the synthesis of compound 23. ¹H NMR (500 MHz, CD₃OD) δ ppm 0.75-0.82 (m, 3H), 2.03-2.65 (m, 12H), 3.21 (dd, J=14.4, 7.2 Hz, 6H), 3.68-3.88 (m, 4H), 4.07-4.21 (m, 3H), 4.58-4.62 (m, 2H), 5.45 (s, 0.5H), 5.56 (s, 0.5H), 7.05 (d, J=18.2 Hz, 1H), 7.26 (t, J=9.1 Hz, 1H), 7.32 (s, 1H), 7.66-7.73 (m, 1H), 9.31 (s, 1H). m/z (ESI): 622.5 [M+H]⁺.

### Synthesis of Compound 30

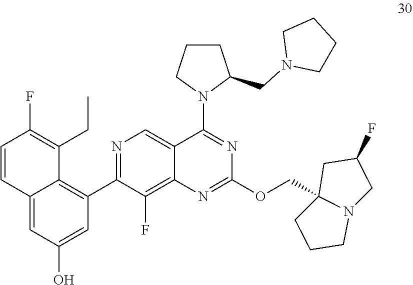 [SMILES: CCc1c(F)ccc2cc(O)cc(-c3ncc4c(N5CCC[C@H]5CN5CCCC5)nc(OC[C@@]56CCCN5C[C@H](F)C6)nc4c3F)c12]

Title compound was synthesized from (S)-(⁺)-1-(2-pyrrolidinylmethyl)pyrrolidine in a manner essentially analogous to the synthesis of compound 23. 1H NMR (500 MHz, CD3OD) δ ppm 0.79 (dd, J=13.8, 7.0 Hz, 3H), 1.87-2.38 (m, 18H), 2.44-2.53 (m, 1H), 2.80-2.82 (m, 4H), 3.01-3.05 (m, 2H), 3.20-3.24 (m, 2H), 4.10 (d, J=8.5 Hz, 1H), 4.21 (s, 1H), 4.28 (d, J=10.6 Hz, 1H), 4.28 (d, J=10.6 Hz, 1H), 5.27 (s, 0.5H), 5.37 (s, 0.5H), 7.05 (d, J=10.4 Hz, 1H), 7.25 (t, J=9.4 Hz, 1H), 7.30 (d, J=2.4 Hz, 1H), 7.68 (dd, J=8.9, 6.0 Hz, 1H), 9.27 (s, 1H). m/z (ESI): 647.5 [M+H]⁺.

### Synthesis of Compound 31

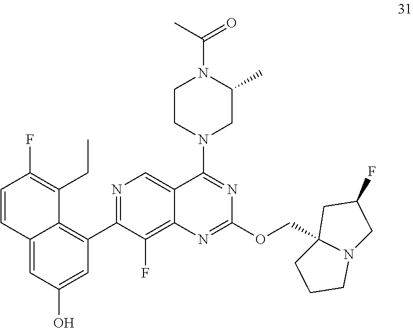 [SMILES: CCc1c(F)ccc2cc(O)cc(-c3ncc4c(N5CCN(C(C)=O)[C@H](C)C5)nc(OC[C@@]56CCCN5C[C@H](F)C6)nc4c3F)c12]

Title compound was synthesized from (R)-1-(2-Methyl-piperazin-1-yl)-ethanone in a manner essentially analogous to the synthesis of compound 3. ¹H NMR (500 MHz, CD₃OD) δ ppm 0.79 (t, J=6.6 Hz, 3H), 1.25-1.29 (m, 3H), 1.90-2.50 (m, 15H), 3.02 (dd, J=11.7, 7.0 Hz, 1H), 3.17-3.24 (m, 3H), 3.84-4.03 (m, 3H), 4.31 (d, J=5.8 Hz, 2H), 5.25 (s, 0.5H), 5.36 (s, 0.5H), 7.05 (d, J=9.7 Hz, 1H), 7.25 (t, J=9.3 Hz, 1H), 7.30 (s, 1H), 7.67 (dd, J=8.8, 5.6 Hz, 1H), 9.14 (s, 1H). m/z (ESI⁺): 635.5 [M+H]⁺.

### Synthesis of Compound 32

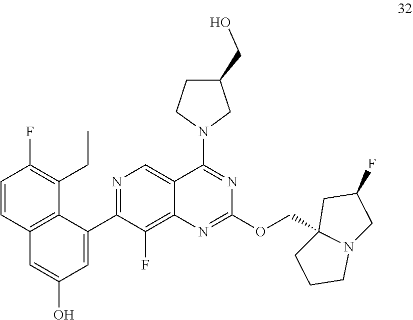 [SMILES: CCc1c(F)ccc2cc(O)cc(-c3ncc4c(N5CC[C@@H](CO)C5)nc(OC[C@@]56CCCN5C[C@H](F)C6)nc4c3F)c12]

Title compound was synthesized from (R)-pyrrolidin-3-ylmethanol in a manner essentially analogous to the synthesis of compound 23. ¹H NMR (500 MHz, CD₃OD) δ ppm 9.35 (d, J=3.6 Hz, 1H), 7.72 (dd, J=9.1, 5.8 Hz, 1H), 7.34 (d, J=2.6 Hz, 1H), 7.29 (t, J=9.3 Hz, 1H), 7.08 (s, 1H), 5.55 (d, J=52.3 Hz, 1H), 4.71-4.56 (m, 2H), 3.94-3.63 (m, 5H), 3.41 (s, 1H), 3.24 (q, J=7.4 Hz, 3H), 2.75-1.94 (m, 8H), 1.44 (s, 1H), 1.40-1.35 (m, 3H), 0.93 (t, J=6.8 Hz, 1H), 0.82 (t, J=7.4 Hz, 3H). m/z (ESI): 594.5 [M+H]⁺.

### Synthesis of Compound 33

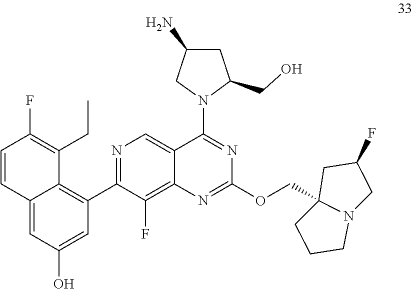 [SMILES: CCc1c(F)ccc2cc(O)cc(-c3ncc4c(N5C[C@@H](N)C[C@H]5CO)nc(OC[C@@]56CCCN5C[C@H](F)C6)nc4c3F)c12]

Title compound was synthesized from (3S,5S)-5-(hydroxymethyl)-3-pyrrolidinyl]-carbamic acid tert-butyl ester in a manner essentially analogous to the synthesis of compound 23. ¹H NMR (500 MHz, CD₃OD) δ ppm 9.18 (d, J=10.0 Hz, 1H), 7.67 (dd, J=9.1, 5.8 Hz, 1H), 7.29 (d, J=2.7 Hz, 1H), 7.24 (t, J=9.4 Hz, 1H), 7.04 (d, J=17.2 Hz, 1H), 5.35 (s, 0.5H), 5.24 (s, 0.5H), 4.39-4.13 (m, 4H), 3.87 (t, J=9.0 Hz, 1H), 3.78 (d, J=11.7 Hz, 1H), 3.64 (q, J=7.2 Hz, 1H), 3.28-3.17 (m, 3H), 3.06-2.97 (m, 1H), 2.55-2.42 (m, 2H), 2.36-1.81 (m, 8H), 0.79 (dt, J=18.4, 7.4 Hz, 3H). m/z (ESI): 609.4 [M+H]⁺.

### Synthesis of Compound 34

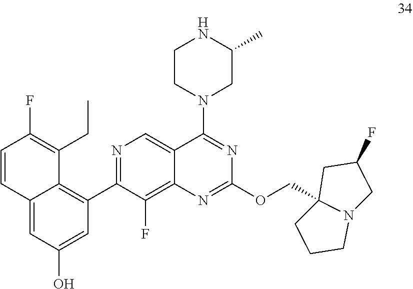 [SMILES: CCc1c(F)ccc2cc(O)cc(-c3ncc4c(N5CCN[C@H](C)C5)nc(OC[C@@]56CCCN5C[C@H](F)C6)nc4c3F)c12]

Title compound was synthesized from (R)-1-N-Boc-2-methylpiperazine in a manner essentially analogous to the synthesis of compound 23. ¹H NMR (500 MHz, CD₃OD) δ ppm 0.80 (t, J=5.8 Hz, 3H), 1.19 (d, J=3.0 Hz, 3H), 1.89-2.47 (m, 8H), 3.01 (t, J=10.5 Hz, 3H), 3.09-3.27 (m, 5H), 3.47-3.55 (m, 1H), 4.28 (dd, J=33.5, 10.4 Hz, 2H), 4.57-4.61 (m, 2H), 5.25 (s, 0.5H), 5.36 (s, 0.5H), 7.05 (s, 1H), 7.25 (t, J=9.3 Hz, 1H), 7.30 (s, 1H), 7.64-7.71 (m, 1H), 9.05 (s, 1H). m/z (ESI): 593.4 [M+H]⁺.

### Synthesis of Compound 35

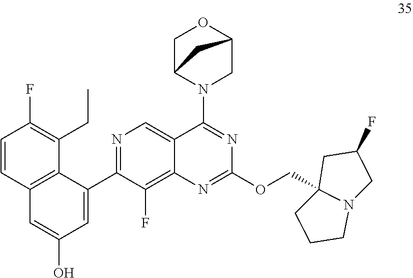 [SMILES: CCc1c(F)ccc2cc(O)cc(-c3ncc4c(N5C[C@@H]6C[C@H]5CO6)nc(OC[C@@]56CCCN5C[C@H](F)C6)nc4c3F)c12]

Title compound was synthesized from (1S, 4S)-2-oxa-5-aza-bicyclo[2.2.1]heptane in a manner essentially analogous to the synthesis of compound 23. ¹H NMR (500 MHz, CD₃OD) δ 9.18 (d, J=5.7 Hz, 1H), 7.71 (dd, J=8.8, 6.0 Hz, 1H), 7.33 (d, J=2.7 Hz, 1H), 7.28 (t, J=9.4 Hz, 1H), 7.08 (s, 1H), 5.63 (s, 1H), 5.34 (d, J=53.8 Hz, 1H), 4.39-4.24 (m, 3H), 4.15-3.99 (m, 3H), 3.31-3.18 (m, 3H), 3.05 (s, 1H), 2.52 (s, 1H), 2.43-1.84 (m, 11H), 0.89-0.78 (m, 3H). m/z (ESI): 592.4 [M+H]⁺.

### Synthesis of Compound 36

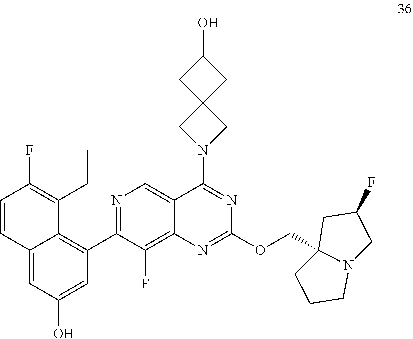 [SMILES: CCc1c(F)ccc2cc(O)cc(-c3ncc4c(N5CC6(CC(O)C6)C5)nc(OC[C@@]56CCCN5C[C@H](F)C6)nc4c3F)c12]

Title compound was synthesized from 2-aza-spiro[3.3]heptan-6-ol in a manner essentially analogous to the synthesis of compound 23. ¹H NMR (500 MHz, CD₃OD) δ ppm 0.78 (t, J=7.0 Hz, 3H), 2.11-2.69 (m, 12H), 3.01 (q, J=7.5 Hz, 2H), 3.34-3.42 (m, 1H), 3.72-3.94 (m, 3H), 4.22 (t, J=8.5 Hz, 1H), 4.42-4.47 (m, 2H), 4.54-4.65 (m, 2H), 5.47 (s, 0.5H), 5.57 (s, 0.5H), 7.02 (s, 1H), 7.25 (t, J=9.5 Hz, 1H), 7.30 (s, 1H), 7.67-7.70 (m, 1H), 8.95 (s, 1H). m/z (ESI): 606.4 [M+H]⁺.

### Synthesis of Compound 37

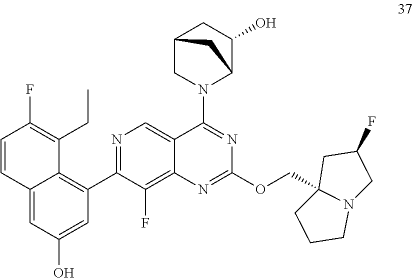 [SMILES: CCc1c(F)ccc2cc(O)cc(-c3ncc4c(N5C[C@H]6C[C@@H]5[C@@H](O)C6)nc(OC[C@@]56CCCN5C[C@H](F)C6)nc4c3F)c12]

Title compound was synthesized from (1R,4S,6S)-2-azabicyclo[2.2.1]heptan-6-ol in a manner essentially analogous to the synthesis of compound 23. ¹H NMR (500 MHz, CD₃OD) δ ppm 9.16 (d, J=3.7 Hz, 1H), 7.66 (dd, J=9.0, 5.7 Hz, 1H), 7.28 (d, J=2.7 Hz, 1H), 7.23 (t, J=9.4 Hz, 1H), 7.02 (d, J=9.6 Hz, 1H), 5.38 (s, 0.5H), 5.27 (s, 0.5H), 5.11-4.97 (m, 1H), 4.41-4.23 (m, 2H), 4.20-4.00 (m, 2H), 3.65-3.50 (m, 1H), 3.42-3.34 (m, 1H), 3.11-3.01 (m, 1H), 2.90-2.76 (m, 1H), 2.54-2.42 (m, 1H), 2.42-2.08 (m, 5H), 2.08-1.74 (m, 7H), 1.71-1.49 (m, 1H), 0.84-0.72 (m, 3H). m/z (ESI): 606.4 [M+H]⁺.

### Synthesis of Compound 38

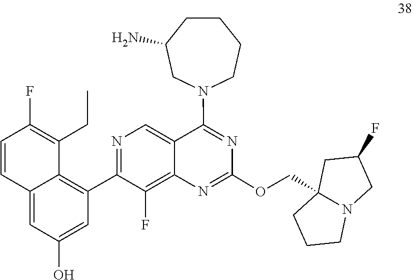 [SMILES: CCc1c(F)ccc2cc(O)cc(-c3ncc4c(N5CCCC[C@@H](N)C5)nc(OC[C@@]56CCCN5C[C@H](F)C6)nc4c3F)c12]

Title compound was synthesized from (R)-tert-butyl azepan-3-ylcarbamate in a manner essentially analogous to the synthesis of compound 23. ¹H NMR (500 MHz, CD₃OD) δ ppm 9.18 (s, 1H), 7.73-7.60 (m, 1H), 7.34-7.21 (m, 2H), 7.05 (d, J=9.0 Hz, 1H), 5.37 (s, 0.6H), 5.26 (s, 0.4H), 4.62 (s, 2H), 4.46-4.09 (m, 4H), 3.61-3.42 (m, 2H), 3.27-3.19 (m, 2H), 3.10-2.97 (m, 1H), 2.56-2.43 (m, 1H), 2.39-1.86 (m, 11H), 1.68-1.44 (m, 2H), 0.85-0.76 (m, 3H). m/z (ESI): 607.4 [M+H]⁺.

### Synthesis of Compound 39

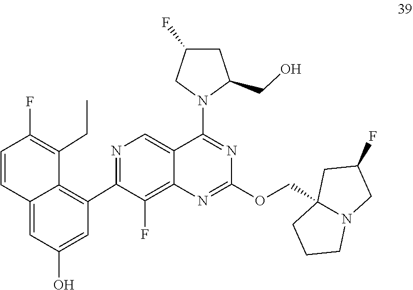 [SMILES: CCc1c(F)ccc2cc(O)cc(-c3ncc4c(N5C[C@H](F)C[C@H]5CO)nc(OC[C@@]56CCCN5C[C@H](F)C6)nc4c3F)c12]

Title compound was synthesized from [(2S,4R)-4-fluoropyrrolidin-2-yl]methanol in a manner essentially analogous to the synthesis of compound 23. ¹H NMR (500 MHz, CD₃OD) δ ppm 9.12 (d, J=8.3 Hz, 1H), 7.67 (dd, J=9.0, 5.8 Hz, 1H), 7.29 (d, J=2.7 Hz, 1H), 7.24 (t, J=9.4 Hz, 1H), 7.05 (d, J=2.9 Hz, 1H), 5.51 (s, 0.5H), 5.41 (s, 0.5H), 5.37 (s, 0.5H), 5.26 (s, 0.5H), 5.02-4.94 (m, 1H), 4.45-4.09 (m, 6H), 3.84-3.77 (m, 1H), 3.38-3.32 (m, 1H), 3.26-3.19 (m, 1H), 3.13-2.97 (m, 1H), 2.56-2.09 (m, 8H), 2.08-1.84 (m, 4H), 0.79 (dt, J=24.5, 7.4 Hz, 3H). m/z (ESI): 612.4 [M+H]⁺.

### Synthesis of Compound 40

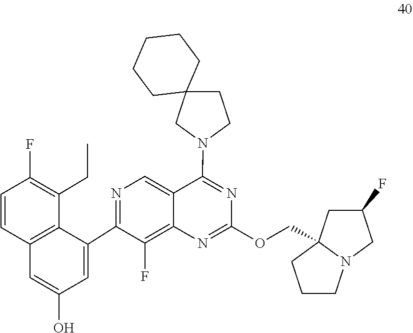 [SMILES: CCc1c(F)ccc2cc(O)cc(-c3ncc4c(N5CCC6(CCCCC6)C5)nc(OC[C@@]56CCCN5C[C@H](F)C6)nc4c3F)c12]

Title compound was synthesized from 2-azaspiro[4.5]decane in a manner essentially analogous to the synthesis of compound 23. ¹H NMR (500 MHz, CD₃OD) δ ppm 9.34 (s, 1H), 7.77-7.64 (m, 1H), 7.34 (s, 1H), 7.29 (t, J=9.5 Hz, 1H), 7.08 (s, 1H), 5.55 (d, J=51.6 Hz, 1H), 4.72-4.54 (m, 2H), 3.86-3.71 (m, 1H), 2.65-2.45 (m, 2H), 2.44-2.36 (m, 1H), 2.30 (s, 2H), 2.22-2.06 (s, 1H), 1.65 (s, 11H), 1.38-1.28 (m, 10H), 0.85-0.79 (m, 3H). m/z (ESI): 632.4 [M+H]⁺.

### Synthesis of Compound 41

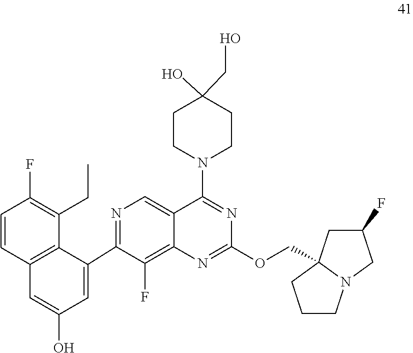 [SMILES: CCc1c(F)ccc2cc(O)cc(-c3ncc4c(N5CCC(O)(CO)CC5)nc(OC[C@@]56CCCN5C[C@H](F)C6)nc4c3F)c12]

Title compound was synthesized from 4-(hydroxymethyl)piperidin-4-ol in a manner essentially analogous to the synthesis of compound 23. ¹H NMR (500 MHz, CD₃OD) δ ppm 9.11 (s, 1H), 7.73 (s, 1H), 7.39-7.21 (m, 2H), 7.11 (s, 1H), 5.35 (d, J=53.0 Hz, 1H), 4.61 (s, 2H), 4.41-4.25 (m, 2H), 3.84 (s, 2H), 3.65 (s, 2H), 3.33-3.15 (m, 3H), 3.06 (s, 1H), 2.53 (s, 1H), 2.39 (s, 1H), 2.32-2.12 (m, 4H), 2.11-1.87 (m, 8H), 0.85 (s, 3H). m/z (ESI): 656.4 [M+H]⁺.

### Synthesis of Compound 42

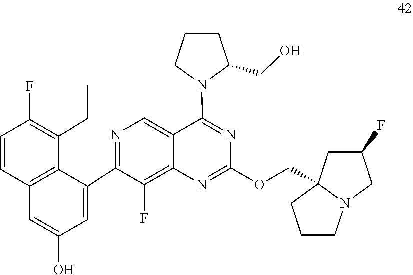 [SMILES: CCc1c(F)ccc2cc(O)cc(-c3ncc4c(N5CCC[C@@H]5CO)nc(OC[C@@]56CCCN5C[C@H](F)C6)nc4c3F)c12]

Title compound was synthesized from (R)-1-pyrrolidin-2-ylmethanol in a manner essentially analogous to the synthesis of compound 23. ¹H NMR (500 MHz, CD₃OD) δ ppm 9.29 (s, 1H), 7.71 (s, 1H), 7.33 (s, 1H), 7.28 (t, J=9.4 Hz, 1H), 7.08 (d, J=10.9 Hz, 1H), 5.34 (d, J=53.9 Hz, 1H), 4.69 (s, 1H), 4.31 (s, 2H), 4.18 (s, 2H), 3.95 (s, 2H), 3.29-3.22 (m, 2H), 3.06 (s, 1H), 2.53 (s, 1H), 2.42-1.98 (m, 11H), 1.92 (s, 1H), 0.87-0.79 (m, 3H). m/z (ESI): 594.4 [M+H]⁺.

### Synthesis of Compound 43

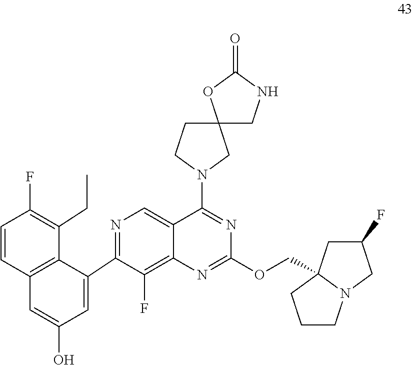 [SMILES: CCc1c(F)ccc2cc(O)cc(-c3ncc4c(N5CCC6(CNC(=O)O6)C5)nc(OC[C@@]56CCCN5C[C@H](F)C6)nc4c3F)c12]

Title compound was synthesized from 1-oxa-3,7-diazaspiro[4.4]nonan-2-one in a manner essentially analogous to the synthesis of compound 23. ¹H NMR (500 MHz, CD₃OD) δ ppm 9.30 (s, 1H), 7.75-7.65 (m, 1H), 7.34 (s, 1H), 7.28 (t, J=9.3 Hz, 1H), 7.09 (d, J=9.7 Hz, 1H), 5.34 (d, J=53.8 Hz, 1H), 4.67 (s, 2H), 4.51-4.10 (m, 4H), 3.83 (dd, J=63.8, 9.6 Hz, 2H), 3.53 (d, J=52.0 Hz, 1H), 3.30-3.22 (m, 2H), 3.05 (s, 1H), 2.53 (s, 1H), 2.43-2.35 (m, 1H), 2.33-2.13 (m, 3H), 2.07 (s, 2H), 2.05-1.98 (m, 2H), 1.98-1.87 (m, 1H), 0.87-0.78 (m, 3H). m/z (ESI): 635.4 [M+H]⁺.

### Synthesis of Compound 44

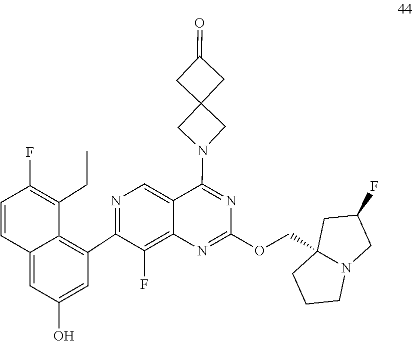 [SMILES: CCc1c(F)ccc2cc(O)cc(-c3ncc4c(N5CC6(CC(=O)C6)C5)nc(OC[C@@]56CCCN5C[C@H](F)C6)nc4c3F)c12]

Title compound was synthesized from 2-azaspiro[3.3]heptan-6-one in a manner essentially analogous to the synthesis of compound 23. ¹H NMR (500 MHz, CD₃OD) δ ppm 0.78 (t, J=7.5 Hz, 3H), 1.36-1.37 (m, 3H), 2.10-2.68 (m, 8H), 3.49-3.51 (m, 3H), 3.72-3.95 (m, 3H), 4.57-4.68 (m, 4H), 5.12 (s, 1H), 5.48 (s, 0.5H), 5.59 (s, 0.5H), 7.03 (s, 1H), 7.26 (t, J=9.5 Hz, 1H), 7.31 (s, 1H), 7.67-7.70 (m, 1H), 8.99 (s, 1H). m/z (ESI⁺): 604.3 [M+H]⁺.

### Synthesis of Compound 45

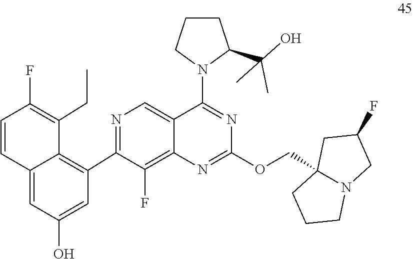 [SMILES: CCc1c(F)ccc2cc(O)cc(-c3ncc4c(N5CCC[C@H]5C(C)(C)O)nc(OC[C@@]56CCCN5C[C@H](F)C6)nc4c3F)c12]

Title compound was synthesized from (S)-2-(pyrrolidin-2-yl)propan-2-ol in a manner essentially analogous to the synthesis of compound 23. ¹H NMR (500 MHz, CD₃OD) δ ppm 9.24 (d, J=6.9 Hz, 1H), 7.79-7.60 (m, 1H), 7.45-7.19 (m, 2H), 7.13-7.00 (m, 1H), 5.48 (s, 0.5H), 5.37 (s, 0.5H), 5.21 (s, 1H), 4.64 (s, 1H), 4.55 (d, J=9.9 Hz, 1H), 4.45-4.33 (m, 1H), 4.30-4.07 (m, 2H), 3.70-3.44 (m, 3H), 3.26-3.15 (m, 1H), 2.57-1.89 (m, 12H), 1.37-1.28 (m, 6H), 0.89-0.75 (m, 3H). m/z (ESI): 622.4 [M+H]⁺.

### Synthesis of Compound 46

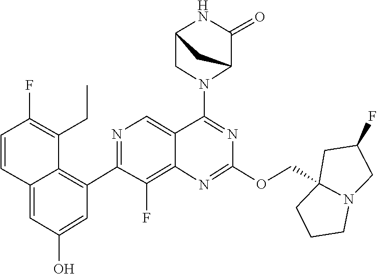 [SMILES: CCc1c(F)ccc2cc(O)cc(-c3ncc4c(N5C[C@H]6C[C@@H]5C(=O)N6)nc(OC[C@@]56CCCN5C[C@H](F)C6)nc4c3F)c12]

Title compound was synthesized from (1R,4R)-2,5-diazabicyclo[2.2.1]heptan-3-one in a manner essentially analogous to the synthesis of compound 23. ¹H NMR (500 MHz, CD₃OD) δ ppm 9.36 (s, 1H), 7.75-7.65 (m, 1H), 7.33 (s, 1H), 7.28 (t, J=9.2 Hz, 1H), 7.08 (d, J=10.7 Hz, 1H), 5.53 (s, 1H), 5.41 (s, 0.5H), 5.30 (s, 0.5H), 4.67 (s, 1H), 4.57-4.38 (m, 3H), 4.29 (d, J=10.7 Hz, 1H), 3.84 (q, J=9.3 Hz, 1H), 3.30-3.23 (m, 2H), 3.11-2.97 (m, 1H), 2.52 (dt, J=18.3, 7.7 Hz, 1H), 2.46-2.23 (m, 3H), 2.23-2.13 (m, 3H), 2.08-1.91 (m, 3H), 0.82 (dt, J=14.2, 7.4 Hz, 3H). m/z (ESI): 605.4 [M+H]⁺.

### Synthesis of Compound 47

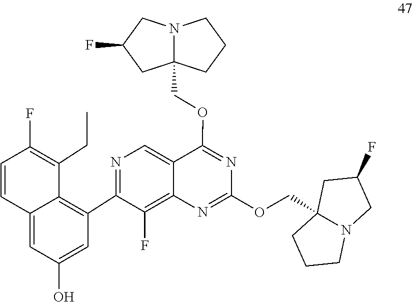 [SMILES: CCc1c(F)ccc2cc(O)cc(-c3ncc4c(OC[C@@]56CCCN5C[C@H](F)C6)nc(OC[C@@]56CCCN5C[C@H](F)C6)nc4c3F)c12]

Title compound was synthesized from compound 1-5 in a manner essentially analogous to the synthesis of compound 3. ¹H NMR (500 MHz, CD₃OD) δ ppm 9.15 (s, 1H), 7.65 (dd, J=9.3, 5.4 Hz, 1H), 7.28 (s, 1H), 7.22 (t, J=9.4 Hz, 1H), 7.00 (s, 1H), 5.42 (d, J=15.3 Hz, 1H), 5.31 (d, J=15.0 Hz, 1H), 4.56-4.43 (m, 3H), 3.56-3.31 (m, 6H), 3.22-3.04 (m, 2H), 2.53-1.86 (m, 15H), 0.79-0.65 (m, 3H). m/z (ESI): 652.4 [M+H]⁺.

### Synthesis of Compound 48

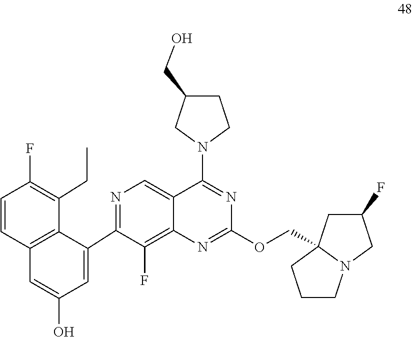 [SMILES: CCc1c(F)ccc2cc(O)cc(-c3ncc4c(N5CC[C@H](CO)C5)nc(OC[C@@]56CCCN5C[C@H](F)C6)nc4c3F)c12]

Title compound was synthesized from (S)-3-pyrrolidin-methanol in a manner essentially analogous to the synthesis of compound 23. ¹H NMR (500 MHz, CD₃OD) δ ppm 9.29 (d, J=4.4 Hz, 1H), 7.70 (dd, J=9.1, 5.7 Hz, 1H), 7.33 (s, 1H), 7.27 (t, J=9.3 Hz, 1H), 7.08 (s, 1H), 5.33 (d, J=54.1 Hz, 1H), 4.42-4.23 (m, 3H), 4.19 (s, 2H), 3.79-3.63 (m, 2H), 3.30-3.16 (m, 3H), 3.12-2.95 (m, 1H), 2.66 (s, 1H), 2.52 (dd, J=15.1, 8.0 Hz, 1H), 2.43-1.81 (m, 10H), 0.88-0.78 (m, 3H). m/z (ESI): 594.3 [M+H]⁺.

### Synthesis of Compound 49

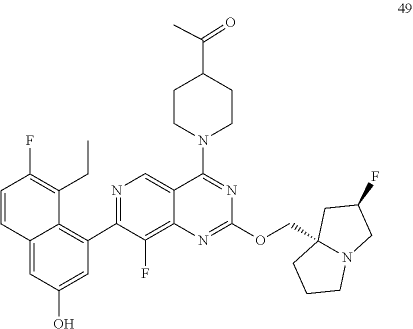 [SMILES: CCc1c(F)ccc2cc(O)cc(-c3ncc4c(N5CCC(C(C)=O)CC5)nc(OC[C@@]56CCCN5C[C@H](F)C6)nc4c3F)c12]

Title compound was synthesized from 1-(piperidin-4-yl)ethan-1-one in a manner essentially analogous to the synthesis of compound 23. ¹H NMR (500 MHz, CD₃OD) δ ppm 0.79 (t, J=7.5 Hz, 3H), 1.81-1.90 (m, 3H), 1.96-2.02 (m, 2H), 2.11-2.20 (m, 5H), 2.23 (s, 3H), 2.25-2.36 (m, 1H), 2.45-2.50 (m, 1H), 2.89-3.03 (m, 2H), 3.16-3.28 (m, 3H), 3.55 (q, J=11.5 Hz, 2H), 4.24-4.33 (m, 2H), 4.63-4.66 (m, 2H), 5.24 (s, 0.5H), 5.35 (s, 0.5H), 7.05 (s, 1H), 7.25 (t, J=9.5 Hz, 1H), 7.30 (s, 1H), 7.66-7.69 (m, 1H), 9.03 (s, 1H). m/z (ESI): 620.4 [M+H]⁺.

### Synthesis of Compound 50

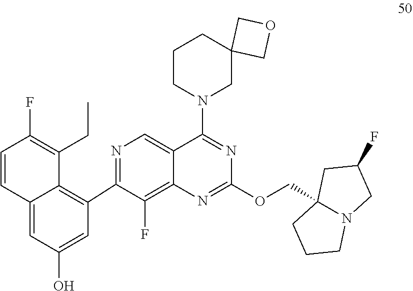 [SMILES: CCc1c(F)ccc2cc(O)cc(-c3ncc4c(N5CCCC6(COC6)C5)nc(OC[C@@]56CCCN5C[C@H](F)C6)nc4c3F)c12]

Title compound was synthesized from 2-Oxa-6-azaspiro[3.5]nonane in a manner essentially analogous to the synthesis of compound 23. ¹H NMR (500 MHz, CD₃OD) δ ppm 0.80 (t, J=7.5 Hz, 3H), 1.79-2.37 (m, 14H), 2.45-2.49 (m, 1H), 3.01-3.03 (m, 1H), 3.92-4.05 (m, 2H), 4.29-4.37 (m, 6H), 4.48 (s, 2H), 5.26 (s, 0.5H), 5.37 (s, 0.5H), 7.06 (s, 1H), 7.26 (t, J=9.5 Hz, 1H), 7.30 (s, 1H), 7.67-7.69 (m, 1H), 9.06 (s, 1H). m/z (ESI): 620.4 [M+H]⁺.

### Synthesis of Compound 51

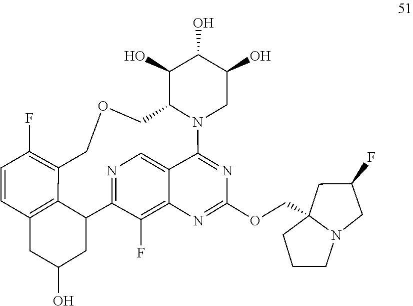 [SMILES: OC1Cc2ccc(F)c3c2C(C1)c1ncc2c(nc(OC[C@@]45CCCN4C[C@H](F)C5)nc2c1F)N1C[C@H](O)[C@@H](O)[C@H](O)[C@H]1COC3]

Title compound was synthesized from 1-Deoxynojirimycin in a manner essentially analogous to the synthesis of compound 23. ¹H NMR (500 MHz, CD₃OD) δ ppm 9.40 (s, 1H), 7.78-7.65 (m, 1H), 7.35 (s, 1H), 7.29 (t, J=9.3 Hz, 1H), 7.08 (s, 1H), 5.34 (d, J=53.7 Hz, 1H), 4.49-4.34 (m, 2H), 3.56 (d, J=13.7 Hz, 1H), 3.43 (t, J=9.4 Hz, 1H), 3.31-3.16 (m, 5H), 3.05 (s, 1H), 2.98 (s, 1H), 2.57 (t, J=11.6 Hz, 1H), 2.47 (s, 1H), 2.41-2.33 (m, 1H), 2.33-1.87 (m, 8H), 0.85-0.75 (m, 3H). m/z (ESI): 656.5 [M+H]⁺.

### Synthesis of Compound 52

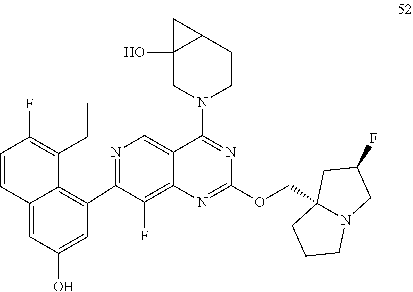 [SMILES: CCc1c(F)ccc2cc(O)cc(-c3ncc4c(N5CCC6CC6(O)C5)nc(OC[C@@]56CCCN5C[C@H](F)C6)nc4c3F)c12]

Title compound was synthesized from 4-azabicyclo[4.1.0]heptan-6-ol in a manner essentially analogous to the synthesis of compound 23. ¹H NMR (500 MHz, CD₃OD) δ ppm 0.63-0.66 (m, 1H), 0.77-0.81 (m, 3H), 1.01-1.04 (m, 1H), 1.44 (s, 1H), 1.87 (s, 1H), 2.15-2.69 (m, 9H), 3.74-3.93 (m, 4H), 4.05 (s, 1H), 4.26 (t, J=14 Hz, 1H), 4.48-4.65 (m, 4H), 5.48 (s, 0.5H), 5.59 (s, 0.5H), 7.04-7.06 (m, 1H), 7.26 (t, J=9.5 Hz, 1H), 7.31 (s, 1H), 7.67-7.69 (m, 1H), 9.16 (s, 1H). m/z (ESI): 606.4 [M+H]⁺.

### Synthesis of Compound 53

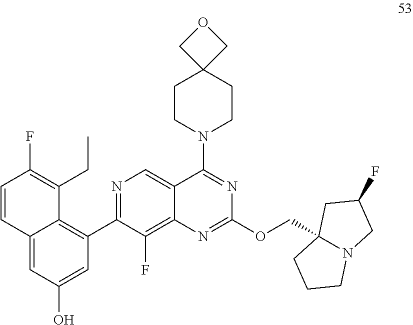 [SMILES: CCc1c(F)ccc2cc(O)cc(-c3ncc4c(N5CCC6(CC5)COC6)nc(OC[C@@]56CCCN5C[C@H](F)C6)nc4c3F)c12]

Title compound was synthesized from 2-Oxa-7-aza-spiro[3.5]nonane in a manner essentially analogous to the synthesis of compound 23. ¹H NMR (500 MHz, CD₃OD) δ ppm 0.80-0.83 (m, 3H), 1.94-2.50 (m, 13H), 3.03-3.11 (m, 1H), 3.25-3.29 (m, 2H), 4.03 (s, 4H), 4.29-4.38 (m, 2H), 4.59 (m, 4H), 5.30 (s, 0.5H), 5.41 (s, 0.5H), 7.07 (s, 1H), 7.28 (t, J=11.0 Hz, 1H), 7.33 (s, 1H), 7.69-7.72 (m, 1H), 9.06 (s, 1H). m/z (ESI): 620.4 [M+H]⁺.

### Synthesis of Compound 54

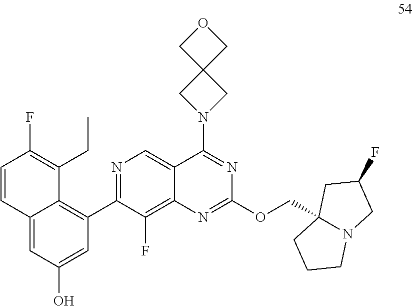 [SMILES: CCc1c(F)ccc2cc(O)cc(-c3ncc4c(N5CC6(COC6)C5)nc(OC[C@@]56CCCN5C[C@H](F)C6)nc4c3F)c12]

Title compound was synthesized from 2-oxa-6-azaspiro[3.3]heptane in a manner essentially analogous to the synthesis of compound 23. ¹H NMR (500 MHz, CD₃OD) δ ppm 0.80 (t, J=7.5 Hz, 3H), 1.93-2.51 (m, 8H), 3.06-3.11 (m, 1H), 3.26-3.42 (m, 5H), 4.30-4.39 (m, 2H), 4.67 (s, 4H), 5.04-5.09 (m, 2H), 5.31 (s, 0.5H), 5.41 (s, 0.5H), 7.04-7.06 (m, 1H), 7.28 (t, J=9.5 Hz, 1H), 7.33 (s, 1H), 7.69-7.72 (m, 1H), 8.97 (s, 1H). m/z (ESI): 592.4 [M+H]⁺.

### Synthesis of Compound 55

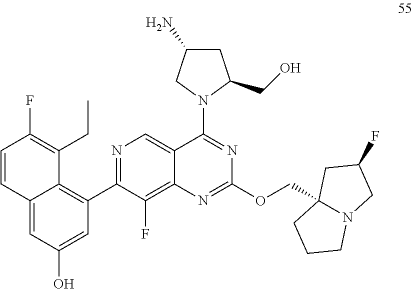 [SMILES: CCc1c(F)ccc2cc(O)cc(-c3ncc4c(N5C[C@H](N)C[C@H]5CO)nc(OC[C@@]56CCCN5C[C@H](F)C6)nc4c3F)c12]

Title compound was synthesized from ((2S,4R)-4-aminopyrrolidin-2-yl)methanol in a manner essentially analogous to the synthesis of compound 23. ¹H NMR (500 MHz, CD₃OD) δ ppm 0.80-0.85 (m, 3H), 1.92-2.53 (m, 11H), 3.02-3.06 (m, 1H), 3.20-3.27 (m, 3H), 3.85-3.95 (m, 3H), 4.09-4.41 (m, 4H), 5.29 (s, 0.5H), 5.39 (s, 0.5H), 7.06-7.09 (m, 1H), 7.28 (t, J=9.5 Hz, 1H), 7.33 (s, 1H), 7.69-7.72 (m, 1H), 9.23 (s, 1H). m/z (ESI): 609.4 [M+H]⁺.

### Synthesis of Compound 56

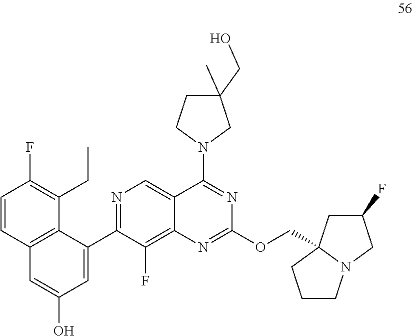 [SMILES: CCc1c(F)ccc2cc(O)cc(-c3ncc4c(N5CCC(C)(CO)C5)nc(OC[C@@]56CCCN5C[C@H](F)C6)nc4c3F)c12]

Title compound was synthesized from (3-Methyl-3-pyrrolidinyl)methanol in a manner essentially analogous to the synthesis of compound 23. ¹H NMR (500 MHz, CD₃OD) δ ppm 9.30 (s, 1H), 7.71 (dd, J=9.0, 5.8 Hz, 1H), 7.34 (d, J=2.7 Hz, 1H), 7.28 (t, J=9.3 Hz, 1H), 7.08 (d, J=2.6 Hz, 1H), 5.36 (d, J=53.7 Hz, 1H), 4.45-4.29 (m, 2H), 4.22-3.96 (m, 1H), 3.58 (s, 2H), 3.49-3.37 (m, 1H), 3.33-3.18 (m, 5H), 3.13-3.02 (m, 1H), 2.59-2.48 (m, 1H), 2.45-2.11 (m, 5H), 2.10-2.01 (m, 2H), 2.00-1.85 (m, 2H), 1.26 (s, 3H), 0.87-0.79 (m, 3H). m/z (ESI): 608.4 [M+H]⁺.

### Synthesis of Compound 57

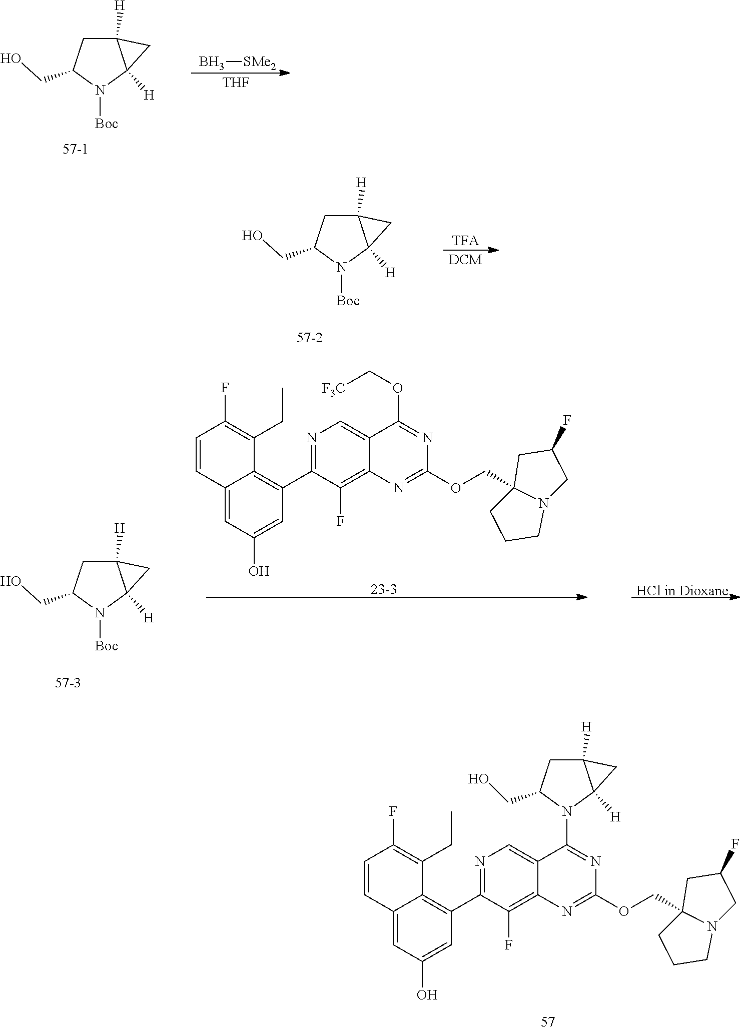

A solution of intermediate 57-1 (50 mg, 220.02 μmol, 1 eq) in tetrahydrofuran (3 mL) was purged with nitrogen gas and cooled to 0° C. Borane-methyl sulfide complex 2M Solution in Tetrahydrofuran (110 μL, 1 eq) was added. The reaction mixture was warmed to 60° C. and stirred for 3 hours. Upon the completion of conversion, the reaction mixture was cooled to 0° C. and quenched by adding methanol. Then the mixture was gradually warmed up to room temperature and the solvent was removed in vacuo at 25° C. to afford crude residue, which was subjected to silica gel chromatography eluted with dichloromethane/methanol (v/v=10/1) to afford compound 57-2 (25 mg, Yield 53.3%).

To a stirred solution of intermediate 57-2 (25 mg, 117.22 μmol, 1 eq) in dichloromethane (1 mL), trifluoroacetic acid (13.37 mg, 117.22 μmol, 8.71 μL, 1 eq) was added in one portion. The reaction mixture was stirred for 5 minutes at room temperature. Upon the completion of conversion, the solvent was removed in vacuo to afford compound 57-3, which was directly subjected to the next transformation without further purification (26 mg, Yield 97.63%)

Compound 57 was synthesized from compounds 57-3 and 23-3 in a manner essentially analogous to the synthesis of compound 23. ¹H NMR (500 MHz, CD₃OD) δ ppm 0.82-0.84 (m, 3H), 2.05-2.76 (m, 10H), 3.24-3.28 (m, 3H), 3.75-4.08 (m, 8H), 4.64-4.73 (m, 2H), 5.54 (s, 0.5H), 5.65 (s, 0.5H), 7.09 (s, 1H), 7.29 (t, J=9.5 Hz, 1H), 7.35 (s, 1H), 7.71-7.73 (m, 1H), 9.95-9.96 (s, 1H). m/z (ESI): 606.3 [M+H]⁺.

### Synthesis of Compound 58

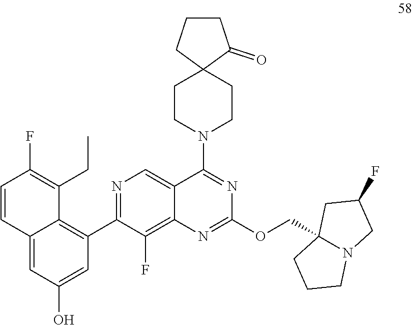 [SMILES: CCc1c(F)ccc2cc(O)cc(-c3ncc4c(N5CCC6(CCCC6=O)CC5)nc(OC[C@@]56CCCN5C[C@H](F)C6)nc4c3F)c12]

Title compound was synthesized from 8-azaspiro[4.5]decan-1-one in a manner essentially analogous to the synthesis of compound 23. ¹H NMR (500 MHz, CD₃OD) δ ppm 0.82 (t, J=7.5 Hz, 3H), 1.70-1.73 (m, 2H), 1.94-2.50 (m, 16H), 3.04 (s, 1H), 3.23-3.32 (m, 3H), 3.87-3.91 (m, 2H), 4.26-4.44 (m, 4H), 5.27 (s, 0.5H), 5.38 (s, 0.5H), 5.51 (s, 1H), 7.07 (s, 1H), 7.27 (t, J=9.5 Hz, 1H), 7.32 (s, 1H), 7.70 (t, J=10.0 Hz, 1H), 9.07 (s, 1H). m/z (ESI): 646.4 [M+H]⁺.

Synthesis of compound 59

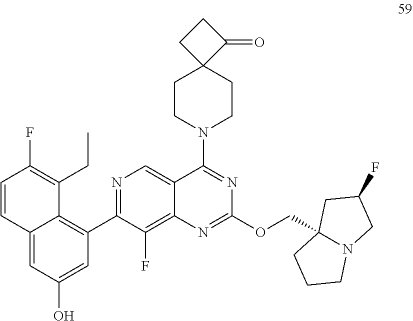 [SMILES: CCc1c(F)ccc2cc(O)cc(-c3ncc4c(N5CCC6(CCC6=O)CC5)nc(OC[C@@]56CCCN5C[C@H](F)C6)nc4c3F)c12]

Title compound was synthesized from 7-azaspiro[3.5]nonan-1-one in a manner essentially analogous to the synthesis of compound 23. ¹H NMR (500 MHz, CD₃OD) δ ppm 0.79 (t, J=6.5 Hz, 3H), 1.68-1.70 (m, 2H), 1.91-2.47 (m, 14H), 3.02 (s, 1H), 3.20-3.29 (m, 3H), 3.84-3.88 (m, 2H), 4.24-4.41 (m, 4H), 5.25 (s, 0.5H), 5.35 (s, 0.5H), 7.05 (s, 1H), 7.24 (t, J=9.5 Hz, 1H), 7.29 (s, 1H), 7.67 (t, J=8.0 Hz, 1H), 9.04 (s, 1H). m/z (ESI): 632.4 [M+H]⁺.

### Synthesis of Compound 60

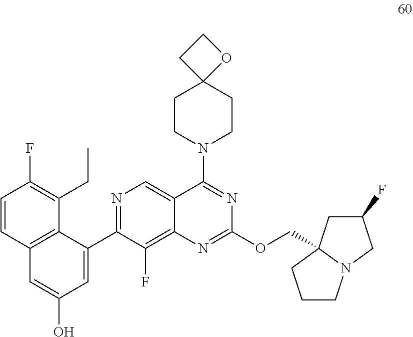 [SMILES: CCc1c(F)ccc2cc(O)cc(-c3ncc4c(N5CCC6(CCO6)CC5)nc(OC[C@@]56CCCN5C[C@H](F)C6)nc4c3F)c12]

Title compound was synthesized from 1-oxa-7-aza-spiro[3,5]nonane in a manner essentially analogous to the synthesis of compound 23. ¹H NMR (500 MHz, CD₃OD) δ ppm 9.12 (s, 1H), 7.71 (dd, J=9.1, 5.8 Hz, 1H), 7.34 (d, J=2.6 Hz, 1H), 7.28 (t, J=9.3 Hz, 1H), 7.09 (s, 1H), 5.63 (s, 1H), 5.38 (d, J=53.6 Hz, 1H), 4.60 (s, 2H), 4.45-4.30 (m, 2H), 4.31-4.11 (m, 2H), 3.73 (t, J=6.5 Hz, 2H), 3.61-3.41 (m, 1H), 3.26-3.17 (m, 1H), 3.11 (s, 1H), 2.60-1.89 (m, 13H), 0.84 (dt, J=7.9, 3.9 Hz, 3H). m/z (ESI): 620.4 [M+H]⁺.

### Synthesis of Compound 61

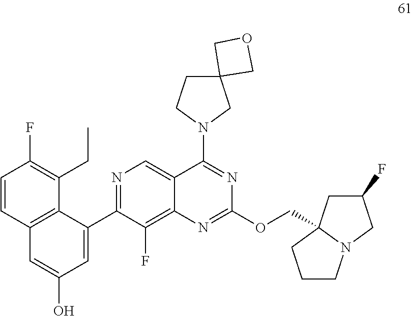 [SMILES: CCc1c(F)ccc2cc(O)cc(-c3ncc4c(N5CCC6(COC6)C5)nc(OC[C@@]56CCCN5C[C@H](F)C6)nc4c3F)c12]

Title compound was synthesized from 2-oxa-6-azaspiro[3.4]octane in a manner essentially analogous to the synthesis of compound 23. ¹H NMR (500 MHz, CD₃OD) δ ppm 9.32 (s, 1H), 7.72 (dd, J=9.0, 5.7 Hz, 1H), 7.52 (t, J=7.7 Hz, 1H), 7.38 (t, J=7.7 Hz, 1H), 7.30 (t, J=9.3 Hz, 1H), 7.08 (s, 1H), 5.44 (d, J=53.1 Hz, 1H), 4.88-4.72 (m, 4H), 4.59-3.80 (m, 5H), 3.69-3.41 (m, 3H), 3.28-3.15 (m, 1H), 2.64-1.92 (m, 11H), 0.87-0.79 (m, 3H). m/z (ESI): 606.4 [M+H]⁺.

### Synthesis of Compound 62

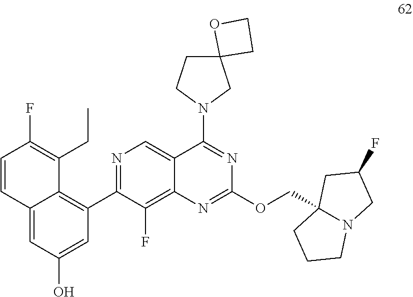 [SMILES: CCc1c(F)ccc2cc(O)cc(-c3ncc4c(N5CCC6(CCO6)C5)nc(OC[C@@]56CCCN5C[C@H](F)C6)nc4c3F)c12]

Title compound was synthesized from 1-oxa-6-azaspiro[3,4]octane in a manner essentially analogous to the synthesis of compound 23. ¹H NMR (500 MHz, CD₃OD) δ ppm 0.76-0.82 (m, 3H), 1.90-2.59 (m, 11H), 2.80-2.85 (m, 1H), 2.94-3.02 (m, 2H), 3.19-3.23 (m, 2H), 4.01-4.41 (m, 6H), 4.61 (t, J=8.5 Hz, 2H), 5.25 (s, 0.5H), 5.36 (s, 0.5H), 7.05 (d, J=11.8 Hz, 1H), 7.25 (t, J=9.3 Hz, 1H), 7.30 (d, J=2.1 Hz, 1H), 7.68 (dd, J=8.8, 5.9 Hz, 1H), 9.26 (d, J=6.5 Hz, 1H). m/z (ESI): 606.3 [M+H]⁺.

### Synthesis of Compound 63

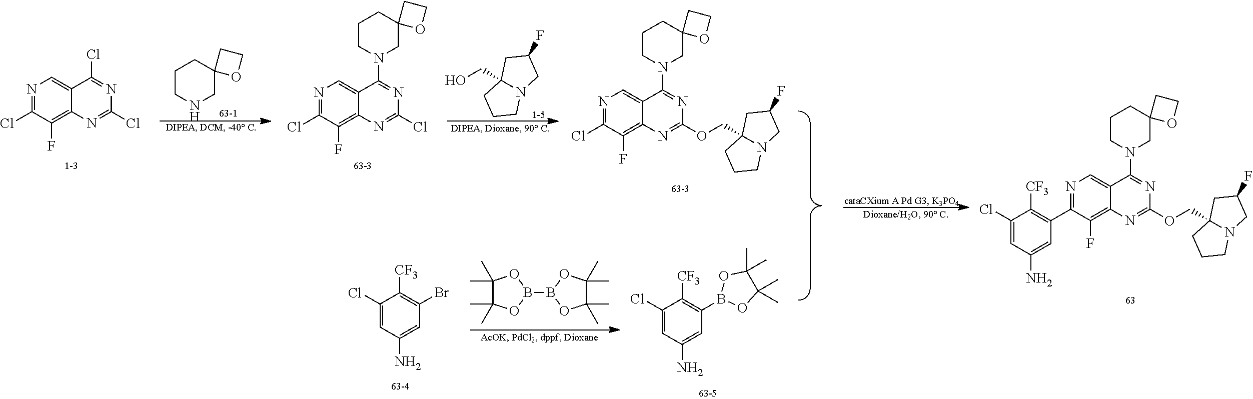

The suspension of intermediate 63-1 (85 mg, 668.32 μmol, 1 eq) in dichloromethane (5 mL) was purged with nitrogen gas and cooled to −40° C. Intermediate 1-3 (168.73 mg, 668.32 μmol, 1 eq) and diisopropylethylamine (345.50 mg, 2.67 mmol, 465.63 μL, 4 eq) were added subsequently. The reaction mixture was stirred at −40° C. for 30 minutes then was gradually warmed to room temperature. The solvent was removed in vacuo to afford crude residue, which was subjected to silica gel chromatography eluted with dichloromethane/methanol (v/v=100/0 to 95/5) to afford compound 63-2 (210 mg, Yield 91.51%).

To a stirred solution of intermediate 63-2 (25 mg, 72.85 μmol, 1 eq) in 1,4-dioxane (5 mL), reagent 1-5 (17.40 mg, 109.27 μmol, 1.5 eq) and Diisopropylethylamine (28.24 mg, 218.54 μmol, 38.07 μL, 3 eq) were added subsequently. The reaction mixture was warmed to 90° C. and stirred overnight. Upon the completion of conversion, the reaction mixture was cooled to room temperature and the solvent was removed in vacuo to afford crude product, which was subjected to silica gel chromatography eluted with dichloromethane/methanol (v/v=95/5) to give purified compound 63-3 (31 mg, Yield 91.33%).

Bis(pinacolato)diboron (277.56 mg, 1.09 mmol, 3 eq) was added to a stirred solution of compound 63-4 (100 mg, 364.34 μmol, 1 eq) in dioxane (5 mL). Potassium acetate (107.27 mg, 1.09 mmol, 3 eq) and 1,1′-Bis(diphenylphosphino)ferrocene palladium (II) dichloride (53.32 mg, 72.87 mol, 0.2 eq) were added subsequently. The reaction mixture was purged with nitrogen gas three times, then warmed to 100° C. and stirred overnight. Upon completion, the reaction mixture was cooled to room temperature, diluted with water and extracted with ethyl acetate. The organic layer was washed with water and brine, dried over sodium sulfate and then filtered. The filtrate was concentrated in vacuo to afford crude residue, which was subjected to silica gel column chromatography eluted with Petroleum Ether/Ethyl Acetate (v/v=100/0 to 80/20) to give compound 63-5 (70 mg, Yield 59.75%).

Compound 63-5 (13.46 mg, 41.85 μmol, 1.3 eq) was added to a stirred solution of compound 63-3 (15 mg, 32.19 μmol, 1 eq) in dioxane/water (3 mL/0.5 mL). Potassium phosphate (20.50 mg, 96.58 μmol, 3 eq) and cataCXium A Pd G3 (4.69 mg, 6.44 μmol, 0.2 eq) were added subsequently. The reaction mixture was purged with nitrogen gas three times, then warmed to 90° C. and stirred for 3 hours. Upon the completion, reaction mixture was cooled to room temperature, then concentrated in vacuo to afford crude residue, which was subjected to a reverse phase preparative HPLC eluted with water/acetonitrile to give the compound 63 (3.6 mg, Yield 11.13%). ¹H NMR (400 MHz, CD₃OD) δ 9.31 (d, J=7.0 Hz, 1H), 6.96 (d, J=2.3 Hz, 1H), 6.55 (d, J=2.3 Hz, 1H), 5.61 (d, J=51.8 Hz, 1H), 4.75 (d, J=13.9 Hz, 2H), 4.67 (dd, J=12.2, 4.1 Hz, 1H), 4.60 (d, J=7.9 Hz, 3H), 3.95 (d, J=16.6 Hz, 3H), 3.81 (dd, J=13.7, 3.7 Hz, 1H), 3.55-3.45 (m, 2H), 2.69-2.44 (m, 5H), 2.40-2.33 (m, 3H), 2.07-1.91 (m, 3H), 1.85 (d, J=12.7 Hz, 1H). m/z (ESI): 625.3 [M+H]⁺.

### Synthesis of Compound 64

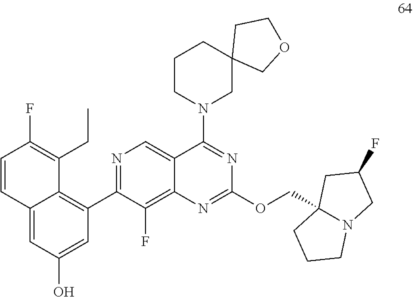 [SMILES: CCc1c(F)ccc2cc(O)cc(-c3ncc4c(N5CCCC6(CCOC6)C5)nc(OC[C@@]56CCCN5C[C@H](F)C6)nc4c3F)c12]

Title compound was synthesized from 2-oxa-7-azaspiro[4.5]decane in a manner essentially analogous to the synthesis of compound 23. ¹H NMR (500 MHz, CD₃OD) δ ppm 0.79-0.82 (m, 3H), 1.77-2.49 (m, 15H), 3.00-3.03 (m, 1H), 3.18-3.28 (m, 2H), 3.42-3.45 (m, 1H), 3.77-4.38 (m, 9H), 5.26 (s, 0.5H), 5.36 (s, 0.5H), 7.06 (t, J=3.5 Hz, 1H), 7.25 (t, J=10.0 Hz, 1H), 7.30 (d, J=2.1 Hz, 1H), 7.68 (dd, J=6.0, 3.5 Hz, 1H), 9.08 (d, J=2.5 Hz, 1H). m/z (ESI): 634.5 [M+H]⁺.

### Synthesis of Compound 65

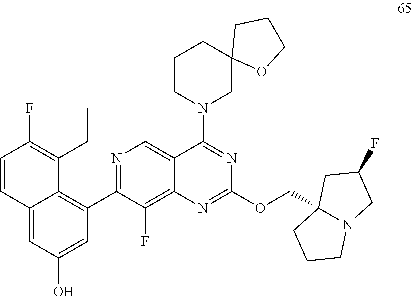 [SMILES: CCc1c(F)ccc2cc(O)cc(-c3ncc4c(N5CCCC6(CCCO6)C5)nc(OC[C@@]56CCCN5C[C@H](F)C6)nc4c3F)c12]

Title compound was synthesized from 1-oxa-7-azaspiro[4.5]decane in a manner essentially analogous to the synthesis of compound 23. ¹H NMR (500 MHz, CD₃OD) δ ppm 0.80 (t, J=8.0 Hz, 3H), 1.77-2.47 (m, 16H), 3.15-3.16 (m, 1H), 3.37-3.55 (m, 5H), 3.67-3.86 (m, 3H), 4.24-4.51 (m, 3H), 5.33 (s, 0.5H), 5.44 (s, 0.5H), 7.05 (s, 1H), 7.25 (t, J=10.0 Hz, 1H), 7.31 (s, 1H), 7.68 (dd, J=5.5, 3.0 Hz, 1H), 9.17 (d, J=13.0 Hz, 1H). m/z, (ESI): 634.5 [M+H]⁺.

### Synthesis of Compound 66

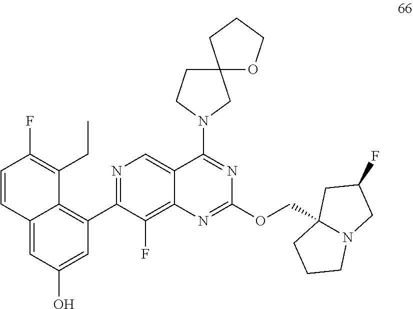 [SMILES: CCc1c(F)ccc2cc(O)cc(-c3ncc4c(N5CCC6(CCCO6)C5)nc(OC[C@@]56CCCN5C[C@H](F)C6)nc4c3F)c12]

Title compound was synthesized from 1-oxa-7-azaspiro[4.5]decane in a manner essentially analogous to the synthesis of compound 23. ¹H NMR (500 MHz, CD₃OD) δ ppm 0.76-0.81 (m, 3H), 1.93-2.49 (m, 15H), 3.07-3.08 (m, 1H), 3.40-3.44 (m, 2H), 3.92-4.41 (m, 8H), 5.29 (s, 0.5H), 5.39 (s, 0.5H), 7.04-7.06 (m, 1H), 7.25 (t, J=9.0 Hz, 1H), 7.30 (d, J=2.5 Hz, 1H), 7.68 (dd, J=6.0, 3.5 Hz, 1H), 9.24 (s, 1H). m/z, (ESI): 620.4 [M+H]⁺.

### Synthesis of Compound 67

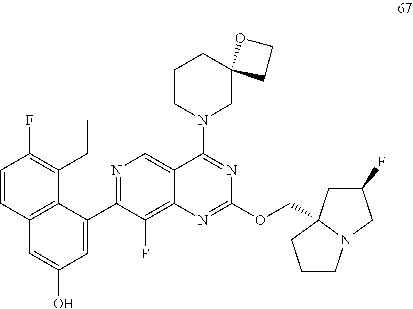 [SMILES: CCc1c(F)ccc2cc(O)cc(-c3ncc4c(N5CCC[C@@]6(CCO6)C5)nc(OC[C@@]56CCCN5C[C@H](F)C6)nc4c3F)c12]

Title compound was synthesized from (R)-1-oxa-6-azaspiro[3.5]nonane in a manner essentially analogous to the synthesis of compound 23. ¹H NMR (500 MHz, CD₃OD) δ ppm 0.76-0.81 (m, 3H), 1.93-2.49 (m, 15H), 3.07-3.08 (m, 1H), 3.40-3.44 (m, 2H), 3.92-4.41 (m, 8H), 5.29 (s, 0.5H), 5.39 (s, 0.5H), 7.04-7.06 (m, 1H), 7.25 (t, J=9.0 Hz, 1H), 7.30 (d, J=2.5 Hz, 1H), 7.68 (dd, J=6.0, 3.5 Hz, 1H), 9.24 (s, 1H). m/z, (ESI): 620.4 [M+H]⁺.

### Synthesis of Compound 68

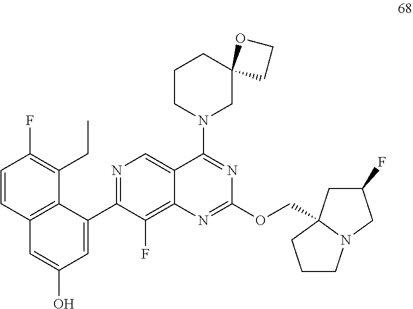 [SMILES: CCc1c(F)ccc2cc(O)cc(-c3ncc4c(N5CCC[C@]6(CCO6)C5)nc(OC[C@@]56CCCN5C[C@H](F)C6)nc4c3F)c12]

Title compound was synthesized from (S)-1-oxa-6-azaspiro[3.5]nonane in a manner essentially analogous to the synthesis of compound 23. ¹H NMR (500 MHz, CD₃OD) δ ppm 0.76-0.81 (m, 3H), 1.93-2.49 (m, 15H), 3.07-3.08 (m, 1H), 3.40-3.44 (m, 2H), 3.92-4.41 (m, 8H), 5.29 (s, 0.5H), 5.39 (s, 0.5H), 7.04-7.06 (m, 1H), 7.25 (t, J=9.0 Hz, 1H), 7.30 (d, J=2.5 Hz, 1H), 7.68 (dd, J=6.0, 3.5 Hz, 1H), 9.24 (s, 1H). m/z, (ESI): 620.4 [M+H]⁺.

### Synthesis of Compound 70

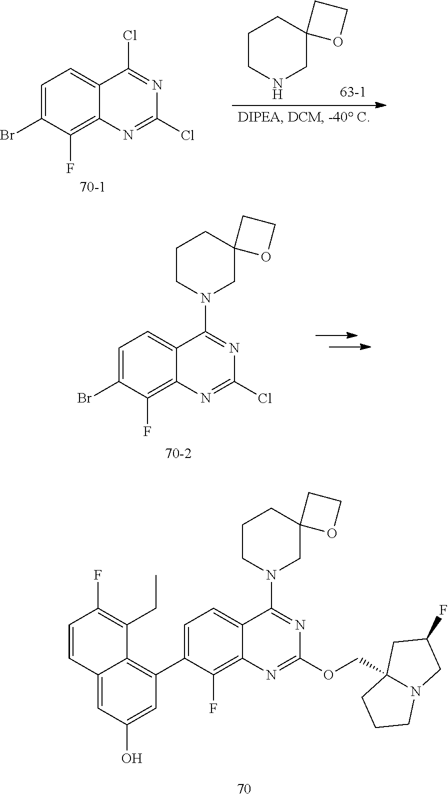 [SMILES: Fc1c(Br)ccc2c(Cl)nc(Cl)nc12 ; C1CNCC2(C1)CCO2 ; Fc1c(Br)ccc2c(N3CCCC4(CCO4)C3)nc(Cl)nc12 ; CCc1c(F)ccc2cc(O)cc(-c3ccc4c(N5CCCC6(CCO6)C5)nc(OC[C@@]56CCCN5C[C@H](F)C6)nc4c3F)c12]

A solution of intermediate 70-1 (100 mg, 168.96 μmol, 1 eq) in dichloromethane (5 mL) was purged with nitrogen gas and cooled to −40° C. Compound 63-1 (21.49 mg, 168.96 μmol, 1 eq) and Diisopropylethylamine (65.51 mg, 506.89 μmol, 3 eq) were added subsequently. The reaction mixture was stirred at −40° C. for 3 hours. Upon the completion of conversion, the reaction mixture was warmed to room temperature and the solvent was removed in vacuo to afford crude product, which was subjected to silica gel chromatography eluted with dichloromethane/methanol (v/v=100/0 to 99/1) to give purified compound 70-2 (49 mg, Yield 75.01%).

Title compound was synthesized from intermediate 70-2 in a manner essentially analogous to the synthesis of compound 3. ¹H NMR (500 MHz, CD₃OD) δ ppm 8.09 (d, J=9.2 Hz, 1H), 7.72-7.65 (m, 1H), 7.48 (s, 1H), 7.31-7.22 (m, 2H), 6.97 (s, 1H), 5.62 (s, 0.5H), 5.51 (s, 0.5H), 4.70 (dt, J=29.8, 15.0 Hz, 3H), 4.61-4.32 (m, 3H), 4.14-3.72 (m, 4H), 3.46 (s, 2H), 2.79-2.25 (m, 10H), 2.24-1.77 (m, 4H), 0.79 (t, J=6.8 Hz, 3H). m/z, (ESI): 619.3 [M+H]⁺.

### Synthesis of Compound 71

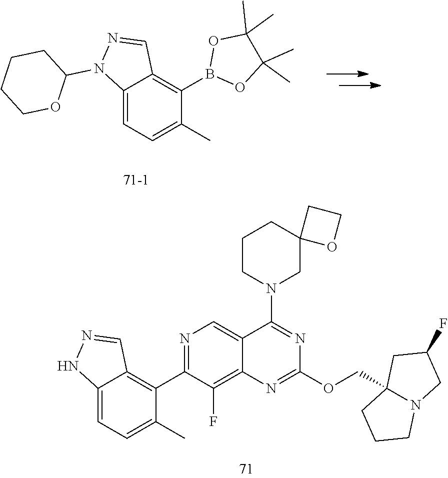 [SMILES: Cc1ccc2c(cnn2C2CCCCO2)c1B1OC(C)(C)C(C)(C)O1 ; Cc1ccc2[nH]ncc2c1-c1ncc2c(N3CCCC4(CCO4)C3)nc(OC[C@@]34CCCN3C[C@H](F)C4)nc2c1F]

Title compound was synthesized from intermediate 71-1 in a manner essentially analogous to the synthesis of compound 63. ¹H NMR (500 MHz, CD₃OD) δ ppm 8.09 (d, J=9.2 Hz, 1H), 7.72-7.65 (m, 1H), 7.48 (s, 1H), 7.31-7.22 (m, 2H), 6.97 (s, 1H), 5.62 (s, 0.5H), 5.51 (s, 0.5H), 4.70 (dt, J=29.8, 15.0 Hz, 3H), 4.61-4.32 (m, 3H), 4.14-3.72 (m, 4H), 3.46 (s, 2H), 2.79-2.25 (m, 10H), 2.24-1.77 (m, 4H), 0.79 (t, J=6.8 Hz, 3H). m/z, (ESI): 619.3 [M+H]⁺.

### Synthesis of Compound 73

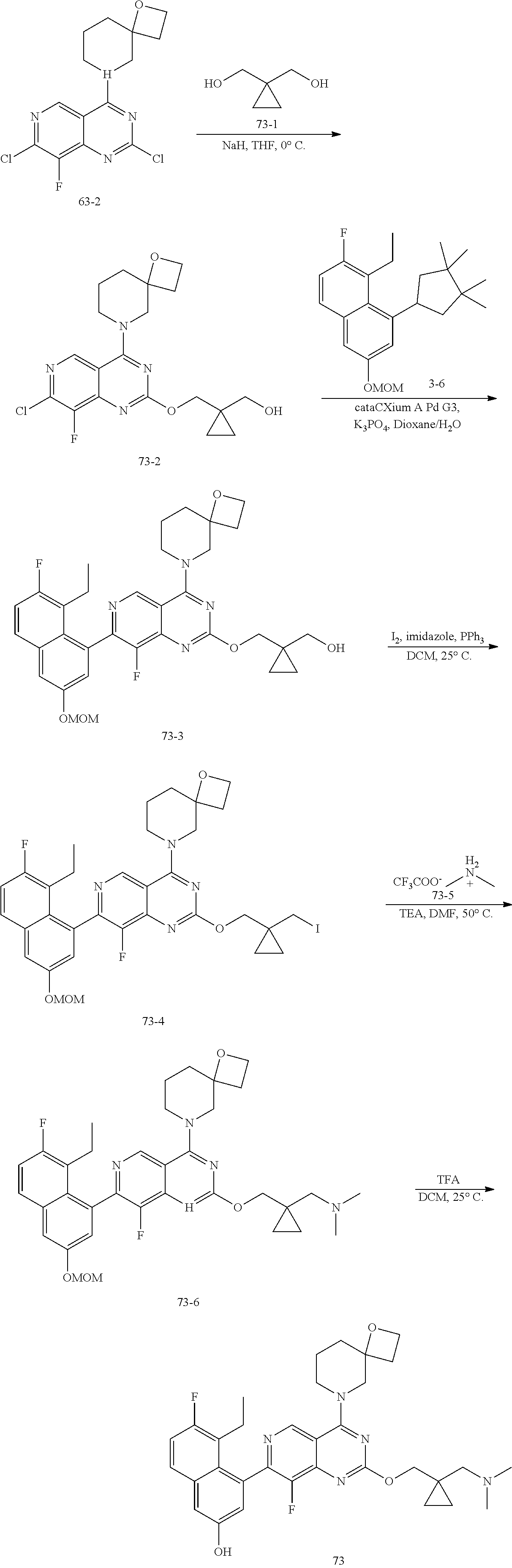

The solution of reagent 73-1 (267.84 mg, 2.62 mmol, 3 eq) in anhydrous tetrahydrofuran (5 mL) was purged with nitrogen gas and cooled to 0° C. 60% sodium hydride (w/w) in mineral oil (104.8 mg, 2.62 mmol, 3 eq) was added at 0° C., and stirred for 30 minutes to reach full deprotonation. Intermediate 63-2 (300 mg, 874.17 μmol, 1 eq) was added and the reaction mixture was stirred at −40° C. for another 1 hour, then was quenched by adding water. Ethyl acetate was poured into the reaction mixture and the organic layer was collected, washed with water and brine, dried over sodium sulfate and then filtered. Filtrate was concentrated in vacuo to give crude residue, which was subjected to silica gel chromatography eluted with dichloromethane/methanol (v/v=100/0 to 97/3) to afford compound 73-2 (180 mg, Yield 50.40%). m/z, (ESI): 409.5 [M+H]⁺

Compound 3-6 (317.18 mg, 880.51 μmol, 2 eq) was added to a stirred solution of compound 73-2 (180 mg, 333.26 μmol, 1 eq) in dioxane/water (10 mL/1 mL). Potassium phosphate (280.36 mg, 1.32 mmol, 3 eq) and cataCXium A Pd G3 (64.04 mg, 88.05 μmol, 0.2 eq) were added subsequently. The reaction mixture was purged with nitrogen gas three times, then warmed up to 90° C. and stirred for 3 hours. Upon completion, the reaction mixture was cooled to room temperature, diluted with water and extracted with ethyl acetate. The organic layer was washed with water and brine, dried over sodium sulfate and then filtered. The filtrate was concentrated in vacuo to afford crude residue, which was subjected to silica gel column chromatography eluted with dichloromethane/methanol (v/v=100/0 to 98/2) to give compound 73-3 (200 mg, Yield 74.90%). m/z, (ESI): 607.9 [M+H]⁺.

To a solution of intermediate 73-3 (267.84 mg, 2.62 mmol, 3 eq) in dichloromethane (10 mL), triphenylphosphine (259.41 mg, 989.02 μmol, 3 eq), imidazole (67.33 mg, 989.02 μmol, 3 eq) and iodine (167.35 mg, 659.35 μmol, 2 eq) were added subsequently at room temperature, and the reaction mixture was stirred for 30 minutes. Upon completion of conversion, the solvent was removed in vacuo to give crude residue, which was subjected to silica gel chromatography eluted with dichloromethane/methanol (v/v=100/0 to 99/1) to afford compound 73-4 (220 mg, Yield 93.10%). m/z, (ESI): 717.3 [M+H]⁺.

To a stirred solution of intermediate 73-4 (60 mg, 83.73 μmol, 1 eq) in dimethylformamide (2 mL), reagent 73-5 (66.61 mg, 418.67 μmol, 5 eq) and triethylamine (84.73 mg, 837.34 μmol, 10 eq) were added subsequently. The reaction mixture was warmed to 50° C. and stirred for 2 hours. Upon the completion of conversion, the reaction mixture was cooled to room temperature, diluted with ethyl acetate. The organic layer was washed with water and brine, dried over sodium sulfate and then filtered. The filtrate was concentrated in vacuo to afford crude residue, which was subjected to silica gel chromatography eluted with dichloromethane/methanol (v/v=100/0 to 91/9) to give purified compound 73-6 (50 mg, Yield 81.60%). m/z, (ESI): 634.5 [M+H]⁺.

To a stirred solution of intermediate 73-6 (40 mg, 63.12 μmol, 1 eq) in dichloromethane (1 mL), trifluoroacetic acid (2 mL) was added in one portion. The reaction mixture was stirred for 25 minutes at room temperature. Upon the completion of conversion, the solvent was removed in vacuo to afford crude residue, which was subjected to a reverse phase preparative HPLC eluted with 0.05% ammonia hydroxide aqueous solution/acetonitrile to give the compound 73 (11.7 mg, Yield 29.5%). ¹H NMR (500 MHz, CD₃OD) δ ppm 9.22 (d, J=6.8 Hz, 1H), 7.62 (dd, J=9.5, 5.3 Hz, 1H), 7.25 (q, J=2.8 Hz, 1H), 7.20 (t, J=9.2 Hz, 1H), 7.02 (s, 1H), 4.70-4.28 (m, 6H), 3.81-3.70 (m, 1H), 3.49-3.30 (m, 1H), 2.89-2.60 (m, 2H), 2.60-2.29 (m, 9H), 2.24 (d, J=12.9 Hz, 1H), 2.18-2.04 (m, 1H), 2.02-1.67 (m, 3H), 0.81-0.70 (m, 5H), 0.60 (s, 2H). m/z, (ESI): 590.3 [M+H]⁺.

### Synthesis of Compound 92

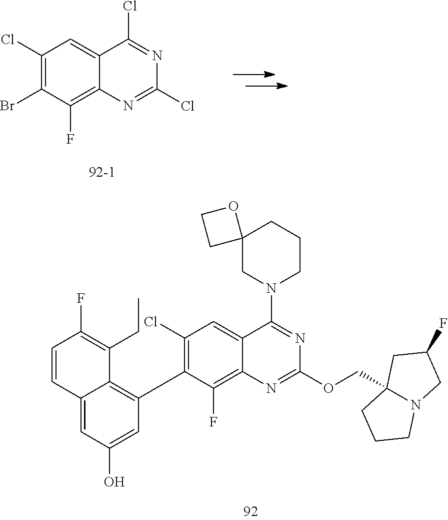 [SMILES: Fc1c(Br)c(Cl)cc2c(Cl)nc(Cl)nc12 ; CCc1c(F)ccc2cc(O)cc(-c3c(Cl)cc4c(N5CCCC6(CCO6)C5)nc(OC[C@@]56CCCN5C[C@H](F)C6)nc4c3F)c12]

Title compound was synthesized from intermediate 92-1 in a manner essentially analogous to the synthesis of compound 70. ¹H NMR (500 MHz, CD₃OD) δ ppm 8.27 (d, J=8.3 Hz, 1H), 7.65-7.60 (m, 1H), 7.23 (d, J=2.8 Hz, 1H), 7.19 (t, J=9.3 Hz, 1H), 6.85 (d, J=3.0 Hz, 1H), 5.55 (s, 0.5H), 5.45 (s, 0.5H), 4.65-4.47 (m, 6H), 4.33 (s, 1H), 3.86-3.79 (m, 2H), 3.58 (t, J=14.7 Hz, 1H), 3.44-3.35 (m, 2H), 2.58-2.42 (m, 4H), 2.40-2.20 (m, 6H), 2.15-1.91 (m, 3H), 1.88-1.71 (m, 2H), 0.78-0.70 (m, 3H). m/z, (ESI): 653.3 [M+H]⁺.

### Synthesis of Compound 96

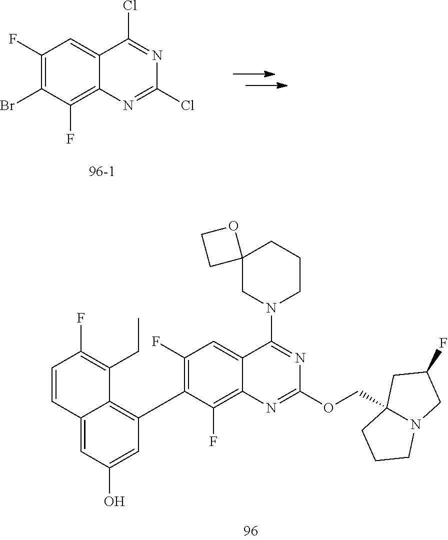 [SMILES: Fc1cc2c(Cl)nc(Cl)nc2c(F)c1Br ; CCc1c(F)ccc2cc(O)cc(-c3c(F)cc4c(N5CCCC6(CCO6)C5)nc(OC[C@@]56CCCN5C[C@H](F)C6)nc4c3F)c12]

Title compound was synthesized from intermediate 96-1 in a manner essentially analogous to the synthesis of compound 70. ¹H NMR (500 MHz, CD₃OD) δ ppm 7.93 (t, J=11.0 Hz, 1H), 7.74-7.65 (m, 1H), 7.31 (s, 1H), 7.27 (t, J=9.2 Hz, 1H), 7.00 (s, 1H), 5.61 (s, 0.5H), 5.51 (s, 0.5H), 5.34 (s, 1H), 4.74-4.49 (m, 4H), 4.31 (s, 1H), 4.10-3.80 (m, 3H), 3.72-3.62 (m, 1H), 3.45 (s, 1H), 2.77-1.98 (m, 12H), 1.96-1.76 (m, 2H), 1.60 (s, 1H), 0.90 (d, J=7.1 Hz, 1H), 0.81 (s, 3H). m/z, (ESI): 637.4 [M+H]⁺.

### Synthesis of Compound 110

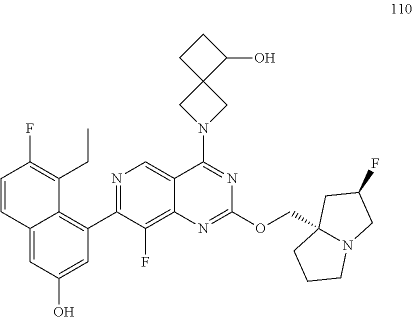 [SMILES: CCc1c(F)ccc2cc(O)cc(-c3ncc4c(N5CC6(CCC6O)C5)nc(OC[C@@]56CCCN5C[C@H](F)C6)nc4c3F)c12]

Title compound was synthesized from 2-azaspiro[3.3]heptan-5-ol in a manner essentially analogous to the synthesis of compound 23. ¹H NMR (500 MHz, CD₃OD) δ ppm 8.89 (d, J=54.7 Hz, 1H), 7.61 (dd, J=9.0, 5.8 Hz, 1H), 7.23 (d, J=2.7 Hz, 1H), 7.18 (t, J=9.4 Hz, 1H), 6.97 (s, 1H), 5.29 (s, 0.5H), 5.18 (s, 0.5H), 4.72-4.59 (m, 1H), 4.37-4.10 (m, 5H), 3.20-3.04 (m, 3H), 3.01-2.89 (m, 1H), 2.52-2.35 (m, 1H), 2.32-1.75 (m, 11H), 1.75-1.62 (m, 1H), 0.72 (t, J=7.5 Hz, 3H). m/z, (ESI): 606.4 [M+H]⁺.

### Synthesis of Compound 112

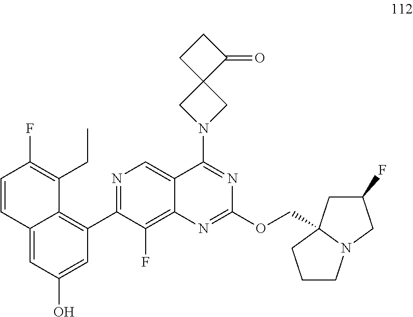 [SMILES: CCc1c(F)ccc2cc(O)cc(-c3ncc4c(N5CC6(CCC6=O)C5)nc(OC[C@@]56CCCN5C[C@H](F)C6)nc4c3F)c12]

Title compound was synthesized from 2-azaspiro[3.3]heptan-5-one in a manner essentially analogous to the synthesis of compound 23. ¹H NMR (500 MHz, CD₃OD) δ ppm 8.88 (s, 1H), 7.61 (dd, J=7.8, 5.8 Hz, 1H), 7.23 (s, 1H), 7.18 (t, J=9.3 Hz, 1H), 6.95 (s, 1H), 5.55 (s, 1H), 5.44 (s, 1H), 5.05-4.91 (m, 1H), 4.67-4.42 (m, 3H), 4.05-3.76 (m, 3H), 3.45-3.32 (m, 2H), 3.13-3.01 (m, 2H), 2.68-1.86 (m, 11H), 0.75-0.65 (m, 3H). m/z, (ESI): 604.2 [M+H]⁺.

### Synthesis of Compound 117

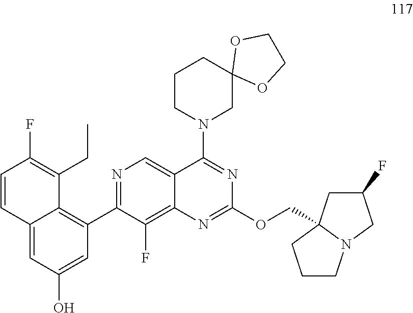 [SMILES: CCc1c(F)ccc2cc(O)cc(-c3ncc4c(N5CCCC6(C5)OCCO6)nc(OC[C@@]56CCCN5C[C@H](F)C6)nc4c3F)c12]

Title compound was synthesized from 1,4-dioxa-7-azaspiro[4.5]decane in a manner essentially analogous to the synthesis of compound 23. ¹H NMR (500 MHz, CD₃OD) δ 0.78-0.82 (m, 3H), 1.89-2.50 (m, 12H), 2.99-3.04 (m, 1H), 3.16-3.28 (m, 3H), 3.90-4.08 (m, 8H), 4.24-4.34 (m, 2H), 5.25 (s, 0.5H), 5.36 (s, 0.5H), 7.07 (s, 1H), 7.25 (t, J=10.0 Hz, 1H), 7.30 (s, 1H), 7.66-7.69 (m, 1H), 9.14 (s, 1H). m/z, (ESI): 636.3 [M+H]⁺.

### Synthesis of Compound 1a

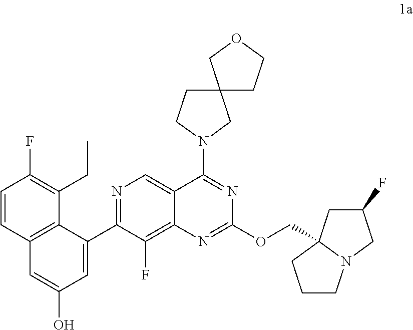 [SMILES: CCc1c(F)ccc2cc(O)cc(-c3ncc4c(N5CCC6(CCOC6)C5)nc(OC[C@@]56CCCN5C[C@H](F)C6)nc4c3F)c12]

Title compound was synthesized from 2-Oxa-7-aza-spiro[4.4]nonane in a manner essentially analogous to the synthesis of compound 23. ¹H NMR (500 MHz, CD₃OD) δ 9.31 (s, 1H), 7.71 (dd, J=9.0, 5.8 Hz, 1H), 7.34 (s, 1H), 7.29 (t, J=9.4 Hz, 1H), 7.08 (s, 1H), 5.42 (d, J=53.2 Hz, 1H), 4.63 (s, 1H), 4.53-4.35 (m, 2H), 4.01 (t, J=7.2 Hz, 2H), 3.88-3.72 (m, 2H), 3.52-3.39 (m, 2H), 3.32 (s, 2H), 3.19 (s, 1H), 2.62-1.89 (m, 15H), 0.83 (t, J=6.3 Hz, 3H). m/z, (ESI): 620.5 [M+H]⁺.

### Synthesis of Compound 2a

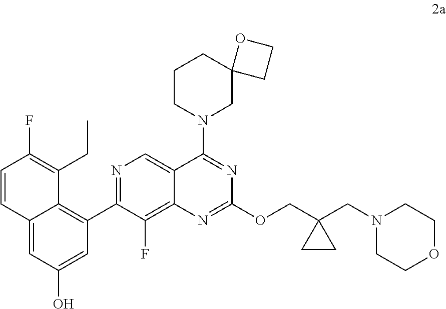 [SMILES: CCc1c(F)ccc2cc(O)cc(-c3ncc4c(N5CCCC6(CCO6)C5)nc(OCC5(CN6CCOCC6)CC5)nc4c3F)c12]

Title compound was synthesized from morpholine in a manner essentially analogous to the synthesis of compound 73. ¹H NMR (500 MHz, CD₃OD) δ ppm 9.26 (d, J=8.2 Hz, 1H), 7.62 (dd, J=9.1, 5.7 Hz, 1H), 7.25 (d, J=2.7 Hz, 1H), 7.20 (t, J=9.3 Hz, 1H), 7.02 (s, 1H), 4.68 (dd, J=37.4, 13.6 Hz, 1H), 4.57-4.31 (m, 5H), 4.12-3.90 (m, 2H), 3.89-3.62 (m, 5H), 3.44-3.27 (m, 3H), 3.19-3.02 (m, 2H), 2.55-2.34 (m, 3H), 2.32-2.20 (m, 1H), 2.20-2.03 (m, 1H), 2.02-1.64 (m, 3H), 0.94 (s, 2H), 0.82 (s, 2H), 0.74 (q, J=7.5 Hz, 3H). m/z, (ESI): 632.3 [M+H]⁺.

### Synthesis of Compound 3a

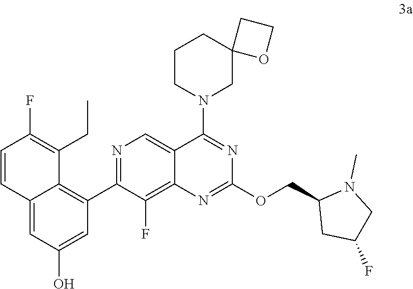 [SMILES: CCc1c(F)ccc2cc(O)cc(-c3ncc4c(N5CCCC6(CCO6)C5)nc(OC[C@@H]5C[C@@H](F)CN5C)nc4c3F)c12]

Title compound was synthesized from [(2S,4R)-4-fluoro-1-methyl-pyrrolidin-2-yl]methanol in a manner essentially analogous to the synthesis of compound 73. ¹H NMR (500 MHz, CD₃OD) δ 0.80-0.81 (m, 3H), 1.80-2.71 (m, 11H), 3.18 (s, 3H), 3.39-3.49 (m, 1H), 3.63-3.85 (m, 2H), 4.03-4.28 (m, 2H), 4.50-4.60 (m, 3H), 4.69-4.79 (m, 2H), 5.43 (s, 0.5H), 5.54 (s, 0.5H), 7.8 (s, 1H), 7.26 (t, J=9.0 Hz, 1H), 7.30 (d, J=2.5 Hz, 1H), 7.67-7.70 (m, 1H), 9.31-9.33 (m, 1H). m/z, (ESI): 594.3 [M+H]⁺.

### Synthesis of Compound 4a

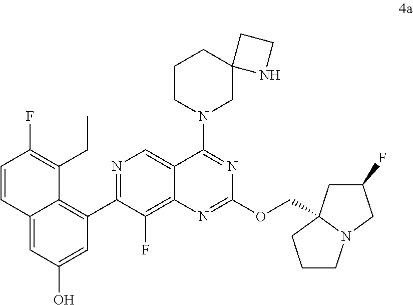 [SMILES: CCc1c(F)ccc2cc(O)cc(-c3ncc4c(N5CCCC6(CCN6)C5)nc(OC[C@@]56CCCN5C[C@H](F)C6)nc4c3F)c12]

Title compound was synthesized from tert-butyl 1,6-diazaspiro[3.5]nonane-1-carboxylate in a manner essentially analogous to the synthesis of compound 23. ¹H NMR (500 MHz, CD₃OD) δ 0.77-0.82 (m, 3H), 1.86-2.48 (m, 15H), 3.03 (s, 1H), 3.21-3.26 (m, 2H), 3.62-3.86 (m, 3H), 3.98-4.11 (m, 2H), 4.27-4.40 (m, 3H), 5.26 (s, 0.5H), 5.37 (s, 0.5H), 7.06 (s, 1H), 7.25 (t, J=9.0 Hz, 1H), 7.31 (s, 1H), 7.66-7.69 (m, 1H), 9.08 (s, 1H). m/z, (ESI): 619.3 [M+H]⁺.

### Synthesis of Compound 5a

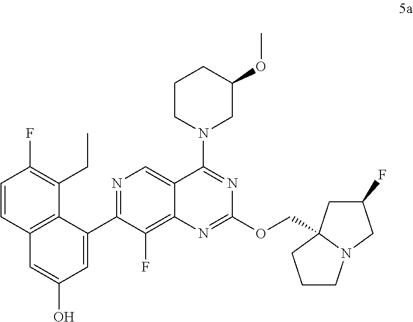 [SMILES: CCc1c(F)ccc2cc(O)cc(-c3ncc4c(N5CCC[C@@H](OC)C5)nc(OC[C@@]56CCCN5C[C@H](F)C6)nc4c3F)c12]

Title compound was synthesized from (R)-3-methoxy-piperidine in a manner essentially analogous to the synthesis of compound 23. ¹H NMR (500 MHz, CD₃OD) δ 9.18 (d, J=13.1 Hz, 1H), 7.70 (dd, J=9.1, 5.7 Hz, 1H), 7.33 (d, J=2.8 Hz, 1H), 7.28 (t, J=9.4 Hz, 1H), 7.10 (dd, J=9.3, 2.8 Hz, 1H), 5.33 (d, J=53.0 Hz, 1H), 4.42-3.97 (m, 5H), 3.90-3.69 (m, 1H), 3.67-3.56 (m, 1H), 3.41 (s, 1H), 3.34 (s, 3H), 3.29-3.19 (m, 2H), 3.04 (td, J=9.7, 5.1 Hz, 1H), 2.59-2.43 (m, 1H), 2.40-1.83 (m, 10H), 1.74 (s, 1H), 0.82 (td, J=7.4, 3.7 Hz, 3H). m/z, (ESI): 608.3 [M+H]⁺.

### Synthesis of Compound 6a

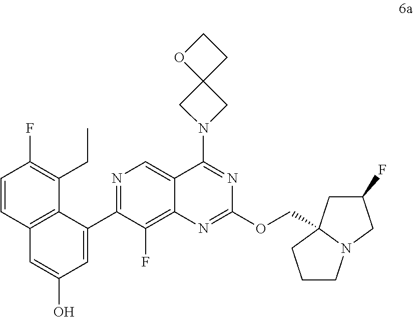 [SMILES: CCc1c(F)ccc2cc(O)cc(-c3ncc4c(N5CC6(CCO6)C5)nc(OC[C@@]56CCCN5C[C@H](F)C6)nc4c3F)c12]

Title compound was synthesized from 2-oxa-6-azaspiro[3.3]heptane in a manner essentially analogous to the synthesis of compound 23. ¹H NMR (500 MHz, CD₃OD) δ 8.94 (s, 1H), 7.70 (dd, J=9.1, 5.6 Hz, 1H), 7.33 (d, J=2.7 Hz, 1H), 7.28 (t, J=9.3 Hz, 1H), 7.07 (s, 1H), 5.33 (d, J=53.7 Hz, 1H), 5.09 (s, 2H), 4.82-4.69 (m, 1H), 4.62 (t, J=7.5 Hz, 3H), 4.40-4.20 (m, 2H), 3.32-3.17 (m, 3H), 3.09-2.98 (m, 3H), 2.49 (s, 1H), 2.40-1.84 (m, 7H), 0.80 (t, J=7.4 Hz, 3H). m/z, (ESI): 592.3 [M+H]⁺.

### Synthesis of Compound 7a

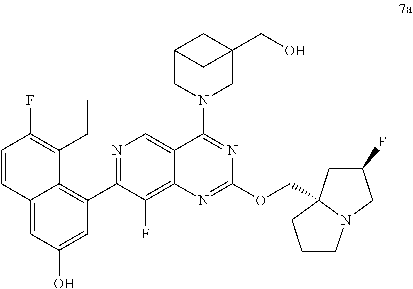 [SMILES: CCc1c(F)ccc2cc(O)cc(-c3ncc4c(N5CC6CC(CO)(C6)C5)nc(OC[C@@]56CCCN5C[C@H](F)C6)nc4c3F)c12]

Title compound was synthesized from {3-azabicyclo[3.1.1]heptan-1-yl}methanol in a manner essentially analogous to the synthesis of compound 23. ¹H NMR (400 MHz, CD₃OD) δ 0.80 (t, J=7.6 Hz, 3H), 1.55-1.58 (m, 2H), 1.93-2.53 (m, 11H), 2.72 (s, 1H), 3.11-3.16 (m, 1H), 3.38-3.42 (m, 2H), 3.57 (s, 2H), 4.23-4.47 (m, 6H), 5.31 (s, 0.5H), 5.45 (s, 0.5H), 7.05 (s, 1H), 7.25 (t, J=9.2 Hz, 1H), 7.30 (s, 1H), 7.66-7.70 (m, 1H), 9.47 (s, 1H). m/z, (ESI): 620.5 [M+H]⁺.

### Synthesis of Compound 8a

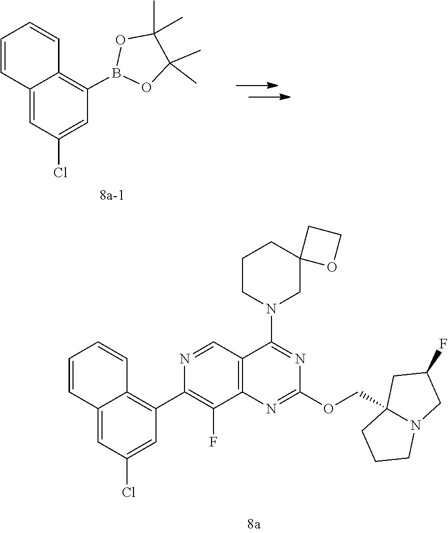 [SMILES: CC1(C)OB(c2cc(Cl)cc3ccccc23)OC1(C)C ; Fc1c(-c2cc(Cl)cc3ccccc23)ncc2c(N3CCCC4(CCO4)C3)nc(OC[C@@]34CCCN3C[C@H](F)C4)nc12]

Title compound was synthesized from intermediate 8a-1 in a manner essentially analogous to the synthesis of compound 63. ¹H NMR (400 MHz, CD₃OD) δ 9.39 (s, 1H), 8.13 (d, J=2.1 Hz, 1H), 8.00 (d, J=8.3 Hz, 1H), 7.73 (d, J=8.6 Hz, 1H), 7.69 (d, J=2.1 Hz, 1H), 7.64 (t, J=7.6 Hz, 1H), 7.55 (t, J=7.6 Hz, 1H), 5.35 (d, J=54.2 Hz, 2H), 4.61 (d, J=8.1 Hz, 1H), 4.48 (d, J=13.5 Hz, 1H), 4.39 (dd, J=10.6, 5.1 Hz, 1H), 4.31 (dd, J=10.6, 4.4 Hz, 1H), 3.87 (d, J=13.6 Hz, 1H), 3.50 (d, J=11.7 Hz, 1H), 3.27 (d, J=21.5 Hz, 2H), 3.14-3.00 (m, 1H), 2.56 (dt, J=16.9, 8.6 Hz, 2H), 2.39-2.26 (m, 3H), 2.18 (d, J=9.6 Hz, 1H), 2.12-1.90 (m, 6H), 1.88-1.81 (m, 1H), 1.32 (s, 1H). m/z, (ESI): 592.3 [M+H]⁺.

### Synthesis of Compound 9a

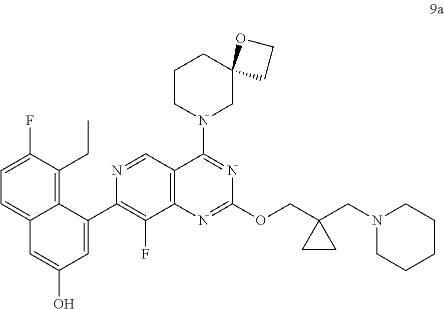 [SMILES: CCc1c(F)ccc2cc(O)cc(-c3ncc4c(N5CCC[C@]6(CCO6)C5)nc(OCC5(CN6CCCCC6)CC5)nc4c3F)c12]

Title compound was synthesized from piperidine in a manner essentially analogous to the synthesis of compound 73. ¹H NMR (400 MHz, d₆-DMSO) δ 9.96 (s, 1H), 9.26 (d, J=3.4 Hz, 1H), 8.79-8.58 (m, 1H), 7.78 (dd, J=9.1, 6.0 Hz, 1H), 7.42-7.28 (m, 2H), 7.08-6.97 (m, 1H), 4.63-4.49 (m, 1H), 4.45-4.12 (m, 5H), 4.00-3.82 (m, 2H), 3.81-3.45 (m, 2H), 3.44-3.31 (m, 1H), 3.25-3.08 (m, 2H), 2.99-2.79 (m, 2H), 2.43-2.27 (m, 3H), 2.20-2.05 (m, 2H), 1.96-1.62 (m, 7H), 1.52-1.16 (m, 3H), 0.98-0.83 (m, 2H), 0.82-0.65 (m, 4H). m/z, (ESI): 630.4 [M+H]⁺.

### Synthesis of Compound 10a

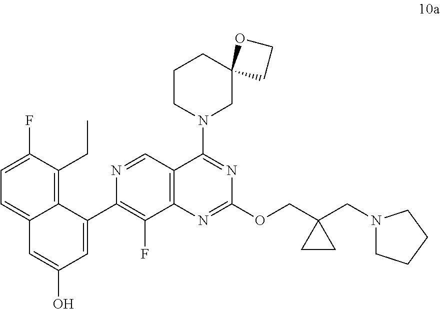 [SMILES: CCc1c(F)ccc2cc(O)cc(-c3ncc4c(N5CCC[C@]6(CCO6)C5)nc(OCC5(CN6CCCC6)CC5)nc4c3F)c12]

Title compound was synthesized from pyrrolidine in a manner essentially analogous to the synthesis of compound 73. ¹H NMR (400 MHz, d₆-DMSO) δ 10.03-9.89 (m, 1H), 9.45-9.29 (m, 1H), 9.27 (d, J=1.9 Hz, 1H), 7.79 (dd, J=9.1, 6.0 Hz, 1H), 7.36 (dd, J=11.6, 6.0 Hz, 2H), 7.10-6.98 (m, 1H), 4.62-4.49 (m, 1H), 4.45-4.18 (m, 5H), 3.99-3.78 (m, 5H), 3.76-3.67 (m, 3H), 3.42-3.33 (m, 1H), 3.30 (d, J=5.6 Hz, 1H), 3.16-3.00 (m, 2H), 2.44-2.29 (m, 3H), 2.21-2.09 (m, 2H), 2.00-1.98 (m, 2H), 1.96-1.82 (m, 3H), 1.79-1.66 (m, 1H), 0.90-0.69 (m, 5H). m/z, (ESI): 616.3 [M+H]⁺.

### Synthesis of Compound 11a

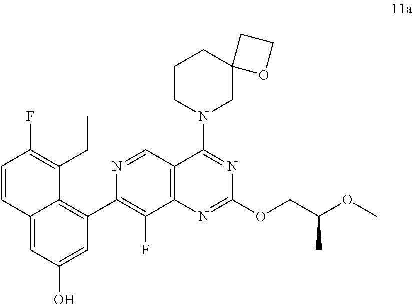 [SMILES: CCc1c(F)ccc2cc(O)cc(-c3ncc4c(N5CCCC6(CCO6)C5)nc(OC[C@H](C)OC)nc4c3F)c12]

Title compound was synthesized from (2S)-2-methoxy-1-propanol in a manner essentially analogous to the synthesis of compound 73. ¹H NMR (400 MHz, CD₃OD) δ 9.30 (d, J=5.9 Hz, 1H), 7.72 (dd, J=9.1, 5.8 Hz, 1H), 7.34 (d, J=2.6 Hz, 1H), 7.29 (t, J=9.4 Hz, 1H), 7.12 (d, J=2.6 Hz, 1H), 4.74-4.56 (m, 3H), 4.50 (d, J=5.2 Hz, 2H), 3.94-3.81 (m, 2H), 3.48 (s, 3H), 2.59-2.49 (m, 3H), 2.33 (d, J=13.1 Hz, 1H), 2.21 (s, 1H), 2.09-1.95 (m, 2H), 1.86 (s, 1H), 1.32 (d, J=6.6 Hz, 5H), 0.85 (q, J=7.0 Hz, 3H). m/z, (ESI): 551.4 [M+H]⁺.

### Synthesis of Compound 12a

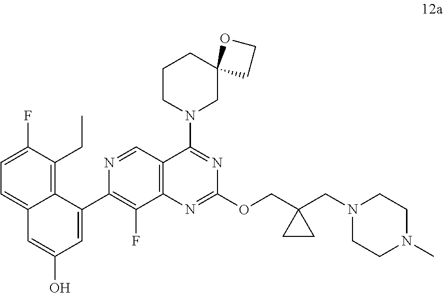 [SMILES: CCc1c(F)ccc2cc(O)cc(-c3ncc4c(N5CCC[C@]6(CCO6)C5)nc(OCC5(CN6CCN(C)CC6)CC5)nc4c3F)c12]

Title compound was synthesized from N-methylpiperazine in a manner essentially analogous to the synthesis of compound 73. ¹H NMR (400 MHz, d₆-DMSO) δ 10.15-9.72 (m, 2H), 9.24 (s, 1H), 7.77 (dd, J=9.1, 6.0 Hz, 1H), 7.35 (dd, J=11.8, 6.1 Hz, 2H), 7.05-6.98 (m, 1H), 4.62-4.04 (m, 11H), 3.98-3.76 (m, 2H), 3.37 (d, J=10.2 Hz, 4H), 3.13-2.94 (m, 2H), 2.71 (d, J=34.1 Hz, 3H), 2.47-2.27 (m, 5H), 2.12 (d, J=11.2 Hz, 2H), 1.86 (s, 3H), 0.73 (dd, J=13.9, 7.0 Hz, 4H), 0.55 (s, 2H). m/z, (ESI): 645.3 [M+H]⁺.

### Synthesis of Compound 13a

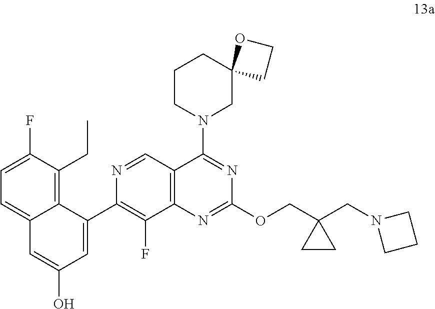 [SMILES: CCc1c(F)ccc2cc(O)cc(-c3ncc4c(N5CCC[C@]6(CCO6)C5)nc(OCC5(CN6CCC6)CC5)nc4c3F)c12]

Title compound was synthesized from azetidine in a manner essentially analogous to the synthesis of compound 73. ¹H NMR (400 MHz, CD₃OD) δ 9.33 (d, J=7.6 Hz, 1H), 7.71 (dd, J=9.1, 5.9 Hz, 1H), 7.35 (d, J=2.7 Hz, 1H), 7.28 (t, J=9.4 Hz, 1H), 7.11 (t, J=2.6 Hz, 1H), 4.80-4.69 (m, 2H), 4.63-4.49 (m, 3H), 4.41 (s, 3H), 4.22 (q, J=9.8 Hz, 2H), 3.91-3.80 (m, 1H), 3.52-3.43 (m, 1H), 3.39 (s, 2H), 2.66 (q, J=10.1 Hz, 1H), 2.57-2.45 (m, 3H), 2.44-2.30 (m, 2H), 2.25-2.16 (m, 1H), 2.09-1.90 (m, 2H), 1.89-1.78 (m, 1H), 0.95-0.79 (m, 7H). m/z, (ESI): 602.3 [M+H]⁺.

### Synthesis of Compound 14a

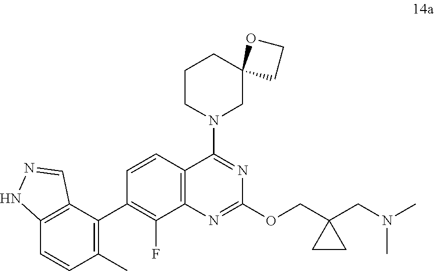 [SMILES: Cc1ccc2[nH]ncc2c1-c1ccc2c(N3CCC[C@]4(CCO4)C3)nc(OCC3(CN(C)C)CC3)nc2c1F]

Title compound was synthesized from intermediate 71-1 in a manner essentially analogous to the synthesis of compound 73. ¹H NMR (400 MHz, CD₃OD) δ 8.19 (s, 1H), 7.79 (s, 1H), 7.72 (dd, J=11.2, 1.6 Hz, 1H), 7.55 (dd, J=8.6, 1.0 Hz, 1H), 7.43 (d, J=8.6 Hz, 1H), 4.51 (s, 2H), 4.37-4.42 (m, 2H), 3.57-3.63 (m, 2H), 3.06 (s, 6H), 2.40-2.43 (m, 5H), 2.30-2.18 (m, 2H), 2.10-1.73 (m, 5H), 1.59-1.66 (m, 1H), 1.07-1.01 (m, 2H), 0.90-0.93 (m, 2H). m/z, (ESI): 531 [M+H]⁺.

### Synthesis of Compound 15a

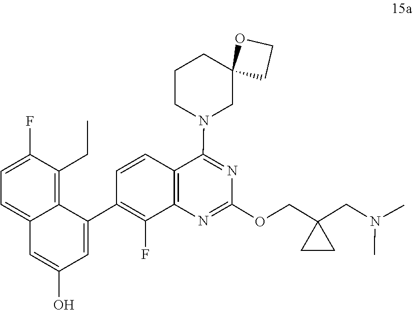 [SMILES: CCc1c(F)ccc2cc(O)cc(-c3ccc4c(N5CCC[C@]6(CCO6)C5)nc(OCC5(CN(C)C)CC5)nc4c3F)c12]

Title compound was synthesized from intermediate 70-1 in a manner essentially analogous to the synthesis of compound 73. ¹H NMR (400 MHz, CD₃OD) δ 8.18 (dd, J=17.1, 8.6 Hz, 1H), 7.70 (dd, J=9.1, 5.9 Hz, 1H), 7.57 (ddd, J=8.4, 7.0, 1.2 Hz, 1H), 7.36-7.24 (m, 2H), 7.00 (d, J=2.6 Hz, 1H), 4.80 (d, J=13.5 Hz, 2H), 4.64-4.50 (m, 5H), 3.90 (dd, J=13.5, 1.8 Hz, 1H), 3.62-3.48 (m, 1H), 3.39 (s, 1H), 3.00 (s, 6H), 2.60-2.42 (m, 4H), 2.34 (dd, J=10.8, 5.4 Hz, 1H), 2.13-1.96 (m, 2H), 1.93-1.82 (m, 1H), 1.04 (d, J=4.1 Hz, 2H), 0.94 (d, J=2.0 Hz, 2H), 0.83 (t, J=7.3 Hz, 3H). m/z, (ESI): 589.3 [M+H]⁺.

### Synthesis of Compound 16a

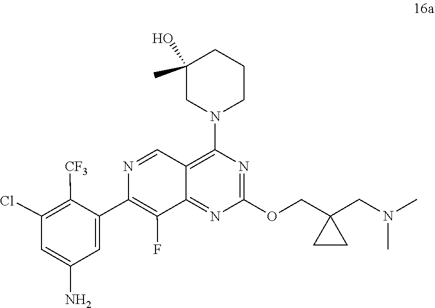 [SMILES: CN(C)CC1(COc2nc(N3CCC[C@@](C)(O)C3)c3cnc(-c4cc(N)cc(Cl)c4C(F)(F)F)c(F)c3n2)CC1]

Title compound was synthesized from (3R)-3-methylpiperidin-3-ol in a manner essentially analogous to the synthesis of compound 63. ¹H NMR (400 MHz, CD₃OD) δ 0.75 (s, 2H), 0.89 (s, 2H), 1.27 (s, 3H), 1.74-1.84 (m, 3H), 2.08-2.16 (m, 1H), 2.75 (s, 7H), 3.00 (t, J=17.2 Hz, 1H), 3.40 (t, J=12.4 Hz, 1H), 3.59 (d, J=13.6 Hz, 1H), 4.25-4.28 (m, 1H), 4.40-4.41 (m, 2H), 4.52-4.56 (m, 1H), 6.50 (s, 1H), 6.91 (s, 1H), 9.17 (s, 1H). m/z, (ESI): 584.3 [M+H]⁺.

### Synthesis of Compound 17a

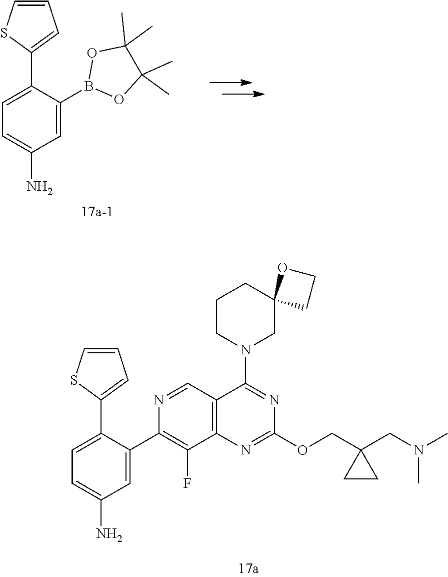 [SMILES: CC1(C)OB(c2cc(N)ccc2-c2cccs2)OC1(C)C ; CN(C)CC1(COc2nc(N3CCC[C@]4(CCO4)C3)c3cnc(-c4cc(N)ccc4-c4cccs4)c(F)c3n2)CC1]

Title compound was synthesized from intermediate 17a-1 in a manner essentially analogous to the synthesis of compound 63. ¹H NMR (400 MHz, CD₃OD) δ 9.19 (s, 1H), 7.42 (d, J=8.2 Hz, 1H), 6.91 (dd, J=3.0, 1.3 Hz, 1H), 6.70 (dd, J=5.0, 1.3 Hz, 1H), 4.68-4.61 (m, 1H), 4.52-4.45 (m, 2H), 4.41-4.34 (m, 1H), 4.33-4.24 (m, 2H), 3.71-3.63 (m, 1H), 3.35-3.27 (m, 1H), 3.16-3.11 (m, 2H), 2.80 (s, 6H), 2.47-2.36 (m, 2H), 2.23-2.19 (m, 1H), 1.96-1.77 (m, 3H), 1.74-1.71 (m, 1H), 1.53-1.47 (m, 1H), 0.88-0.81 (m, 2H), 0.75-0.72 (m, 2H). m/z, (ESI⁺): 585.3 [M+H]⁺.

### Synthesis of Compound 18a

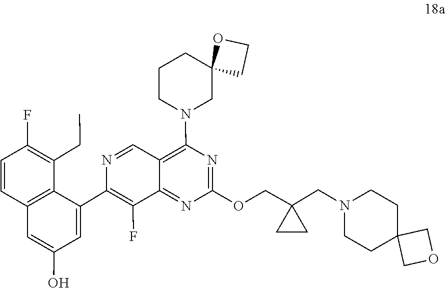 [SMILES: CCc1c(F)ccc2cc(O)cc(-c3ncc4c(N5CCC[C@]6(CCO6)C5)nc(OCC5(CN6CCC7(CC6)COC7)CC5)nc4c3F)c12]

Title compound was synthesized from 2-oxa-7-aza-spiro[3.5]nonane in a manner essentially analogous to the synthesis of compound 73. ¹H NMR (400 MHz, CD₃OD) δ 0.78-0.83 (m, 3H), 0.86 (s, 2H), 1.00 (s, 2H), 1.78-2.54 (m, 13H), 2.96 (t, J=12.8 Hz, 2H), 3.22-3.48 (m, 2H), 3.74-3.84 (m, 3H), 4.41-4.77 (m, 10H), 7.07-7.09 (m, 1H), 7.26 (t, J=9.6 Hz, 1H), 7.32-7.33 (m, 1H), 7.65-7.71 (m, 1H), 9.29-9.31 (m, 1H). ¹⁹F NMR (376 MHz, CD₃OD) δ −139.19 (d, J=54.1 Hz 1H), −120.92, (s, 1H), −77.15 (s, 6H). m/z, (ESI): 672.6 [M+H]⁺.

### Synthesis of Compound 19a

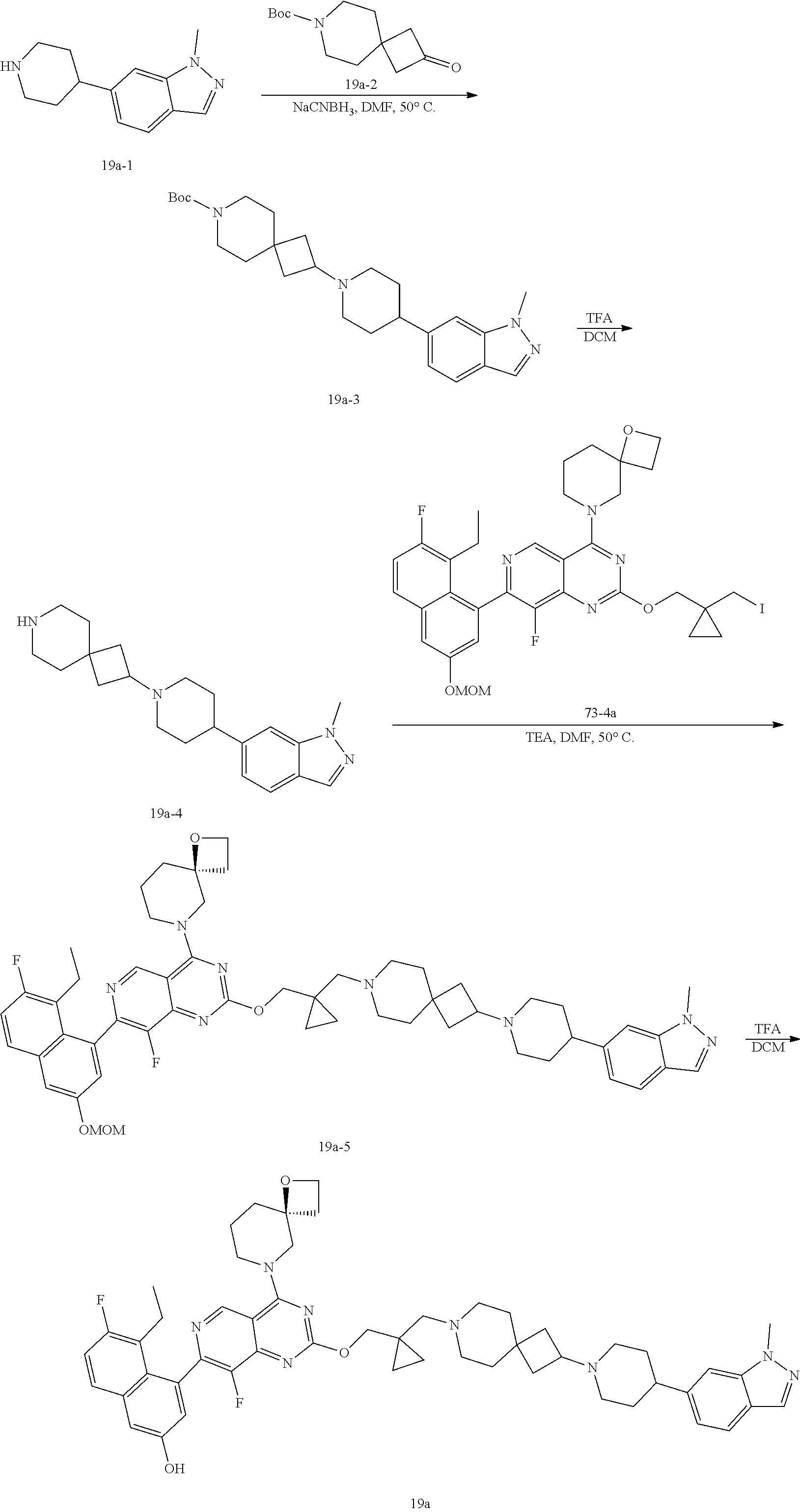

To a stirred solution of intermediate 19a-1 (175.40 mg, 696.72 μmol, 1 eq) in dimethylformamide (2 mL), triethylamine (108.3 mg, 1.07 mmol, 1.1 eq) and intermediate 19a-2 (250.10 mg, 1.05 mmol, 1.5 eq) were added subsequently. The reaction mixture was stirred at 50° C. for 1 hour. Sodium cyanoborohydride was then added to the reaction mixture, and stirring continued for another 16 hours. Upon the completion of conversion, the reaction mixture was diluted with ethyl acetate. The organic layer was washed with water and brine, dried over sodium sulfate and then filtered. The filtrate was concentrated in vacuo to afford crude product, which was subjected to silica gel chromatography eluted with dichloromethane/methanol (v/v=100/0 to 97/3) to give purified compound 19a-3 (130 mg, Yield 42.50%).

To a stirred solution of intermediate 19a-3 (130 mg, 296.39 μmol, 1 eq) in dichloromethane (2 mL), trifluoroacetic acid (33.79 mg, 296.39 μmol, 1 eq) was added in one portion. The reaction mixture was stirred for 10 minutes at room temperature. Upon the completion of conversion, the solvent was removed in vacuo to afford compound 19a-4, which was directly subjected to the next transformation without further purification (130 mg, Yield 96.90%).

To a stirred solution of intermediate 19a-4 (31.58 mg, 69.78 μmol, 1 eq) in dimethylformamide (2 mL), intermediate 73-4a (50 mg, 69.78 μmol, 1 eq) and triethylamine (173.40 mg, 1.34 mmol, 233.70 μL, 2 eq) were added subsequently. The reaction mixture was warmed to 50° C. and stirred for 16 hours. Upon the completion of conversion, the reaction mixture was cooled to room temperature and diluted with ethyl acetate. The organic layer was washed with water and brine, dried over sodium sulfate and then filtered. The filtrate was concentrated in vacuo to afford crude product, which was subjected to silica gel chromatography eluted with dichloromethane/methanol (v/v=100/0 to 99/1) to give purified compound 19a-5 (34 mg, Yield 52.56%).

To a stirred solution of intermediate 19a-5 (34 mg, 36.67 μmol, 1 eq) in dichloromethane (2 mL), trifluoroacetic acid (4.18 mg, 36.67 μmol, 1 eq) was added in one portion. The reaction mixture was stirred for 10 minutes at room temperature. Upon the completion of conversion, the solvent was removed in vacuo to afford compound 19a-4, which was subjected to a reverse phase preparative HPLC eluted with 0.1% trifluoroacetic acid aqueous solution/acetonitrile to give the compound 19a (13.3 mg, Yield 25.81%). ¹H NMR (400 MHz, CD₃OD) δ 0.79-0.83 (m, 3H), 0.88 (s, 2H), 1.00 (s, 2H), 1.82-2.54 (m, 21H), 2.94-3.14 (m, 5H), 3.37-3.49 (m, 2H), 3.60-3.63 (m, 2H), 3.74-3.86 (m, 4H), 4.05 (s, 3H), 4.44-4.74 (m, 6H), 7.07-7.11 (m, 2H), 7.25 (t, J=9.2 Hz, 1H), 7.31-7.32 (m, 1H), 7.41 (s, 1H), 7.66-7.74 (m, 2H), 7.96 (s, 1H), 9.28-9.30 (m, 1H). ¹⁹F NMR (376 MHz, CD₃OD) δ −139.34 (d, J=55.2 Hz 1H), −120.97, (s, 1H), −77.03 (s, 12H). m/z, (ESI): 883.9 [M+H]⁺.

### Synthesis of Compound 20a

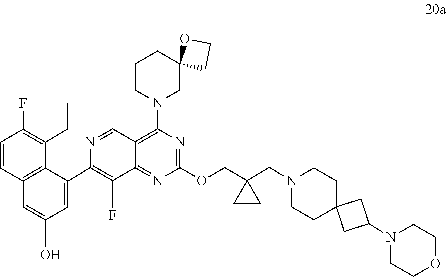 [SMILES: CCc1c(F)ccc2cc(O)cc(-c3ncc4c(N5CCC[C@]6(CCO6)C5)nc(OCC5(CN6CCC7(CC6)CC(N6CCOCC6)C7)CC5)nc4c3F)c12]

Title compound was synthesized from morpholine in a manner essentially analogous to the synthesis of compound 19a. ¹H NMR (400 MHz, CD₃OD) δ 0.63-0.90 (m, 7H), 1.64-1.81 (m, 6H), 1.91-2.03 (m, 4H), 2.17-2.34 (m, 6H), 2.45-3.53 (m, 3H), 2.74 (t, J=8.0 Hz, 2H), 3.38-3.50 (t, J=5.2 Hz, 4H), 3.83 (t, J=14 Hz, 1H), 4.40-4.71 (m, 12H), 7.07 (s, 1H), 7.25 (t, J=9.6 Hz, 1H), 7.30-7.31 (m, 1H), 7.66-7.70 (m, 1H), 9.25-9.27 (m, 1H). m/z, (ESI): 755.8 [M+H]⁺.

### Synthesis of Compound 21a

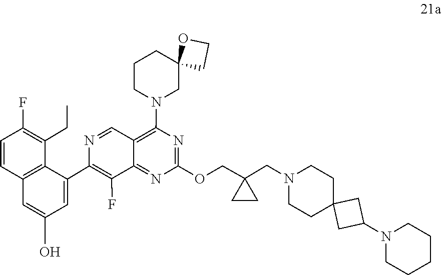 [SMILES: CCc1c(F)ccc2cc(O)cc(-c3ncc4c(N5CCC[C@]6(CCO6)C5)nc(OCC5(CN6CCC7(CC6)CC(N6CCCCC6)C7)CC5)nc4c3F)c12]

Title compound was synthesized from piperidine in a manner essentially analogous to the synthesis of compound 19a. ¹H NMR (400 MHz, CD₃OD) δ 0.77-0.82 (m, 3H), 0.86 (s, 2H), 0.99 (s, 2H), 1.44-1.54 (m, 1H), 1.66-2.51 (m, 23H), 2.71 (t, J=12.4 Hz, 2H), 2.98-3.13 (m, 2H), 3.41-3.47 (m, 3H), 3.66-3.84 (m, 4H), 4.42-4.76 (m, 6H), 7.04-7.07 (m, 1H), 7.26 (t, J=9.2 Hz, 1H), 7.30-7.31 (m, 1H), 7.66-7.70 (m, 1H), 9.27-9.29 (m, 1H). ¹⁹F NMR (376 MHz, CD₃OD) δ −139.23 (d, J=54.8 Hz 1H), −120.99, (s, 1H), −77.05 (s, 9H). m/z, (ESI): 753.6 [M+H]⁺.

### Synthesis of Compound 22a

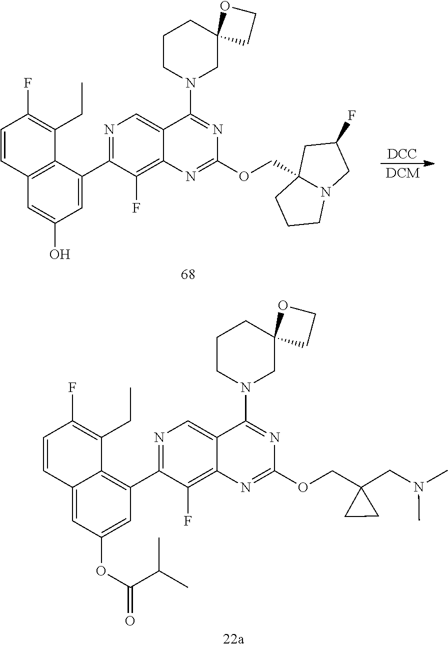

To a stirred solution of compound 68 (35.00 mg, 59.35 μmol, 1 eq) in dichloromethane (5 mL), isobutyric acid (5.23 mg, 59.35 μmol, 1 eq) and Dicyclohexylcarbodiimide (12.25 mg, 59.35 mol, 1 eq) were added subsequently. The reaction mixture was stirred at room temperature for 16 hours. Upon the completion of conversion, the reaction mixture was filtered and insoluble solid was discarded. The filtrate was diluted with ethyl acetate, then washed with water and brine, dried over sodium sulfate and filtered. Filtrate was concentrated in vacuo to afford crude product, which was subjected to silica gel chromatography eluted with dichloromethane/methanol (v/v=20/1) to give purified compound 1-6 (180 mg, Yield 49.68%). ¹H NMR (400 MHz, CD₃OD) δ ppm 9.28 (s, 1H), 7.92 (dd, J=9.0, 5.8 Hz, 1H), 7.81 (d, J=2.6 Hz, 1H), 7.41 (t, J=9.4 Hz, 1H), 7.30 (d, J=2.3 Hz, 1H), 4.66-4.53 (m, 3H), 4.50-4.33 (m, 3H), 3.90-3.75 (m, 1H), 3.57-3.42 (m, 1H), 2.95-2.79 (m, 1H), 2.58-2.46 (m, 4H), 2.39-2.25 (m, 8H), 2.08-1.88 (m, 2H), 1.88-1.74 (m, 1H), 1.72-1.53 (m, 1H), 1.33 (d, J=7.0 Hz, 6H), 0.88-0.79 (m, 3H), 0.75 (d, J=4.9 Hz, 2H), 0.59-0.52 (m, 2H); ¹⁹F NMR (376 MHz, CD₃OD) δ ppm −116.66 (t, J=7.3 Hz, 1F), −138.95 (d, J=44.7 Hz, 1F). m/z, (ESI): 661.1 [M+H]⁺.

### Synthesis of Compound 23a

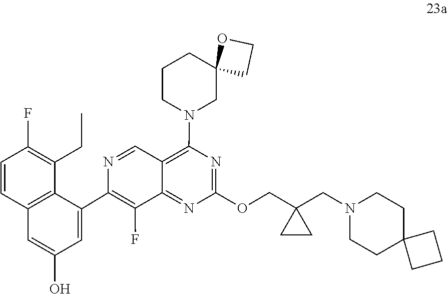 [SMILES: CCc1c(F)ccc2cc(O)cc(-c3ncc4c(N5CCC[C@]6(CCO6)C5)nc(OCC5(CN6CCC7(CCC7)CC6)CC5)nc4c3F)c12]

Title compound was synthesized from 7-aza-spiro[3.5]nonane in a manner essentially analogous to the synthesis of compound 73. ¹H NMR (400 MHz, CD₃OD) δ 0.79-0.84 (m, 3H), 0.86 (s, 2H), 0.99 (s, 2H), 1.84-2.06 (m, 14H), 2.19-2.52 (m, 5H), 2.97 (t, J=12.4 Hz, 2H), 3.22-3.49 (m, 2H), 3.70-3.85 (m, 3H), 4.42-4.78 (m, 6H), 7.08 (t, J=3.2 Hz, 1H), 7.27 (t, J=9.2 Hz, 1H), 7.33-7.36 (m, 1H), 7.68-7.72 (m, 1H), 9.29-9.30 (m, 1H). ¹⁹F NMR (376 MHz, CD₃OD) δ −139.18 (d, J=55.2 Hz 1H), −120.98, (s, 1H), −77.23 (s, 6H). m/z, (ESI): 670.5 [M+H]⁺.

### Synthesis of Compound 24a

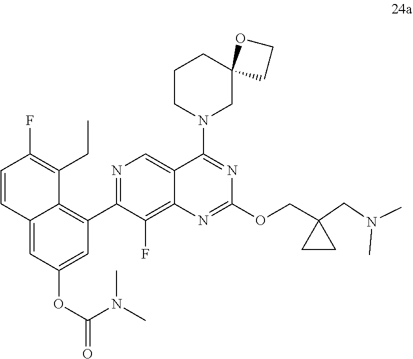 [SMILES: CCc1c(F)ccc2cc(OC(=O)N(C)C)cc(-c3ncc4c(N5CCC[C@]6(CCO6)C5)nc(OCC5(CN(C)C)CC5)nc4c3F)c12]

Title compound was synthesized from dimethylcarbamoyl chloride in a manner essentially analogous to the synthesis of compound 22a. ¹H NMR (400 MHz, CD₃OD) δ ppm 9.27 (s, 1H), 7.90 (dd, J=9.0, 5.8 Hz, 1H), 7.81 (d, J=2.6 Hz, 1H), 7.39 (t, J=9.4 Hz, 1H), 7.34 (d, J=2.6 Hz, 1H), 4.69-4.49 (m, 4H), 4.49-4.33 (m, 3H), 3.84 (dd, J=17.9, 13.6 Hz, 1H), 3.54-3.38 (m, 1H), 3.22-3.10 (m, 3H), 3.08-2.94 (m, 3H), 2.63-2.40 (m, 5H), 2.40-2.20 (m, 8H), 2.12-1.88 (m, 2H), 1.88-1.73 (m, 1H), 0.88-0.79 (m, 3H), 0.79-0.72 (m, 2H), 0.62-0.45 (m, 2H); ¹⁹F NMR (376 MHz, CD₃OD) δ ppm −116.95 (t, J=7.4 Hz, 1F), −138.94 (d, J=41.7 Hz, 1F). m/z, (ESI): 662.1 [M+H]⁺.

### Synthesis of Compound 25a

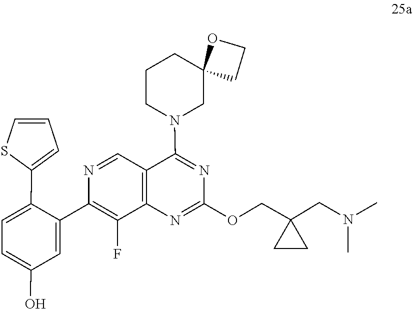 [SMILES: CN(C)CC1(COc2nc(N3CCC[C@]4(CCO4)C3)c3cnc(-c4cc(O)ccc4-c4cccs4)c(F)c3n2)CC1]

Title compound was synthesized from 2-(5-(methoxymethoxy)-2-(thiophen-2-yl)phenyl)-4,4,5,5-tetramethyl-1,3,2-dioxaborolane in a manner essentially analogous to the synthesis of compound 73. ¹H NMR (400 MHz, CD₃OD) δ 9.27 (s, 1H), 7.42 (d, J=8.4 Hz, 1H), 7.20-7.16 (m, 1H), 7.00 (d, J=8.4 Hz, 1H), 6.98-6.91 (m, 2H), 6.78 (dd, J=5.0, 1.3 Hz, 1H), 4.73-4.67 (m, 1H), 4.60-4.55 (m, 2H), 4.51-4.44 (m, 1H), 4.43-4.34 (m, 2H), 3.80-3.74 (m, 1H), 3.50-3.36 (m, 2H), 3.25-3.10 (m, 3H), 2.92-2.86 (m, 4H), 2.58-2.47 (m, 2H), 2.34-2.27 (m, 1H), 2.03-1.90 (m, 2H), 1.84-1.77 (m, 1H), 0.98-0.89 (m, 2H), 0.88-0.79 (m, 2H). m/z (ESI): 576 [M+H]⁺.

### Synthesis of Compound 26a

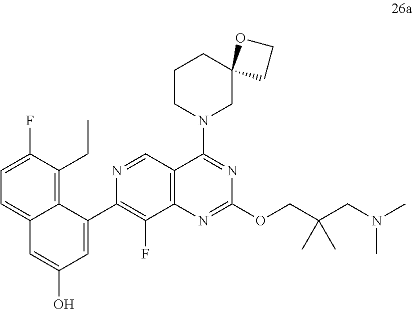 [SMILES: CCc1c(F)ccc2cc(O)cc(-c3ncc4c(N5CCC[C@]6(CCO6)C5)nc(OCC(C)(C)CN(C)C)nc4c3F)c12]

Title compound was synthesized from 2,2-dimethylpropane-1,3-diol in a manner essentially analogous to the synthesis of compound 73. ¹H NMR (400 MHz, CD₃OD) δ 9.34 (d, J=6.6 Hz, 1H), 7.71 (dd, J=9.0, 5.8 Hz, 1H), 7.34 (d, J=2.7 Hz, 1H), 7.28 (t, J=9.4 Hz, 1H), 7.10 (t, J=2.5 Hz, 1H), 4.65-4.50 (m, 3H), 4.42 (s, 2H), 3.39 (s, 2H), 3.04 (s, 6H), 2.57-2.45 (m, 3H), 2.38-2.31 (m, 1H), 2.26-2.15 (m, 2H), 2.12-2.02 (m, 2H), 2.01-1.94 (m, 2H), 1.91-1.79 (m, 2H), 1.36 (s, 3H), 1.29-1.26 (m, 5H). m/z (ESI): 592 [M+H]⁺.

### Synthesis of Compound 27a

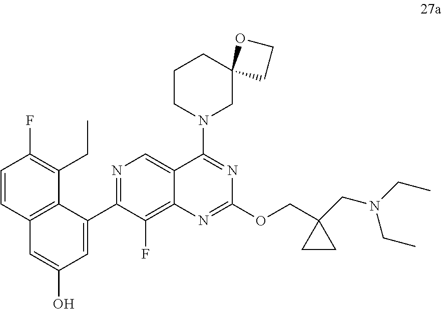 [SMILES: CCc1c(F)ccc2cc(O)cc(-c3ncc4c(N5CCC[C@]6(CCO6)C5)nc(OCC5(CN(CC)CC)CC5)nc4c3F)c12]

Title compound was synthesized from diethylamine in a manner essentially analogous to the synthesis of compound 73. ¹H NMR (400 MHz, CD₃OD) δ 9.20 (d, J=7.0 Hz, 1H), 7.59 (dd, J=9.1, 5.8 Hz, 1H), 7.29-7.08 (m, 2H), 6.97 (t, J=2.7 Hz, 1H), 4.69-4.54 (m, 2H), 4.51-4.31 (m, 4H), 3.66-3.74 (m, 1H), 3.45-3.23 (m, 4H), 2.47-2.32 (m, 2H), 2.25-2.16 (m, 1H), 2.16-2.03 (m, 1H), 1.97-1.67 (m, 3H), 1.29-1.16 (m, 6H), 0.93-0.65 (m, 6H). m/z (ESI): 618 [M+H]⁺.

### Synthesis of Compound 28a

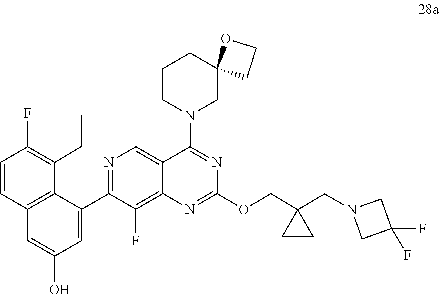 [SMILES: CCc1c(F)ccc2cc(O)cc(-c3ncc4c(N5CCC[C@]6(CCO6)C5)nc(OCC5(CN6CC(F)(F)C6)CC5)nc4c3F)c12]

Title compound was synthesized from 3,3-difluoroazetidine in a manner essentially analogous to the synthesis of compound 73. ¹H NMR (400 MHz, CD₃OD) δ 9.20 (d, J=7.1 Hz, 1H), 7.59 (dd, J=9.1, 5.8 Hz, 1H), 7.27-7.11 (m, 2H), 6.98 (t, J=2.8 Hz, 1H), 4.81 (d, J=11.0 Hz, 3H), 4.70-4.56 m, 1H), 4.53-4.24 (m, 5H), 3.76-3.65 (m, 1H), 3.46 (s, 2H), 3.40-3.26 (m, 1H), 2.50-1.59 (m, 9H), 0.89-0.58 (m, 7H). m/z (ESI): 638 [M+H]⁺.

### Synthesis of Compound 29a

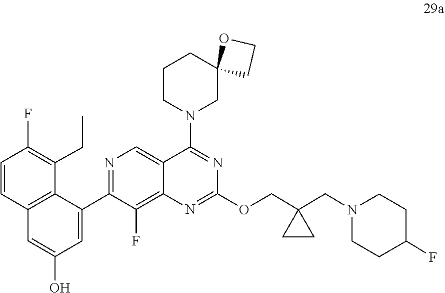 [SMILES: CCc1c(F)ccc2cc(O)cc(-c3ncc4c(N5CCC[C@]6(CCO6)C5)nc(OCC5(CN6CCC(F)CC6)CC5)nc4c3F)c12]

Title compound was synthesized from 4-fluoropiperidine in a manner essentially analogous to the synthesis of compound 73. ¹H NMR (400 MHz, CD₃OD) δ 9.32 (d, J=7.5 Hz, 1H), 7.71 (dd, J=9.1, 5.8 Hz, 1H), 7.37-7.24 (m, 2H), 7.10 (t, J=2.8 Hz, 1H), 5.12-4.94 (m, 1H), 4.84-4.68 (m, 2H), 4.59-4.46 (m, 4H), 4.0-3.8 (m, 3H), 3.52-3.40 (m, 1H), 3.40-3.35 (m, 2H), 3.28-3.13 (m, 2H), 2.59-2.43 (m, 3H), 2.40-1.89 (m, 8H), 1.87-1.75 (m, 1H), 1.08-0.96 (m, 2H), 0.94-0.86 (m, 2H), 0.85-0.76 (m, 3H). m/z (ESI): 648 [M+H]⁺.

### Synthesis of Compound 30a

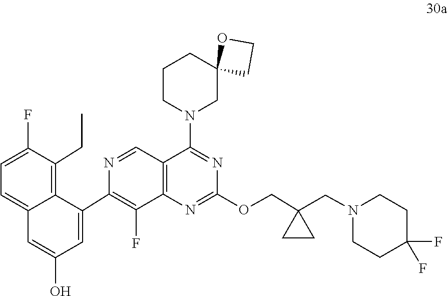 [SMILES: CCc1c(F)ccc2cc(O)cc(-c3ncc4c(N5CCC[C@]6(CCO6)C5)nc(OCC5(CN6CCC(F)(F)CC6)CC5)nc4c3F)c12]

Title compound was synthesized from 4,4-difluoropiperidine in a manner essentially analogous to the synthesis of compound 73. ¹H NMR (400 MHz, CD₃OD) δ 9.31 (d, J=6.5 Hz, 1H), 7.71 (dd, J=9.1, 5.8 Hz, 1H), 7.36-7.23 (m, 2H), 7.09 (t, J=3.0 Hz, 1H), 4.84-4.65 (m, 2H), 4.62-4.46 (m, 4H), 4.16-3.67 (m, 4H), 3.51-3.40 (m, 3H), 2.60-1.72 (m, 13H), 1.10-0.75 (m, 7H). m/z (ESI): 666 [M+H]⁺.

### Synthesis of Compound 31a

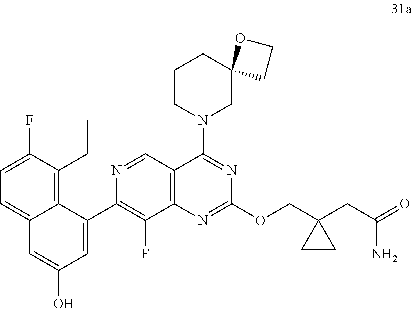 [SMILES: CCc1c(F)ccc2cc(O)cc(-c3ncc4c(N5CCC[C@]6(CCO6)C5)nc(OCC5(CC(N)=O)CC5)nc4c3F)c12]

Title compound was synthesized from 2-(1-(hydroxymethyl)cyclopropyl)acetonitrile in a manner essentially analogous to the synthesis of compound 73. ¹H NMR (400 MHz, CD₃OD) δ 9.35 (d, J=10.1 Hz, 1H), 7.72 (dd, J=9.1, 5.9 Hz, 1H), 7.42-7.21 (m, 2H), 7.12 (d, J=2.6 Hz, 1H), 4.86-4.72 (m, 3H), 4.67-4.45 (m, 4H), 3.94-3.83 (m, 1H), 3.61-3.47 (m, 1H), 2.73-2.14 (m, 7H), 2.12-1.80 (m, 3H), 0.95-0.56 (m, 6H). m/z (ESI): 590 [M+H]⁺.

### Synthesis of Compound 32a

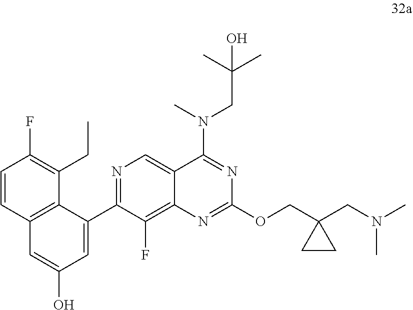 [SMILES: CCc1c(F)ccc2cc(O)cc(-c3ncc4c(N(C)CC(C)(C)O)nc(OCC5(CN(C)C)CC5)nc4c3F)c12]

Title compound was synthesized from 2-methyl-1-(methylamino)propan-2-ol in a manner essentially analogous to the synthesis of compound 73. ¹H NMR (400 MHz, CD₃OD) δ 9.44 (s, 1H), 7.71 (dd, J=9.1, 5.8 Hz, 1H), 7.34 (d, J=2.7 Hz, 1H), 7.29 (t, J=9.4 Hz, 1H), 4.46 (d, J=1.8 Hz, 2H), 4.23 (d, J=14.0 Hz, 1H), 4.09 (d, J=14.0 Hz, 1H), 3.82 (s, 3H), 3.03 (d, J=10.9 Hz, 6H), 2.56-2.17 (m, 3H), 1.97 (s, 1H), 1.35 (d, J=4.3 Hz, 6H), 1.06-0.97 (m, 2H), 0.94-0.88 (m, 2H), 0.836-0.77 (m, 3H). m/z (ESI): 566 [M+H]⁺.

### Synthesis of Compound 33a

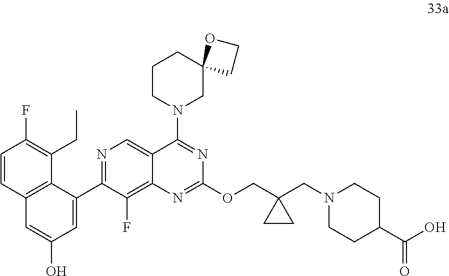 [SMILES: CCc1c(F)ccc2cc(O)cc(-c3ncc4c(N5CCC[C@]6(CCO6)C5)nc(OCC5(CN6CCC(C(=O)O)CC6)CC5)nc4c3F)c12]

Title compound was synthesized from methyl piperidine-4-carboxylate in a manner essentially analogous to the synthesis of compound 73. ¹H NMR (400 MHz, CD₃OD) δ 9.18 (d, J=5.0 Hz, 1H), 7.59 (dd, J=9.1, 5.8 Hz, 1H), 7.22 (d, J=2.7 Hz, 1H), 7.16 (t, J=9.4 Hz, 1H), 7.01-6.93 (m, 1H), 4.66-4.56 (d, J=13.7 Hz, 1H), 4.52-4.29 (m, 5H), 3.92-3.76 (m, 2H), 3.75-3.56 (m, 2H), 2.98-2.84 m, 2H), 2.63-1.65 (m, 15H), 0.95-0.64 (m, 7H). m/z (ESI): 674 [M+H]⁺.

### Synthesis of Compound 34a

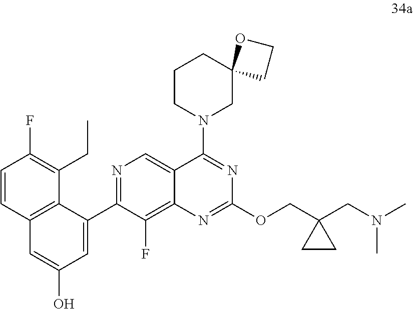 [SMILES: CCc1c(F)ccc2cc(O)cc(-c3ncc4c(N5CCC[C@]6(CCO6)C5)nc(OCC5(CN(C)C)CC5)nc4c3F)c12]

Title compound was synthesized from (S)-1-oxa-6-azaspiro[3.5]nonane in a manner essentially analogous to the synthesis of compound 73. ¹H NMR (400 MHz, CD₃OD) δ 9.34 (d, J=8.5 Hz, 1H), 7.71 (dd, J=9.1, 5.8 Hz, 1H), 7.37-7.25 (m, 2H), 7.10 (t, J=2.7 Hz, 1H), 4.85-4.70 (m, 2H), 4.63-4.44 (m, 5H), 3.88-3.80 (m, 1H), 3.54-3.36 (m, 2H), 3.02 (s, 6H), 2.61-2.44 (m, 3H), 2.37-1.79 (m, 6H), 1.04-0.98 (m, 2H), 0.94-0.88 (m, 2H), 0.86-0.79 (m, 3H). m/z (ESI): 590 [M+H]⁺.

### Synthesis of Compound 35a

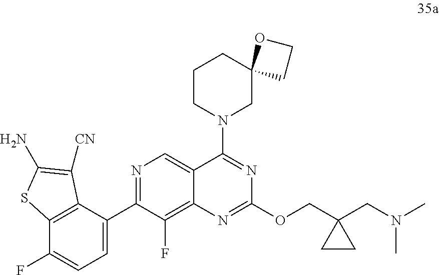 [SMILES: CN(C)CC1(COc2nc(N3CCC[C@]4(CCO4)C3)c3cnc(-c4ccc(F)c5sc(N)c(C#N)c45)c(F)c3n2)CC1]

Title compound was synthesized from 2-amino-4-(5,5-dimethyl-1,3,2-dioxaborinan-2-yl)-7-fluorobenzo[b]thiophene-3-carbonitrile in a manner essentially analogous to the synthesis of compound 73. ¹H NMR (400 MHz, CD₃OD) δ 9.31 (s, 1H), 7.45 (dd, J=8.4, 5.1 Hz, 1H), 7.15-7.03 (m, 1H), 4.74-4.33 (m, 5H), 3.90-3.77 (m, 1H), 3.52-3.42 (m, 1H), 3.38-3.31 (m, 5H), 3.01 (s, 4H), 2.59-2.36 (m, 2H), 2.33-2.21 (m, 1H), 2.07-1.73 (m, 3H), 1.16-0.94 (m, 2H), 0.93-0.75 (m, 2H). m/z (ESI): 592 [M+H]⁺.

### Synthesis of Compound 36a

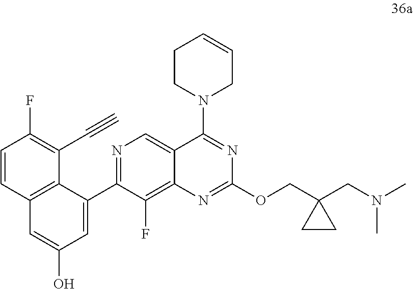 [SMILES: C#Cc1c(F)ccc2cc(O)cc(-c3ncc4c(N5CC=CCC5)nc(OCC5(CN(C)C)CC5)nc4c3F)c12]

Title compound was synthesized from 1,2,3,6-tetrahydropyridine in a manner essentially analogous to the synthesis of compound 1. ¹H NMR (400 MHz, CD₃OD) δ 9.13 (s, 1H), 7.91 (dd, J=9.2, 5.7 Hz, 1H), 7.42-7.33 (m, 2H), 7.27 (d, J=2.6 Hz, 1H), 6.12-6.01 (m, 1H), 5.90-5.80 (m, 1H), 4.65-4.41 (m, 4H), 4.34-4.12 (m, 2H), 3.50-3.47 (m, 1H), 3.39-3.34 (m, 1H), 3.30-3.25 (m, 1H), 3.00 (s, 6H), 2.60-2.45 (m, 2H), 1.04-0.85 (m, 4H). m/z (ESI): 542 [M+H]⁺.

### Synthesis of Compound 37a

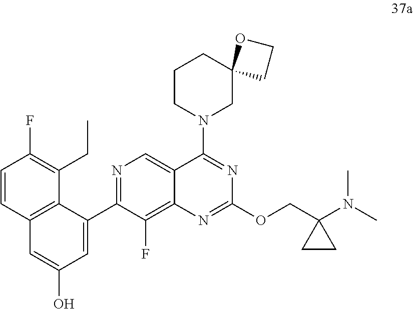 [SMILES: CCc1c(F)ccc2cc(O)cc(-c3ncc4c(N5CCC[C@]6(CCO6)C5)nc(OCC5(N(C)C)CC5)nc4c3F)c12]

Title compound was synthesized from (1-(dimethylamino)cyclopropyl)methanol in a manner essentially analogous to the synthesis of compound 23. ¹H NMR (400 MHz, CD₃OD) δ 9.31 (s, 1H), 7.45 (dd, J=8.4, 5.1 Hz, 1H), 7.15-7.03 (m, 1H), 4.74-4.33 (m, 5H), 3.90-3.77 (m, 1H), 3.52-3.42 (m, 1H), 3.38-3.31 (m, 5H), 3.01 (s, 4H), 2.59-2.36 (m, 2H), 2.33-2.21 (m, 1H), 2.07-1.73 (m, 3H), 1.16-0.94 (m, 2H), 0.93-0.75 (m, 2H). m/z (ESI): 576 [M+H]⁺.

### Synthesis of Compound 38a

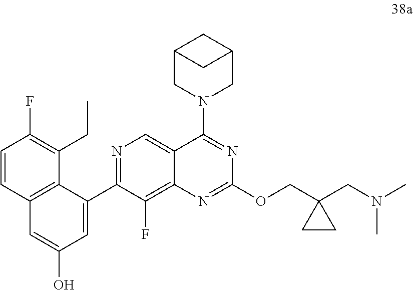 [SMILES: CCc1c(F)ccc2cc(O)cc(-c3ncc4c(N5CC6CC(C6)C5)nc(OCC5(CN(C)C)CC5)nc4c3F)c12]

Title compound was synthesized from 3-azabicyclo[3.1.1]heptane in a manner essentially analogous to the synthesis of compound 73. ¹H NMR (400 MHz, CD₃OD) δ 9.50 (s, 1H), 7.91 (dd, J=9.2, 5.7 Hz, 1H), 7.43-7.33 (m, 2H), 7.26 (d, J=2.6 Hz, 1H), 4.61-4.29 (m, 6H), 3.49-3.46 (m, 1H), 3.40-3.35 (m, 1H), 3.31-3.25 (m, 1H), 3.01 (s, 6H), 2.80-2.70 (m, 2H), 2.43-2.30 (m, 2H), 1.64-1.56 (m, 2H), 1.03-0.85 (m, 4H). m/z (ESI): 560 [M+H]⁺.

### Synthesis of Compound 39a

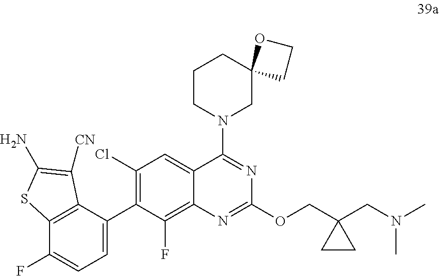 [SMILES: CN(C)CC1(COc2nc(N3CCC[C@]4(CCO4)C3)c3cc(Cl)c(-c4ccc(F)c5sc(N)c(C#N)c45)c(F)c3n2)CC1]

Title compound was synthesized from 2-amino-4-(5,5-dimethyl-1,3,2-dioxaborinan-2-yl)-7-fluorobenzo[b]thiophene-3-carbonitrile in a manner essentially analogous to the synthesis of compound 73. ¹H NMR (400 MHz, CD₃OD) δ 8.32-8.21 (m, 1H), 7.28-7.19 (m, 1H), 7.10-7.02 (m, 1H), 4.67-4.47 (m, 3H), 4.39-4.20 (m, 2H), 3.82-3.57 (m, 2H), 3.40-3.34 (m, 1H), 3.24-3.17 (m, 1H), 2.98 (s, 6H), 2.53-2.33 (m, 2H), 2.32-2.23 (m, 1H), 2.17-1.73 (m, 4H), 1.03-0.77 (m, 4H). m/z (ESI): 625 [M+H]⁺.

### Synthesis of Compound 40a

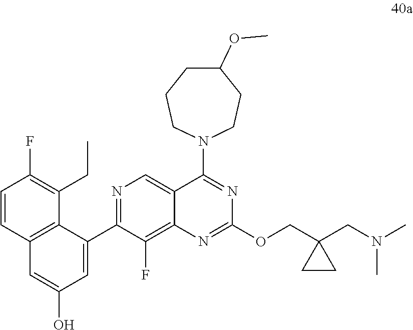 [SMILES: CCc1c(F)ccc2cc(O)cc(-c3ncc4c(N5CCCC(OC)CC5)nc(OCC5(CN(C)C)CC5)nc4c3F)c12]

Title compound was synthesized from 4-methoxyazepane in a manner essentially analogous to the synthesis of compound 73. ¹H NMR (400 MHz, CD₃OD) δ 9.22 (s, 1H), 7.71 (dd, J=9.1, 5.8 Hz, 1H), 7.35 (d, J=2.7 Hz, 1H), 7.29 (t, J=9.4 Hz, 1H), 7.09 (t, J=2.9 Hz, 1H), 4.56-4.42 (m, 2H), 4.26-4.04 (m, 4H), 3.63-3.56 (m, 1H), 3.38-3.34 (dd, J=9.8, 2.5 Hz, 5H), 3.02 (s, 6H), 2.57-2.43 (m, 1H), 2.32-2.10 (m, 4H), 2.02-1.77 (m, 3H), 1.03-0.87 (m, 4H), 0.85-0.81 (m, 3H). m/z (ESI): 592 [M+H]⁺.

### Synthesis of Compound 41a

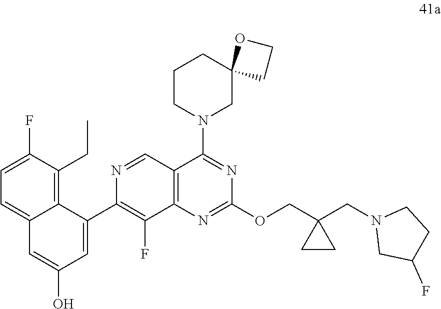 [SMILES: CCc1c(F)ccc2cc(O)cc(-c3ncc4c(N5CCC[C@]6(CCO6)C5)nc(OCC5(CN6CCC(F)C6)CC5)nc4c3F)c12]

Title compound was synthesized from 3-fluoropyrrolidine in a manner essentially analogous to the synthesis of compound 73. ¹H NMR (400 MHz, CD₃OD) δ 9.31 (dd, J=6.9, 2.7 Hz, 1H), 7.71 (dd, J=9.0, 5.8 Hz, 1H), 7.36-7.24 (m, 2H), 7.09 (t, J=2.9 Hz, 1H), 5.58-5.40 (m, 2H), 4.83-4.42 (m, 3H), 3.88-3.80 (m, 1H), 3.65-3.35 (m, 4H), 3.25-3.14 (m, 1H), 2.59-1.77 (m, 12H), 1.05-0.75 (m, 9H). m/z (ESI): 634 [M+H]⁺.

### Synthesis of Compound 42a

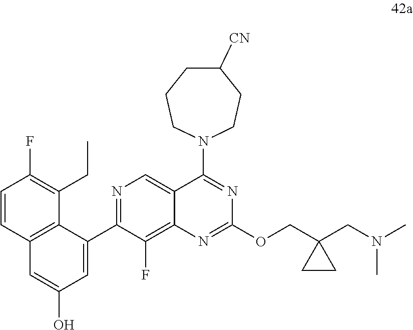 [SMILES: CCc1c(F)ccc2cc(O)cc(-c3ncc4c(N5CCCC(C#N)CC5)nc(OCC5(CN(C)C)CC5)nc4c3F)c12]

Title compound was synthesized from azepane-4-carbonitrile in a manner essentially analogous to the synthesis of compound 73. ¹H NMR (400 MHz, CD₃OD) δ 9.23 (s, 1H), 7.71 (dd, J=9.0, 5.8 Hz, 1H), 7.39-7.24 (m, 2H), 7.09 (dd, J=6.6, 2.6 Hz, 1H), 4.49 (s, 2H), 4.39-4.12 (m, 4H), 3.02 (s, 6H), 2.53-1.98 (m, 10H), 1.07-0.77 (m, 8H). m/z (ESI): 587 [M+H]⁺.

### Synthesis of Compound 43a

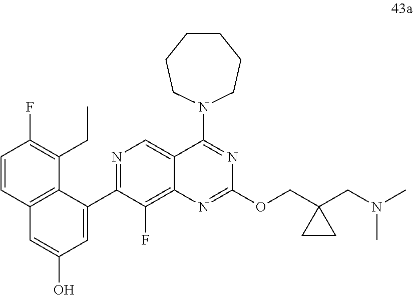 [SMILES: CCc1c(F)ccc2cc(O)cc(-c3ncc4c(N5CCCCCC5)nc(OCC5(CN(C)C)CC5)nc4c3F)c12]

Title compound was synthesized from azepane in a manner essentially analogous to the synthesis of compound 73. ¹H NMR (400 MHz, CD₃OD) δ 9.22 (s, 1H), 7.71 (dd, J=9.1, 5.8 Hz, 1H), 7.35 (d, J=2.6 Hz, 1H), 7.29 (t, J=9.4 Hz, 1H), 7.09 (d, J=2.7 Hz, 1H), 4.60-4.43 (m, 2H), 4.15 (t, J=5.8 Hz, 4H), 3.34 (d, J=2.8 Hz, 2H), 3.01 (s, 6H), 2.54-2.41 (m, 1H), 2.25 (ddd, J=11.5, 7.3, 3.6 Hz, 1H), 2.07 (s, 4H), 1.73 (d, J=4.6 Hz, 4H), 1.07-0.78 (m, 7H). m/z (ESI): 562 [M+H]⁺.

### Synthesis of Compound 44a

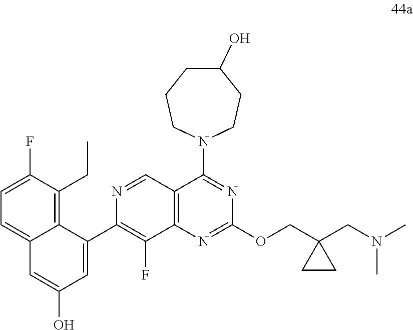 [SMILES: CCc1c(F)ccc2cc(O)cc(-c3ncc4c(N5CCCC(O)CC5)nc(OCC5(CN(C)C)CC5)nc4c3F)c12]

Title compound was synthesized from azepan-4-ol in a manner essentially analogous to the synthesis of compound 73. ¹H NMR (400 MHz, CD₃OD) δ 9.22 (s, 1H), 7.71 (dd, J=9.1, 5.8 Hz, 1H), 7.40-7.22 (m, 2H), 7.09 (d, J=2.6 Hz, 1H), 4.49 (dd, J=5.6, 2.4 Hz, 2H), 4.30-3.92 (m, 5H), 3.35 (s, 2H), 3.02 (s, 6H), 2.50 (q, J=7.4 Hz, 1H), 2.24 (ddd, J=14.4, 7.3, 3.0 Hz, 3H), 2.16-1.71 (m, 4H), 1.04-0.77 (m, 7H). m/z (ESI): 578 [M+H]⁺.

### Synthesis of Compound 45a

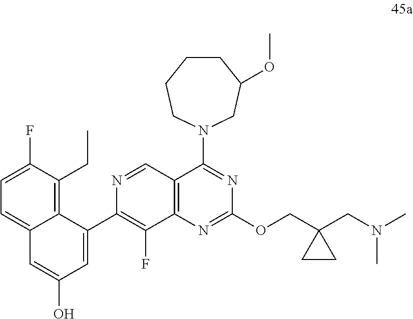 [SMILES: CCc1c(F)ccc2cc(O)cc(-c3ncc4c(N5CCCCC(OC)C5)nc(OCC5(CN(C)C)CC5)nc4c3F)c12]

Title compound was synthesized from 3-methoxyazepane in a manner essentially analogous to the synthesis of compound 73. ¹H NMR (400 MHz, CD₃OD) δ 9.35 (d, J=10.7 Hz, 1H), 7.70 (dd, J=9.1, 5.8 Hz, 1H), 7.36-7.24 (m, 2H), 7.08 (d, J=2.6 Hz, 1H), 4.54-4.32 (m, 3H), 4.27-4.17 (m, 1H), 4.14-4.00 (m, 2H), 3.88-3.80 (m, 1H), 3.46 (d, J=1.6 Hz, 3H), 3.39-3.32 (m, 3H), 3.01 (s, 6H), 2.55-2.41 (m, 1H), 2.29-2.16 (m, 1H), 2.11-1.90 (m, 4H), 1.87-1.73 (m, 1H), 1.61-1.48 (m, 1H), 1.02-0.95 (m, 2H), 0.93-0.88 (m, 2H), 0.86-0.78 (m, 3H). m/z (ESI): 592 [M+H]⁺.

### Synthesis of Compound 46a

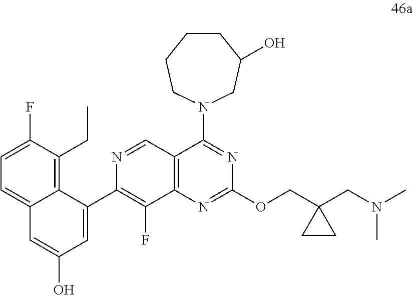 [SMILES: CCc1c(F)ccc2cc(O)cc(-c3ncc4c(N5CCCCC(O)C5)nc(OCC5(CN(C)C)CC5)nc4c3F)c12]

Title compound was synthesized from azepan-3-ol in a manner essentially analogous to the synthesis of compound 73. ¹H NMR (400 MHz, CD₃OD) δ 9.33 (d, J=8.7 Hz, 1H), 7.71 (dd, J=9.1, 5.8 Hz, 1H), 7.36-7.24 (m, 2H), 7.08 (t, J=3.2 Hz, 1H), 4.50-4.39 (m, 2H), 4.19-4.13 (m, 2H), 3.9-3.8 (m, 1H), 3.02 (s, 6H), 2.55-2.45 (m, 1H), 2.25-1.94 (m, 5H), 1.71-1.30 (m, 5H), 1.08-0.78 (m, 8H). m/z (ESI): 578 [M+H]⁺.

### Synthesis of Compound 47a

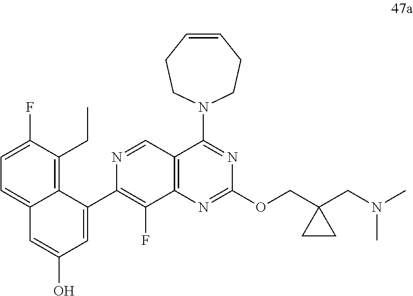 [SMILES: CCc1c(F)ccc2cc(O)cc(-c3ncc4c(N5CCC=CCC5)nc(OCC5(CN(C)C)CC5)nc4c3F)c12]

Title compound was synthesized from 2,3,6,7-tetrahydro-1H-azepine in a manner essentially analogous to the synthesis of compound 73. ¹H NMR (400 MHz, CD₃OD) δ 9.18 (s, 1H), 7.90 (dd, J=9.2, 5.7 Hz, 1H), 7.41-7.32 (m, 2H), 7.25 (d, J=2.5 Hz, 1H), 5.81 (t, J=2.9 Hz, 2H), 4.54 (d, J=12.0 Hz, 1H), 4.42-4.25 (m, 5H), 3.47-3.41 (m, 1H), 3.30-3.22 (m, 2H), 3.00 (s, 6H), 2.80-2.68 (m, 4H), 1.02-0.95 (m, 2H), 0.93-0.86 (m, 2H). m/z (ESI): 556 [M+H]⁺.

### Synthesis of Compound 48a

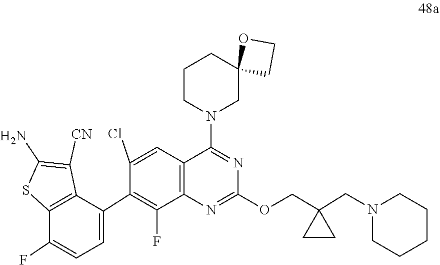 [SMILES: N#Cc1c(N)sc2c(F)ccc(-c3c(Cl)cc4c(N5CCC[C@]6(CCO6)C5)nc(OCC5(CN6CCCCC6)CC5)nc4c3F)c12]

Title compound was synthesized from 2-amino-4-(5,5-dimethyl-1,3,2-dioxaborinan-2-yl)-7-fluorobenzo[b]thiophene-3-carbonitrile and piperidine in a manner essentially analogous to the synthesis of compound 73. ¹H NMR (400 MHz, CD₃OD) δ 8.28-8.19 (m, 1H), 7.27-7.18 (m, 1H), 7.05 (dd, J=9.5, 8.4 Hz, 1H), 4.65-4.43 (m, 3H), 4.40-4.31 (m, 1H), 4.26-4.15 (m, 1H), 3.887-3.71 (m, 2H), 3.68-3.59 (m, 1H), 3.53-3.44 (m, 1H), 3.30-3.21 (m, 4H), 3.20-3.08 (m, 1H), 3.04-2.88 (m, 2H), 2.55-2.38 (m, 2H), 2.31-2.20 (m, 1H), 2.10-1.74 (m, 6H), 1.60-1.48 (m, 1H), 1.07-0.91 (m, 2H), 0.89-0.73 (m, 2H). m/z (ESI): 665 [M+H]⁺.

### Synthesis of Compound 49a

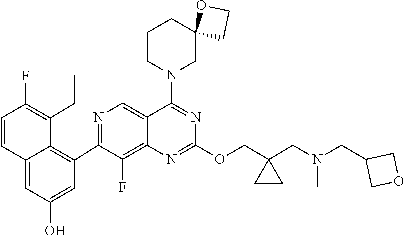 [SMILES: CCc1c(F)ccc2cc(O)cc(-c3ncc4c(N5CCC[C@]6(CCO6)C5)nc(OCC5(CN(C)CC6COC6)CC5)nc4c3F)c12]

Title compound was synthesized from N-methyl-1-(oxetan-3-yl)methanamine in a manner essentially analogous to the preparation of compound 73. ¹H NMR (400 MHz, CD₃OD) δ 9.30 (d, J=4.5 Hz, 1H), 7.72 (dd, J=9.0, 5.8 Hz, 1H), 7.35 (d, J=2.7 Hz, 1H), 7.29 (t, J=9.4 Hz, 1H), 7.16-7.07 (m, 1H), 4.77 (t, J=7.2 Hz, 2H), 4.71-4.38 (m, 10H), 3.87 (dd, J=16.1, 13.4 Hz, 1H), 3.49 (dt, J=23.9, 12.5 Hz, 1H), 2.60-2.16 (m, 10H), 2.09-1.94 (m, 2H), 1.85 (s, 1H), 0.85 (tt, J=12.1, 5.8 Hz, 6H), 0.61 (s, 2H). ¹⁹F NMR (376 MHz, CD₃OD) δ −121.09 (s, 1F), −139.16 (1, 1F). m/z (ESI): 646 [M+H]⁺.

### Synthesis of Compound 50a

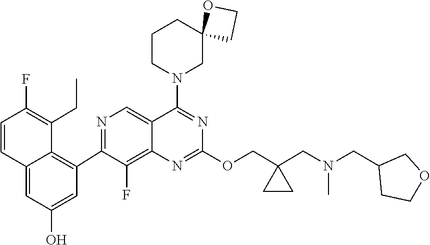 [SMILES: CCc1c(F)ccc2cc(O)cc(-c3ncc4c(N5CCC[C@]6(CCO6)C5)nc(OCC5(CN(C)CC6CCOC6)CC5)nc4c3F)c12]

Title compound was synthesized from N-methyl-1-(tetrahydrofuran-3-yl)methanamine in a manner essentially analogous to the preparation of compound 73. ¹H NMR (400 MHz, CD₃OD) δ 9.29 (d, J=4.9 Hz, 1H), 7.72 (dd, J=9.0, 5.8 Hz, 1H), 7.34 (d, J=2.6 Hz, 1H), 7.29 (t, J=9.4 Hz, 1H), 7.12 (d, J=2.6 Hz, 1H), 4.68-4.37 (m, 7H), 3.89 (ddd, J=16.9, 13.6, 2.7 Hz, 1H), 3.77 (t, J=7.0 Hz, 2H), 3.66 (q, J=7.4 Hz, 1H), 3.55-3.43 (m, 2H), 2.58-2.29 (m, 11H), 2.20 (td, J=10.0, 8.7, 5.9 Hz, 1H), 2.08-1.95 (m, 3H), 1.85 (s, 1H), 1.61 (s, 1H), 0.85 (td, J=7.3, 5.2 Hz, 3H), 0.76 (s, 2H), 0.55 (s, 2H). ¹⁹F NMR (376 MHz, CD₃OD) δ −121.12 (t, J=7.9 Hz, 1F), −139.04 (d, J=39.4 Hz, 1F). m/z (ESI): 660 [M+H]⁺.

### Synthesis of Positive Control A1.

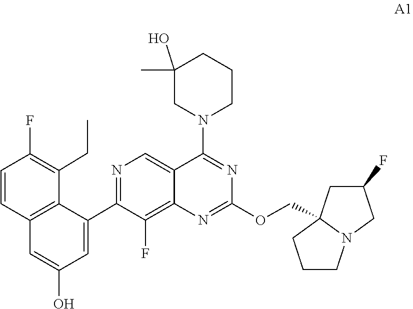 [SMILES: CCc1c(F)ccc2cc(O)cc(-c3ncc4c(N5CCCC(C)(O)C5)nc(OC[C@@]56CCCN5C[C@H](F)C6)nc4c3F)c12]

A1 was prepared as described in International PCT Application Publication No. WO 2022/132200.

### Biological Assays

### Example 1. KRAS Binding Assay

This example illustrates that exemplary compounds of the present invention bind to KRAS and are capable of displacing a fluorophore labeled tracer ligand occupying the KRAS binding site. KRAS^{G12D} was used in this assay.

The ability of a test compound to bind to KRAS^{G12D} was measured by using an HTRF KRAS^{G12D} GTP Binding Kit (Cisbio, 63ADK000CB27PEG). 100×His-tagged Human KRAS^{G12D} stock solution was diluted to 1× by adding PPI Europium detection buffer and then transferring the diluted solution to a 384-well plate (5 μL/well, PerkinElmer, 6008289). 5 μL of compound solution was added to each well after serial dilution, followed by addition of 5 μL of GDP (50 nM) solution. After 30 minutes incubation at room temperature, 1×6His Eu cryptate antibody solution and GTP-Red tracer were added to the plate, then incubated for another 30 minutes. The HTRF signal was measured with a VICTOR Nivo multimode plate reader according to the manufacturer's instructions. The percent inhibition was calculated using the following formula:

$$\% inhibition = 100 - [ \frac{( Test compound signal - Minimum Signal )}{( Maximum Signal - Minimum Signal )} ] \times 100$$

(where the “Maximum Signal” are wells containing DMSO without inhibitor; the “Minimum Signal” are wells containing reference inhibitory component (GDP); the “Test compound signal” are wells containing test compound (inhibitor) at established concentrations).

IC50 was determined by fitting percent inhibition at each inhibitor concentration to the four-parameter nonlinear logistic equation using GraphPad Prism. IC50 (nM) values of selected compounds are depicted in Table 3.

TABLE 3
IC50 values for test compounds for binding to KRAS^{G12D}.

| Compound | IC50 (nM) | Compound | IC50 (nM) |
|---|---|---|---|
| 1 | 261.3 | 67 | 415.3 |
| 2 | 184.9 | 68 | 6.3 |
| 3 | 101.6 | 70 | 67.4 |
| 4 | >1000 | 71 | >1000 |
| 6 | 1392 | 73 | 10.7 |
| 7 | >1000 | 92 | 416.2 |
| 8 | >1000 | 96 | 127.6 |
| 10 | 881 | 110 | >1000 |
| 11 | >1000 | 112 | >1000 |
| 12 | >1000 | 117 | 373.0 |
| 13 | 979 | 1a | 751.5 |
| 14 | >1000 | 2a | 18.8 |
| 15 | >1000 | 3a | 141.2 |
| 16 | >1000 | 4a | >1000 |
| 17 | >1000 | 5a | 652.0 |
| 18 | >10000 | 6a | >1000 |
| 19 | >10000 | 7a | >1000 |
| 20 | >10000 | 8a | >1000 |
| 21 | >10000 | 9a | 7.9 |
| 22 | >10000 | 10a | 6.9 |
| 23 | 1353 | 11a | >1000 |
| 24 | >10000 | 12a | 12.1 |
| 25 | 43.6 | 13a | 13.2 |
| 26 | 1534 | 14a | >1000 |
| 27 | >10000 | 15a | 19.6 |
| 28 | >10000 | 16a | 384.9 |
| 29 | >10000 | 17a | >1000 |
| 30 | >10000 | 18a | 4.5 |
| 31 | >10000 | 19a | 16.1 |
| 32 | >1000 | 20a | 4.7 |
| 33 | >1000 | 21a | 6.8 |
| 34 | >1000 | 22a | >1000 |
| 35 | >1000 | 23a | 31.2 |
| 36 | >1000 | 24a | >1000 |
| 37 | >1000 | 25a | 638.7 |
| 38 | 162.8 | 26a | 13.3 |
| 39 | >1000 | 27a | 12.7 |
| 40 | >1000 | 28a | 158.8 |
| 41 | >1000 | 29a | 4.3 |
| 42 | >1000 | 30a | 41.6 |
| 43 | >1000 | 31a | 518.4 |
| 44 | >1000 | 32a | 481.4 |
| 45 | >1000 | 33a | 10.3 |
| 46 | >1000 | 34a | 3.1 |
| 47 | >1000 | 35a | >1000 |
| 48 | >1000 | 36a | >1000 |
| 49 | >1000 | 37a | 121.3 |
| 50 | 71.02 | 38a | >1000 |
| 51 | >1000 | 39a | >1000 |
| 52 | >1000 | 40a | 424.2 |
| 53 | >1000 | 41a | 14.2 |
| 54 | >1000 | 42a | >1000 |
| 55 | >1000 | 43a | 556.7 |
| 56 | >1000 | 44a | 234.0 |
| 57 | 510.1 | 45a | >1000 |
| 58 | >1000 | 46a | 683.5 |
| 59 | >1000 | 47a | >1000 |
| 60 | >1000 | 48a | >1000 |
| 61 | >1000 | 49a | 4.05 |
| 62 | >1000 | 50a | 6.76 |
| 63 | >1000 | | |
| 64 | >1000 | | |

### Example 2. Cell Proliferation Assay

A cell proliferation assay was used to evaluate inhibition by exemplary compounds. Proliferation assays used human tissue derived cancer cell lines, for example, AsPC-1 (Cobioer, CBP60546), GP2D (Cobioer, CBP60683), NCI-H358 (Cobioer, CBP60544), NCI-H727 (Cobioer, CBP60182), MKN-1 (Cobioer, CBP60486), HCT116 (ATCC, CCL-247), and HT-29 (Cobioer, CBP60011). Signal reflecting cell proliferation was detected with the CellTiterGlo kit (Promega, G7573).

Cells were cultured in the growth phase and 3×10³ cells per well were plated in a 96-well plate (Greiner, 655090). Cells were incubated 24 hours at 37° C. and 5% carbon dioxide in a humidity chamber. Serially diluted exemplary compounds were added to the plate which was then incubated for another 72 hours. Plates were brought to room temperature and an equal volume of CellTiterGlo reagent was added. After 10 minutes incubation at room temperature to stabilize the signal, the luminescent signal was measured on the VICTOR Nivo multimode plate reader according to the manufacturer's instructions. The signal was converted to percent inhibition using the following formula:

$$\% inhibition = 100 - [ \frac{( Test compound signal - Minimum Signal )}{( Maximum Signal - Minimum Signal )} \times 100 ]$$

(where the “Maximum Signal” are wells containing DMSO without inhibitor; the “Minimum Signal” are signal of wells recorded at Day 0 prior to commencement of the experiment; the “Test compound signal” are wells containing inhibitor at established concentrations).

IC50 was determined by fitting percent inhibition at each inhibitor concentration to the four-parameter nonlinear logistic equation using GraphPad Prism. IC50 (nM) values of selected compounds are depicted in Table 4. “-”: means without measuring; and “ND” means not detected.

TABLE 4
IC50 values for test compounds for inhibition in cell proliferation assay.

| | Cell line | | | | | | |
|---|---|---|---|---|---|---|---|
| | AsPC-1 | GP2D | NCI-H358 | NCI-H727 | HCT-116 | MKN-1 | HT-29 |
| Cmpd | IC50(nM) | IC50(nM) | IC50(nM) | IC50(nM) | IC50(nM) | IC50(nM) | IC50(nM) |
| 1 | — | 4222 | 3211 | 1869 | — | 4087 | — |
| 2 | — | 1322 | 6319 | >10000 | — | >10000 | — |
| 3 | — | 1570 | 1656 | 1077 | — | 1775 | — |
| 4 | — | 1627 | 6601 | 4043 | — | 7879 | — |
| 6 | — | N/A | >10000 | >10000 | — | >10000 | — |
| 7 | — | N/A | 7984 | 5313 | — | >10000 | — |
| 25 | 98.1 | 8.7 | 92.4 | 36.3 | 1050 | 95.7 | 7978 |
| 68 | 41.3 | 1.1 | 11.0 | 16.4 | — | — | — |
| 9a | 20.1 | 1.6 | 18.5 | 15.0 | — | — | — |
| 10a | 16.0 | 1.8 | 18.7 | 17.0 | — | — | — |
| 18a | — | 1.9 | 23.0 | — | — | — | — |
| 19a | — | 14.1 | 15.2 | — | — | — | — |
| 20a | — | 4.7 | 124.6 | — | — | — | — |
| 21a | — | 16.3 | 92.2 | — | — | — | — |
| 26a | 30.8 | 4.5 | 98.5 | 37.4 | — | — | — |
| 27a | 32.0 | 3.07 | 40.5 | 24.8 | — | — | — |
| 29a | 25.0 | 2.3 | 25.1 | 14.7 | — | — | — |
| 33a | N/A | 421 | >10000 | N/A | — | — | — |
| 34a | 21.6 | 1.4 | 23.7 | 13.7 | — | — | — |
| A1 | 160.9 | 15.74 | 152.3 | 129.5 | 1431 | 143.7 | 7731 |

As illustrated in Table 3, multiple exemplary compounds of the disclosure demonstrated anti proliferation activity against cancer cell lines that carried KRas mutations, or wild type. Several exemplary compounds exhibited comparable or increased anti cellular proliferation potency compared to reference compound A1.

### Example 3. Cellular Phospho-ERK Inhibition

The ability of exemplary test compounds to inhibit the phosphorylation of p-ERK1/2, which is the downstream effector of KRas in multiple human cancer cell lines such as AsPC-1 (Cobioer, CBP60546), GP2D (Cobioer, CBP60683), NCI-H358 (Cobioer, CBP60544), NCI-H727 (Cobioer, CBP60182), MKN-1 (Cobioer, CBP60486), HCT116 (ATCC, CCL-247), and HT-29 (Cobioer, CBP60011), was measured. Signal reflecting cell proliferation was detected with the Advanced phospho-ERK (Thr202/Tyr204) cellular kit (Cisbio, 64AERPEG).

Cells were cultured in the growth phase and 3×10⁴ cells per well were plated in a 96-well plate (Corning, 3599). Cells were incubated 24 hours at 37° C. and 5% carbon dioxide in a humidity chamber. Serially diluted exemplary compounds were added to the cell plate which was then incubated for another 4 hours in a humid tray at 37° C. and 5% carbon dioxide. 1× Lysis buffer (Cisbio, 64AERPEG) was prepared at ambient temperature. Cell culture medium was removed, then lysis buffer (50 μL/well) was added to the cell plate and the plate was incubated at room temperature for 30 minutes on a shaker. The cell lysate (16 μL/well) was transferred to another 384-well plate (PerkinElmer, 6008289). For p-ERK detection, Phospho-ERK1/2 Eu Cryptate antibody (10 μL, Cisbio 64AERPEG) and Phospho-ERK1/2 d2 antibody (10 μL, Cisbio 64AERPEG) were mixed and diluted with 380 μL detection buffer to give detection solution. Detection solution was then added to the cell lysate plate (4 μL/well) and the plate was sealed with foil and incubated at room temperature for 2 hours. The luminescent signal was measured on the VICTOR Nivo multimode plate reader according to the manufacturer's instructions. The signal was converted to percent inhibition using the following formula:

$$\% inhibition = 100 - [ \frac{( Test compound signal - Minimum Signal )}{( Maximum Signal - Minimum Signal )} ] \times 100$$

(where the “Maximum Signal” are wells containing DMSO without inhibitor; the “Minimum Signal” are wells containing lysis buffer without cells; the “Test compound signal” are wells containing inhibitor at established concentrations).

IC50 was determined by fitting percent inhibition at each inhibitor concentration to the four-parameter nonlinear logistic equation using GraphPad Prism. IC50 (nM) values of selected compounds are depicted in Table 5.

TABLE 5
IC50 values for test compounds for inhibition in cellular phospho-ERK assay.

| | Cell line | | | | | | |
|---|---|---|---|---|---|---|---|
| | AsPC-1 | GP2D | NCI-H358 | NCI-H727 | HCT-116 | MKN-1 | HT-29 |
| Cmpd | IC50(nM) | IC50(nM) | IC50(nM) | IC50(nM) | IC50(nM) | IC50(nM) | IC50(nM) |
| 25 | 17.43 | 3.357 | 83.66 | 109.8 | 172.6 | 8.249 | >1000 |

### Example 4. Microsomal Stability Assay

A microsomal stability assay was used to measure the rate of disappearance of a test compound over time in microsomal incubations, and these data were used to calculate intrinsic clearance. Test results from this assay were used to determine the metabolic liabilities of test compounds and allow focus on the improvement of drug candidates through structure-activity relationships. Pooled mouse/rat/human liver microsomes were used for this assay

In vitro mouse (CD-1, BioIVT)/rat (SD, RILD)/human (Corning) liver microsomal suspensions at 0.5 mg/mL protein and 1.3 mM NADPH, 3.3 mM MgCl₂ were pre incubated at 37° C. for 3 mins prior to addition of 0.1 mM test compound solution to give a final incubation concentration of 1 μM. Samples were incubated for 0, 15, 30, 60 minutes at 37° C. on a shaker (the positive control substance was cultured for 0 or 60 minutes). At the end of each incubation time both incubated and control samples were quenched with ice-cold stop solution containing internal standard. All samples were mixed by a plate shaker at 800 rpm for 5 minutes, then centrifuged at 4100 rpm for 15 minutes at 4° C. to precipitate protein. Supernatant was transferred to another 96-well plate, dilution solution was added to each supernatant well, and then each bioanalysis plate was sealed. The samples were mixed by plate shaker at 800 rpm for 5 minutes and then centrifuged at 4° C., 4100 rpm for 5 minutes. All samples were stored at 4° C. before LC-MS/MS analysis.

The peak area ratio was converted to the remaining percentages (% Remaining) using the following formula:

$$\% Remaining = \frac{Peak area ratio compound to internal standard at each time point}{Peak area ratio of analyte to internal standard at 0 minute} \times 100 \%$$

where % Remaining is the compound ratio (compound peak area/internal standard peak area) as a percentage of the 0 minute time point.

The ln remaining percent of test articles were plotted against time and the gradient of the line determined; the gradient was converted to elimination rate constant by using the following formula:

Elimination rate constant(kₑ)=−gradient

Half time (T_{1/2}) was determined using following formula:

$${T}_{1 / 2} = \frac{Ln 2}{{k}_{e}} = \frac{0.693}{{k}_{e}}$$

Hepatic intrinsic clearance (CLᵢₙₜ₍ₗᵢᵥₑᵣ₎) was determined by using the following formulas:

$$C {L}_{int ( mic )} = \frac{0.693}{half time \times mg microsome protein per mL}$$

$$C {L}_{int ( liver )} = C {L}_{int ( mic )} \times \frac{mg microsomes}{g liver} \times \frac{g liver}{kg body weight}$$

The Intrinsic clearance of selected compounds is depicted in Table 6.

TABLE 6

| Compound | Mouse CLᵢₙₜ₍ₗᵢᵥₑᵣ₎ | Rat CLᵢₙₜ₍ₗᵢᵥₑᵣ₎ | Human CLᵢₙₜ₍ₗᵢᵥₑᵣ₎ |
|---|---|---|---|
| ID | (mL/min/kg) | (mL/min/kg) | (mL/min/kg) |
| 68 | 2268 | 311 | 124 |
| 67 | 1282 | 288 | 83.5 |
| 73 | 361 | 51.1 | 32.4 |
| 2a | 2629 | 887 | 402 |
| 10a | 671 | 46.4 | 42.5 |
| 15a | 371 | 52.9 | 31.8 |
| 29a | 1042 | 410 | 181 |
| 34a | 255 | 29.2 | 26.3 |
| A1 | 1181 | 111 | 46.0 |

The contents of all documents and references cited herein are hereby incorporated by reference in their entirety.

Although this invention is described in detail with reference to embodiments thereof, these embodiments are offered to illustrate but not to limit the invention. It is possible to make other embodiments that employ the principles of the invention and that fall within its spirit and scope as defined by the claims appended hereto.

What is claimed is:

1. A compound having the structure of Formula (I) or a pharmaceutically acceptable salt, ester, hydrate, solvate or stereoisomer thereof: 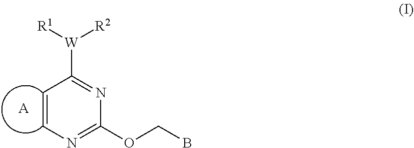
wherein:
the A ring is a substituted or unsubstituted aromatic ring, substituted or unsubstituted heteroaromatic ring, substituted or unsubstituted carboatomic ring or substituted or unsubstituted carbon heteroatomic ring;
the B ring is a substituted or unsubstituted alkyl, substituted or unsubstituted alkylaminoacyl, substituted or unsubstituted carboatomic ring, substituted or unsubstituted carbon heteroatomic ring, substituted or unsubstituted aromatic ring, substituted or unsubstituted heteroaromatic ring, substituted or unsubstituted condensed ring, a group shown below: 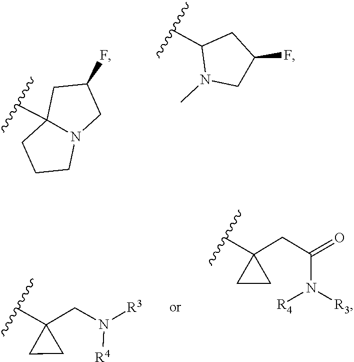
or a combination thereof;
wherein R³ and R⁴ are independently hydrogen (H) or substituted or unsubstituted alkyl, wherein the substitutions in substituted alkyl are halogen, C₁-C₄ alkyl, —OH, C₁-C₄ alkoxy, C₁-C₄ carboxyl, C₁-C₄ ester group, or C₁-C₄ acylamino group, or substituted or unsubstituted carbon heteroatomic ring, wherein the heteroatom is N, O or S; or,
R³ and R⁴ and the N atom linked to them form a substituted or unsubstituted heterocyclic ring, substituted or unsubstituted spiro-heterocycle, or substituted or unsubstituted heterobridged ring; or,
R³ and R⁴ and the N atom linked to them form a substituted or unsubstituted C₃-C₁₂ heteroaryl;
W is C, O or N, wherein:
if W is O, then R¹ is absent, and R² is independently H, substituted or unsubstituted alkyl, or substituted or unsubstituted cycloalkyl;
if W is C, then R¹ and R² are independently H, hydroxyl, halogen, alkyl, alkoxy, or alkanoyl, or R¹ and R² together form substituted or unsubstituted C₅-C₈ aryl or substituted or unsubstituted C₅-C₈ bicyclic;
if W is N, then R¹ and R² are independently H, substituted or unsubstituted alkyl, spiro ring or alkanoyl, or R and R² and the W linked to them form a substituted or unsubstituted aryl, heteroaryl, cycloalkyl, heterocycloalkyl or group shown below: 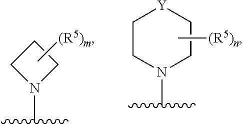
where Y is O, N, —CH₂—, —CH₂CH₂—, —CH═CH—, —OCH₂— or absent, and the H on Y and the substitutable sites on the ring are optionally arbitrarily substituted by R⁵;
m is an integer of 0 to 6;
n is an integer of 0 to 8; and
R⁵ is independently H, alkyl, hydroxyl, halogen, amino, —CF₃, —NH(C₁-C₃ alkyl), —N(C₁-C₃ alkyl)₂, ═O, —CN, —O—(C₁-C₃ alkyl), —(C₁-C₃ alkyl)-OH, —C(═O)OH, —C(═O)(C₁-C₃ alkyl), —C(═O)O(C₁-C₃ alkyl), aryl, arylalkyl, cycloalkyl or heterocycloalkyl; or,
two arbitrary R⁵s linked to one atom and the ring linked to them form a spiro ring, wherein the spiro ring is optionally substituted by alkyl, hydroxyl, halogen, amino, ═O or —CN.

2. The compound of claim 1, wherein the compound has the structure of Formula (I-b) or a pharmaceutically acceptable salt, ester, hydrate, solvate or stereoisomer thereof: 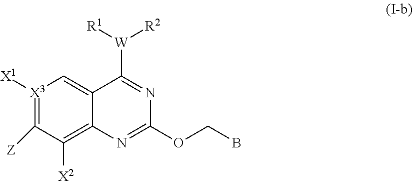
wherein:
X¹ and X² are independently H, OH, halogen, CF₃, NH₂, substituted or unsubstituted C₁-C₄ alkyl, or absent;
Z is a substituted or unsubstituted cycloalkyl, substituted or unsubstituted aryl or substituted or unsubstituted polycycloaryl;
X³ is C or N;
B is 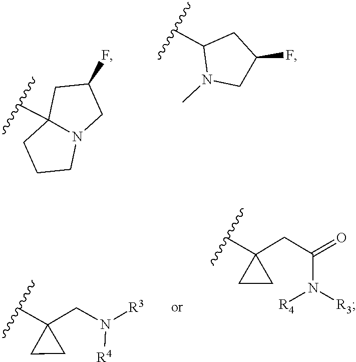
and
R¹, R², and W are as defined in claim 1.

3. The compound of claim 2, wherein Z is: 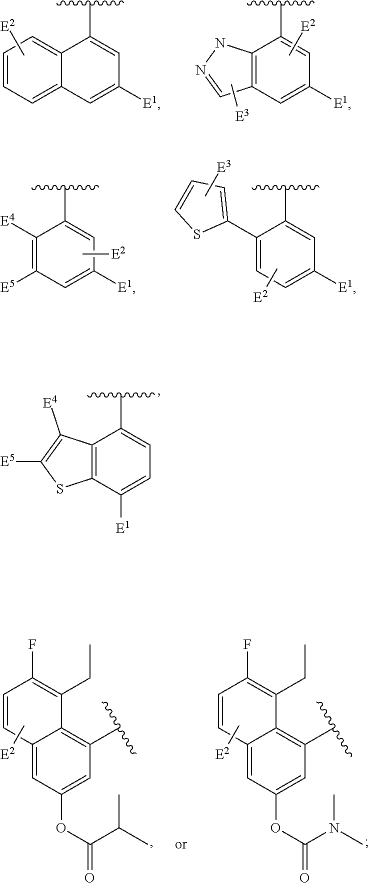
wherein E¹, E², E³, E⁴ and E⁵ are independently H, halogen, CF₃, NH₂, OH, CN, substituted or unsubstituted C₁-C₄ hydrocarbyl, or absent;
wherein E² and E³ are optionally substituted at any substitutable site on the ring;
wherein, if E² and E³ are absent or H, then E¹ is Cl or methyl.

4. The compound of claim 1, wherein the compound has the structure of Formula (II-a) or a pharmaceutically acceptable salt, ester, hydrate, solvate or stereoisomer thereof: 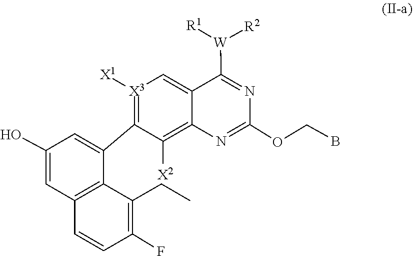
wherein X¹ and X² are independently H, OH, F, Cl, CF₃, NH₂, or substituted or unsubstituted C₁-C₄ alkyl, and X³ is C or N;
wherein, when X³ is N, X¹ is absent.

5. The compound of claim 1, wherein
B has the structure of 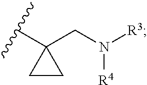
and
R³ and R⁴ and the N atom linked to them form a substituted or unsubstituted six-membered heterocyclic ring, substituted or unsubstituted five-membered heterocyclic ring or substituted or unsubstituted four-membered heterocyclic ring, wherein the heterocyclic ring has one of the following structures:

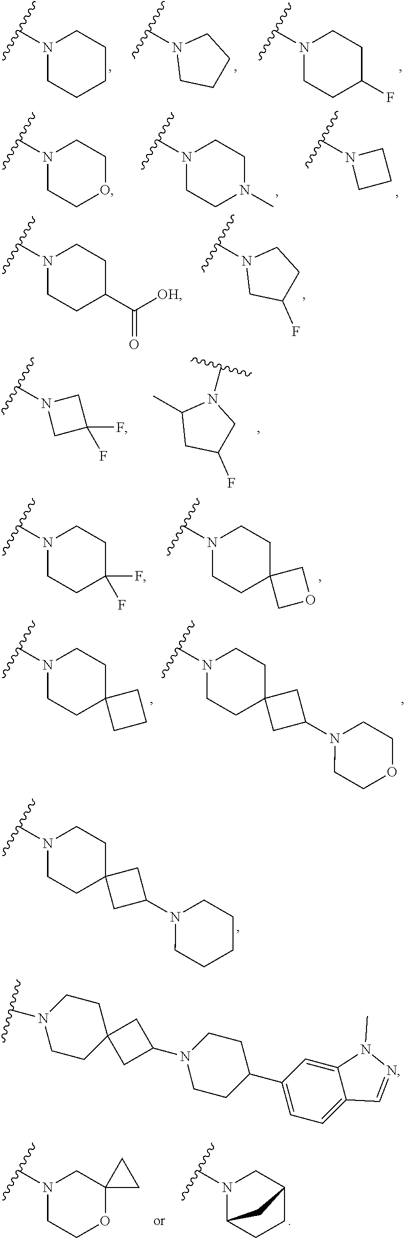

6. The compound of claim 1, wherein
W is N, R¹ or R², and the W linked to them form 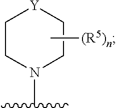
wherein Y is O, N, —CH₂—, —CH₂CH₂—, —OCH₂— or absent; and
R⁵ is H, —OH, halogen, amino, —CH₂OH, —NH(C₁-C₃ alkyl), —N(C₁-C₃ alkyl)₂, ═O, —CN, —O—(C₁-C₃ alkyl), —(C₁-C₃ alkyl)-OH, —C(═O)NH₂, —C(═O)OH, —C(═O) (C₁-C₃ alkyl), or —C(═O)O(C₁-C₃ alkyl); and
R⁵ and the six-membered ring linked to it form one of the following structures:

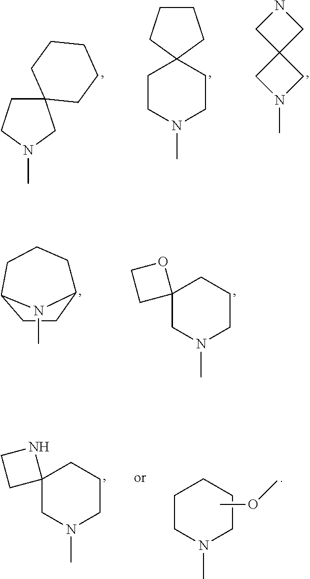

7. The compound of claim 1, wherein the compound has the structure of Formula (IV-c) or a pharmaceutically acceptable salt, ester, hydrate, solvate or stereoisomer thereof:

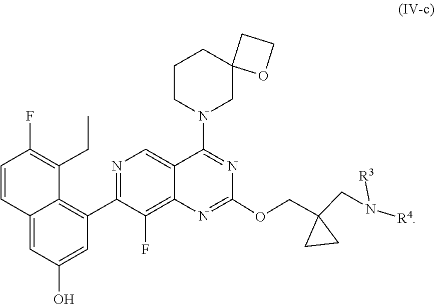

8. A compound which is: 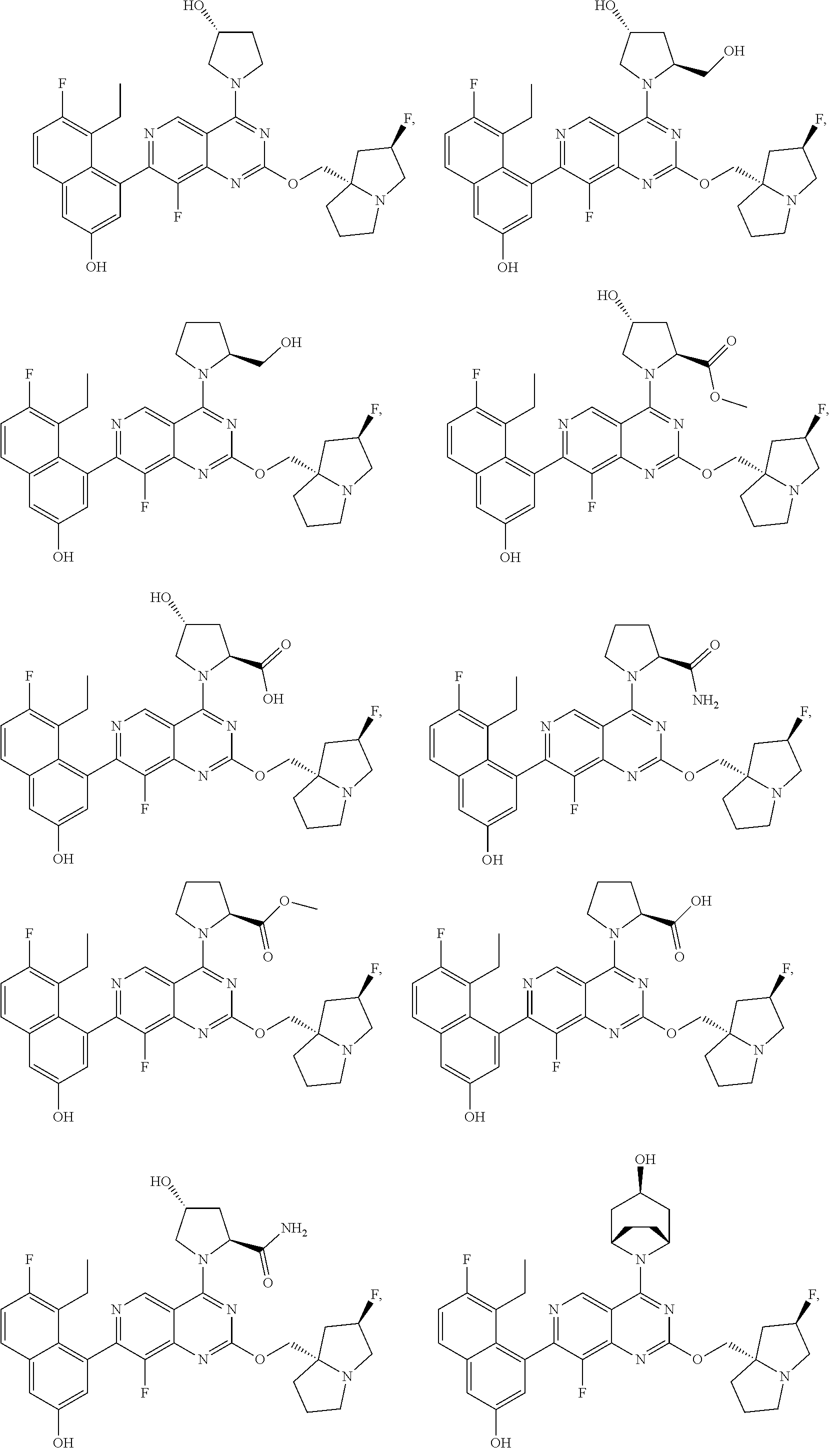 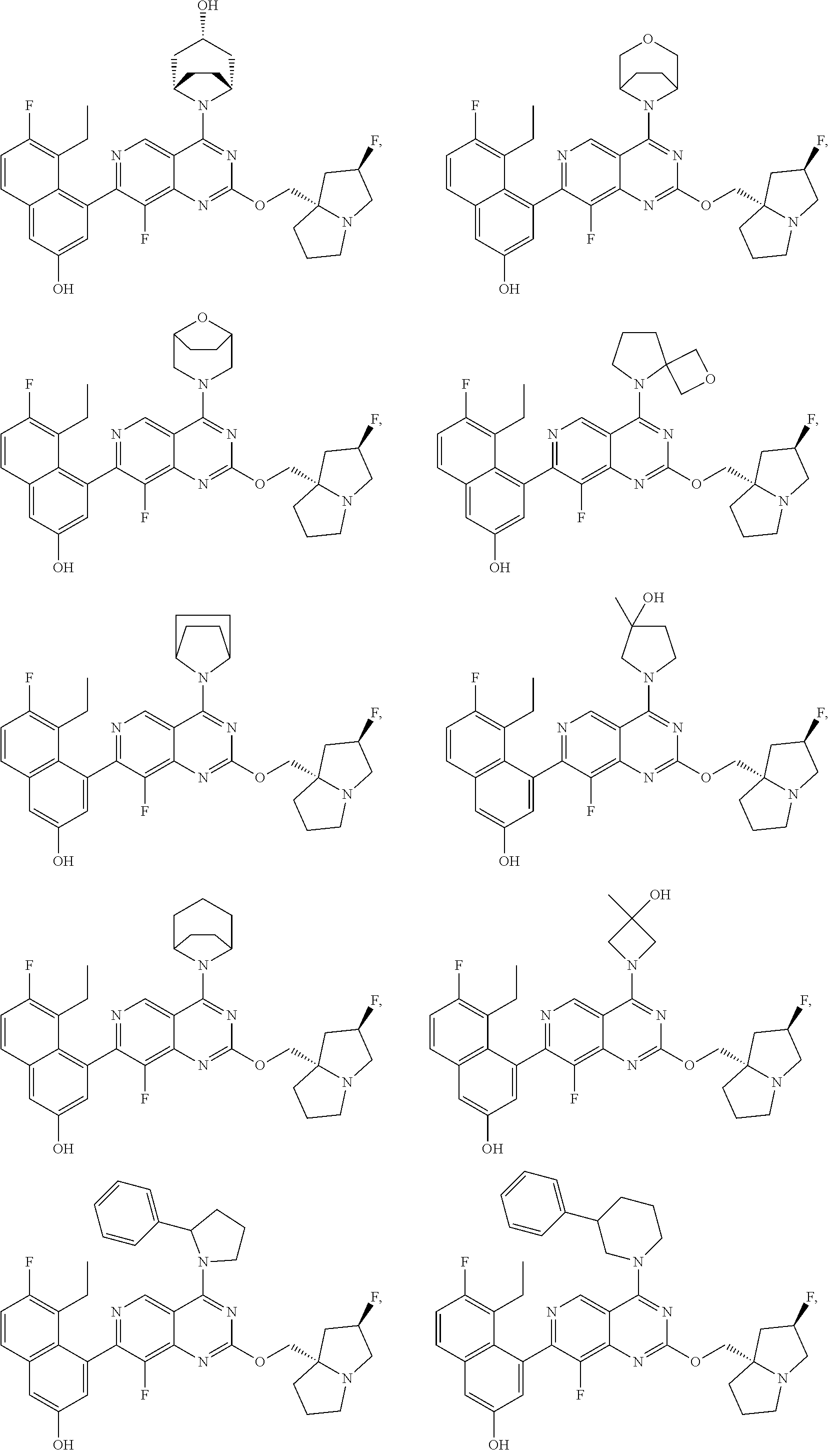 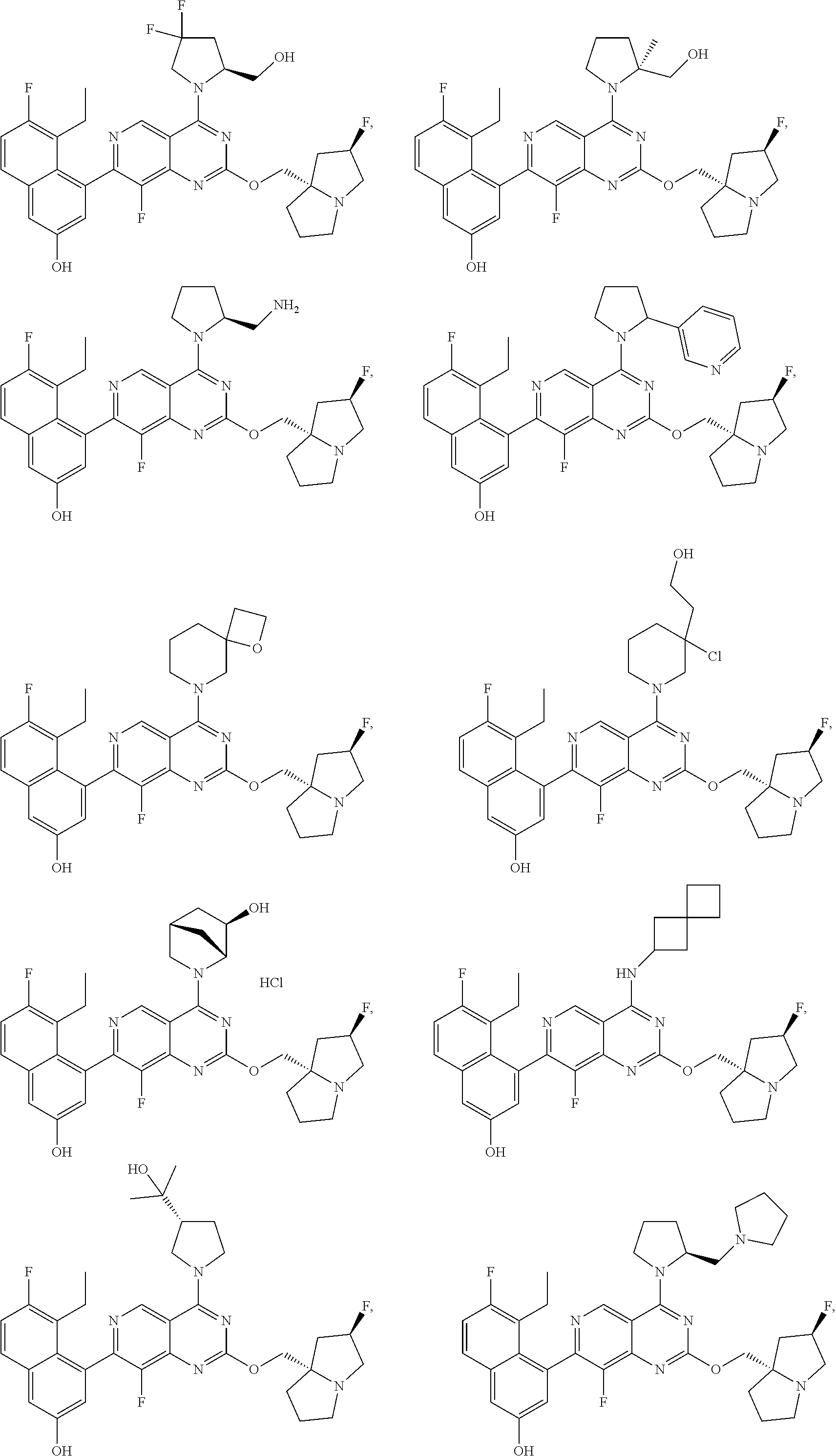 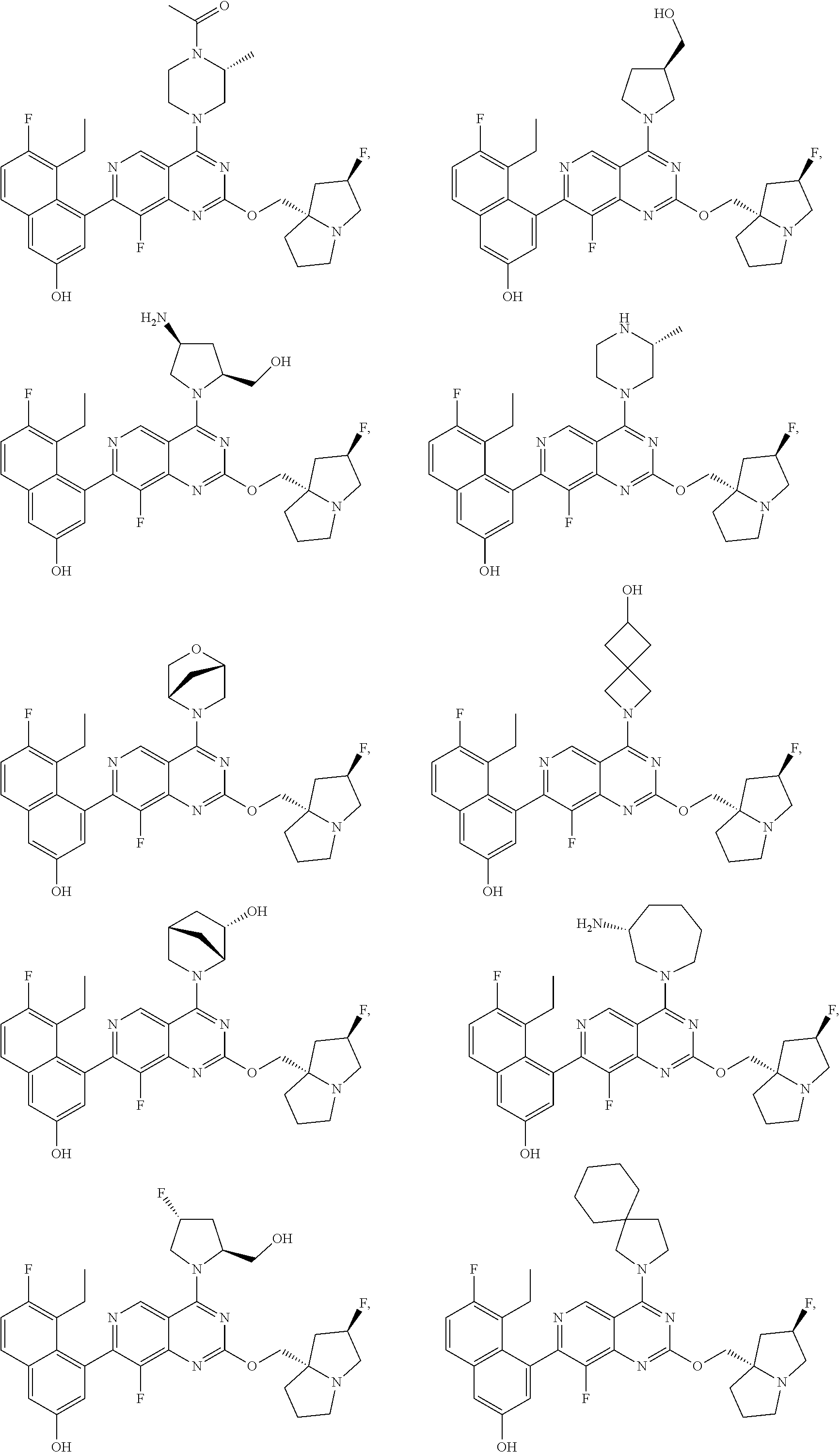 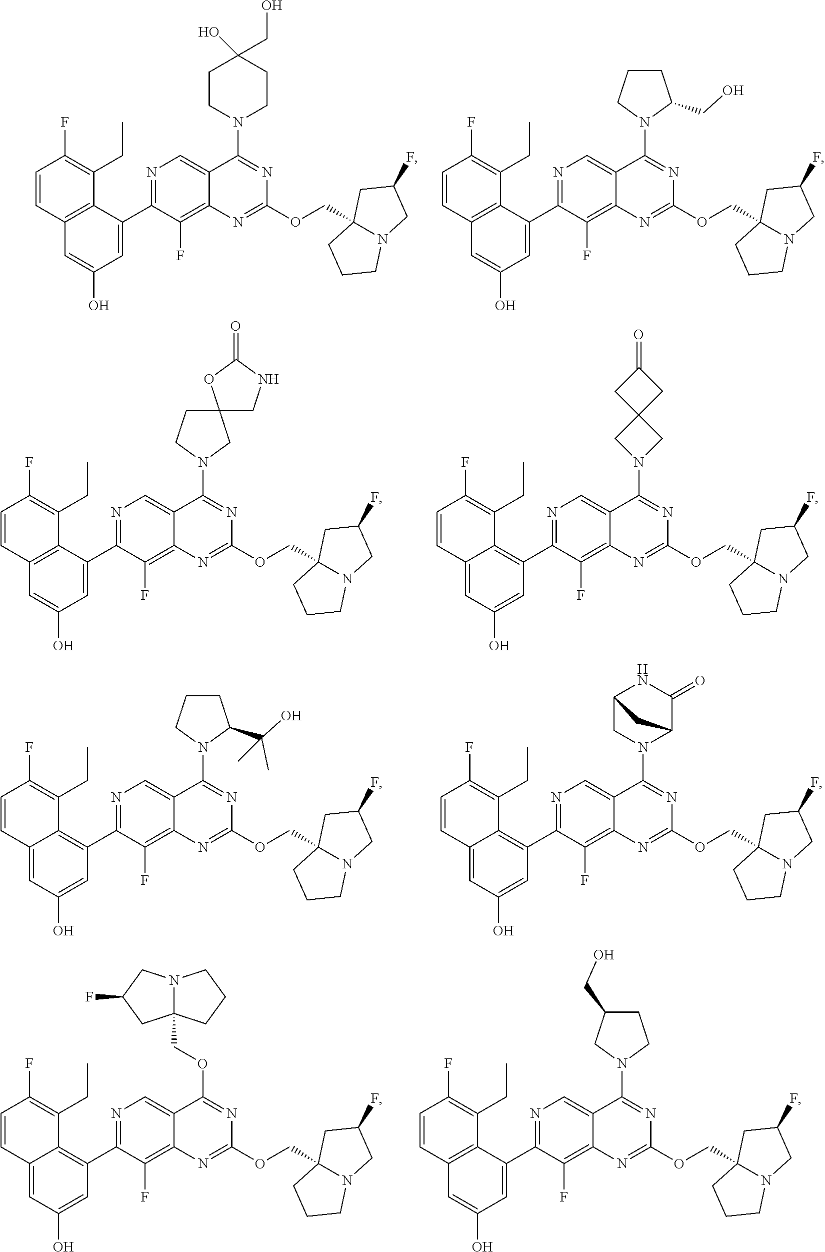 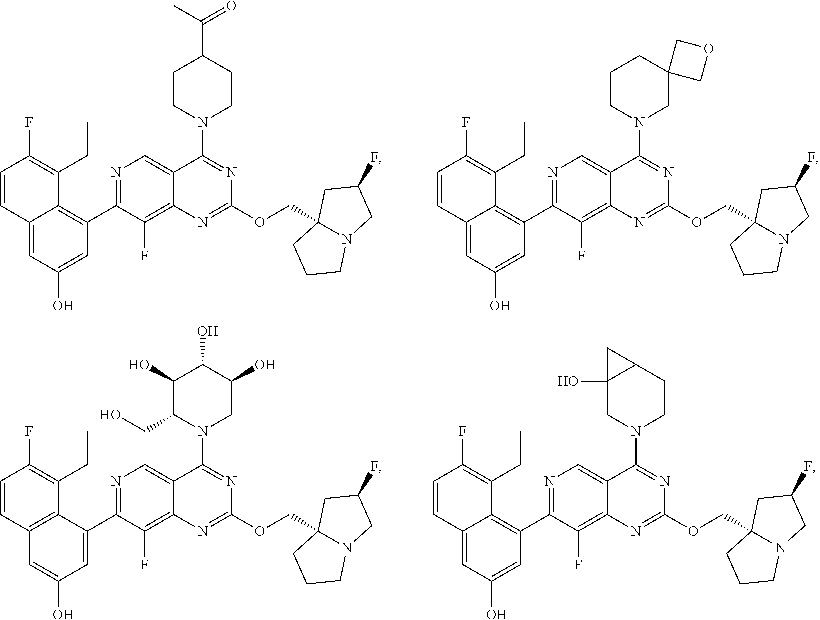 [SMILES: CCc1c(F)ccc2cc(O)cc(-c3ncc4c(N5CCC(C(C)=O)CC5)nc(OC[C@@]56CCCN5C[C@H](F)C6)nc4c3F)c12 ; CCc1c(F)ccc2cc(O)cc(-c3ncc4c(N5CCCC6(COC6)C5)nc(OC[C@@]56CCCN5C[C@H](F)C6)nc4c3F)c12 ; CCc1c(F)ccc2cc(O)cc(-c3ncc4c(N5C[C@H](O)[C@@H](O)[C@H](O)[C@H]5CO)nc(OC[C@@]56CCCN5C[C@H](F)C6)nc4c3F)c12 ; CCc1c(F)ccc2cc(O)cc(-c3ncc4c(N5CCC6CC6(O)C5)nc(OC[C@@]56CCCN5C[C@H](F)C6)nc4c3F)c12] 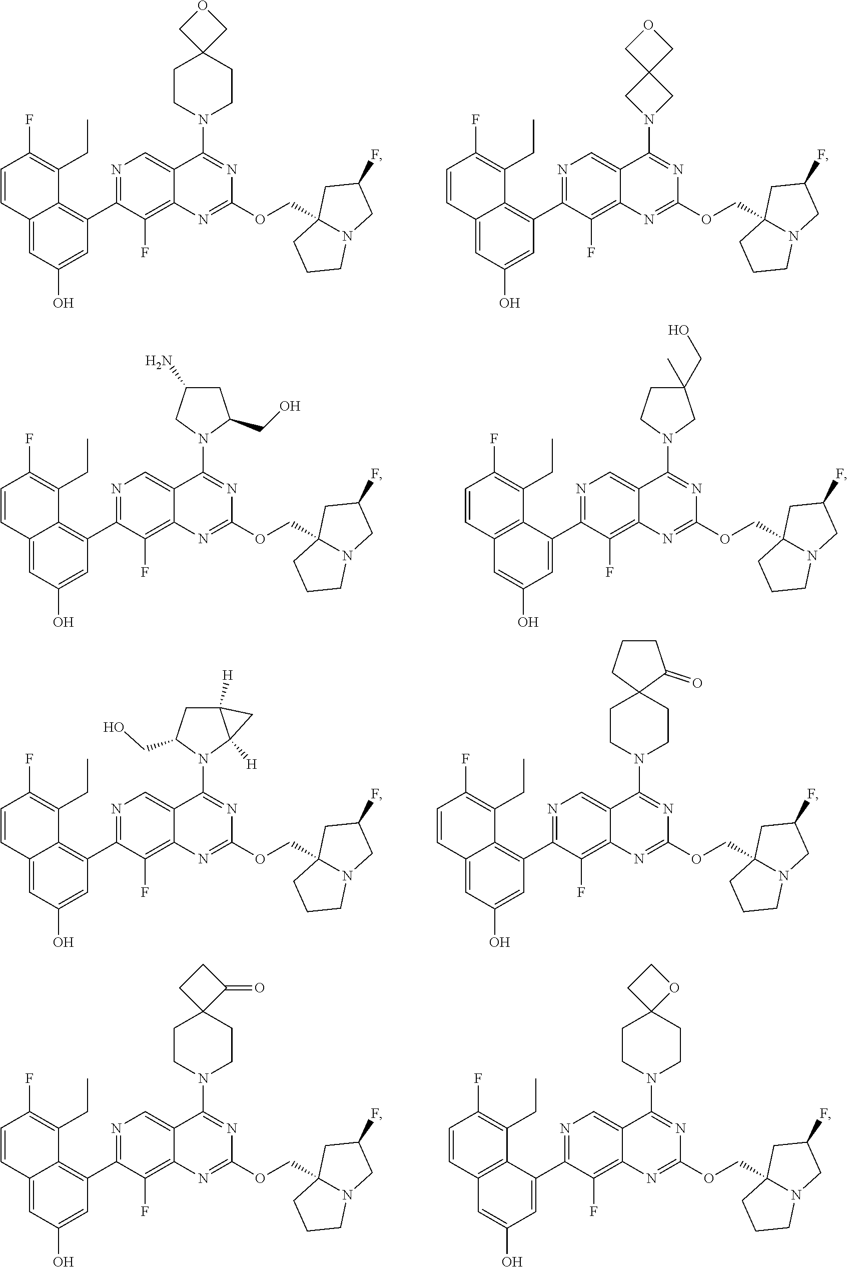 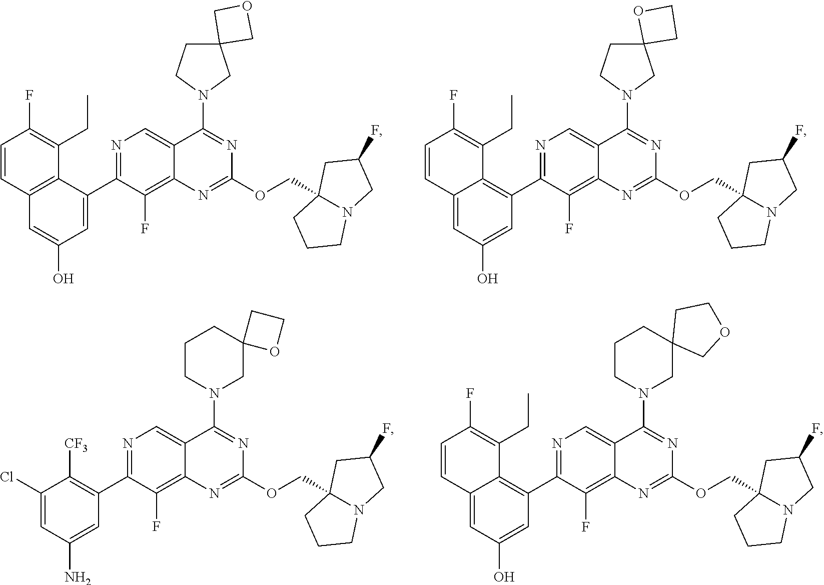 [SMILES: CCc1c(F)ccc2cc(O)cc(-c3ncc4c(N5CCC6(COC6)C5)nc(OC[C@@]56CCCN5C[C@H](F)C6)nc4c3F)c12 ; CCc1c(F)ccc2cc(O)cc(-c3ncc4c(N5CCC6(CCO6)C5)nc(OC[C@@]56CCCN5C[C@H](F)C6)nc4c3F)c12 ; Nc1cc(Cl)c(C(F)(F)F)c(-c2ncc3c(N4CCCC5(CCO5)C4)nc(OC[C@@]45CCCN4C[C@H](F)C5)nc3c2F)c1 ; CCc1c(F)ccc2cc(O)cc(-c3ncc4c(N5CCCC6(CCOC6)C5)nc(OC[C@@]56CCCN5C[C@H](F)C6)nc4c3F)c12] 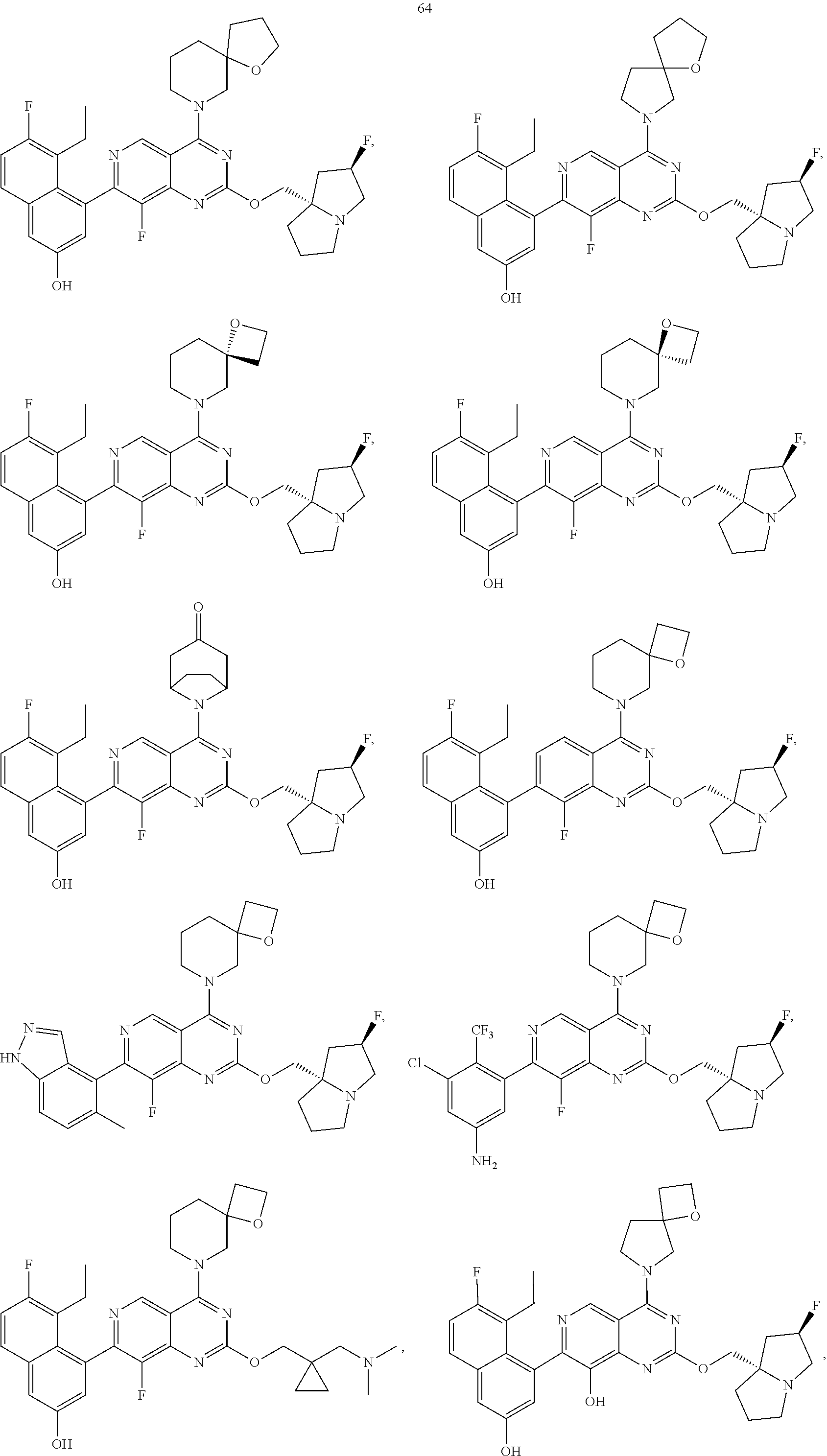 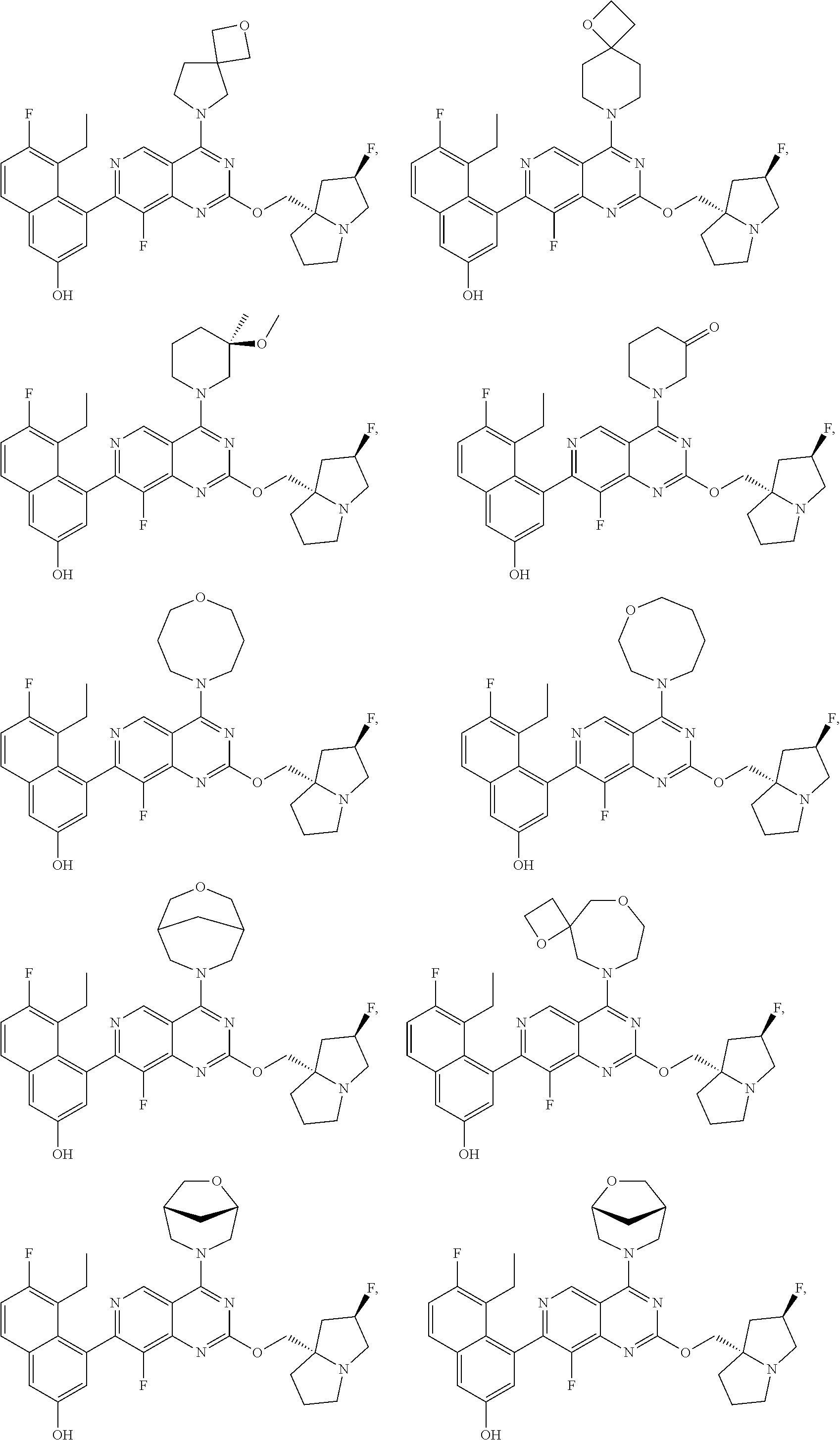 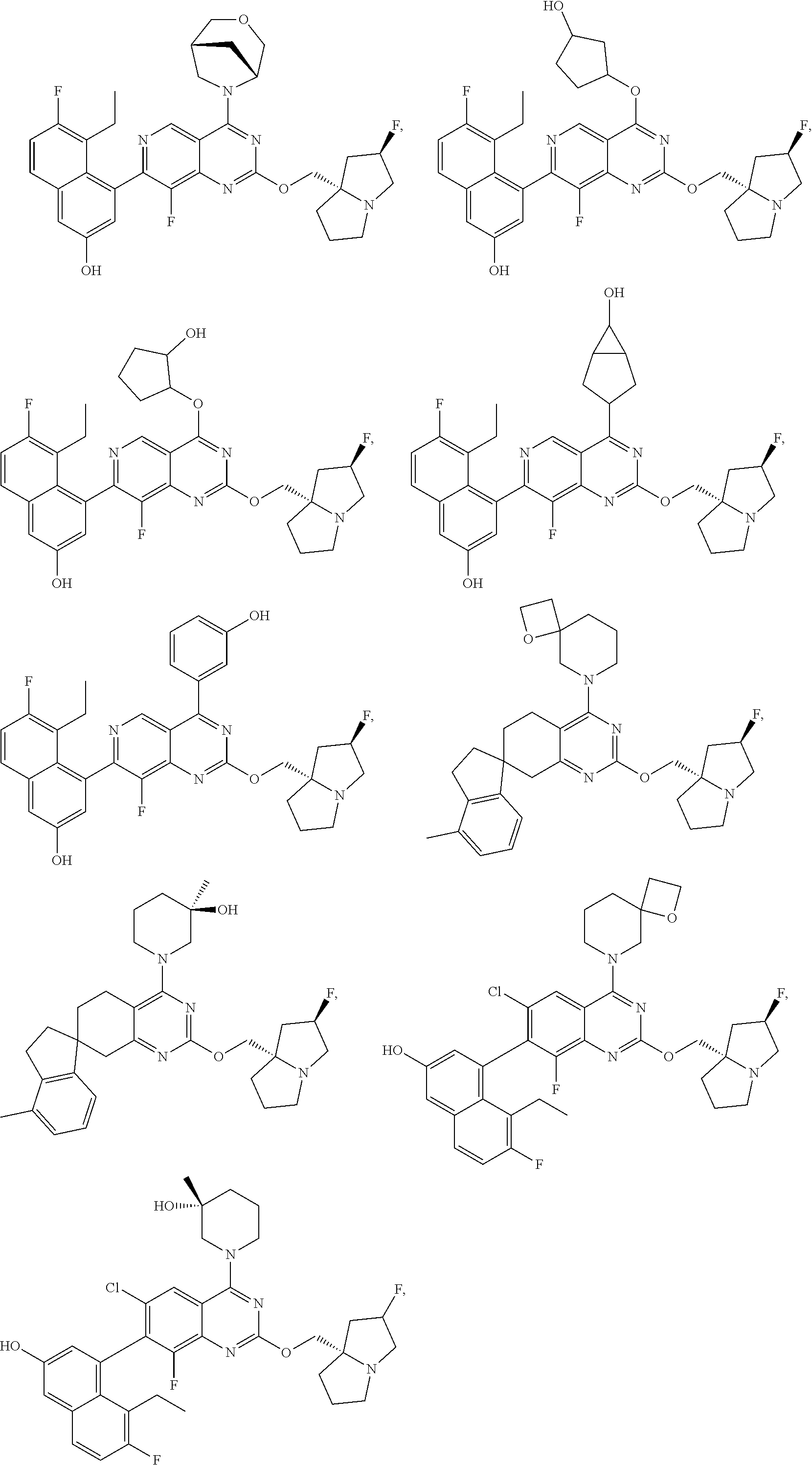 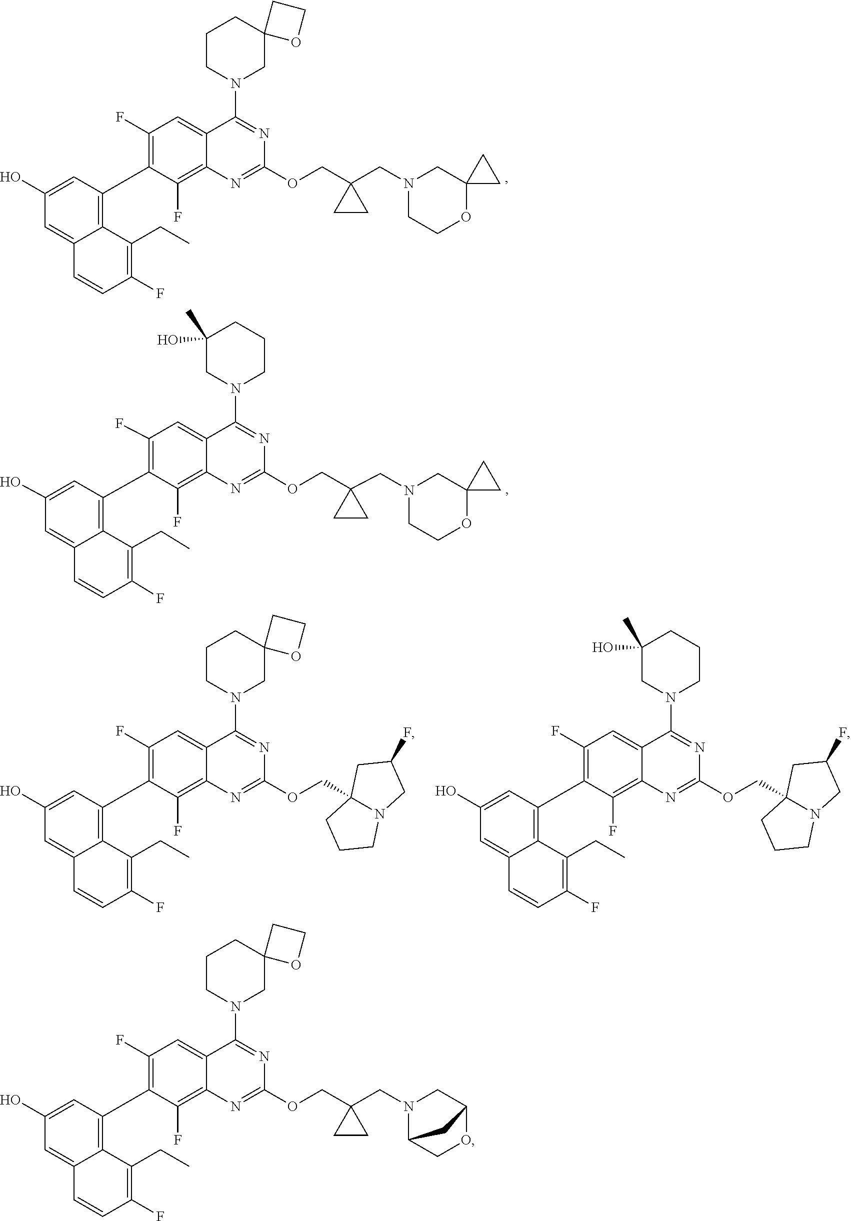 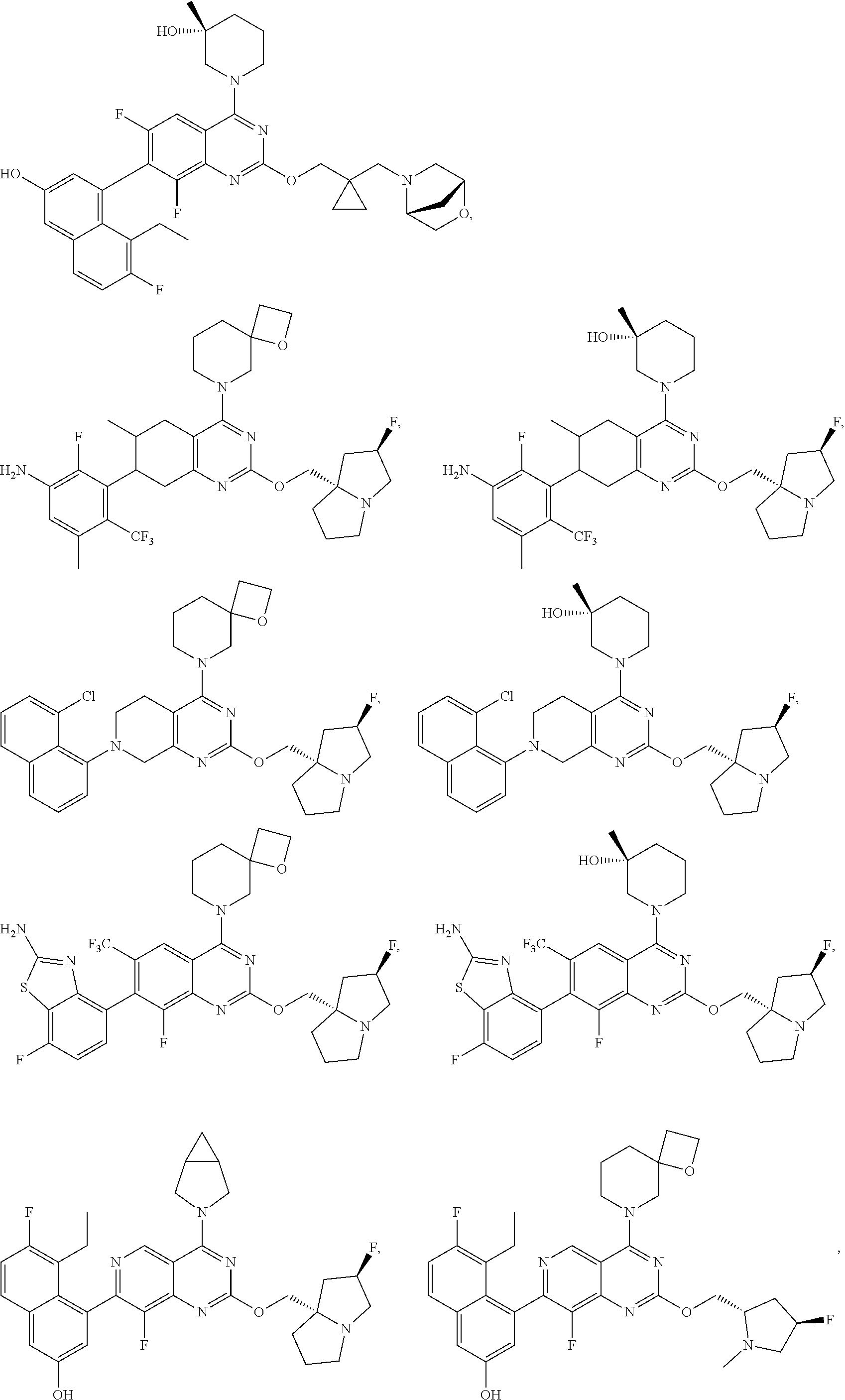 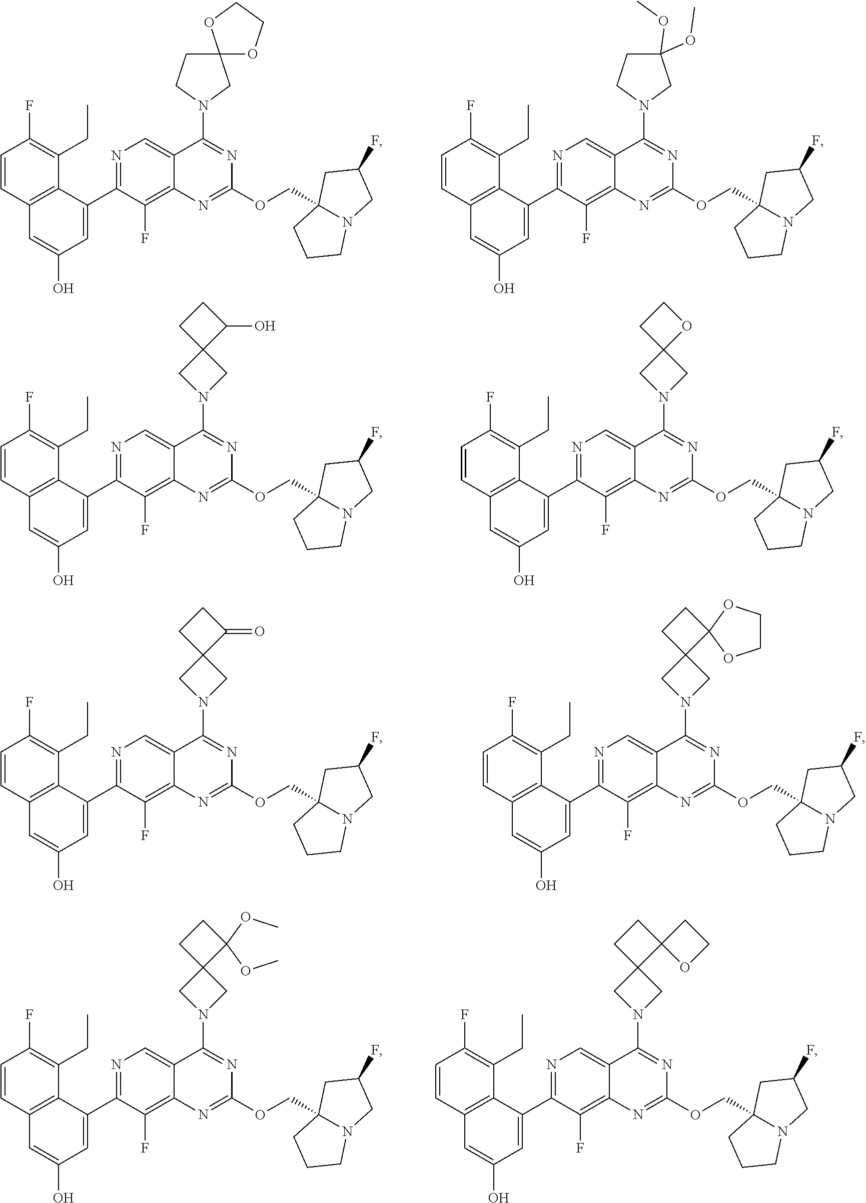 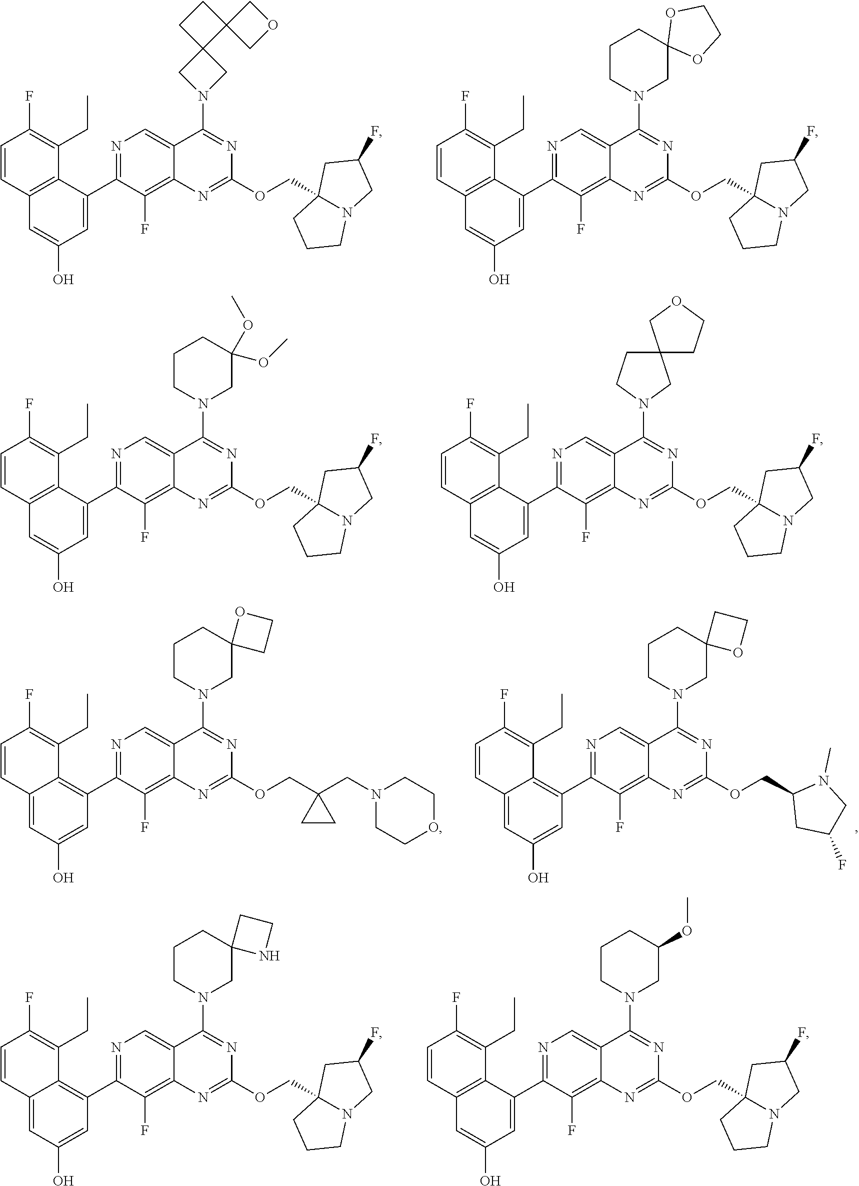 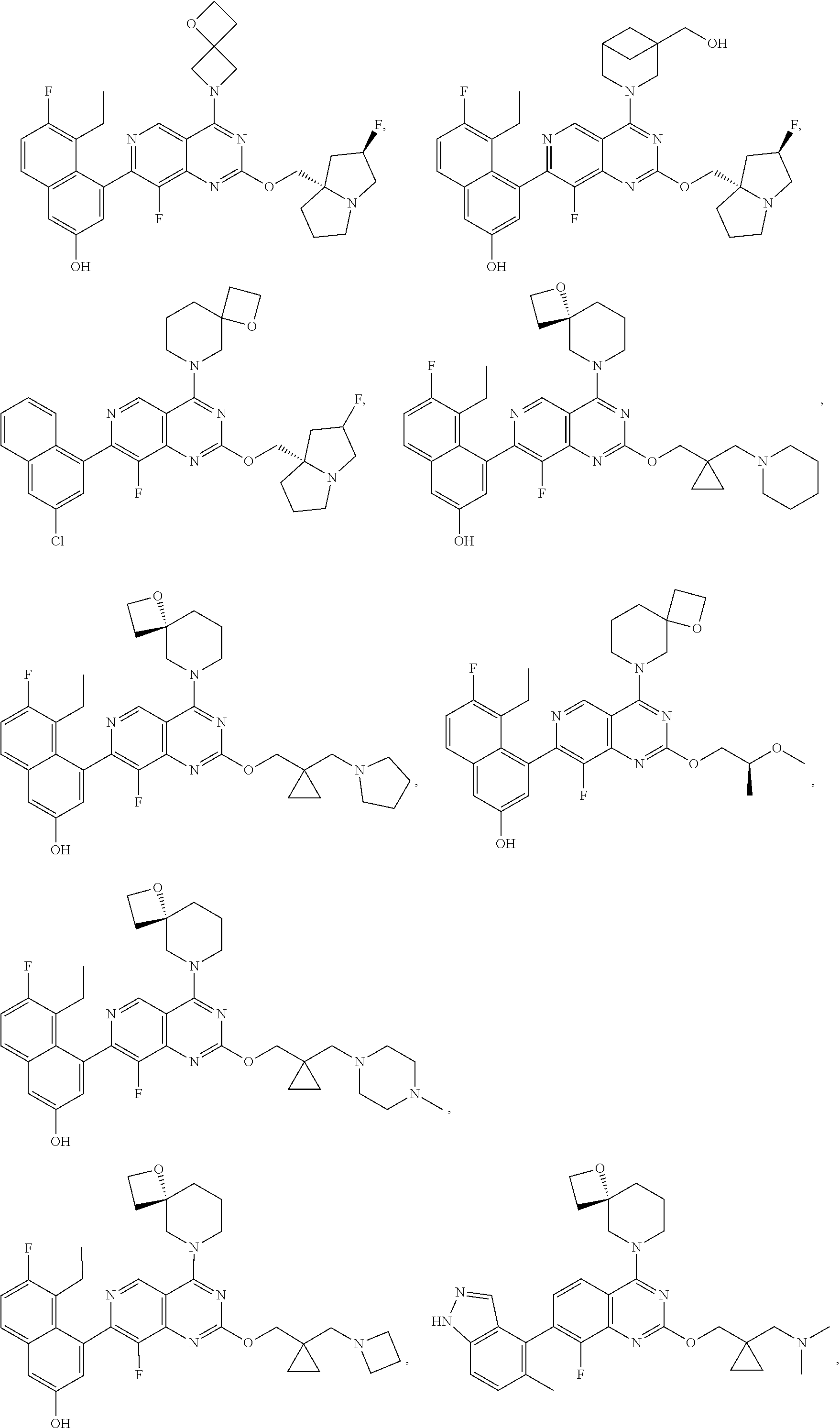 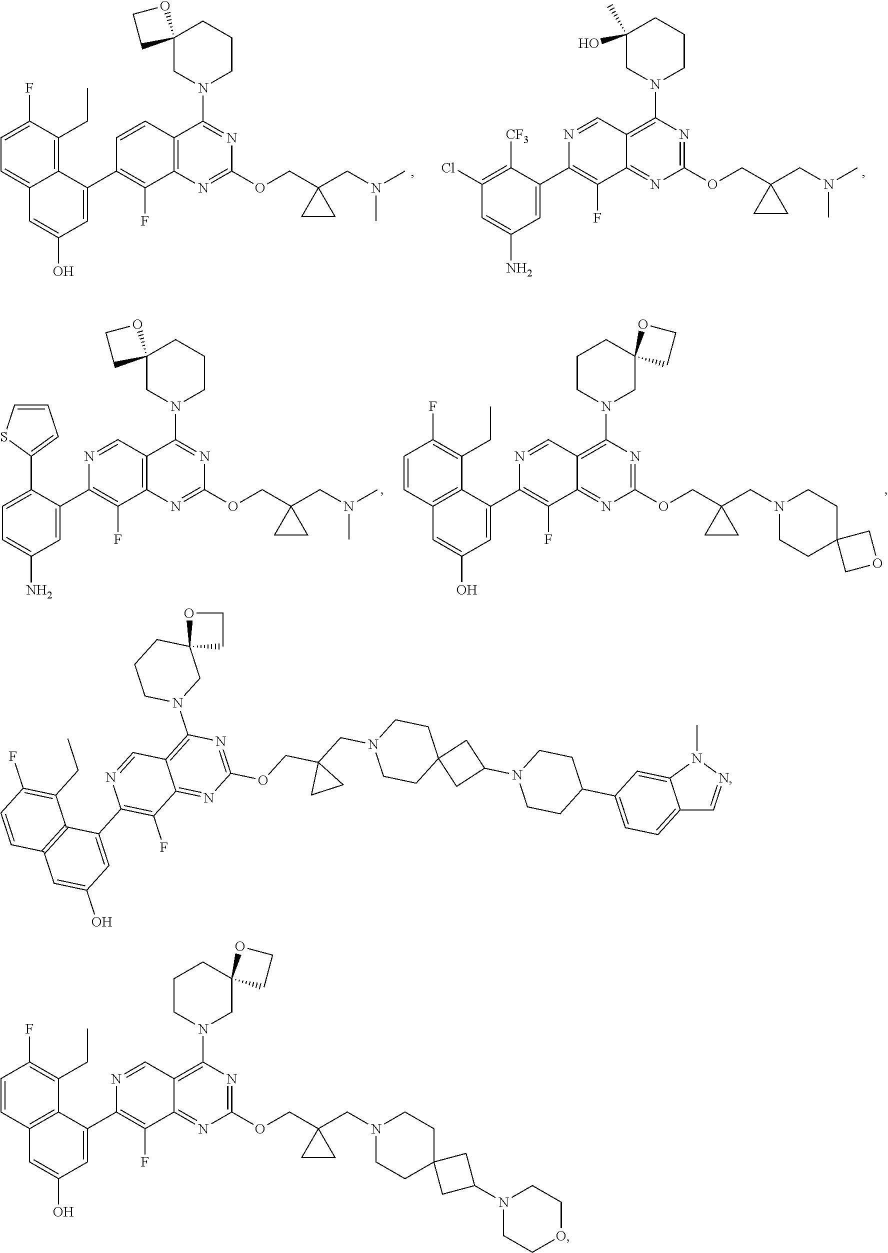 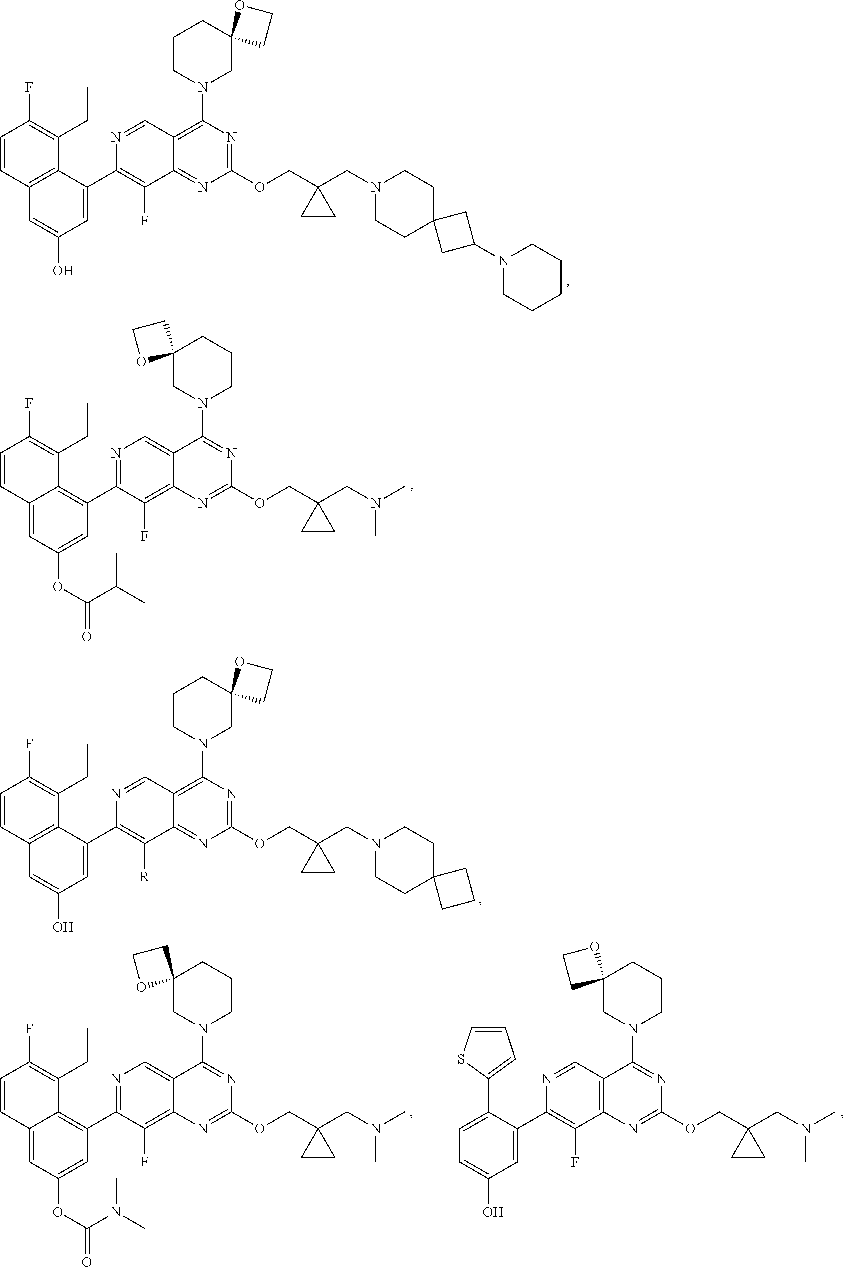 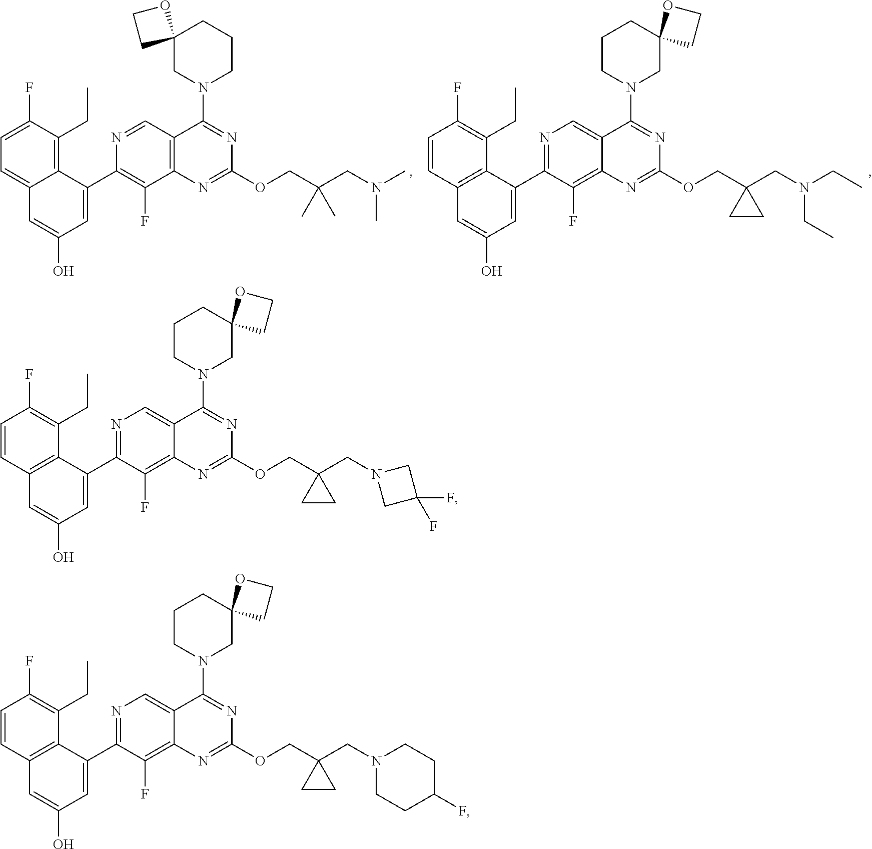 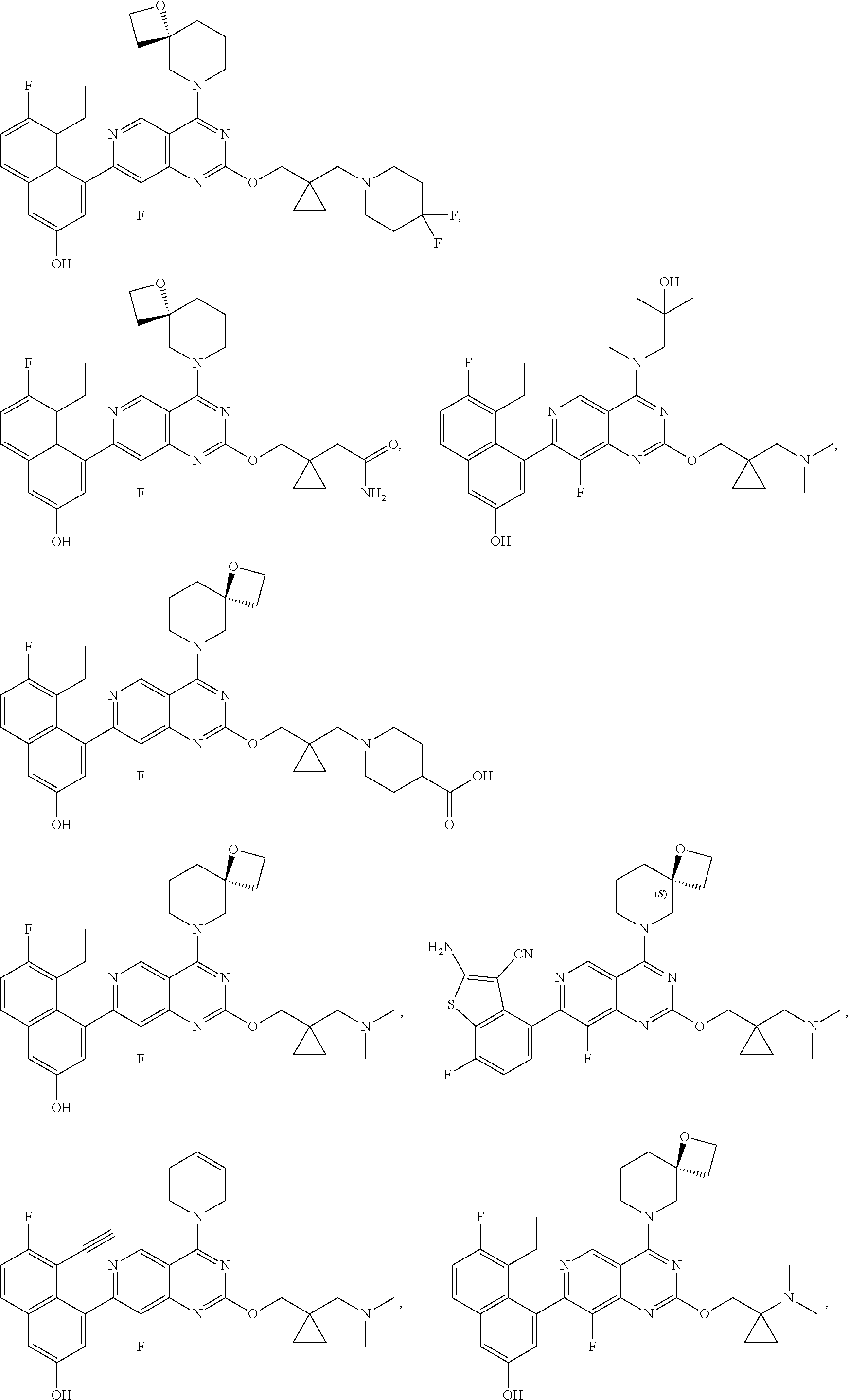 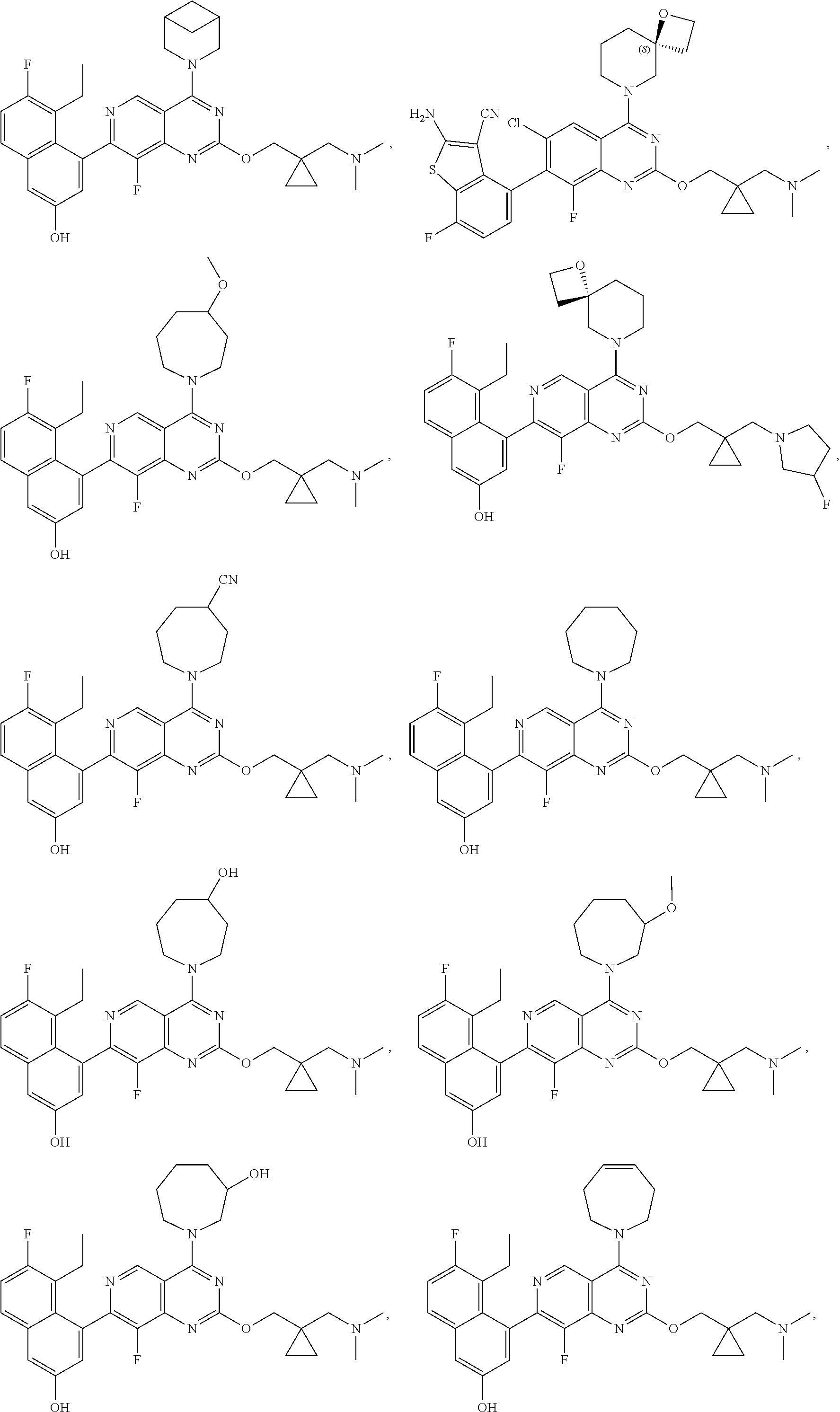 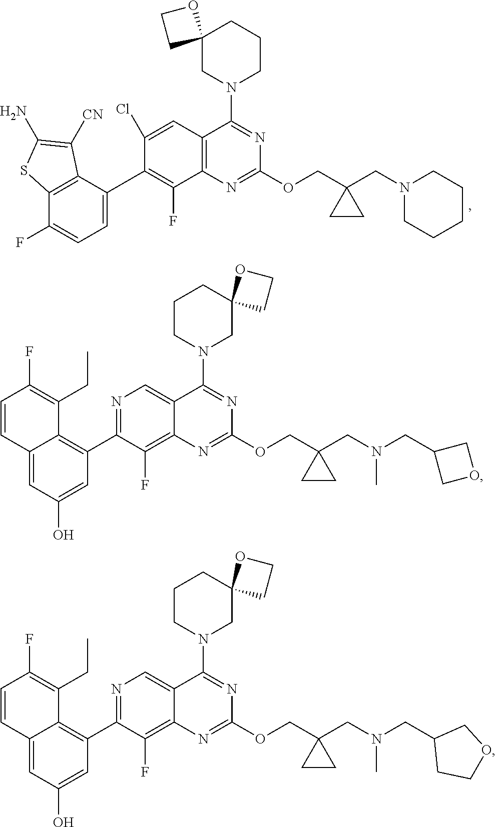 [SMILES: N#Cc1c(N)sc2c(F)ccc(-c3c(Cl)cc4c(N5CCC[C@]6(CCO6)C5)nc(OCC5(CN6CCCCC6)CC5)nc4c3F)c12 ; CCc1c(F)ccc2cc(O)cc(-c3ncc4c(N5CCC[C@]6(CCO6)C5)nc(OCC5(CN(C)CC6COC6)CC5)nc4c3F)c12 ; CCc1c(F)ccc2cc(O)cc(-c3ncc4c(N5CCC[C@]6(CCO6)C5)nc(OCC5(CN(C)CC6CCOC6)CC5)nc4c3F)c12]
or a pharmaceutically acceptable salt, ester, hydrate, solvate, stereoisomer and/or diastereomer thereof.

9. A pharmaceutical composition comprising the compound or the pharmaceutically acceptable salt, ester, hydrate, solvate or stereoisomer thereof of claim 1 and at least one pharmaceutically acceptable excipient, carrier or diluent.

10.-12. (canceled)

13. The pharmaceutical composition of claim 9, wherein the composition is for parenteral, intraperitoneal, intradermal, intracardiac, intraventricular, intracranial, cerebrospinal, intrasynovial, or intrathecal administration, or for intramuscular injection, intravitreal injection, or intravenous injection, or for intra-arterial, oral, intraoral, sublingual, transdermal, intratracheal, intrarectal, subcutaneous or topical administration.

14.-45. (canceled)

46. A method for inhibiting, treating and/or preventing a hyperproliferative disorder in a subject comprising administering an effective amount of the compound or the pharmaceutically acceptable salt, ester, hydrate, solvate, or stereoisomer thereof of claim 1 to the subject.

47. The method of claim 46, wherein the hyperproliferative disorder is a KRAS-associated cancer or tumor.

48. The method of claim 47, wherein the hyperproliferative disorder is related to or associated with a KRAS mutation selected from KRAS G12A, KRAS G12C, KRAS G12D, KRAS G12R, KRAS G12S, KRAS G12V, KRAS G13D, KRAS Q61H, and combinations thereof.

49. The method of claim 46, wherein the cancer or tumor is a cardiac, lung, gastrointestinal, genitourinary tract, liver, biliary tract, small intestine, large intestine, bone, nervous system, gynecological, hematologic, skin, or adrenal gland cancer or tumor.

50. A method for treating or preventing a malignant or hyperplastic disorder in a subject in need thereof, comprising administering an effective amount of the compound or the pharmaceutically acceptable salt, ester, hydrate, solvate, or stereoisomer thereof of claim 1 to the subject, such that the malignant or hyperplastic disorder is treated or prevented in the subject.

51. The method of claim 50, wherein the malignant or hyperplastic disorder is associated with a KRAS mutation.

52. The method of claim 51, wherein the KRAS mutation is selected from KRAS G12A, KRAS G12C, KRAS G12D, KRAS G12R, KRAS G12S, KRAS G12V, KRAS G13D, KRAS Q61H, and combinations thereof.

53. The method of claim 50, wherein the malignant or hyperplastic disorder is non-small cell lung cancer (NSCLC), small cell lung cancer, pancreatic cancer, colorectal cancer, colon cancer, bile duct carcinoma, cervical cancer, bladder cancer, liver cancer or breast cancer.

54.-57. (canceled)

58. A method for treating a cancer in a subject, comprising administering to the subject an effective amount of the compound or the pharmaceutically acceptable salt, ester, hydrate, solvate, or stereoisomer thereof of claim 1 and an immune checkpoint inhibitor to the subject, such that cancer is treated in the subject.

59.-63. (canceled)

64. The method of claim 58, wherein said immune checkpoint inhibitor is selected from the group consisting of ipulimumab, nivolumab and lambrolizumab.
